SEQUENCE LISTING (28 sequences)

SEQ ID NO: 1 - DNA, 102211 bp, synthetic construct
  source 1..102211: mol_type = other DNA
gcggccgcaccatcatgatatcccacaagtatggaagatacataagtagatggccagactatacaggaaagaagcaaaacttgggctaacagaagagcaaacacagagctcagacagaactacctggcaatgcgactgggcacactgaaagcactgggcatcagcactgagccccaaatatgcactcaggatcctctgcataataatgtgacataacaggaaggttagaacaggccaaaagaggaaacagaacaaatgcccccaaccaaagaagtataaacaaattgggaagagtaaagaaggattgtaaggattgagtaccacacagaacatgctcttaatggcctcaatgctgaagctaggaagaactaagttaaaagaaacatgttcaacgggattccctgtcactggacttcacaacaagcaaaattcaatctttctgttaaggagatgagaagagaatatctgaaccttgtgttgacagtgccccaccccgactgtcaggctgtgggaaatgccagagcaatcactaggaacacacaaggatgagggagacgagggttaggacacaattctcgagaagttcctattctctagaaagtataggaacttcctgatcactcgatctcgtgctacgataacttcgtatagcatacattatacgaagttatcgcgccgcacacaaaaaccaacacacagatcatgaaaataaagctcttttattggtaccgaattcgccagggagctctcagacgtcgcttggtcggtctttattcgaaccccagagtcccgctcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtttagttcctcaccttgtcgtattatactatgccgatatactatgccgatgattaattgtcaacacgtgctgctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtggaaggtggcgcgaaggggccaccaaagaacggagccggttggcgcctaccggtggatgtggaatgtgtgcgagccagaggccacttgtgtagcgccaagtgcccagcggggctgctaaagcgcatgctccagactgccttgggaaaagcgcctcccctacccggtaggaagttcctattctctagaaagtataggaacttcaatttcttgaaaaaaatcagtgctattttggggtcagaacaatcctcaaaaatggtccccggaaagaagcccaaatttaattgcaccagattgttgaacgatttatgctacaaaacagtgtcacatgtcagaaatgagcacagtgtaacatctacatggtttgttaagagacacaaatggtcaagtagaacaatcaggtaattaggctgtcgaaggcaacactgccaaatgacagcatttccgtggaaactgcgtgtacatccaggactgcacctgtgaacgatgacatcatacccttcacagtgtcgaggaaagagacatcactcaaacagacaagccaagggacttcagaaaatataaggggaaatacagtgtgcaaatatgtaaaaaatgcaataagatgattactcctaaatgaatatcaagacacaatcacataatatgaaattaaattttcctgaatgataggattactaccaatcaccccccaggacaccctcatctactctgtgcacagccttctcgtcaggcgtcccagcccagaccttgctatgtagcagaagacatgcaaataagacccccctttttgctgatgaaaagcagcccagccctgaccctgcagctctgggagaggagctccagccttgggattcccagctgtctccactcggtgatcggcactgaatacaggagactcaccatggagtttgggctgagctgggttttccttgttgctattttaaaaggtgattcatggggaactagagatactgagtgtgagtggacatgagtgagagaaacagtggacgtgtgtggcactttctgaccagggtgtctctgtgtttgcaggtgtccagtgtgaggtgcagctggtggagtctgggggaggcttggtccagcctggggggtccctgaaactctcctgtgcagcctctgggttcaccttcagtggctctgctatgcactgggtccgccaggcttccgggaaagggctggagtgggttggccgtattagaagcaaagctaacagttacgcgacagcatatgctgcgtcggtgaaaggcaggttcaccatctccagagatgattcaaagaacacggcgtatctgcaaatgaacagcctgaaaaccgaggacacggccgtgtattactgtactagacacacagtgaggggaggtcagtgtgagcccggacacaaacctccctgcaggggcgcgcggggctaccagggggcgctcgggactcactgagggcgggacaggtcccaggaacaggtgcagcggtaggttttctttctcctcagctggagaagtcaggtttgtgttttcagaactctggagtcttacaggttgctacattttcatacagttattagtatgtatttattatcattggtatttaagttttaataattttaaaccttttatgtagtgttattttttaaaactgtttactttcatttgcagttattcttccagagtttcattaacatctattgctatgagcaactacatagctatgagagcataaatttacacctgtagacgtaggtctaaatgccacaaacctgtgcataaatgtatagtgacttatatttaacattataataagataatttttaaaatatatcctaaacgatcaaacttactgatgaactaaatataaattatcagagtaatgcataattgatttcaataattttatattgtttatattaattaatatctatttctttactgaaacataatatattggtcatttcaaaatagctacgatacatttcaaatggccttgatgctaataatgaaaagattttgaggtgattaatatgctaattagttatatttaattattccatattgggtcccacacactgttaaacaaccaaatctcatgagaactcatttactatcatagcaaggggaccgtgctaaccattattcataaatccggacccacgatacaaccaccgggccccgcctcaaacattagggaagcaaaatcacatacagttgttatgacaggtgggacatcctgaaaacctctctaggcatgtcccacacggccctggagctgtctcaggggagcagtctcctccagtgtttagacgcacaggcacggataatagggctaagtctggccagatgtgtgatattcaacacattgcacaactgctctgttctgtatgtaatttatcttctctacaaatgtaacattgatggttgcattaaatatattctgcaaatatgtaaaaataaaataagatgatgattgctaaatgattatcaaggcacaatcacataatccgaagttacaatttcctgagagataggattactgcccatgcttaccaggacactcacatctgctctgggcactgccttctcctcaggcgtcccaccccagagcttgctatatagtaggagacatgcaaatagggtcctccctctgctgatgaaaaccagcccagccctgaccctgcagctctgagagcggagccccagccccagaattcccaggtgttttcatttggtgatcagcactgaacacagaggactcaccatggagtttgggctgagctgggttttccttgttgttattttacaaggtgatttatggagaactagagatgttaagtgtgagtggacgtgagtgagagaaacagtggatttgtgtgacagtttctgaccagggtgtctctgtgtttgcaggtgtccagtgtgaggtgcagctggtggagtctgggggaggcttggtccagcctggagggtccctgagactctcctgtgcagcctctggattcaccttcagtgaccactacatggactgggtccgccaggctccagggaaggggctggagtgggttggccgtactagaaacaaagctaacagttacaccacagaatacgccgcgtctgtgaaaggcagattcaccatctcaagagatgattcaaagaactcactgtatctgcaaatgaacagcctgaaaaccgaggacacggccgtgtattactgtgctagagacacagcgaggggaggtcagtgtgagcccggacacaaacctccctgcaggggcgcgtggggccaccagggggcggtcgggaaccactgaggacgggacaggtcccaggagcaggtgcagggggcggtttcctgtcagctgcaggaggcgggtttgtttttgcaggactctggagccttatgaggttgcactattttattatggttatttatcatgtgttttttattgatatttgtgttttagtaatttttacatttatatgtaggggtattttttaaaaatacataacttcaagaaataattcttcctagtaatttacacctattcctctaaagttttattaacacctgttgatatcagcaactacatagctatgcaacattaatttacatctataaacatatgtgtgaatacatgaaactatgcatacatgtgtagtcatttatatttaacattataataaaataataaattaaataaaataattgaattaaattaaaagaattaaacttaatgatgaactaaatataaattagagtaatccataattgatttcaatccttctctatggtttacataaatcgatgtctacttgtaagcttaagtatagtgaattggtcattttaaaatagccaagaacaaatttcaaatgttcttgtcaccaaaaaatgataaggatttgaagatttgaggtgatatataaggagtcttccaattcccttgtccaaagcatccgctcattcatgggctgagaaaagggaagtcattcatgcagtctactctctccacagaactgacggggcacaaggacaacatcgatttttcatggaacatgcctctaggaatgcagctgtgtgcacacacactgctaagcacacacttctttacataattacttgtaactgtattttcttatttattctctccaatttttttacacaaattcatcacttttccccataatcaaagaggattttgatcagaatgcttgtggggagccccttgcctgccagatgcccaacatcactactcttgaagggaggaggaacggcagctctcttgatttctactctaatcctctaggactaaaaccagaaggttgcatgtccagtgcgggagcatcgaagaagatcctgtctgtagaagcaggagcgtcaagacttgactgagagccatggtgctgaaatgagatagattccctgatggagagcacacgtggacccccacacctgagggctcactgctcctcaccacagatgcactcccctactgagtcctgagacctgagtgcaccccatagagtagggctcagatgaggggatgcaaatctccaccagctccaccctcccctgggttcaaaagacgaggacagggcctcgctcagtgaatcctgctctccaccatggacatactttgttccacgctcctgctactgactgtcccgtcctgtgagtgctgtggtcaggtagtacttcagaagcaaaaaatctattctctcctttgtgggcttcatcttcttatgtcttctccacaggggtcttatcccaggtcaccttgaaggagtctggtcctgcgctggtgaaacccacacagaccctcacactgacctgcaccttctctgggttctcactcagcactagtggaatgcgtgtgagctggatccgtcagcccccagggaaggccctggagtggcttgcacgcattgattgggatgatgataaattctacagcacatctctgaagaccaggctcaccatctccaaggacacctccaaaaaccaggtggtccttacaatgaccaacatggaccctgtggacacggccgtgtattactgtgcacggataccacagagacacagcccagggcgcctcctgtacaagaacccaggctgcttctcagtggtgctccctccccacctctgcagaacaggatagtgtggctgagatgccatttcctgccagggcctgcgtttcccatccccatctgactcagagccttgttttcctccctcttctttactaataaatggcatgtcccctgttagtggttcgtgcaagcagaagctgtatcctgtttgacaaagattcagcatgaaaggttcctgttacctaaaaaaaaatagacagatgagacttaattaacctaaataatttttttcacaacaacagagtgaatacacaatttgcagaatgacagaaaacttttgcacactttgtctgtgacagggaactaatatgaagaatttgcaaggaactcaaacaactctacaacaacaacagcaacgagaaccaaataaccccattaaaatgagcaaagaacatgagtagacattttcaaaagaacacatagaaatggataataaatatataaacaatgctcaacatcactaaccatcagggaaatgcaaattaaaaccacaataagatatcatcttccaccagtcacaatgactgttactaaaaactcaaataatatcagatgttgctgaggatgggaaataaaggcaactcttagacattgttgatgaggatgtagacgagtacaaactcaaattcctatttctatttcaacccctgattcctactgtcaatgggagggaagtctcagaaccaatcacacatcagacggcaaatctgtcaaccaagagtctttccactgaaggacctgggaggtcaggaccctcaggaaagtgctggggaccctgtcttgggagtgcccagcagatctcagaactctccatgggtcctgctggacactcatgtagggtaacgagtggccaccttttcagtgttaccagtgagctctgagtgttcctaatgggaccaggatgggtctaggtgcctgctcaatgtcagagacagcaatggtcccacaaaaaacccaggtaatctttaggccaataaaatgtgggttcacagtgaggagtgcatcctggggttggggtttgttctgcagcgggaagagcgctgtgcacagaaagcttagaaatggggcaagagatgcttttcctcaggcaggatttagggcttggtctctcagcatcccacacttgtacagctgatgtggcatctgtgttttctttctcatcctagatcaggctttgagctgtgaaataccctgcctcatgcatatgcaaataacctgaggtcttctgagataaatatagatatattggtgccctgagagcatcacataacaaccacattcctcctctgaagaagcccctgggagcacagctcatcaccatggactggacctggaggttcctctttgtggtggcagcagctacaggtaaggggcttcctagtcctaaggctgaggaagggatcctggtttagttaaagaggattttattcacccctgtgtcctctccacaggtgtccagtcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagacacagtgtgaaaacccacatcctgagagtgtcagaaaccctgagggagaaggcagctgtgccgggctgaggagatgacaggggttattaggtttaaggctgtttacaaaatgggttatatatttgagaaaaaaagaacagtagaaacaagtacatactctaattttaagataaatattccattcaagagtcgtaatataagccaaattcacagagtggaaaaggccacactctataacgttgatacaaacattccatgaaggtgctactgtgaacaagttttcaaattggatgaatacatgatttggagcaaggttatttgatcatgtggtgagactaagaatgattcttaaaaagtgccaaaagtttccttcaaatgtttctgtcactccttatcataaagtttattttacagcagttttaggattacaaagaaattgcacaggaggcgtgagaattcccatgactccctgccctacacaggcacagcctcctccactacgaccatcctgcaccgcagtcacaaatcagttacaatggaggaatctccaaggacgcttggttctttctttttctggtgatctcctaatataacaagcctaagtatctcaagattccacggttttttcagtgttttctagaactgatattagtcagagggaaagtgggtaaggctattactatttgaactctttcttccatacatattttcaatcagaagtgtttagaggccaggacatatcttcacggtcacacattgagaaggatgtagatatgtcccactaccttctcctgagatctcagacagaatcccagatttcaaaaggacacagaaggacagctctcaggtgcttttaaaaaatgacccacttccagggacagggagcttccctataaccatggtggatgttctgaactacaataaacattggatggatccaggattgtttgaagtcactgtcattattacattcagctgctgtttcaatgtgtctgaagtagtaaatgacaatttagatgacaatttatatgaatcttcaagggtagaacaatattgaccatattccaaaatctgtccttgatccatgatcacactcatctcccagaccaggtccttcagcacgtctctttacctgaaagaagaggactctgggcttggagaggggagaccccaagaagacaactgagttctcaaagggcacagccagcatcctactcccagggcgagcccaaaagactggggcctccctcctcctttttcacctctccgtacaaaggcaccacccacatgcaaatccttacttaagcacccacaggaaaccaccacacatttccttaaattcaggttccagctcacatgggaaatactttctgagagtcctggacctcctgtgcaagaacatgaaacacctgtggttcttcctcctcctggtggcagccccgagatgtgagtgtctcagggatccagacatgggggtatgggaggtgcctctgatcccagggctcactgtgggtctctctgttcacaggggtcctgtcccaggtgcagctgcaggagtcgggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagacacagtgaggggaggtgagtgtgagcccagacacaaacctccctgcatggacgcggaggggaccggcgcaggtgctgctcaggaccagcaggtggcgcgcggggcccccagagcatgaggccgggtcaggagcaggtgcagggagggcggggcttcctcatctgctcagtggtctccgtcctcgccagcacctcgctgtcaccagggctcctctttctttattatctgtggttctgcttcctcacattcttgtgccaggaaagaaacgaggaagacaaattttcgtctatagttgaagcttcaccaattactaggaacttgcctacaagttcctgcatgacccattataacttatcgattaaaaaatatatattctaatgcttctcaccatctcttgatttgtatcatcaactgaattgtaccctctttgaaattcatatgatgaaaccttaaattcaatggatctatattggaattttaatgaaataattaaggttaaatgtggtcataattgtaagaccctaatgcaatagacgtgttgtctttataagaagaggaagagacaccagagacctctcacttttcacgtgcaggcagagaagaggccatgtggagacatagtgcactagaaggtggcccagtgcaagccaggaagaagccgcgccaagaaccagccctgccagcacactaatcttcaacattgggtgcagcaatgggcatcccttcaggtccatcagcaagtcagagacctaaacaaatattccctattcatgttgaattggcagagtgtaaaaatctagaagacaaaatggccaggaaaaatgtgcgattttgcatgggtgaaggtctggcaagttaatcatcttgaggactgttgaggtcctgaggtggtcctgcccttgcttgatgcccaggccgtagtccaactcacacgaaattgccagggaatagacagtagtttttttagtagttcttgttatcaggcataagtgcatttgaattttctcttcatggcctttcctggcactatttctcattttttttaacacacatagtttcaactagatttatcaccttcacagggtcacagagaagggtggaagaagggaggccctgtatgggtctcgaagaaacatggaaaagagtggagagggacaatagcagggtgtaaggaattattgagaccttactctgcccctcccaggaggctcaggccagcctttttctgcatttgaggttctgggttataaacgctgtagactcctcccttcagggcagggtgacaactatgcaaatgcaagtgggggcctccccacttaaacccagggctcccctccacagtgagtctccctcactgcccagctgggatctcagggcttcattttctgtcctccaccatcatggggtcaaccgccatcctcgccctcctcctggctgttctccaaggtcagtcctgccgagggcttgaggtcacagaggagaacgggtggaaaggagcccctgattcaaattttgtgtctcccccacaggagtctgttccgaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacacacagtgagagaaaccagccccgagcccgtctaaaaccctccacaccgcaggtgcagaatgagctgctagagactcactccccaggggcctctctattcatccggggaggaaacactggctgtttgtgtcctcaggagcaagaaccagagaacaatgtgggagggttcccagcccctaaggcaactgtataggggacctgaccatgggaggtggattctctgacggggctcttgtgtgttctacaaggttgttcatggtgtatattagatggttaacatcaaaaggctgcctaacaggcacctctccaatatgacagtattttaattagtgaaaattttacacagttcatcattgcttgcttgccttcctccctcctgtccactctcactcactccttcttttattttctacttaattttacaaaatcatttaacccctttttgaactattaataggttatctttgtttggtgattgttttcctttcaataatatgtactgaataattcatctttgtgccaattcataagtattctggtgtaataaagacttctttcataaaaattggataaattaaaataaagataaatttttaaaaacatacgatctatcaaaactgaaccataaagaaataaaaactctgggttgggtgtgtttgctcattcctgtaatcccagcactttgggaggccatggccggtttacaagcaaaaattgaaagttagacaagaaggagtgatagaaagagaaaatgtatattaaatttcagaaatatttaagaatgtatctgcctgaaccctagttctcaccatatctttaggtgaatgctaaaatgcagcaaaatcacgcatgttctcactacagaaagtgggttctacaaaccacactcggcacatttagctttgtcctggagttggttcagggagttattggggccagtgatgaggagcacaggccaagataccagcgatcacttatcccaaacatgagctctaacatacacacttagtcccttttccgtgtgtggtttacttccacatctgtacatggagagaccactgactgacaaaatataatttatacaaatatgtaaaattaaatagggtgatcagttcaaggtgtttatcacagcataattttacaataagacagcatatttcccaaataccatcattgtcaccaaactccttcaaggcacagtcatcttatctgggccccgtcctctcctcaggtgtcccaccccagagcttggtatatagtaggagacatgcaaataaggccctccctctgctgatgaaaatgagcccagccctgaccctgcagctctgggagaggagccccagccgtgagattcccaggagtttccacttggtgatcagcactgaacacagaccaccaaccatggagtttgggcttagctgggttttccttgttgctattttaaaaggtaattcatggtgtactagagatactgagtgtgaggggacatgagtggtagaaacagtggatatgtgtggcagtttctgaccttggtgtttctgtgtttgcaggtgtccaatgtgaggtgcagctggtggagtctgggggaggcttggtacagccagggcggtccctgagactctcctgtacagcttctggattcacctttggtgattatgctatgagctggttccgccaggctccagggaaggggctggagtgggtaggtttcattagaagcaaagcttatggtgggacaacagaatacaccgcgtctgtgaaaggcagattcaccatctcaagagatggttccaaaagcatcgcctatctgcaaatgaacagcctgaaaaccgaggacacagccgtgtattactgtactagagacacagtgaggggaggtcagtgtgagcccagacacaaacctccctgcaggggcgcacagagccaccagggggcgctagggaccgactgagtacgggacaggtcccaggagcaggtgcagggggaggtttcctttttccttggctggaaaagtcacctttatcttcccaggactcgagccttctaggctgtgatattttattacttgtatttactgttcatttattatcattagtttttaaattttggtaatttttacaactctatggatatatttttaagtgtatactttcaagaaataaacattcctaattatttgcactgattctcccagagttttattaacatttgttgacatcagcaactacatagctatagggacaaacacttttaacgatagacagttgtttaggcctgaaaccccgtttatactaaaaatttacaaaaattagcctggcgtggtgaggggcgcctgtaatcccagctactcgggaggctgagcaaggagaattgcttgaatccgggaagcggaagtcacagtgagtggagtggactgccactacactccagcctggcgacagagcgagactttgtatgaaagaaagaaaagaaaaggaagaaaggaagaaaggaaggaaggaaggaaggaaggaaggaaggaaggaaggaaggaaggaaggaaaagagagaaagaaagagagagaatgtttgtcaaatggtgaacatattgtgtgaagattatatgtctgtattacttcattaaagctattataaataaaagtctaatgtggtagaaaaagatgaagagagaaataaaaataatacaagaaaagtcatgaactcctgaatgaattaacccttagtttttctctattacttataaaaacaccaagatacagccaaataatatcacgatatcattataagaagagtgttttgtaaacctcactgggaatttatagctctttcctagagttaattttgggaacagttggatccaattgtgagaaatgcaggctggacactgagactggctcttatgagatgtgagctcttgtctatgtcacatggtccttccatacttgggggtttacattcacatctgtaaatgaaggaaacattgactctcaaagaacatatttcatgtgcatgtaaaagtatgaatgctagtgagaattaattacttatgaagtataatcacccacatccactcttggacacagcccactctgaggcatctgttacagaactcattatatagtaggagacatgcaaatagggtcctccctctgctgatgaaaaccagcccagccctgaccctgcagctctgggagaggagccccagccctgagattcccaggtgtttccattcggtgatcagcactgaacacagagaacgcaccatggagtttggactgagctgggttttccttgttgctattttaaaaggtgattcatggataaatagagatgttgagtgtgagtgaacatgagtgagagaaacagtggatatgtgtggcagtgtctgaccagggtgtctctgtgtttgcaggtgtccagtgtgaagtgcagctggtggagtctgggggagtcgtggtacagcctggggggtccctgagactctcctgtgcagcctctggattcacctttgatgattataccatgcactgggtccgtcaagctccggggaagggtctggagtgggtctctcttattagttgggatggtggtagcacatactatgcagactctgtgaagggccgattcaccatctccagagacaacagcaaaaactccctgtatctgcaaatgaacagtctgagaactgaggacaccgccttgtattactgtgcaaaagatacacagtgaggggaagtcagcgagagcccagacaaaaaccttgctgcaggaagacaggaggggcctgggctgcagaggccactcaagacacactgagcatagggttaactctgggacaagttgctcaggaaggttaagagctggtttcctttcagagtcttcacaatttctccatctaacagtttccccaggaaccctgtctagatctgtgatctggatctgctgaaactgcctgtgtcaccttcctcacctgtgactttgggggagctgattgtggacactccagtgtgtgggatttcttggtgacagcaattgtgtcttctgtctaggcatgtctagggctggccatcaggaagggcaggctggaatttttggaaagaggcgcacctgccatccaccaggaaattttgttgtcttttgttctgctagaattaaatcagacacaccaaggttaactagcactatcttcctagcttgagaaacttgatggcaggcttgaataacacctgtatgaagccatcagagcaacaactagattaatgtctgctagaattaaatcaggcacaccaaggttaactagcactatcttcctagctcgagaaacttgatagcaggcttgaataacacttgtatgaaaccatcagagcaacacctagaatagtgtctgatttaataattgagactatggtctagccaaggagcagaagtgtttagaggccaggacacatcttcaaggtcacacattgagaaggatggagatatgtcccactaccttctcctacgatctcagacagaatcccaaatttcaaaaggacacagaaggacagctctcaggtgcttttaaaaaatgacccacttccagggacagtgagcttccctgtaaccatggtggatgttctgaactacaataaacattggatggatccagtattgtttgaagtcactgtcattattacattcagctgttgtttcaatgtgtctgaaagggtaaatgactatttagatggcctgggtgtgtggttggttttatatgaatctttaagggttgaacagtactgaccctattccaaaatctgtccttgatccaggatcacactcatctctcagaccagctccttcagcacatctctttacctggaagaagaggactctgggcttggagaggggaggccccaagaagagaactgagttctcaaagggcacagccagcattctcctcccagggtgagctcaaaagactggcgcctctctcatcccttttcactgctccgtacaaacgcaccacccccatgcaaatcctcacttaggcgcccacaggaagccaccacacatttccttaaattcaggtccaactcataagggaaatgctttctgagagtcatggatctcatgtgcaagaaaatgaagcacctgtggttcttcctcctgctggtggcggctcccagatgtgagtgtttctaggatgcagacatggagatatgggaggctgcctctgatcccagggctcactgtgggtttttctgttcacaggggtcctgtcccagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagacacacagtgaggggaggtgagtgtgagcccagacaaaaacctccctgcagggaggctgagggggcgggcgcaggtgcagctcagggccagcagggggcgtgcggagctcacggaatacaaggccgggtcaggagcaggtgcagggtgagcggggcttgctcatcttctcagagatcatcatctccctcctcgccagcacctcagctttccgtagaggtcctctttctttattgtgtgtggttctacttcctcacatccttgtgccaggaaagaaaggagtaaggcaaattttcctgttacaattgaagtttcaccaattactaagaactttcctgcaagtacctgcacagcccattataccttatttatatatgtatatattctaatgcttctcaccatctcttgatttgtgtcatcaatttaattgtgccctttttgaaattcatatgctgaaactttaaatccaatggatctatatcggaattttaatggtataattaatgttaaatgtggtcataaatgagaccctaatgcaatagagctgttgtctttataagaagaggaagagacaccagatacctctcacttctcacatgcactcagagaagaggccacgtggagacatagtgcactagaaggtgggcctctgcaagccaggaagaagccgcaccaagaaccaaccctgccagcaccttgatcttctttcattacaggaagaaagttctgaaaatgtcactggggtgaaccacattgtgctgggcttggttcagggagcagtcaggcccagtgttgtgaccttcaaccacagaatcctgaggctgcctcaaagtccccatcagttcccggactcgctatgtttctcgatcgtatcagtgcatccggagctccctggtggctttagtgattccttgcttgccatgctgaggtctcgctgtagattatgttgggttttctaagaccgttttgctattgtaagatccactccctgggacagagcgattccctctaaacctgatggaggttctgaactacaaataacgttaagtgaatcctggtgtgtctgaactcaagtgattgttacattaagctgctgttgcaatctgtttcctcacctgggaaaagaggagccaggacatagtgagttgaggccccaggaagataactgaattctcagagggcacagccagcatcctcttcccagggagagtctaaaagactggggcctccctcatcccttttcacctgtccatacagaggcaccacccacatgcaaatctcacttaggcacccacagaaaaccaccacacatttccttaaattcagggtcctgctcacatgggaaatactttctgagagtcctggacctcctgtgcaagaacatgaaacacctgtggttcttcctcctgctggtggcagctcccagatgtgagtgtctcaaggctgcagacatggagatatgggaggtgcctctgatcccagggctcactgtgtgtctctctgttcacaggggtcctgccccaggtgcagctgcaggagtcgggcccaggactggtgaagccttcacagaccctgtccctcacctgtactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagagacacaatgaggggaggtgagtgtgagcccagacacaaacctccctgcagggaggcggaggggaccggcgcagttgctgctcaggaccagcagggggtgcgcggggcccacagagcatgaggccgggtcaggagcaggtgcagggagggcggggcttcctcatctgctcagtgctctccctcctcgccagcacctcagctgtccccagggctcctctttctttattatctgtggttctgcttcctcacatccttgtggtaggaaaggaaagaggaaggcaaattttcctcttagagtcaaagtgtcactaattactaggaactttcctacaagttcctgaatgtcccatttttccttcttaattaaaaaaaatatatattctaatacttctcaccatctcttgatttgtgtcatcagttgaattgtgctgtctttgaaattcaaatgctgaaaccttaaatccaattgatctatattggaattttaaggatggaattaaggttaaatgtgatcataagtctgagattctaatgcaatagatctgttgtctttataagaagtggaagagtcaccagagacctctcacttttcccgtgcacgcagagaagaggccatgtggagacatagtggactagaaggtgcaagccaagaagaagccgcaccaagaaccaaccctgccagcacctggaccttggacattcatgctcactcatgcgctggaagagggatgtcagagatgcagtctactcttcctaaagggctgacagggcataaagacaagactgattatccatggaacattcctataggaatgcacctatgtgcagacacacaaataagctgtgtgtcttgcagtgtgtaaatggctgagatgccatttacacttctttacacaatacatttttaaatgttttgttgatgtattctctatcattcaaaaaaatcatcactttccccctaaacaaagaggatttttatcagaacacttatgggaagccccttgctctcccagattcccaccctcatttctcctgaaggaagaaagaaaaccatctctattgatttccactctcatcctctaggactaaaaccagaaagctgcatgctccctgggtgagccttagagaagaccctgtctgtaggagcaggaatctcagagccttggctgagaggcatggtcctgaaatgagatggagtcccccatggagatcccacgtggacgcccacacctgagggctcactgctcctcaccacagatgcactcccctactgagtcctgagacctgagtgcaccccatagagtaggactcagatgagggaatgcaaatctccaccagctccaccctcctctgggttcaaaagctgagcacggggcctcgctcagtgactcctgtgccccaccatggacacactttgctacacactcctgctgctgaccaccccttcctgtgagtgctgtggtcagggacttcctcagaagtgaaacatcagttgtctcctttgtgggcttcatcttcttatgtcttctccacaggggtcttgtcccaggtcaccttgaaggagtctggtcctgtgctggtgaaacccacagagaccctcacgctgacctgcaccgtctctgggttctcactcagcaatgctagaatgggtgtgagctggatccgtcagcccccagggaaggccctggagtggcttgcacacattttttcgaatgacgaaaaatcctacagcacatctctgaagagcaggctcaccatctccaaggacacctccaaaagccaggtggtccttaccatgaccaacatggaccctgtggacacagccacatattactgtgcacggataccacagagacacagcccaggatgcctcctgtacaagaacctagctgcatctcagtggtgctccctccctacctctgcagaacaggaaagtgtgactgagatgccatttcctgccagggcttgtgtttcctataccaaccagactcagagccctgtctgttttcctattctgtactattaaatggcatgttccctgttagtgattcacgcaagcagaggctgtatcctgtttgacaaagattgagcatgaaagattcccattacctgggccacatgcatcactgatatgtggccacttatttcttgagctcatatccttccaaggggctgaatacaaatatctataacataggccatcttaaactgcaggaagtaatgttggagaagaatgtggagataaaagagcaggatgattaattaaaatccagataccatctttttttgcagagaaaaatttacactaataaagtaattttatgaaacaacaagaaagatggaatgtgtcctcatcatggagtgtctcacttgcagtgcacaaataaatttctaaaaattttatagggaaggtgtgatagatgaggctgatttccacacagggagtgattaaatacccaggtaagtataaaatccaacacttggaaaggaaaatgcctcagcttgacatcagaaacccagctcagtaagtctgaggagcaatgcggacagggagcacccagcacaggacccagccctggggcaggtgcacaggaggctggggcagggttttctctcaggaattgaatgttccttatttcaaagcaataatgacctaacatttaaataagaatttagcaagtactgatgtgcctgtaagtattttattgtatacgcagcctatacctaacccaataatttaatgcaaaccggatttaaaaggagaaatgtctaggtctttcaaatgtatttctagttaggaattgaggggtggttttattaattcaatgggtgttactgtccggagacacactcatcccagaagttagatgtgcagagggcaaggctcaggaaaagttcaggtagacagtgagccatatgaacagggaatcacattgaggaccatgtcctgtgagattctgggtttctgtgagaggagttctgtcttcatggacttctgggcatgtcagaggacaaatattaaacaaagttcagggcagggagctcagcatcccactgtggcgtggtccacatgtcacctatcttcttcctcagggtggcgtggcctgagctatgaaaaacctgcctcatgaatatgcaaatgcactgctgtctaccgaggtaaatacagatctttccttgcccagagaccatcacacaacagccacatccctcccctacagaagcccccagagcgcagcacctcaccatggactgcacctggaggatcctcttcttggtggcagcagctacaggcaagagaatccttagttccagggctgatgaggggactgggtccagttaagtggtgtctcatccactcctctgtcctctccacaggcacccacgcccaggtccagctggtacagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaacagacacagtgtgaaaacccacatcctgagagcgtcagaaaccctgaggaatgaggcagctgtgctgaggctgaggagatgacaggatttatgaagtttaaatctgtttagaaaatgggttgagtaattgagtaaaacagcaatggaaagatgtgtacactgtcattatgtaggaaatagtattttcaactgtcaccctataagtaacactcacagagtgggaaaggcagccatcaatcaagctgatgcaaacattcccatgggggctttgtggggacatacattttataattggatctataaatcattagaaacaggattgcttgatctcatggtaagactaaatataatttccaagaagtggccgaaatttcttccaaaatgtctttgcccttccttttacactgaatttattttaaaggagttttaggatcacaacaagtttgagtagaagaaacagagttcccatgcactcctgccccccgacgctcagccttctctgctatcaacaccctgcaccagtgtcataaatcggttacagaggatgaatctccacagacacattggttgtttccttttctggtggtccctggtataacaagcctcaactatcttgaagcaccccaggttcaagtggattcttcaagtgggttactgggaatgatgccaggtagaaggaaagtgggtgggaccattcctctttgtgcttttctttatatttgttaggtaatcaccacagtgtgtacacatcacaccatgttcccattacagagaaaaggttctgcgaacctcacgagctgtgacccctgtgtgctgggcttggttcagggagaagtcaggtccagtggtgagaagcacaggcccagatgcccaggctcactctgaccaaatgtgagcactggggacattgtaaaacccacctgtgcttttgctgataatttttcatctttaacatggaaataatattgatactatataccatggtttctctgcgtatgtaaaaataaaagatgattggtgctaactttaaaaatatgcagtttatgtagatctatggtacctcaataaaactgttttaaaataaaaattacaaaattataagatttttaggttttaaggtttaagtttatcacaaaacaaactgacaataggaaagcacaatttcccaatgctttcaatatcacagatctccccgaggacattctgacatgctctgagccccactatctccaaaggcctctcaccccagagcttactatatagtaggagatatgcaaatagagccctccgtctgctgatgaaaaccagcccagccctgaccctgcagctctgagagaggagcccagccctgggattttcaggtgttttcatttggtgatcaggactgaacagagagaactcaccatggagtttgggctgagctggctttttcttgtggctaaaataaaaggtaattcatggagaaatagaaaaattgagtgtgaatggataagagtgagagaaacagtggatacgtgtggcagtttctgaccagggtttctttttgtttgcaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagacacagtgaggggaagtcattgtgagcccagacacaaacctccctgcaggaacgatgggggggaaatcagcggcagggggcgctcaggacccgctgatcagagtcatccccagaggcaggtgcagatggaggctgtttcctgtcagggtgtgggacttcatcttcttctgacagtttctctagtgaacctctctaacctcagaattctgtgcttactaatgtcatctctacgtattttttaaaagatcattttaatatgagcacctattctcacacgcaccaaatgcagattgacgcttacagagatgaaaagttctcaaccatggtcaccaggatgagagtcctgaggaaactcaggggtgcctggtgactcttccgcaatcagtcctgggacagaaacctcagggacagcctttatgagttttgatcacatctaacagagaggatgggccagggccagagtcatgtagaacctcacaggttccacgtctgacccttctcctgacactaaagccaatcagcatcagcactgacctggtgctccttttgctcccaatccatgttctttctttggagagtttcttctcccttttttatttgcttttcctgtttcctgaaaaagaaacagatggtccccttggtccacattccagggctcaaggcattttcttggagctcaggtggggctcaggctgtggctcagcaaacaaatgaaaacttgcacaagaaataagtgacataaagatagaaaaaatattaaatttcagaaacacctaataatttatcttcgtgaaccctagttctcaccatatttttaggtgaatgctagaatgcagcaaaattacacatgctctcaatacagaaagtgggtttcacaaaccacactaggcatgctcagctctgtcctggagttgggttagggagtaatatagggccagtggatgaggagcacaggcctagatactggggctcactaacctcaggtatgagctcttagatacatacaaagcccctccacgcatgggtttacttccccatctgtaaattgagaaaccattgacccctaaaaatatgatttacacaaatatgtaaaaatgtaagagagtgattagtgcaaagtgtttatcacagcacaatttcataacaagacagcaagttttccaaacagcatcattgtcattagattcctgcagggcatcattaccttatctgggccctgccctctgctcaggcatcccaccccagagcttgctatatagtaggtgacatgcaaatagggccctccctctcctgatgaaaaccagcccagtcctgaccctgcagctctgggagaggagccccagccttgggattcccaagtgttttcattcagtgatcaggactgaacacagaggactcaccatggagtttgggctgagctggattttccttgctgctattttaaaaggtgatttatggagaactagagagattaagtgtgagtggacgtgagtgagagaaacagtggatatgtgtggcagtttctgatcttagtgtctctgtgtttgcaggtgtccagtgtgaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagacacagtgaggggaggtcagtgtgagcccggacacaaacctccctgcaggggcgcgcggggccaccagggggcgctcgtgacccactgagggcgggacaggtcccaggagcaggtgccgggagaggtttcctttctcctcagctggaaaagtcaggtttatcttcgcaggactctggagtcttctaggctgtgatattttgttacttatatttattatgaattttatcattaatacttaaattttagtaattattaacattctacatattattatatttttaagtatatactttcaagaaataaacattcctaattgcataataattattgtaacataattattgtaacatataattatatcctaaatgcataataattattgtaacaatcacattctcattttgtatttgaattttcatatttattttactaagattttaattttaatataaaatatttttctacaattgtctgttttccatttttgtgagataaaattagcatatacaaaaatgcacagattctccaggtacagttagagggtttcaggcaaatgtgcacatacttgtctacagcagctaagtgagggtgaggaacaggtgaggtccatctccccacaaagtgtcctcttagtgcttccagttagttctcacataaggatttttttttatttcaactttataatttagatacagagggttcaaattttgaatgtattgtttgaagattgttatttgtcaatattacctcattaaaactattaaaaatggaagtgtataatttggtagaaaaggattaaaagaaagataaaaataatacaagaaaagtcacagactcctgaatccacatgtgaattagcactcagtttttctatattatttataaaaacactaagatacagccaaataatatcatgatatccttataagaagagtgtttcgtaaacctcactgggaatttctagctctgtcctagagttaatttagggagcagtcggatccagtcctgagcaacacaggccagacactgagacttaccacatcttaccagatgtgagctcttagacacgtcacatggcctctccatgcttgggggtttaccttcacatctgtaaatgaaggaaacattgactgccacagaacatacttcatgtgcatctatacagtagaaatgctaataagaattaattgtttatgaagtgtaatcactctgggacacagcccactcagaggcatcccttccagaacccgctatatagtaggagacatgcaaatagggccctccctctgctgatgaaaaccagcccagccctgaccctgcagctctgggagaggagccccagccctgagattcccaggtgtttccattcagtgatcagcactgaacacagaggactcaccatggagttgggactgagctggattttccttttggctattttaaaaggtgattcatggagaaatagagagattgagtgtgagtggacatgagtggatttgtgtggcagtttctgaccttggtgtctctgtgtttgcaggtgtccagtgtgaagtgcagctggtggagtctgggggaggcttggtacagcctggcaggtccctgagactctcctgtgcagcctctggattcacctctgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatacacagtgaggggaagtcagcgagagcccagacaaaaacctcctgcaggaagacaggaggggcctgggctgcagagggcactcaagacacactgaaaacacggttaacactgggacaagttgctcaggaaggttaagggctggtttcctttcagagtctttgctgtttctgtatctaacagtttccccaggaaccctgtctagatttgcgatcttgatctgcttaaactgtctctgtgtcatttttcttacctgtgactttaggggagctgatcgtggacactccagtgtgtggagatttcctggtgacagcaattgtgtgttctgtctaagcatgtctagggccggtcatcaggaaggacaggaaggacaggctggaattcttggaaacagctgcacctgccatccaccagggaactttattgtcttctgttctgctaaaattaaatcaggcacaccaagtttaactagcactatcttcctagcttaagaaaattgatggcaggtttcaataacacctatattaaaccttcagagcaacacctacattagtgtctgatttgataattgagactatgttctagccaagcagacacataaaactggtgattgccatgcttagttcatatttcatatttatcaatagaatctggctggtgttataaacagatttgccaacacatgtgtgttagtgtttgtcttcaaattcctatttctatttaaactcccgattcctactctcagtgggagggaaaactcacagccaatcacacatcacaggacaaatctgtaaacgaagagtcattcctctgaaggtcctgggtgttcaggactctcaggcaggtgctgaggaccctgtcttgggagtgcccagcagatctcagaaccctacatggggcctgctggacactcatgtgggataactagtcgccacttattcagagttaccagtgagctttgactgttccgaatgggaccagcatggagtcaaggtgcctgctcaatgtcagagacagcgatggtctcagaaacaatccaggtaatctctaggccaataaaatgtggattcacagtgagaagtacatcctggaggtggagcttgttcttcagtgggaagagtgctgtgcacagaaagcttagaaatggggaagggggtgcgtttcctcaggcaggattagggcttcgtccctcagcgtcccactcttgtatggctgatgtggcatctgtgttttctttctcatactagataaggctttgagctgtgaaataccctgcctcatgaatatgcaaataacctgagctcttctgaggtaaatataggtatattggtgccctgagagcatcactcaacaaccacatctgtcctctagagaaaaccctgtgagcacagctcctcaccatggactggacctggaggatcctcttcttggtggcagcagctacaagtaaggggcttcctagtctcaaagctgaggaacggatcctggttcagtcaaagaggattttattctctcctgtgttctctccacaggtgcccactcccaggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagaggcacagtgtgaaaaaccacatcctcagagagtcagaaaccctaggggagaaggcagctgtgctgggctgaggagatgacaggggttatcaggtttaaggcttttttgaaaatgggttatatatttgagaaaaaaataacaatagaaacaagtacacactctaattttaagagatatattcaattcaagaattgtagaagccgaattcacagtgggaaaggccacactcaataaagttgataaaaacattccaggaaggtgctactgtgaacaagttttcaaattagatgaataaataatttggagcaatgttatttgatcatgaggtgagacagtatgattcttaaaaagtggaaaaaattctttcaaatgtttctgccactctttatcataaaattaattttagagcagttttaggattacaatgaaattgcacagaaggcatgagaattcccatgactccctgccctacacaggcagagcctcctccactatgaccatccagcaccacagtcacaaatcagttacaatggaggaatctccaaggacatttgattctttcttttttggtgatgccctaatataacaagcctaatttatctcggaataccacaagttttgttcagtggatttctaggaatcatattagttagagggaaagttggtgaggctcttactatttgaactcattcttccaaaatccacaatggaaaaggtaaatgtggtctccgtgacctcaattcaagggctgaagccctttccctgtagctcagctggggctcaggctgtggctactgcagccatgtggaagaggctgaagggactttcttcactctccttgctcaggaccatccactgtattgtgtataggcttctctggaaatgcaagtggccatttgtagtgaaagaaatatgtttgtctggttaaaatgggaggtggatgtagagttaattggctgctacataaactgtccttctccaccagtgcttttaggatgagattgtgaaatttgtaagaatcaaaatggagtcacatatgttaaaaccctgacaaatggattcaggaagtgtagggagaattcttacacacatatccctgacaacaagaactatcataaaatagttcttgcaaaaagaccaacatgacctcataatcatgacttctgcaaagacttctactcagaatctacttgcccagccttagattaatgccatctgaattacactgatcatgttactatcactgctcctcaccacagatgcaacaccctcctgagtcctgaaacctgactccatcccatagagtagggcacagatgaggggaatgcaaatctccaccagctccaccctcctctgggttgaaaaagccgagcacaggtcccagctcagtgactcctgtgccccaccatggacacactttgctccacgctcctgctgctgaccatcccttcatgtgagtgctgtggtcagggactccttcacgggtgaaacatcagttttcttgtttgtgggcttcatcttcttatgctttctccacaggggtcttgtcccagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattgggatgatgataagcgctacagcccatctctgaagagcaggctcaccatcaccaaggacacctccaaaaaccaggtggtccttacaatgaccaacatggaccctgtggacacagccacatattactgtgcacacagaccacaaagacacagcccagggcacctcctgtacaaaaacccaggctgcttctcattggtgctccctccccacctctgcagaacaggaaagtgcagctgagatacgttttcctgccagggcctgcatttcccatccccattagactcagagccctgtcttcctccttcttctttaataataaatggcatgactcctgttaatagttcatagaagcagaagctgagtcctgtttgtcaaacattcagcatgaaatgttcatgttacctgggccagatgcatcactggtatgtggccgccagtttaatgggctcatactactccaagcggccgaatacaaaacagcagaacatatgcctttattaaactgcaggaaatgacactggagaaaaatgtggggataagggagcaggaggaattgtgaaaattcagataatagcttttctcgtagagagaaaggtgcgttaataaaataattttataaaacaataaaacagatggaatgtgtccccatcacggagtgtctcacagtaacagtgcagaaataaatttctaaaaatgtcgtagggtaggtgtgatagacaaggcttatttccacacagggagcccataaatacccatgtaattaaaaatccagctcttagaaagggagtgcctcaacttgacatcagaaacccacgtcagtgagtctgaggagcaatgtgctctgcagtgcaagggctgggctgcaggggacgctcaaagcccacccagtacaagtttcagccccagagcaggtgcacaggaggctggggagggattcctctcgagatctgtgtcattctttaaaaaatctaaaatacatatttgacaactgaacattctataaatatcctattcaattgcgagcatttatcaaacttgatgttgtaatgagaaccacttttacactggagatttctaacccttctagatatcttaatagtatgcagctggaggttaaggaaactacttttcttttctataaataagtgaaacttttggagaaacacactcatcccccaaataatacattcatgtattaaagcctagaaatgcttatattacctctgagccatttgagtgtgggttcccagtgaagtcctgttctagggaaaattgttagccaatgggagaagctctgtcaacatagagcttagggatatggcagggctcacatggcctctaaggggattacagcttgaaccttgagcatcctccgtgtgtcatctctctgctctttctcatgcaatatcaggtatgaaataggatcactcatgaatatgcaaataactgaggtaaatatagctatctttgggccctgagagcatcacccaacaaccacacccctcctaagaagaagcccctagaccacagctccacaccatggactggacctggaggatcctcttcttggtggcagcagcaacaggtaaggggctccccagtcactgggctgagggagaaaccagcacagtcaagtgagacttcatgcactcccatctcctctccacaggtgcccactcccaggtgcagctggtgcaatctgggtctgagttgaagaagcctggggcctcagtgaaggtttcctgcaaggcttctggatacaccttcactagctatgctatgaattgggtgcgacaggcccctggacaagggcttgagtggatgggatggatcaacaccaacactgggaacccaacgtatgcccagggcttcacaggacggtttgtcttctccttggacacctctgtcagcacggcatatctgcagatcagcagcctaaaggctgaggacactgccgtgtattactgtgcgagagacacagtgtggaaacccacatcctgagagtgtcaaaaaccctgtgggaggaggcagctgtgctgagctgaggcagtgacagggacaacgtggctgcaccctttgtagggttataaacatacatatatatgcacccaacagtggagtacccagatatatcaagcaaacattattagagctaaacagagagatagacccaatacagtaaaagctggagactttaacaccccaccctcagcattggacagatcttccagacagaaaattaacaatgaaatcttagactaaatgtacatgacagaccaaatggacataatggatattgacagaacacttcatccaatggctgtagagtgcacagtcttttcctaaacatatggatcagtctcaaggatagaccatatagtagaccagaaaattagtcattttttaattgagaagatatcaagtaccttctctgatcataatggaagaaaactaaattaataagaggaatcttggaaaactatgcaaacacatggaaattaaacaatatgctccctaattaaacaatgtgctcccaaattgaaccacatgctccaaaatgggttaataaagacataaaaaattttaaaatttattgaaagaaatgataaagaagacaaagcatatgaaaacctatgaaatacagtgaaagcagtattaagaggaaagtttatagctcacaggctgcagccctggagcaggtgcaagggaggctggggaggggttcctcccagggtctgatgtcttccttttctcggacaaacatgctttaataagttaaacaagactttagtaaagactattgatgtgtctttgtgtctttcagtatacagttctatttgtaggatttatctaacctaacaagtcaatgagaatcacatgtaaaaggagaaatttctaggattttcagatatcttaataggtaggagatggagaaaagggatggttttattaattcagtgcttgccaatcttaacagagacagtagtaagacatgcagaaagcaaagcccagaaaagtatgaaggtgtcaaagtgccatttaagtatgggttcacttggaggaccatgttctgcgggaacttgttttcagcagacaatctattttagcagagttctgggcatacaaggggacacacatcattaaacaaggattgggacagggacttcagcgtcccactgttgcatggcccataaattatgtgtgttctctttctcatcttggatcaagtctagagctatgaaatagtatccctcatgaatatgcaaataacctgagatttactgaagtaaatacagatctgtcctgtgccctgagagcatcacccagcaaccacatctgtcctctagagaatcccctgagagctccgttcctcaccatggactggacctggaggatcctcttcttggtggcagcagccacaggtaagaggctccctagtcccagtgatgagaaagagattgagtccagtccagggagatctcatccacttctgtgttctctccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccagtaccagggacacgtccatcagcacagcctacatggagctgagcaggctgagatctgacgacacggtcgtgtattactgtgcgagagacacagtgtgaaaacccacatcctgagggtgtcagaaaccccagggaggaggcagctgtgctggggctgagaaatgaaagggattactatttttaatgttgtttacagtatgtcattaataaattgaaaaaaagtaacaatagaagtatatactctaattatatgggaactttgttttttcagttttttcatttttttttttttttttggtttgtttgtgacagagtctcactctgccacccaggctggagtgtaacggcacaatctcagctcactacaacctccacctcccaggttcaagcaattctcctgcctcggcctccagagtagttgggattacaggcacccgccaccatgcccggtgaatttttgtatttttagtagagacggggtttcaccatgttagctaggctggtctcaaactgctgatctcaggtgatctaccctcctcagcctcccaaagtcctgggattacaggcgtgagccactgcgcctggcccaattatatgggaattgtttatataattatcaccctataagcaaaattcatggaggaggaaaagctctactgaagaaagctgataccggcattcccatgaaagtatctgtgtagaagtaagtattaaaatcagttgaataggcaaggcatggtggctcacgcctataatcccagcactttgggagaccgaggcaggtggatccccttggggtttgggatgagctgcccttgcccactgtgctctgtggacctccctttagaagctcacagctccctgcactcggctccatcctgccccaccacacagaagcaaaacccctctcctttccactgcaggcttttcctggaccagaatgctgacctgctgcccttcactcccgaagtggtgggactgcctggggtggtgtgggtgttgagccttcttactctagggacctggcacctggccccaggggcacagggatggtgcatctgcctagggatgcctcctcatgccagggggtgggggttagtaccatcggccctcaggatttgttgcatgaatgagtgaatgggtgaataaatgaaggggatctgatctatgaataagggtatatagactttggttgatgtaggacgccaaatgctggaatttcagagtcatcacacccaggggccctgcctctgagctcctctttgcatccaatctgctgaagaacatggctctagggaaacccagttgtagacctgagggccccggctcttcaatgagccatctccgtcccggggccttatatcagcaagtgacgcacacatgcaaatgccagggtgtggtttcctgtttaaatgtagcctcccccgctgcagaactgcagagcctgctgaattctggctgaccagggcagtcaccagagctccagacaatgtctgtctccttcctcatcttcctgcccgtgctgggcctcccatggggtcagtgtcagggagatgccgtattcacagcagcattcacagactgaggggtgtttcactttgctgtttccttttgtctccaggtgtcctgtcacaggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagagacacagtgaggggaagtcagtgtgagcccagacacaaacctccctgcagggatgctcaggaccccagaaggcacccagcactaccagcgcagggcccagaccaggagcaggtgtggagttaagcaaaaatggaacttcttgctgtgtcttaaactgttgttgttttttttttttttttggctcagcaacagagatcatagaaaaccctttttcatatttttgaaatctgttcttagtctaatggagattctctaatatgtgacaatgtttttctcttgctgtttttggaattctttgtctttgacttttgacaacttgacttttgacagtgtgcctcaaagaagttctattttgggttctgtgaacctcctggatctgggaagttttcagctatgatttcattaaacgtgttttctacaccatttccctactcttttggaatacccataatgcaaatatttgttcacttaattgtgtcccataaatgctggggattttcttcattcctttttactctttttttctttttattcatctgcctgaattatttcaaaagatctgtcttcaacttcagaaactcttttgcttggcctagtctaatcttgaaggtctcaattgtacttttaatttcattcattgaattcttcaactctggaatttctgttggttcttttttatgatacttatctctttgttgaattcgaacacaaaatagtgacagcagagatgacggcaagttggcatttttcttttctagcaataaaacttaaagctgactcaaggagaaatggaaatcataattggaacagtaatcctcaagaaagcattaagattattaaataattgccctcacagatgacttcaggccaagatggctttatgggtgaagtttagactttcacaaaactaatcagttcccataagaactgctccaggatttggaggaacatgggaaagtctattaaagggatcacaattcacagtccccagagtaaaacatgggctaacttgcattttggcaaagagccaaatgttataaatgacatcctagaaggccaaattctgtccatctcgttgaacaaggacttacaccaggaatttagaactatttatagctcatcccaccactcaggccaatgatgacccatgatcatctcaccagaaatggaaagactcagatgattaatagagtctcaatttctctgagacatctaagagcccagcccaagcccagacccaggagggcacccaggcctggacagagaacactgatatcacaccagccctccagagggaagcagagactccttcaagctctggaaacacaggcccagacagctgcctaaagttgggcaggcttcactgcaaacccaaatcatgaagctaggtaacacctttacagattctttacatttaaaaatcatcaaaacaagagtaaataataaactcaaataatattaatctaatatgtaaaggtcttgtaccattattatgcaaacaacatacataagctaataagaaaaagaacaaatcccttaagaaatcggcaaaaaggatataacacaatttctaaaagaaaacaaatggctagcacacataaggaaaacactttgtgaacagacattcttcagaacattatttataattataaaatagttgaaagcaagatagtgcctgaagaaattatggtgcatacattagtgggactattctgcaaacattcccaattatacttgtcacatatctgtgataacgtgacagccagcattcatggggtgacctcatttggtaaaagggtgcaaagctcaacacgcattgtgagatgactgtggtgtaaaattagtgggattattccgcaaacattcccaattatacttaccgcatatctgtgataacatgacagcattcatggggtgacctcatttggtaaaagggtgcaaagctcaacacgcattgtgagatgactggtgtaaatacaaagaccaaactgtgaaaaggagtccatcaattaatcgatgcttaccttcagttttgggctaatttttaaagtatgctataagcatatgctcctgttataacagaatggagggattatgagagatgatgcaggtgtgtcctgggcctcccctggcccactgggccctagagatgccttcccaggcatcgctgtcagggcttccctcagagggagtcctgtattgacctcaccaccaaggtctggagcaggggatccttagatattggttggggttatctcaccttaggtctgaatttggggttgtcttagactgttttgtgctgttagaatagaatacccaagactgggaaatttatactgaacggaaatttatttctcacagttctagaggctgtgaagtccaagagcacaggtgccagagcaagtccaagagcaagggaaagtccaaagcaagtccaggagcatctggcgaggaccttcttgctgtgtcatcacatggcggaaggcaagaaagagagcaagagggggccgaactcacccttttataacagcaccaatcccacccatgaggtggggaccttatgacctaatcactcttcatactgttacaatggcaatgaaatttcaacatgagttttggaggagagaagcattcaaaccacagcaagggtgctcctacctcctctctcagggcatctgcagaaagagctgcaactgcacgtccttcctccgtccatcctccatcccttcccaatgtccgtgcatatcctgtgacccaggaggtctggcatagggggtgctcctgccttaggtctgaggccctgtctgaagaggggtaggtgaggaggccatctgatggtctgggccaagacagtcacaggacgcatcatttatcatcaaggaggctgagggttgagtctccaggtccagggaactccccacaaagtgggaaccctgcccagctccacacagcctctgctgggggaccctgctctggtgcagagcctggggacaggtcttgagctcagccagagtctgcctccctgtcatttaggaactaaaccaagcggcaggatgctggagcccagcccccatctgaccttacagggccaaggctggggccctgggttcccctcaaggcgcagcaggactggagccccaggcagtgcaggagtggccaaagctggggcttcctccagagcccccaagcatcacggcaccaagaagggtaggaccctggcctgaggaattggcaccaaagccccagaaactaccctggacaccatggagagaggcctggaggggaagcaccaggcactgcctccccttctgatcccacctgaggtggctgccaagcccagagagccgctctgatgtcccccagccctgcagcccagggatacctgtactgtgcccctgggggacccctggccagtctgtgcaaagaagtcaccaccctacactcagagacagtgggggtcctcgtcccacatcctcagagcatggcccggctgctgcagggatggtctcctggtcctcagagcatggcccggctgctgcagggatggtctcctggtcctcagagcatggcccagctgctgcagggatggtctcctggaggccccccagtgctctattgtcagggctccctccaccccccgcaccaagagagagccagaccccagcaaggcttccagtggcttcaggtcacacccctaggctgaccccagccccattaacacctgcctgagaaagctccacgcaccagaactgaccgtctgctccaactcttgacctcccgttctcagggcgtctgctgaaaaggctgcaactgcacatccttcctccgtccgttcccgatgtccgtgtgtctcctgtggccaggaaggtctttctcgggacctgagagccgctccctgaagtgtccccattgggaaggatggggcctgtgtctccaggctctgggaggacagaatcctgacctcaacagtggccggcacggacacaactggccccatcccggggacgctgaccagcgctgggcaacttttcccttccccgacgactgagccccgagcaccctccctgctcccctaccacctccctttacaaggctgtggcctctgcacagatgataatggagcttggctcattcccctagagtcggtagggagttaaggacaaaactcagtttcctccacctgaactcaagtctgcctatgtttacctaatcacacctggtggacagtttggacaaacttgcacactcagagacacagacacttctagaaatcattatctccctgccccggggaccccactccagcagaagtctgctaggcactggcctgggccctcctgctgtcctaggaggctgctgacctcctgcctggctcctgtccccaggtccagagtcagagcagactccagggacgctgcaggctaggaagccgccccctccaggcgagggtctagtgcaggtgcccaggacaagaaagattgtgaatgcaggaatgactgggccacacccctcccgtgcacgccccctcctgccctgcaccccacagcccagccccccgtgctggatgcccccccacagcagaggtgctgttctgtgatcccctgggaaagacgccctcaacctccaccctgtcccacggcccaaggaagacaagacacaggccctctcctcacagtctccccacctggctcctgctgggaccctcaaggtgtgaacagggaggatggttgtctgggtggcccctaggagcccagatcttcactccacagaccccaacccaagcacccccttctgcagggcccagctcatccccctcctcctccctctgctctcctctcgtcgcctctacgggaaatccgggactcagcagtaaccctcaggaagcagggcccaggcgccgtttaataggaggcttcctcacaatgaaacttttagaaagccttgactacaatgatgaccttggtgtggctgtgaacactgtcagctcccacagctgctgcagcaaaaaatgtccatagacagggtgggggcccggggtcgtctgctgtcctgctcagcccacagcacgcatggaggatctgaggtgccacacctgacgcccaggccagaacatgcctccctccagggtgacctgccatgtcctgcattgctggagggacaggggcagcctatgaggatctggggccaggagatgaatcctattaacccagaggaaaactaacaggacccaagcaccctccccgttgaagctgacctgcccagaggggcctgggcccaccccacacaccggggcggaatgtgtacaggccccggtctctgtgggtgttccgctaactggggctcccagtgctcaccccacaactaaagcgagccccagcctccagagcccccgaaggagatgccgcccacaagcccagcccccatccaggaggccccagagctcagggcgccggggcagattctgaacagccccgagtcacggtgggtacaactggaacgacaggatattgtactggtggtgtatgctatacccaccgtgagaaaaactgtgtccaaaactctctcctggcccctgctggaggccgcgccagagaggggagcagccgccccgaacctaggtcctgctcagctcacacgacccccagcagccagagcacagtggagtccccactgaaccccactgaatggtgaggacggggaccagggctccagggggtcatggaaggggctggaccccatcctactgctatggtcccagtgctcctggccagaactgaccctaccaccgacaagagtccctcagggaaacgggggtcactggcacctcccagcatcaaccccaggcagcacaggcataaaccccacatccagagccgactccaggagcagagacaccccagtaccctgggggacaccgaccctgatgactccccactggaatccaccccagagtccaccaggaccaaagaccccgccccggtctctgtccctcactcaggacctgctgcggggcgggccatgagaccagactcgggcttagggaacaccactgtggccccaacctcgaccaggccacaggcccttccttcctgccctgcggcagcacagactttggggtctgtgcagagaggaatcacagaggccccaggctgaggtggtgggggtggaagacccccaggaggtggcccacttcccttcctcccagctggaacccaccatgaccttcttaagataggggtgtcatccgaggcaggtcctccatggagctcccttcaggctcctccctggtcctcactaggcctcagtcccggctgtgggaatgcagccaccacaggcacaccaggcagcccagacccagccagcctgcagtgcccaagcccacattctggagcagagcaggctgtgtctgggagagtctgggctccccaccgccccccgcacaccccacccacccctgtccaggccctatgcaggagggtcagagccccccatggggtatggacttagggtctcactcacgcggctcccctcctgggtgaaggggtctcatgcccagatccccacagcagagctggtcaaaggtggaggcagtggccccagggccaccctgacctggaccctcaggctcctctagccctggctgccctgctgtccctgggaggcctggactccaccagaccacaggtccagggcaccgcccataggtgctgcccacactcagttcacaggaagaagataagctccagacccccaagactgggacctgccttcctgccaccgcttgtagctccagacctccgtgcctcccccgaccacttacacacgggccagggagctgttccacaaagatcaaccccaaaccgggaccgcctggcactcgggccgctgccacttccctctccatttgctcccagcacctctgtgctccctccctcctccctccttcaggggaacagcctgtgcagcccctccctgcaccccacaccctggggaggcccaaccctgcctccagccctttctcccccgctgctcttcctgcccatccagacaaccctggggtcccatccctgcagcctacaccctggtctccacccagacccctgtctctccctccagacacccctcccaggccaaccctgcacatgcaggccctccccttttctgctgccagagcctcagtttctaccctctgtgcctaccccctgcctcctcctgcccacaactcgagctcttcctctcctggggcccctgagccatggcactgaccgtgcactcccacccccacactgcccatgccctcaccttcctcctggacactctgaccctgctcccctcttggacccagccctggtatttccaggacaaaggctcacccaagtcttccccatgcaggcccttgccctcactgcccggttacacggcagcctcctgtgcacagaagcagggagctcagcccttccacaggcagaaggcactgaaagaaatcggcctccagcaccctgatgcacgtccgcctgtgtctctcactgcccgcacctgcagggaggctcggcactccctgtaaagacgagggatccaggcagcaacatcatgggagaatgcagggctcccagacagcccagccctctcgcaggcctctcctgggaagagacctgcagccaccactgaacagccacggagcccgctggatagtaactgagtcagtgaccgacctggagggcaggggagcagtgaaccggagcccagaccatagggacagagaccagccgctgacatcccgagcccctcactggcggccccagaacaccgcgtggaaacagaacagacccacattcccacctggaacagggcagacactgctgagcccccagcaccagccctgagaaacaccaggcaacggcatcagagggggctcctgagaaagaaaggaggggaggtctccttcaccagcaagtacttcccttgaccaaaaacagggtccacgcaactcccccaggacaaaggaggagccccctgtacagcactgggctcagagtcctctccaacacaccctgagtttcagacaaaaaccccctggaaatcatagtatcagcaggagaactagccagagacagcaagaggggactcagtgactcccgcggggacaggaggattttgtgggggctcgtgtcactgtgaggatattgtagtagtaccagctgctatgccgtattacgatttttggagtggttattatacccacagtgacacagccccattcccaaagccctgctgtaaacgcttccacttctggagctgaggggctggggggagcgtctgggaagtagggcctaggggtggccatcaatgcccaaaacgcaccagactcccccccagacatcaccccactggccagtgagcagagtaaacagaaaatgagaagcagctgggaagcttgcacaggccccaaggaaagagctttggcgggtgtgcaagaggggatgcgggcagagcctgagcagggccttttgctgtttctgctttcctgtgcagatagttccataaactggtgttcaagatcgatggctgggagtgagcccaggaggacagtgtgggaagggcacagggaaggagaagcagccgctatcctacactgtcatctttcaagagtttgccctgtgcccacaatgctgcatcatgggatgcttaacagctgatgtagacacagctaaagagagaatcagtgaaatggatttgcagcacagatctgaataaattctccagaatgtggagccacacagaagcaagcacaaggaaagtgcctgatgcaagggcaaagtacagtgtgtaccttcaggctgggcacagacactctgaaaagccttggcaggaactccctgcaacaaagcagagccctgcaggcaatgccagctccagagccctccctgagagcctcatgggcaaagatgtgcagaacatatgtttgtcatagccccaaactgagaatgaagcaaacagccatctgaaggaaaacaggcaaataaacgatggcaggttcatgaaatgcaaacccagacagccagaaggacaacagtgagggttacaggtgactctgtggttgagttcatgacaatgctgagtaattggagtaacaaaggaaagtccaaaaaatactttcaatgtgatttcttctaaataaaatttacagccggcaaaatgaactatcttcttaagggataaactttccactaggaaaactataaggaaaatcaagaaaaggatgatcacataaacacagtggtcgttacttctactggggaaggaagagggtatgaactgagacacacagggttggcaagtctcctaacaagaagagaacaaatacattacagtaccttgaaaacagcagttaaaattctaaattgcaagaagaggaaaatgcacacagctgtgtttagaaaattctcagtccagcactgttcataatagcaaagacattaacccaggttggataaataaacgatgacacaggcaattgcacaatgatacagacatacattcagtatatgagacattgatgatgtatccccaaagaaatgactttaaagagaaaaggcctgatatgtggtggcactcacctccctgggcatccccggacaggctgcaggcacactgtgtggcagggcaggctggtacctgctggcagctcctggggcctgatgtggagcaggcacagagccgtatccccccgaggacatatacccccaaggacggcacagttggtacattccggagacaagcaactcagccacactcccaggccagagcccgagagggacgcccatgcacagggaggcagagcccagctcctccacagccagcagcacctgtgcaggggccgccatctggcaggcacagagcatgggctgggaggaggggcagggacaccaggcagggttggcaccaactgaaaattacagaagtctcatacatctacctcagccttgcctgacctgggcctcacctgacctggacctcacctggcctggacctcacctggcctagacctcacctctgggcttcacctgagctcggcctcacctgacttggaccttgcctgtcctgagctcacatgatctgggcctcacctgacctgggtttcacctgacctgggcttcacctgacctgggcctcatctgacctgggcctcactggcctggacctcacctggcctgggcttcacctggcctcaggcctcatctgcacctgctccaggtcttgctggaacctcagtagcactgaggctgcaggggctcatccagggttgcagaatgactctagaacctcccacatctcagctttctgggtggaggcacctggtggcccagggaatataaaaagcctgaatgatgcctgcgtgatttgggggcaatttataaacccaaaaggacatggccatgcagcgggtagggacaatacagacagatatcagcctgaaatggagcctcagggcacaggtgggcacggacactgtccacctaagccaggggcagacccgagtgtccccgcagtagacctgagagcgctgggcccacagcctcccctcggtgccctgctacctcctcaggtcagccctggacatcccgggtttccccaggcctggcggtaggtttggggtgaggtctgtgtcactgtggtattacgatttttggagtggttattatacccacagtgtcacagagtccatcaaaaacccatccctgggaaccttctgccacagccctccctgtggggcaccgctgcgtgccatgttaggattttgactgaggacacagcaccatgggtatggtggctaccgcagcagtgcagcctgtgacccaaacacacagggcagcaggcacaacagacaagcccacaagtgaccaccctgagctcctgcctgccagccctggagaccatgaaacagatggccaggattatcccataggtcagccagacctcagtccaacaggtctgcatcgctgctgccctccaataccagtccggatggggacagggccggcccacattaccatttgctgccatccggccaacagtcccagaagcccctccctcaaggctgggccacatgtgtggaccctgagagccccccatgtctgagtaggggcaccaggaaggtggggctggccctgtgcactgtcactgcccctgtggtccctggcctgcctggccctgacacctgggcctctcctgggtcatttccaagacagaagacattcccaggacagctggagctgggagtccatcatcctgcctggccatcctgagtcctgcgcctttccaaacctcacccgggaagccaacagaggaatcacctcccacaggcagagacaaagaccttccagaaatctctgtctctctccccagtgggcaccctcttccagggcagtcctcagtgatatcacagtgggaacccacatctggatcgggactgcccccagaacacaagatggcccacagggacagccccacagcccagcccttcccagacccctaaaaggcgtcccaccccctgcatctgccccagggctcaaactccaggaggactgactcctgcacaccctcctgccagacatcacctcagcccctcctggaagggacaggagcgcgcaagggtgagtcagaccctcctgccctcgatggcaggcggagaagattcagaaaggtctgagatccccaggacgcagcaccactgtcaatgggggccccagacgcctggaccagggcctgcgtgggaaaggcctctgggcacactcaggggctttttgtgaagggtcctcctactgtggtggatatagtggctacgattactgactacggtgactaccacagtgatgaacccagcagcaaaaactgaccggactcccaaggtttatgcacacttctccgctcagagctctccaggatcagaagagccgggcccaagggtttctgcccagaccctcggcctctagggacatcttggccatgacagcccatgggctggtgccccacacatcgtctgccttcaaacaagggcttcagagggctctgaggtgacctcactgatgaccacaggtgccctggccccttccccgccagctgcaccagaccccgtcctgacagatgccccgattccaacagccaattcctggggccaggaatcgctgtagacaccagcctccttccaacacctcttgccaattgcctggattcccatcccggttggaatcaagaggacagcatcccccaggctcccaacaggcaggactcccacaccctcctctgagaggccgctgtgttccgtagggccaggctgcagacagtccccctcacctgccactagacaaatgcctgctgtagatgtccccacctggaaaagaccactcatggagcccccagccccaggtacagccatagagagagtctctgaggcccctaagaagtagccatgcccagttctgccgggaccctcggccaggctgacaggagtggacgctggagctgggcccacactgggccacataggagctcaccagtgagggcaggagagcacatgccggggagcacccagcctcctgctgaccagaggcccgtcccagagcccaggaggctgcagaggcctctccagggggacactgtgcatgtctggtccctgagcagccccccatgtccccagtcctgggggcccctggcacagctgtctggaccctctctattccctgggaagctcctcctgacagccccgcctccagttccaggtgtggttattgtcagggggtgtcagactgtggtggatacagctatggttacgtattactatgatagtagtggttattactaccacagtggtgctgcccatagcagcaaccaggccaagtagacaggcccctgctgtgcagccccaggcctccagctcacctgcttctcctggggctctcaaggctgctgttttctgcactctcccctctgtggggagggttccctcagtgggagatctgttctcaacatcccagggcctcattcctgcaaggaaggccaatggatgggcaacctcacatgccgcggctaagatagggtgggcagcctggcggggacaggacatcctgctggggtatctgtcactgtgcctagtggggcactggctcccaaacaacgcagtcctcgccaaaatccccacggcctcccccgctaggggctggcctgatctcctgcagtcctaggaggctgctgacctccagaatggctccgtccccagttccagggcgagagcagatcccaggccggctgcagactgggaggccaccccctccttcccagggttcactgcaggtgaccagggcaggaaatggcctgaacacagggataaccgggccatcccccaacagagtccaccccctcctgctctgtaccccgcacccccaaggccagcccatgacatccgacaaccccacaccagagtcactgcccggtgctgccctagggaggacccctcagcccccaccctgtctagaggactggggaggacaggacacgccctctccttatggttcccccacctggctctggctgggacccttggggtgtggacagaaaggacgcttgcctgattggcccccaggagcccagaacttctctccagggaccccagcccgagcacccccttacccaggacccagccctgcccctcctcccatctgctctcctctcatcaccccatgggaatccagaatccccaggaagccatcaggaagggctgagggaggaagtggggccactgcaccaccaggcaggaggctccgtctttgtgaacccagggaggtgccagcctcctagagggtatggtccaccctgcctatggctcccacagtggcaggctgcagggaaggaccagggacggtgtgggggagggctcagggccccgcgggtgctccatcttggatgagcccatctctctcacccacggactcacccacctcctctccaccctggccacacgtcgtccacaccatcctaagtcccacctacaccagagccggcacagccagtgcagacagaggctggggtgcaggggggccgccagggcagctttggggagggaaggatggaggaaggggagttcagtgaagaggcccccctcccctgggtccaggatcctcctctgggacccccggatcccatcccctccaggctctgggaggagaagcaggatgggagaatctgtgcgggaccctctcacagtggaatacctccacagcggctcaggcaagacccaaaagcccctcagtgagccctccactgcagtcctgggcctgggtagcagcccctcccacagaggatgaacccagcaccccgaggatgtcctgccagggggagctcagagccatgaaggagcaggatatgggacccccgatacaggcacagacctcagctccattcaggactgccacgtcctgccctgggaggaacccctttctctagtccctgcaggccaggaggcagctgactcctgacttggacgcctattccagacaccagacagaggggcaggccccccagaaccagggatgaggacgccccgtcaaggccagaaaagaccaagttgtgctgagcccagcaagggaaggtccccaaacaaaccaggaagtttctgaaggtgtctgtgtcacagtggagtatagcagctcgtccgtattactatggttcggggagttattataaccacagtgacactcgccaggccagaaaccccatcccaagtcagcggaatgcagagagagcagggaggacatgtttaggatctgaggccgcacctgacacccaggccagcagacgtctcctgtccatggcaccctgccatgtcctgcatttctggaagaacaagggcaggctgaagggggtccaggaccaggagatgggtcccctctacccagagaaggagccaggcaggacacaagccccctccccattgaggctgacctgcccagagggtcctgggcccaccccacacaccggggcggaatgtgtgcaggcctcggtctctgtgggtgttccgctagctggggctcacagtgctcaccccacacctaaaacgagccacagcctcagagcccctgaaggagaccccgcccacaagcccagcccccacccaggaggccccagagcacagggcgccccgtcggattctgaacagccccgagtcacagtggagtatagcagctcgtccgggtatagcagtggctggtaccactgtgagaaaagcttcgtccaaaacggtctcctggccacagtcggaggccccgccagagaggggagcagccaccccaaacccatgttctgccggctcccatgaccccgtgcacctggagccccacagtgtccccactggatgggaggacaagggccgggggctccggcgggtcggggcaggggcttgatggcttccttctgccgtggctccagtgcccctggctggagttgacccttctgacaagtgtcctcagagagtcagggatcagtggcacctcccaacatcaaccccacgcagcccaggcacaaaccccacatccagggccaactccaggaacagagacaccccaataccctgggggaccccaaccctgatgactcccgtcccatctctgtccctcacttggggcctgctgcggggcgagcacttgggagcaaactcaggcttaggggacaccactgtgggcctgacctcgagcaggccacagacccttccctcctgccctggtgcagcacagactttggggtctgggcagggaggaacttctggcaggtcaccaagcacagagcccccaggctgaggtggccccagggggaaccccagcaggtggcccactacccttcctcccagctggaccccatgtcttccccaagataggggtgccatccaaggcaggtcctccatggagcccccttcaggctcctctccagaccccactgggcctcagtccccactctaggaatgcagccaccacgggcacaccaggcagcccaggcccagccaccctgcagtgcccaagcccacaccctggaggagagcagggtgcgtctgggaggggctgggctccccacccccacccccacctgcacaccccacccacccttgcccgggccccctgcaggagggtcagagcccccatgggatatggacttagggtctcactcacgcacctcccctcctgggagaaggggtctcatgcccagatccccccagcagcgctggtcacaggtagaggcagtggccccagggccaccctgacctggcccctcaggctcctctagccctggctgccctgctgtccctgggaggcctgggctccaccagaccacaggtctagggcaccgcccacactggggccgcccacacacagctcacaggaagaagataagctccagacccccaggcccgggacctgccttgctgctacgacttcctgccccagacctcgttgccctcccccgtccacttacacacaggccaggaagctgttcccacacagaccaaccccagacggggaccacctggcactcaggtcactgccatttccttctccattcacttccaatgcctctgtgcttcctccctcctccttccttcgggggagcaccctgtgcagctcctccctgcagtccacaccctggggagacccgaccctgcagcccacaccctggggagacctgaccctcctccagccctttctcccccgctgctcttgccacccaccaagacagccctggggtcctgtccctacagcccccacccagttctctacctagacccgtcttcctccctctaaacacctctcccaggccaaccctacacctgcaggccctcccctccactgccaaagaccctcagtttctcctgcctgtgcccacccccgtgctcctcctgcccacagctcgagctcttcctctcctagggcccctgagggatggcattgaccgtgccctcgcacccacacactgcccatgccctcacattcctcctggccactccagccccactcccctctcaggcctggctctggtatttctgggacaaagccttacccaagtctttcccatgcaggcctgggcccttaccctcactgcccggttacagggcagcctcctgtgcacagaagcagggagctcagcccttccacaggcagaaggcactgaaagaaatcggcctccagcgccttgacacacgtctgcctgtgtctctcactgcccgcacctgcagggaggctcggcactccctctaaagacgagggatccaggcagcagcatcacaggagaatgcagggctaccagacatcccagtcctctcacaggcctctcctgggaagagacctgaagacgcccagtcaacggagtctaacaccaaacctccctggaggccgatgggtagtaacggagtcattgccagacctggaggcaggggagcagtgagcccgagcccacaccatagggccagaggacagccactgacatcccaagccactcactggtggtcccacaacaccccatggaaagaggacagacccacagtcccacctggaccagggcagagactgctgagacccagcaccagaaccaaccaagaaacaccaggcaacagcatcagagggggctctggcagaacagaggaggggaggtctccttcaccagcaggcgcttcccttgaccgaagacaggatccatgcaactcccccaggacaaaggaggagccccttgttcagcactgggctcagagtcctctccaagacacccagagtttcagacaaaaaccccctggaatgcacagtctcagcaggagagccagccagagccagcaagatggggctcagtgacacccgcagggacaggaggattttgtgggggctcgtgtcactgtgaggatattgtactaatggtgtatgctatacccacagtgacacagccccattcccaaagccctactgcaaacgcattccacttctggggctgaggggctgggggagcgtctgggaaatagggctcaggggtgtccatcaatgcccaaaacgcaccagactcccctccatacatcacacccaccagccagcgagcagagtaaacagaaaatgagaagcaagctggggaagcttgcacaggccccaaggaaagagctttggcgggtgtgtaagaggggatgcgggcagagcctgagcagggccttttgctgtttctgctttcctgtgcagagagttccataaactggtgttcgagatcaatggctgggagtgagcccaggaggacagcgtgggaagagcacagggaaggaggagcagccgctatcctacactgtcatctttcgaaagtttgccttgtgcccacactgctgcatcatgggatgcttaacagctgatgtagacacagctaaagagagaatcagtgagatggatttgcagcacagatctgaataaattctccagaatgtggagcagcacagaagcaagcacacagaaagtgcctgatgcaaggacaaagttcagtgggcaccttcaggcattgctgctgggcacagacactctgaaaagccctggcaggaactccctgtgacaaagcagaaccctcaggcaatgccagccccagagccctccctgagagcctcatgggcaaagatgtgcacaacaggtgtttctcatagccccaaactgagagcaaagcaaacgtccatctgaaggagaacaggcaaataaacgatggcaggttcatgaaatgcaaacccagacagccacaagcacaaaagtacagggttataagcgactctggttgagttcatgacaatgctgagtaattggagtaacaaagtaaactccaaaaaatactttcaatgtgatttcttctaaataaaatttacaccctgcaaaatgaactgtcttcttaagggatacatttcccagttagaaaaccataaagaaaaccaagaaaaggatgatcacataaacacagtggtggttacttctgctggggaaggaagagggtatgaactgagatacacagggtgggcaagtctcctaacaagaacagaacgaatacattacagtaccttgaaaacagcagttaaacttctaaattgcaagaagaggaaaatggacacagttgtgtttagaaaattctcagtccagcactgttcataatagcaaagacattaacccaggtcggataaataagcgatgacacaggcaattgcacaatgatacagacatatatttagtatatgagacatcgatgatgtatccccaaataaacgactttaaagagataaagggctgatgtgtggtggcattcacctccctgggatccccggacaggttgcaggctcactgtgcagcagggcaggcgggtacctgctggcagttcctggggcctgatgtggagcaagcgcagggccatatatcccggaggacggcacagtcagtgaattccagagagaagcaactcagccacactccccaggcagagcccgagagggacgcccacgcacagggaggcagagcccagcacctccgcagccagcaccacctgtgcacgggccaccaccttgcaggcacagagtgggtgctgagaggaggggcagggacaccaggcagggtgagcacccagagaaaactgcagacgcctcacacatccacctcagcctcccctgacctggacctcactggcctgggcctcacttaacctgggcttcacctgaccttggcctcacctgacttggacctcgcctgtcccaagctttacctgacctgggcctcaactcacctgaacgtctcctgacctgggtttaacctgtcctggaactcacctggccttggcttcccctgacctggacctcatctggcctgggcttcacctggcctgggcctcacctgacctggacctcatctggcctggacctcacctggcctggacttcacctggcctgggcttcacctgacctggacctcacctggcctcgggcctcacctgcacctgctccaggtcttgctggagcctgagtagcactgagggtgcagaagctcatccagggttggggaatgactctagaagtctcccacatctgacctttctgggtggaggcagctggtggccctgggaatataaaaatctccagaatgatgactctgtgatttgtgggcaacttatgaacccgaaaggacatggccatggggtgggtagggacatagggacagatgccagcctgaggtggagcctcaggacacaggtgggcacggacactatccacataagcgagggatagacccgagtgtccccacagcagacctgagagcgctgggcccacagcctcccctcagagccctgctgcctcctccggtcagccctggacatcccaggtttccccaggcctgccggtaggtttagaatgaggtctgtgtcactgtggtggatatagtggctacgattacgtggatacagctatggttaccacagtgtcacagagtccatcaaaaacccatgcctggaagcttcccgccacagccctccccatggggccctgctgcctcctcaggtcagccccggacatcccgggtttccccaggctgggcggtaggtttggggtgaggtctgtgtcactgtggggtatagcagcagctggtacgggtatagcagtggctggtaccacagtgtcacagagtccatcaaaaacccatccctgggagcctcccgccacagccctccctgcaggggaccggtacgtgccatgttaggattttgatcgaggagacagcaccatgggtatggtggctaccacagcagtgcagcctgtgacccaaacccgcagggcagcaggcacgatggacaggcccgtgactgaccacgctgggctccagcctgccagccctggagatcatgaaacagatggccaaggtcaccctacaggtcatccagatctggctccgaggggtctgcatcgctgctgccctcccaacgccagtccaaatgggacagggacggcctcacagcaccatctgctgccatcaggccagcgatcccagaagcccctccctcaaggctgggccacatgtgtggacactgagagccctcatgtctgagtaggggcaccaggagggaggggctggccctgtgcactgtccctgcccctgtggtccctggcctgcctggccctgacacctgagcctctcctgggtcatttccaagacagaagacattcctggggacagccggagctgggcgtcgctcatcctgcccggccgtcctgagtcctgctcatttccagacctcaccggggaagccaacagaggactcgcctcccacattcagagacaaagaaccttccagaaatccctgcctctctccccagtggacaccctcttccaggacagtcctcagtggcatcacagcggcctgagatccccaggacgcagcaccgctgtcaataggggccccaaatgcctggaccagggcctgcgtgggaaaggtctctggccacactcgggctttttgtgaagggccctcctgctgtgaggatattgtagtagtaccagctgctatgccgggtatagcagcagctggtaccatagtgatgaacccagtggcaaaaactggctggaaacccaggggctgtgtgcacgcctcagcttggagctctccaggagcacaagagccgggcccaaggatttgtgcccagaccctcagcctctagggacacctgggccatctcagcctgggctggtgccctgcacaccatcttcctccaaataggggcttcagagggctctgaggtgacctcactcatgaccacaggtgacctggcccttccctgccagctataccagaccctgtcttgacagatgccccgattccaacagccaattcctgggaccctgaatagctgtagacaccagcctcattccagtacctcctgccaattgcctggattcccatcctggctggaatcaagaaggcagcatccgccaggctcccaacaggcaggactcccgcacaccctcctctgagaggccgctgtgttccgcagggccaggccctggacagttcccctcacctgccactagagaaacacctgccattgtcgtccccacctggaaaagaccactcgtggagcccccagccccaggtacagctgtagagagagtcctcgaggcccctaagaaggagccatgcccagttctgccgggaccctcggccaggccgacaggagtggacgctggagctgggcccacactgggccacataggagctcaccagtgagggcaggagagcacatgccggggagcacccagcctcctgctgaccagaggcctgccccagagcccaggaggctgcagaggcctctccagggagacactgtgcatgtctggtacctaagcagccccccacgtccccagtcctgggggcccctggctcagctgtctggaccctccctgttccctgggaagctcctcctgacagccccgcctccagttccaggtgtggttattgtcaggcgatgtcagactgtggtggatatagtggctacgattacgggtatagcagtggctggtaccacagtggtgccgcccatagcagcaaccaggccaagtagacaggcccctgctgcgcagccccaggcatccacttcacctgcttctcctggggctctcaaggctgctgtctgtcctctggccctctgtggggagggttccctcagtgggaggtctgtgctccagggcagggatgattgagatagaaatcaaaggctggcagggaaaggcagcttcccgccctgagaggtgcaggcagcaccacggagccacggagtcacagagccacggagcccccattgtgggcatttgagagtgctgtgcccccggcaggcccagccctgatggggaagcctgtcccatcccacagcccgggtcccacgggcagcgggcacagaagctgccaggttgtcctctatgatcctcatccctccagcagcatcccctccacagtggggaaactgaggcttggagcaccacccggccccctggaaatgaggctgtgagcccagacagtgggcccagagcactgtgagtaccccggcagtacctggctgcagggatcagccagagatgccaaaccctgagtgaccagcctacaggaggatccggccccacccaggccactcgattaatgctcaaccccctgccctggagacctcttccagtaccaccagcagctcagcttctcagggcctcatccctgcaaggaaggtcaagggctgggcctgccagaaacacagcaccctccctagccctggctaagacagggtgggcagacggctgtggacgggacatattgctggggcatttctcactgtcacttctgggtggtagctctgacaaaaacgcagaccctgccaaaatccccactgcctcccgctaggggctggcctggaatcctgctgtcctaggaggctgctgacctccaggatggctccgtccccagttccagggcgagagcagatcccaggcaggctgtaggctgggaggccacccctgcccttgccggggttgaatgcaggtgcccaaggcaggaaatggcatgagcacagggatgaccgggacatgccccaccagagtgcgccccttcctgctctgcaccctgcaccccccaggccagcccacgacgtccaacaactgggcctgggtggcagccccacccagacaggacagacccagcaccctgaggaggtcctgccagggggagctaagagccatgaaggagcaagatatggggcccccgatacaggcacagatgtcagctccatccaggaccacccagcccacaccctgagaggaacgtctgtctccagcctctgcaggtcgggaggcagctgacccctgacttggacccctattccagacaccagacagaggcgcaggccccccagaaccagggttgagggacgccccgtcaaagccagacaaaaccaaggggtgttgagcccagcaagggaaggcccccaaacagaccaggaggtttctgaaggtgtctgtgtcacagtggggtatagcagcagctggtacagcatattgtggtggtgattgctattcccacagtgacactcacccagccagaaaccccattccaagtcagcggaagcagagagagcagggaggacacgtttaggatctgagactgcacctgacacccaggccagcagacgtctcccctccagggcaccccaccctgtcctgcatttctgcaagatcaggggcggcctgagggggggtctagggtgaggagatgggtcccctgtacaccaaggaggagttaggcaggtcccgagcactctccccattgaggctgacctgcccagagagtcctgggcccaccccacacaccggggcggaatgtgtgcaggcctcggtctctgtgggtgttccgctagctggggctcacagtgctcaccccacacctaaaatgagccacagcctccggagcccccgcaggagaccccgcccacaagcccagcccccacccaggaggccccagagctcagggcgccccgtcggattccgaacagccccgagtcacagcggtggatacagctatggttacgtattactatggttcggggagttattataaccactgtcagaatagctacgtcaaaaactgtccagtggccactgccggaggccccgccagagagggcagcagccactctgatcccatgtcctgccggctcccatgacccccagcacgcggagccccacagtgtccccactggatgggaggacaagagctggggattccggcgggtcggggcaggggcttgatcgcatccttctgccgtggctccagtgcccctggctggagttgacccttctgacaagtgtcctcagagagacaggcatcaccggcgcctcccaacatcaaccccaggcagcacaggcacaaaccccacatccagagccaactccaggagcagagacaccccaataccctgggggaccccgaccctgatgacttcccactggaattcgccgtagagtccaccaggaccaaagaccctgcctctgcctctgtccctcactcaggacctgctgccgggcgaggccttgggagcagacttgggcttaggggacaccagtgtgaccccgaccttgaccaggacgcagacctttccttcctttcctggggcagcacagactttggggtctgggccaggaggaacttctggcaggtcgccaagcacagaggccacaggctgaggtggccctggaaagacctccaggaggtggccactccccttcctcccagctggaccccatgtcctccccaagataagggtgccatccaaggcaggtgctccttggagccccattcagactcctccctggaccccactgggcctcagtcccagctctggggatgaagccaccacaagcacaccaggcagcccaggcccagccaccctgcagtgcccaagcacacactctggagcagagcagggtgcctctgggaggggctgagctccccaccccacccccacctgcacaccccacccacccctgcccagcggctctgcaggagggtcagagccccacatggggtatggacttagggtctcactcacgtggctcccatcatgagtgaaggggcctcaagcccaggttcccacagcagcgcctgtcgcaagtggaggcagaggcccgagggccaccctgacctggtccctgaggttcctgcagcccaggctgccctgctgtccctgggaggcctgggctccaccagaccacaggtccagggcaccgggtgcaggagccacccacacacagctcacaggaagaagataagctccagacccccagggccagaacctgccttcctgctactgcttcctgccccagacctgggcgccctcccccgtccacttacacacaggccaggaagctgttcccacacagaacaaccccaaaccaggaccgcctggcactcaggtggctgccatttccttctccatttgctcccagcgcctctgtcctccctggttcctccttcgggggaacagcctgtgcagccagtccctgcagcccacaccctggggagacccaaccctgcctggggcccttccaaccctgctgctcttactgcccacccagaaaactctggggtcctgtccctgcagtccctaccctggtctccacccagacccctgtgtatcactccagacacccctcccaggccaaccctgcacctgcaggccctgtcctcttctgtcgctagagcctcagtttctcccccctgtgcccacaccctacctcctcctgcccacaactctaactcttcttctcctggagcccctgagccatggcattgaccctgccctcccaccacccacagcccatgccctcaccttcctcctggccactccgaccccgccccctctcaggccaagccctggtatttccaggacaaaggctcacccaagtctttcccaggcaggcctgggctcttgccctcacttcccggttacacgggagcctcctgtgcacagaagcagggagctcagcccttccacaggcagaaggcactgaaagaaatcggcctccagcaccttgacacacgtccccccgtgtctctcactgcccgcacctgcagggaggctccgcactccctctaaagacaagggatccaggcagcagcatcacgggagaatgcagggctcccagacatcccagtcctctcacaggcctctcctgggaagagacctgcagccaccaccaaacagccacagaggctgctggatagtaactgagtcaatgaccgacctggagggcaggggagcagtgagccggagcccataccatagggacagagaccagccgctgacatcccgagctcctcaatggtggccccataacacacctaggaaacataacacacccacagccccacctggaacagggcagagactgctgagcccccagcaccagccccaagaaacaccaggcaacagtatcagagggggctcccgagaaagagaggaggggagatctccttcaccatcaaatgcttcccttgaccaaaaacagggtccacgcaactcccccaggacaaaggaggagccccctatacagcactgggctcagagtcctctctgagacaccctgagtttcagacaacaacccgctggaatgcacagtctcagcaggagaacagaccaaagccagcaaaagggacctcggtgacaccagtagggacaggaggattttgtgggggctcgtgtcactgtgaggatattgtagtggtggtagctgctactccgtattactatgatagtagtggttattactaccacagtgacacagacccattcccaaagccctactgcaaacacacccactcctggggctgaggggctgggggagcgtctgggaagtagggtccaggggtgtctatcaatgtccaaaatgcaccagactgcccgccaaacaccaccccaccagccagcgagcagggtaaacagaaaatgagaggctctgggaagcttgcacaggccccaaggaaagagctttggcgggtgtgcaagaggggatgcaggcagagcctgagcagggccttttgctgtttctgctttcctgtgcagagagttccataaactggtgttcaagatcagtggctgggaatgagcccaggagggcagtctgtgggaagagcacagggaaggaggagcagccgctatcctacactgtcatctttcaaaagtttgccttgtgaccacactattgcatcatgggatgcttaagagctgatgtagacacagctaaagagagaatcagtgagatgaatttgcagcatagatctgaataaactctccagaatgtggagcagtacagaagcaaacacacagaaagtgcctgatgcaaggacaaagttcagtgggcaccttcaggcattgctgctgggcacagacactctgaaaagccctggcaggatctccctgcgacaaagcagaaccctcaggcaatgccagccccagagccctccctgagagcgtcatggggaaagatgtgcagaacagctgattatcatagactcaaactgagaacagagcaaacgtccatctgaagaacagtcaaataagcaatggtaggttcatgcaatgcaaacccagacagccaggggacaacagtagagggctacaggcggctttgcggttgagttcatgacaatgctgagtaattggagtaacagaggaaagcccaaaaaatacttttaatgtgatttcttctaaataaaatttacaccaggcaaaatgaactgtcttcttaagggataaactttcccctggaaaaactacaaggaaaattaagaaaacgatgatcacataaacacagttgtggttacttctactggggaaggaagagggtatgagctgagacacacagagtcggcaagtctccaagcaagcacagaacgaatacattacagtaccttgaatacagcagttaaacttctaaatcgcaagaagaggaaaatgcacacagctgtgtttagaaaattctcagtccagcactattcataatagcaaagacattaacccaggttggataaataaatgatgacacaggcaattgcacaatgatacagacatacatttagtacatgagacatcgatgatgtatccccaaagaaatgactttaaagagaaaaggcctgatgtgtggtggcactcacctccctgggatccccggacaggttgcaggcacactgtgtggcagggcaggctggtacatgctggcagctcctggggcctgatgtggagcaagcgcagggctgtatacccccaaggatggcacagtcagtgaattccagagagaagcagctcagccacactgcccaggcagagcccgagagggacgcccacgcacagggaggcagagcccagctcctccacagccaccaccacctgtgcacgggccaccaccttgcaggcacagagtgggtgctgagaggaggggcagggacaccaggcagggtgagcacccagagaaaactgcagaagcctcacacatccacctcagcctcccctgacctggacctcacctggtctggacctcacctggcctgggcctcacctgacctggacctcacctggcctgggcttcacctgacctggacctcacctggcctccggcctcacctgcacctgctccaggtcttgctggaacctgagtagcactgaggctgcagaagctcatccagggttggggaatgactctggaactctcccacatctgacctttctgggtggaggcatctggtggccctgggaatataaaaagccccagaatggtgcctgcgtgatttgggggcaatttatgaacccgaaaggacatggccatggggtgggtagggacatagggacagatgccagcctgaggtggagcctcaggacacagttggacgcggacactatccacataagcgagggacagacccgagtgttcctgcagtagacctgagagcgctgggcccacagcctcccctcggtgccctgctgcctcctcaggtcagccctggacatcccgggtttccccaggccagatggtaggtttgaagtgaggtctgtgtcactgtggtattatgattacgtttgggggagttatcgttatacccacagcatcacacggtccatcagaaacccatgccacagccctccccgcaggggaccgccgcgtgccatgttacgattttgatcgaggacacagcgccatgggtatggtggctaccacagcagtgcagcccatgacccaaacacacagggcagcaggcacaatggacaggcctgtgagtgaccatgctgggctccagcccgccagccccggagaccatgaaacagatggccaaggtcaccccacagttcagccagacatggctccgtggggtctgcatcgctgctgccctctaacaccagcccagatggggacaaggccaaccccacattaccatctcctgctgtccacccagtggtcccagaagcccctccctcatggctgagccacatgtgtgaaccctgagagcaccccatgtcagagtaggggcagcagaagggcggggctggccctgtgcactgtccctgcacccatggtccctcgcctgcctggccctgacacctgagcctcttctgagtcatttctaagatagaagacattcccggggacagccggagctgggcgtcgctcatcccgcccggccgtcctgagtcctgcttgtttccagacctcaccagggaagccaacagaggactcacctcacacagtcagagacaaagaaccttccagaaatccctgtctcactccccagtgggcaccttcttccaggacattcctcggtcgcatcacagcaggcacccacatctggatcaggacggcccccagaacacaagatggcccatggggacagccccacaacccaggccttcccagacccctaaaaggcgtcccaccccctgcacctgccccagggctaaaaatccaggaggcttgactcccgcataccctccagccagacatcacctcagccccctcctggaggggacaggagcccgggagggtgagtcagacccacctgccctcgatggcaggcggggaagattcagaaaggcctgagatccccaggacgcagcaccactgtcaatgggggccccagacgcctggaccagggcctgcgtgggaaaggccgctgggcacactcaggggctttttgtgaaggcccctcctactgtggtggatatagtggctacgattacaggatattgtagtggtggtagctgctactcccacagtgatgaaactagcagcaaaaactggccggacacccagggaccatgcacacttctcagcttggagctctccaggaccagaagagtcaggtctgagggtttgtagccagaccctcggcctctagggacaccctggccatcacagcggatgggctggtgccccacatgccatctgctccaaacaggggcttcagagggctctgaggtgacttcactcatgaccacaggtgccctggccccttccccgccagctacaccgaaccctgtcccaacagctgccccagttccaacagccaattcctggggcccagaattgctgtagacaccagcctcgttccagcacctcctgccaattgcctggattcacatcctggctggaatcaagagggcagcatccgccaggctcccaacaggcaggactcccgcacaccctcctctgagaggccgctgtgttccgcagggccaggccctggacagttcccctcacctgccactagagaaacacctgccattgtcgtccccacctggaaaagaccactcgtggagcccccagccccaggtacagctgtagagagactccccgagggatctaagaaggagccatgcgcagttctgccgggaccctcggccaggccgacaggagtggacactggagctgggcccacactgggccacataggagctcaccagtgagggcaggagagcacatgccggggagcacccagcctcctgctgaccagaggcccgtcccagagcccaggaggctgcagaggcctctccagggggacactgtgcatgtctggtccctgagcagccccccacgtccccagtcctgggggcccctggcacagctgtctggaccctccctcttccctgggaagctcctcctgacagccccgcctccagttccaggtgtggttattgtcagggggtgtcagactgtgaggatattgtagtggtggtagctgctactccgtagagatggctacaattaccacagtggtgctgcccatagcagcaaccaggccaagtagacaggcccctgctgtgcagccccaggcctccagctcacctgcttctcctggggctctcaaggtcactgttgtctgtactctgccctctgtggggagggttccctcagtgggaggtctgttctcaacatcccagggcctcatgtctgcacggaaggccaatggatgggcaacctcacatgccgcggctaagatagggtgggcagcctggcgggggacagtacatactgctggggtgtctgtcactgtgcctagtggggcactggctcccaaacaacgcagtcctcgccaaaatccccacagcctcccctgctaggggctggcctgatctcctgcagtcctaggaggctgctgacctccagaatgtctccgtccccagttccagggcgagagcagatcccaggccggctgcagactgggaggccaccccctccttcccagggttcactggaggtgaccaaggtaggaaatggccttaacacagggatgactgcgccatcccccaacagagtcagccccctcctgctctgtaccccgcaccccccaggccagtccacgaaaaccagggccccacatcagagtcactgcctggcccggccctggggcggacccctcagcccccaccctgtctagaggacttggggggacaggacacaggccctctccttatggttcccccacctgcctccggccgggacccttggggtgtggacagaaaggacacctgcctaattggcccccaggaacccagaacttctctccagggaccccagcccgagcacccccttacccaggacccagccctgcccctcctcccctctgctctcctctcatcaccccatgggaatccggtatccccaggaagccatcaggaagggctgaaggaggaagcggggccgtgcaccaccgggcaggaggctccgtcttcgtgaacccagggaagtgccagcctcctagagggtatggtccaccctgcctggggctcccaccgtggcaggctgcggggaaggaccagggacggtgtgggggagggctcagggccctgcgggtgctcctccatcttcggtgagcctcccccttcacccaccgtcccgcccacctcctctccaccctggctgcacgtcttccacaccatcctgagtcctacctacaccagagccagcaaagccagtgcagacaaaggctggggtgcaggggggctgccagggcagcttcggggagggaaggatggagggaggggaggtcagtgaagaggcccccttcccctgggtccaggatcctcctctgggacccccggatcccatcccctcctggctctgggaggagaagcaggatgggagaatctgtgcgggaccctctcacagtggaatatccccacagcggctcaggccagacccaaaagcccctcagtgagccctccactgcagtcctgggcctgggtagcagcccctcccacagaggacagacccagcaccccgaagaagtcctgccagggggagctcagagccatgaaagagcaggatatggggtccccgatacaggcacagacctcagctccatccaggcccaccgggacccaccatgggaggaacacctgtctccgggttgtgaggtagctggcctctgtctcggaccccactccagacaccagacagaggggcaggccccccaaaaccagggttgagggatgatccgtcaaggcagacaagaccaaggggcactgaccccagcaagggaaggctcccaaacagacgaggaggtttctgaagctgtctgtatcacagtggggtatagcagtggctggtacggtatagtgggagctactaccacagtgacactcgccaggccagaaaccccgtcccaagtcagcggaagcagagagagcagggaggacacgtttaggatctgaggccgcacctgacacccagggcagcagacgtctcccctccagggcaccctccaccgtcctgcgtttcttcaagaataggggcggcctgagggggtccagggccaggcgataggtcccctctaccccaaggaggagccaggcaggacccgagcaccgtccccattgaggctgacctgcccagacgggcctgggcccaccccacacaccggggcggaatgtgtgcaggccccagtctctgtgggtgttccgctagctggggcccccagtgctcaccccacacctaaagcgagccccagcctccagagccccctaagcattccccgcccagcagcccagcccctgcccccacccaggaggccccagagctcagggcgcctggtcggattctgaacagccccgagtcacagtgaggatattgtagtggtggtagctgctactccgtggatacagctatggttaccaccgtgagaaaaactgtgtccaaaactgactcctggcagcagtcggaggccccgccagagaggggagcagccggcctgaacccatgtcctgccggttcccatgacccccagcacccagagccccacggtgtccccgttggataatgaggacaagggctgggggctccggtggtttgcggcagggacttgatcacatccttctgctgtggccccattgcctctggctggagttgacccttctgacaagtgtcctcagaaagacagggatcaccggcacctcccaatatcaaccccaggcagcacagacacaaaccccacatccagagccaactccaggagcagagacaccccaacactctgggggaccccaaccgtgataactccccactggaatccgccccagagtctaccaggaccaaaggccctgccctgtctctgtccctcactcagggcctcctgcagggcgagcgcttgggagcagactcggtcttaggggacaccactgtgggccccaactttgatgaggccactgacccttccttcctttcctggggcagcacagactttggggtctgggcagggaagaactactggctggtggccaatcacagagcccccaggccgaggtggccccaagaaggccctcaggaggtggccactccacttcctcccagctggaccccaggtcctccccaagataggggtgccatccaaggcaggtcctccatggagcccccttcagactcctcccgggaccccactggacctcagtccctgctctgggaatgcagccaccacaagcacaccaggaagcccaggcccagccaccctgcagtgggcaagcccacactctggagcagagcagggtgcgtctgggaggggctaacctccccaccccccaccccccatctgcacacagccacctaccactgcccagaccctctgcaggagggccaagccaccatggggtatggacttagggtctcactcacgtgcctcccctcctgggagaaggggcctcatgcccagatccctgcagcactagacacagctggaggcagtggccccagggccaccctgacctggcatctaaggctgctccagcccagacagcactgccgttcctgggaagcctgggctccaccagaccacaggtccagggcacagcccacaggagccacccacacacagctcacaggaagaagataagctccagaccccagggcgggacctgccttcctgccaccacttacacacaggccagggagctgttcccacacagatcaaccccaaaccgggactgcctggcactagggtcactgccatttccctctccattccctcccagtgcctctgtgctccctccttctggggaacaccctgtgcagcccctccctgcagcccacacgctggggagaccccaccctgcctcgggccttttctacctgctgcacttgccgcccacccaaacaaccctgggtacgtgaccctgcagtcctcaccctgatctgcaaccagacccctgtccctccctctaaacacccctcccaggccaactctgcacctgcaggccctccgctcttctgccacaagagcctcaggttttcctacctgtgcccaccccctaacccctcctgcccacaacttgagttcttcctctcctggagcccttgagccatggcactgaccctacactcccacccacacactgcccatgccatcaccttcctcctggacactctgaccccgctcccctccctctcagacccggccctggtatttccaggacaaaggctcacccaagtcttccccatgcaggcccttgccctcactgcctggttacacgggagcctcctgtgcgcagaagcagggagctcagctcttccacaggcagaaggcactgaaagaaatcggcctccagtgccttgacacacgtccgcctgtgtctctcactgcctgcacctgcagggaggctccgcactccctctaaagatgagggatccaggcagcaacatcacgggagaatgcagggctcccagacagcccagccctctcgcaggcctctcctgggaagagacctgcagccaccactgaacagccacggaggtcgctggatagtaaccgagtcagtgaccgacctggagggcaggggagcagtgaaccggagcccataccatagggacagagaccagccgctaacatcccgagcccctcactggcggccccagaacaccccgtggaaagagaacagacccacagtcccacctggaacagggcagacactgctgagcccccagcaccagccccaagaaacactaggcaacagcatcagagggggctcctgagaaagagaggaggggaggtctccttcaccatcaaatgcttcccttgaccaaaaacagggtccacgcaactcccccaggacaaaggaggagccccctgtacagcactgggctcagagtcctctctgagacaggctcagtttcagacaacaacccgctggaatgcacagtctcagcaggagagccaggccagagccagcaagaggagactcggtgacaccagtctcctgtagggacaggaggattttgtgggggttcgtgtcactgtggtggatatagtggctacgattacgtattactatgatagtagtggttattactaccacagtgacacaaccccattcctaaagccctactgcaaacgcacccactcctgggactgaggggctgggggagcatctgggaagtatggcctaggggtgtccatcaatgcccaaaatgcaccagactctccccaagacatcaccccaccagccagtgagcagagtaaacagaaaatgagaagcagctgggaagcttgcacaggccccaaggaaagagctttggcaggtgtgcaagaggggatgtgggcagagcctcagcagggccttttgctgtttctgctttcctgtgcagagagttccataaactggtattcaagatcaatggctgggagtgagcccaggaggacagtgtgggaagagcacagggaaggaggagcagccgctatcctacactgtcatcttttgaaagtttgccctgtgcccacaatgctgcatcatgggatgcttaacagctgatgtagacacagctaaagagagaatcagtgaaatgcatttgcagcacagatctgaataaatcctccagaatgtggagcagcacagaagcaagcacacagaaagtgcctgatgccaaggcaaagttcagtgggcaccttcaggcattgctgctgggcacagacactctgaaaagcactggcaggaactgcctgtgacaaagcagaaccctcaggcaatgccagccctagagcccttcctgagaacctcatgggcaaagatgtgcagaacagctgtttgtcatagccccaaactatggggctggacaaagcaaacgtccatctgaaggagaacagacaaataaacgatggcaggttcatgaaatacaaactaggacagccagaggacaacagtagagagctacaggcggctttgcggttgagttcatgacaatgctgagtaattggagtaacagaggaaagcccaaaaaatacttttaatgtgatttcttctaaataaaatttacacccggcaaaatgaactatcttcttaagggataaactttcccctggaaaaactataaggaaaatcaagaaaacgatgatcacataaacacagtggtggttacttctactggggaaggaagagggtatgagctgagacacacagagtcggcaagtctcctaacaagaacagaacaaatacattacagtaccttgaaaacagcagttaaacttctaaatcgcaagaagaggaaaatgcacacacctgtgtttagaaaattctcagtccagcactgttcataatagcaaagacattaacccaggttggataaataagcgatgacacaggcaattgcacaatgatacagacatacattcagtatatgagacatcgatgatgtatccccaaagaaatgactttaaagagaaaaggcctgatgtgtggtggcaatcacctccctgggcatccccggacaggctgcaggctcactgtgtggcagggcaggcaggcacctgctggcagctcctggggcctgatgtggagcaggcacagagctgtatatccccaaggaaggtacagtcagtgcattccagagagaagcaactcagccacactccctggccagaacccaagatgcacacccatgcacagggaggcagagcccagcacctccgcagccaccaccacctgcgcacgggccaccaccttgcaggcacagagtgggtgctgagaggaggggcagggacaccaggcagggtgagcacccagagaaaactgcagaagcctcacacatccacctcagcctcccctgacctggacctcacctggcctgggcctcacctgacctggacctcacctggcctgggcttcacctggcctgggcttcacctgacctggacctcacctggcctcgggcctcacctggcctgggcttcacctggcctgggcttcacctgacctggacctcacctggcctgggcctcacctgacctggacctcacctggcctgggcttcacctggcctgggcttcacctggcctgggcttcacctgacctggacctcacctggcctgggcttcacctgacctggacctcacctggcctcgggcctcacctgcacctgctccaggtcttgctggagcctgagtagcactgaggctgtagggactcatccagggttggggaatgactctgcaactctcccacatctgacctttctgggtggaggcacctggtggcccagggaatataaaaagccccagaatgatgcctgtgtgatttgggggcaatttatgaacccgaaaggacatggccatggggtgggtagggacagtagggacagatgtcagcctgaggtgaagcctcaggacacaggtgggcatggacagtgtccacctaagcgagggacagacccgagtgtccctgcagtagacctgagagcgctgggcccacagcctcccctcggggccctgctgcctcctcaggtcagccctggacatcccgggtttccccaggcctggcggtaggtttgaagtgaggtctgtgtcactgtgaggatattgtagtggtggtagctgctactcctgactacggtgactaccacagtgtcacagagtccatcaaaaactcatgcctgggagcctcccaccacagccctccctgcgggggaccgctgcatgccgtgttaggattttgatcgaggacacggcgccatgggtatggtggctaccacagcagtgcagcccatgacccaaacacacggggcagcagaaacaatggacaggcccacaagtgaccatgatgggctccagcccaccagccccagagaccatgaaacagatggccaaggtcaccctacaggtcatccagatctggctccaaggggtctgcatcgctgctgccctcccaacgccaaaccagatggagacagggccggccccatagcaccatctgctgccgtccacccagcagtcccggaagcccctccctgaacgctgggccacgtgtgtgaaccctgcgagccccccatgtcagagtaggggcagcaggagggcggggctggccctgtgcactgtcactgcccctgtggtccctggcctgcctggccctgacacctgagcctctcctgggtcatttccaagacattcccagggacagccggagctgggagtcgctcatcctgcctggctgtcctgagtcctgctcatttccagacctcaccagggaagccaacagaggactcacctcacacagtcagagacaatgaaccttccagaaatccctgtttctctccccagtgagagaaaccctcttccagggtttctcttctctcccaccctcttccaggacagtcctcagcagcatcacagcgggaacgcacatctggatcaggacggcccccagaacacgcgatggcccatggggacagcccagcccttcccagacccctaaaaggtatccccaccttgcacctgccccagggctcaaactccaggaggcctgactcctgcacaccctcctgccagatatcacctcagccccctcctggaggggacaggagcccgggagggtgagtcagacccacctgccctcaatggcaggcggggaagattcagaaaggcctgagatccccaggacgcagcaccactgtcaatgggggccccagacgcctggaccagggcctgtgtgggaaaggcctctggccacactcaggggctttttgtgaagggccctcctgctgtggtggatacagctatggttacgggtatagcagcagctggtaccacagtgatgaaaccagcagcaaaaactgaccggactcgcagggtttatgcacacttctcggctcggagctctccaggagcacaagagccaggcccgagggtttgtgcccagaccctcggcctctagggacacccgggccatcttagccgatgggctgatgccctgcacaccgtgtgctgccaaacaggggcttcagagggctctgaggtgacttcactcatgaccacaggtgccctggtcccttcactgccagctgcaccagaccctgttccgagagatgccccagttccaaaagccaattcctggggccgggaattactgtagacaccagcctcattccagtacctcctgccaattgcctggattcccatcctggctggaatcaagagggcagcatccgccaggctcccaacaggcaggactcccacacaccctcttctgagaggccgctgtgttccgcagggccaggccgcagacagttcccctcacctgcccatgtagaaacacctgccattgtcgtccccacctggcaaagaccacttgtggagcccccagccccaggtacagctgtagagagagtcctcgaggcccctaagaaggagccatgcccagttctgctgggaccctcggccaggccgacaggagtggacgctggagctgggcccacactgggccacataggagctcaccagtgagggcaggagagcacatgccggggagcacccagcctcctgctgaccagagacccgtcccagagcccaggaggctgcagaggcctctccagggggacacagggcatgtctggtccctgagcagcccccaggctctctagcactgggggcccctggcacagctgtctggaccctccctgttccctgggaagctcctcctgacagccccgcctccagttccaggtgtggttattgtcagggggtgccaggccgtggggtatagcagtggctggtacgtattactatgatagtagtggttattactaccacagtggtgccgcccatagcagcaaccaggccaagtagacagacccctgccacgcagccccaggcctccagctcacctgcttctcctggggctctcaaggctgctgtctgccctctggccctctgtggggagggttccctcagtgggaggtctgtgctccagggcagggatgactgagatagaaatcaaaggctggcagggaaaggcagcttcccgccctgagaggtgcaggcagcaccacagagccatggagtcacagagccacggagcccccagtgtgggcgtgtgagggtgctgggctcccggcaggcccagccctgatggggaagcctgccccgtcccacagcccaggtccccaggggcagcaggcacagaagctgccaagctgtgctctacgatcctcatccctccagcagcatccactccacagtggggaaactgagccttggagaaccacccagccccctggaaacaaggcggggagcccagacagtgggcccagagcactgtgtgtatcctggcactaggtgcagggaccacccggagatccccatcactgagtggccagcctgcagaaggacccaaccccaaccaggccgcttgattaagctccatccccctgtcctgggaacctcttcccagcgccaccaacagctcggcttcccaggccctcatccctccaaggaaggccaaaggctgggcctgccaggggcacagtaccctcccttgccctggctaagacagggtgggcagacggctgcagataggacatattgctggggcatcttgctctgtgactactgggtactggctctcaacgcagaccctaccaaaatccccactgcctcccctgctaggggctggcctggtctcctcctgctgtcctaggaggctgctgacctccaggatggcttctgtccccagttctagggccagagcagatcccaggcaggctgtaggctgggaggccacccctgtccttgccgaggttcagtgcaggcacccaggacaggaaatggcctgaacacagggatgactgtgccatgccctacctaagtccgcccctttctactctgcaacccccactccccaggtcagcccatgacgaccaacaacccaacaccagagtcactgcctggccctgccctggggaggacccctcagcccccaccctgtctagaggacttggggggacaggacacaggccctctccttatggttcccccacctggctcctgccgggacccttggggtgtggacagaaaggacgcctgcctaattggcccccaggaacccagaacttctctccagggaccccagcccgagcacccccttacccaggacccagccctgcccctcctcccctctgctctcctctcatcactccatgggaatccagaatccccaggaagccatcaggaagggctgaaggaggaagcggggccgctgcaccaccgggcaggaggctccgtcttcgtgaacccagggaagtgccagcctcctagagggtatggtccaccctgcctggggctcccaccgtggcaggctgcggggaaggaccagggacggtgtgggggagggctcaggtccctgcaggtgctccatcttggatgagcccatccctctcacccaccgacccgcccacctcctctccaccctggccacacgtcgtccacaccatcctgagtcccacctacaccagagccggcagagccagtgcagacagaggctggggtgcaggggggccgccagggcagctttggggagggaggaatggaggaaggggaggtcagtgaagaggcccccctcccctgggtctaggatccacctttgggacccccggatcccatcccctccaggctctgggaggagaagcaggatgggagaatctgtgcgggaccctctcacagtggaatacctccacagcggctcaggccagatacaaaagcccctcagtgagccctccactgcagtgctgggcctgggggcagcccctcccacagaggacagacccagcaccccgaagaagtcctgccagggggagctcagagccatgaaggagcaagatatggggaccccaatactggcacagacctcagctccatccaggcccaccaggacccaccatgggtggaacacctgtctccggcccctgctggctgtgaggcagctggcctctgtctcggacccccattccagacaccagacagagggacaggccccccagaaccagtgttgagggacacccctgtccagggcagccaagtccaagaggcgcgctgagcccagcaagggaaggcccccaaacaaaccaggaggtttctgaagctgtctgtgtcacagtcaggatattgtagtggtggtagctgctactccagcatattgtggtggtgattgctattcccacaatgacactgggcaggacagaaaccccatcccaagtcagccgaaggcagagagagcaggcaggacacatttaggatctgaggccacacctgacactcaagccaacagatgtctcccctccagggcgccctgccctgttcagtgttcctgagaaaacaggggcagcctgaggggatccagggccaggagatgggtcccctctaccccgaggaggagccaggcgggaatcccagccccctccccattgaggccatcctgcccagaggggcccggacccaccccacacacccaggcagaatgtgtgcaggcctcaggctctgtgggtgccgctagctggggctgccagtcctcaccccacacctaaggtgagccacagccgccagagcctccacaggagaccccacgcagcagcccagcccctacccaggaggccccagagctcagggcgcctgggtggattctgaacagccccgagtcacggtggtattacgatttttggagtggttattataccgtattactatggttcggggagttattataaccactgtgagaaaagctatgtccaaaactgtctcccggccactgctggaggcccagccagagaagggaccagccgcccgaacatacgaccttcccagccctcatgacccccagcacttggagctccacagtgtccccattggatggtgaggacgggggccggggccatctgcacctcccaacatcacccccaggcagcacaggcacaaaccccaaatccagagccgacaccaggaacacagacaccccaataccctgggggaccctggccctggtgacttcccactgggatccacccccgtgtccacctggatcaaagaccccaccgctgtctctgtccctcactcagggcctgctgaggggcgggtgctttggagcagactcaggtttaggggccaccattgtggggcccaacctcgaccaggacacagatttttctttcctgccctggggcaacacagactttggggtctgtgcagggaggaccttctggaaagtcaccaagcacagagccctgactgaggtggtctcaggaagacccccaggagggggtttgtgccccttcctctcatgtggaccccatgccccccaagataggggcatcatgcagggcaggtcctccatgcagccaccactaggcaactccctggcgccggtccccactgcgcctccatcccggctctggggatgcagccaccatggccacaccaggcagcccgggtccagcaaccctgcagtgcccaagcccttggcaggattcccagaggctggagcccacccctcctcatccccccacacctgcacacacacacctaccccctgcccagtccccctccaggagggttggagccgcccatagggtgggcgctccaggtctcactcactcgcttcccttcctgggcaaaggagcctcgtgccccggtcccccctgacggcgctgggcacaggtgtgggtactgggccccagggctcctccagccccagctgccctgctctccctgggaggcctgggcaccaccagaccaccagtccagggcacagccccagggagccgcccactgccagctcacaggaagaagataagcttcagaccctcagggccgggagctgccttcctgccaccccttcctgccccagacctccatgccctcccccaaccacttacacacaagccagggagctgtttccacacagttcaaccccaaaccaggacggcctggcactcgggtcactgccatttctgtctgcattcgctcccagcgcccctgtgttccctccctcctccctccttcctttcttcctgcattgggttcatgccgcagagtgccaggtgcaggtcagccctgagcttggggtcacctcctcactgaaggcagcctcagggtgcccaggggcaggcagggtgggggtgaggcttccagctccaaccgctccactagccgagactaaggaagtgagaggcagccagaaatccagaccattccatagcaaatggatttcattaaagttaccagacttcagtgtaagtaacatgagccccatgcacaacaatcccttatgaaggggaagtcagtgtcgcctcggatttcttgaaaaacacaaaaacttatcaatgcctgtaaaagtctgttggaaagaaaatatgattcaagaatgttatgcccaacaaagctggcatattttctacccggacacactcagggaatgtggtcccttgagtgcttctctcactgcgtaaatcctacgtggtgtttaagcatattcataaatgtgtatgtctatttttatgtgtaagatggttcatttttattttatttattcaatatgtacaataaagaatattgacaaataggctggacatggtggctcccacctgtaatcccagccctttgggaggccgaggcgggcagatcacctgaggtctggagttcgagaccagcctggccaacatgatgaaaacccatctctactaaaaatacaaagattagccaggcatggtggtgcatgcctgtaatcccagccactcaggaggctgagacaggagaaatgcgtgaacccggaaggcggaggttgcagtgagccgagatcacaccactgcactccagcctggcgacagagcaagattccatctcaaaaaaaaaaagacaaagaaatttgtttttttgaataaagacaaatttcatcacacgaagataaagatgcaaagctccagacaggaaggcacggacagcacagtgaagcccggagcgggcgctggggggccaggggcatggcgggggtgccagcgtctctcggtgcctaccatggccactccagcctgtgttctcacgaggatggctgtgcaatgctaggagcgtgttcgaagctctagggcaaccactggaagtgaggctgaggagcagagcccagaggcccgtggagctgatgaaaagaaagctggagaaagtgtttgctgcctcccaacatggtaagaaaagatagaaagagagagcacacggcaaagggagcttgctgagggactctttacaatggcttgcacagagctcagggggtctgggaggctagggccctgcgcagggcagtcaccccagcctgctgaccaaggtttgctgcaggcagctctgggggtggttgaggcgcggtccctggagccacccctcaagggaacgaggcagcagagtgggccaaggcccaggtcggctgcaaggctgcccaggacttggggtccttacatcagcagccactgatgcagctggcccagagagaggcgccgagcaggttgcctccaggggacaaaccaggtcggagagggtgaggcagtggatggagccacaacaaccccgggcacgggtgacacgcacgttcatgcacatctgacccttcctccctcaccaaacaggtccccctgccttccccatggttgcgaaaaagcaaaatgtagacgttttttcttttttaattcatgttttaattgacaaatgaagccgtatatatttattgtgtacaacatgatgctttaaaatatgtatacatcgtggaacagcaacgttgagctaatttaacacgcattacttcacatacttgtcatcttttgtggcgagaatgcttaaaatccactctcttagtattttttaagaatgcaatacattgttgtcaactgtggtcaccgtcatgcatagccaagctcccgacctcaccctcctgccagctcaggctgtgcatcctttcaccagcatcccccaccccggcccctggccctggtaactaccactctatactctacgtatgagttcagctttttaagattccacagatgaatgagatcatacagtatttgctttctatgcctggcttattttagttaacacactgtcctccagatccatccgttgttgcaaatgacagggtttcattctttttaaagtctaaagagtattccattgtgtcaatggacctcatttgctttatccatgcatcaactatggacatttaggttgattccatttcttagctgttgtggatggtgctgcagtaaacatggggctgcagatgtctcttcaacatactgacatcatgtcctttggataaatacccagtagtgggatcgctggatcacaatgtacagttttttttttaatggaaactttcattttttggtgaaattaggaaaacagataaaacccacagaatccaaaatatatgtgaagatgccaaaaacagttgacattgggcagaggtcacatggaaggaagtgaatacatgacggggtgtgagggcccagaggcagctgaaatacgctttctaaacacaaggacctcttctgagagggcagaagttttatcctgcacatgcaatgaccagcacagctaaaatacactttctaaacatgaggacctcttctgagagggcagctttatcctgcaaatgcaatgaccagcacaggacccagaataaagagagttgccagcggacgcctggtgtccatgtgtccaggtgagttcgagatgcggacggcgctggccagccagtcacaccctaagtcaatctgctgcatgcatttgtccttgccacagcagaaaacgagaaagcctttgggctgcaaagcttcacaggctcctcttctcccgactccatggaaacagctacaaagagcaggcccagtagagcttaattcatgaaaatgagtaataaacttgaactggaacagtatcgactttttagaaacggcagcaaagtgtataaaaaatattcaccagaacaatatttccaaacgatgagatgagaatttcagccaagtaatcctccatggatagaaaataatgaagggattggatttatgaaggaaaatcatggagctcaaatacaagagaagagaatcaaaaatgaacaggaggagataaaatatggtttggccaaagttacaaaataaattttttaaaaacccttcatcatggcaagtagaaagagcgagaggaaaaacagatcccgtggaagacacaaataggacatggggagaaaaatgaatgagatgaaacagagcagaaataaaattttacggaactaaagacaagtgatctgaacctgcctggggcctgggggacctcgccaccctgaagggaaagaacatgcctggctggctttgccacctgctcattgcagagccccacagcttgcaacaaacataggcggtagccagggagtggttacagcaggccttgagcaagacccagtgttgtgctgacttcaggtctgacccagcactgtcatagtggtggtgtccatagtggtagtgggggtgcttgtgtcactccacccccatctccaggaggctcagaacagacagagagagactccatttgtttgggagaaagtaagggatgagaacaagagtctctgcctggtaatccagagaattattctagatcttggccaagattatcaaagcagtacctctatgagtcttttgggcttggagtccccctaaagcagatatagctaagatcacaacacccaagtccttttgaatatgtgggaagacttcccaaggacaggagcaaacaaacaagcccagactgcaaaaaaacaagcccagactgcaataaacacctcactcttcaatgcccaggcactgaagaacatctcctagcagcaacaccatccaggaaaacatggcctcaaccagtgaactaaataaggcaccagggaccagtctcggagaaatagaggtatgttatctttcagagaattcaaagtagctttgttgaggaaactcaaagaaattcaagataacacagtgaaggaattcagaatcctatccgataaatttaacagagattgaagcaattaaaaagaattaagcagaaattatggagctgaaaaatgcaattggcatactgaaaaatgcatcagagtattttcatagcctcttatatcaagtagaagaaagaattagtgagcttgaaaacaggctatttggaaaagcacgataaaaggagacaaaagagaaaagaataaataacaatgaagcatatctacaggatctagaaaatagcctcaaaaggccaaatctaagaattattagccttaaagaggaggtagagaaagagggatggagagtttattcaaagggataataacagaaaacttcccaaacctagagaaagatatcaatatccaaatgcaagaaggatgtagtacaccaaggagatttaatgcaaagaagactacctcaaggcattcaatactcaaactcccatatgacaaggactttaaaaagatcctaaaagcagcaaaagaaaagaaatgaataaaatactatggagctccaatatgtctggcagcagacttttcagtgaagactttatatgccaggagagagtgtcataatggatttaaagtgctgaaggaaaaaacttttaccctcgaacagtatagctggtgaaattatccttcaaacatgaaggagaaataatttgtttccagacaaatgttgagggatttcatgaacaccagacctgtcttttaagaaatgctaaagggagtacttcaatcagaaagaaacacgttagtgaacaataagaaatcatctgaaggcacaaaactcaccggtaatagtaagtacacagaaaaacacagaatattataacactgtaactgtggtgtgtaaactccttttgtttgtttgtttgtttgtttgtttgtttgtttgtttttgtttttagacggagttttgctccagcccaggctggagtgcaatggcacaatctcagctcactgcaacttccacctcccgggttcaagcaattctcctgcctcagcctcccaagtagctgggattacaggcatgtgctaccatgtccagctaattttgtattttagtagagacggtgtttcaccatgttggtcaggctagccttatcttgagtagaaaaactaaatgatgaagcaatgaaaaataataactacaacttttcaagacatagtacaataagatataaatcataacaaaaagttaaaaggtggagggatgaagttaaggcaaagagtctttattagttttctttttacttgtctgtttatgcaaacagtgttaagttgtcatcagtttaaaataatgggtcataagatactatttgcaagcctcatggtaacgtcaaaccaaaagcaatacaacagatacacaaaaaacaaaaagcaagaagctaaattacgtcatcagagaaaatcaccttcactaaaaggaagacggagaaaagaatgaagagagagaagaccaaaagcaaatagcaatatggcaggagtaagtccttacttatcaataataccattgaatgtaaatggactaaactctccaatcaaaagacatagagtggctgaatcaattaaagaaaaaacaagacccattgatctgttgtccacaagaaacacactttatctataaagacacacatagactgaaaacaaagggatggaaaaagatactccacgccaatggaaaccaaagaaagagcaggagtagctacacttatatcaggcaaaatagatttcaagacaaaaactataagaagagacaaggtcactaatgataaacaggtcaattcagcaagaggatataacaattgtaaatatatatgcacccaatgctggagcacccagatatataaagcaagtatttactagagctaaagagagaaatagactccaatgcaataatagctggagatttcaacatcccactttcaacattgaacagatcctccagatagaaaatcaacaaagaaatattggacttaatctgcactatcgaccaaatggatctaacagatatttacagaacatttcatccaacagctgcagaacacacattcttttcctcagcacatagatcattctcaaggatagaccatatgttgggtcacaaaacaagttttaaaatattcaaatacattgaaataatatcaagcatcttctgtgaccacaatggactaaaactagaaatcaataacaagaggaattttggaaactatataaatatatggaaattaatgaatgctgagtgggtcaatgaagcaattaagaaggaaactgaaatttttcttggaacgaatgatcatggaaacagaaaataccaaaacctatgggatacagcaaaagcagtactaagagggaagtttacagctacaaatgcttacattaaaaaagaagaaaaacttcaataaaaaaacctaacaatgcatcttaaagaactagaaaagcaagaggaaatcaaatccaaaattagtagaagaaaacagtaaaggtcagagcagaaataagtaaaattgaaatgaagaaaacaatacaaaagatcaataaaacaacaggttgttttcttgaaaagttaaacaaaattgacaaacctttagccagactaagaaaaaaagacagaagatccaaataaataaaatcagagatgaaaaaggtgacattacaacttacaccacagaaattcaaaggatcattagtggctactataagcaactatatgccaataaattggaaaatctagaagaaatgcagaaattcctagacacatacaacctcccaagattaaaccaagaagaaattcaaaacctgaacagactgataacaagtaatgagatcaaagccgtaataaaaagcctcccagtaaagagaagcccaggacccgacggcttcactgctgaattctaccaaacatttaaagtagaactaataccaatcctactcaaactattccaaaaaatagaggtggaaggaatacttcaaaactcattatacgaggccagtattaacctgacaccaaaactagacaaagacacatgaaaaaaagaaaactacaggccaatatgtctgatgaatattgacacaaaaatcctcaacaaaatactagcaaaccaaattcaactacacattagaaagttcactcatcatgaccaagtggaatttatctaacttgggatgcaaagatggttcaacatatgcaaatcaatcaatgtgatacatcatatcaacagaatgaacaacaaaaaccatttgatcatttaattgatactgaaaaagcatttgataaaattcaacattccttcataataaaaattctcttctatactaggtacaaaagaaacttacctcaacataataaagccatatatgacagtcccacagtatgatactaaatgaggaaaaactgagagcctttcctctacgatctggaacatgacaaagatgcccactttcatcactgttattcaacatagtactggaagtcctagctggagcgatcagacaagagaaagatataaaagacatccaaattggaaaggaataagtcaaattatcctcatttgcatatggtatgatcttctatttagagctaactaaagactccaccaaaaaaagttattagaactgacgaacaaattcagtaaagctgcaggatacaaaatcaacatacaaaaatcagtagcatttctatatgccaacaatgaccaatgtgaaaaagaaattaaaaagtaaccctatttacaataaccacaaataaacacctaggaattaaccaaagaggtaaaagatttctgtaatgaaaactataaaaactgatgaaagaaattgaagagtacaccaaaaaatggaaagcaattgcatgttcatggattagaagaatcagtgttgttataatgtccatactatccaaagcaatctacagattcaatgcaatccttatcaaaataccaatgacatcattcacagaaatagaaaaaaaaaatcctaaaatttacgtggaaccacaaagacccagaatagccaaagctctcctaagcaaaaagaacgaaactgtaggaatgacattgcctgtcttcaaattctactacagagctatagatagtaaccaaaacagcgtggtactagcataaaaacagacacagagacaaacagaacaaaatttaaaaacccagaaataaatccacacacctacagcaaattcatttttgacaaagttgccaagaacatactctggggaatagataatgatatctcttcaataaataatgtggggaaaactggatatccatatacataacagtgaaactagacccctctctctctcactatatacaaaaatcaaatcaaaattgtttaaggacttaaatctaagacctcatactatgaaaccactgcaagacaaccttggcggaaactctccaagacatcagtccaggcaaagatttcttgagtaatatcccacaagcacagacaaccaaagcaaaaatggacaaatgggatcacatcaagttaaaaagcttctgcacagtaagggaaacaaccaacaaaatgaagagacaacccacagaatgggagaaaatatttgaaaaatacccatctggcaagggattaaaaaccagaatatatgcagaatatataaggagctcaaacagtgctatagaaaaaaaaatctaataatctgatttaaaaatgggaaaaatgttagaatagacatttcttaaaataagacatacagatggcaaaccgacatggaacggtgctcaacatcatggattatcacagaaacacaatcaatcaaaactaaaactaaaatgtgctatcatctcaccccagttaaaatggctgatatccagaagacaggcaataacaaatgctggcaaggatgtggggaaaagggagcccccatacactgttgctgggattgtaaattagtacaaccactgtggagagcagcatgaaagttcctcaaaaaactgaaagaaagctaccataggatccagcaatcccactgctgtgtatatactacaaaagaaaggaagtcagtatatgaagaggtatctgcactcccatgtttgttgcagccctgttcacaacagccaagatttggaagcaacctaagtgtccatcagcagttgaatgtataaagaaaatgtggtgcatatacacaatggagtattattcaataataaaaaggaatgagattgagtcatttgcaacaacatggatggaactggagatcattatgtgaagtgaaataagccaggcacagaaagacaaacattacaatgttcttacttattaatgagatctaaaaatcaaaacaattgcacccatgttcataaagagtaaaaggatggttaccagatgctgagaacggtggtggggggatagggaaaggtggcagtggttaacgggtacaaaaaaatagaaagaatgaataagacttactacttgatagcacagcaaggtggctatagtcagtaatttagttgtatatttttaataatgaaaggtgtataattggattgtttctaacacaaaggataatgcttaagaggatggataccccattttccatgatgtgattatttcacattgcacgcctagatcaaaacatccaatgtaccccataaatatatacatcttctatgtacccataaaaattctgtaaaataaaatatataaaaagaggtgacagatatggaagacaggcaaagaagagacgacatccacataatccgagtacctaagaaagaatggagtccagtgcatctcaggagccaccattctaagccaattttctctggttctctcagtcaccctactaatacgtgggcaatcttgttttatttcaggatagagtttttgaaattatagatttaagtatgctttctgttctattacttttggtaattaattttagaaagaactaatttgggcacaaatttgaaaaaattctaaatccaaaaaaaaaaagaaaaaaacacacacacaatcatctataagggggatgatgaccagtcctagatttctcaccagccacattcaagatcagtaaatggtaggacaaaacctgtagggtccttaagggggaaagaagtagtggatagtccagagtctatatacagccaactgttcttgaagaaaaaaggctgctgaaaaggagttccaaacattctataatccataatctcatgatgaaactactagaggaagaccaccagccatcaaaaggtgcttggagaacccagggccaagaaccaaaagtaaatattaagtgtccttaactgcgagactaagatagaaatgactgtgggggaccatgtggcctcaacagaggtgaaatggtgtctgcctgacaaagtggacattttacaatgatcaaaacacagaatatgagatagagagcacttctgaattactgcctcactccaaataactctcagccaaaggacttcagtaaaaccaaattgggcatattagacagtacaaacaaattctaagaaaataatattactgattacaatcacatgatgctagagatggaggggaaaaggaagaggaaaccaggtaatttcatactcgtatatagtaaagaactaaagtacattgtccaaagaagaacaaagaatattttggaaagttataaaggtagccactacacatagaagatagcaaagaacaagaaaacttaagatggaaaactttttggaagcataaaaatagaaaatataaactactaagataagattgaagccaaacagatctatgaaaacaacaaacatcaatggccttaacttgcctattaaaaggaagagactttcaaattggaccacaagataaaacccaactctatatagcatatgagtattacacacaaaatgggaaaagctgaaaaaacttgggcaaaattcaccccaagcaaattccactgtttcctttgggacaaaatgccaagctccatgccagggaagatgattctcctcagaccttctcctcactctcccagtcctcttagggaaggaattgggtgttagaggagggagactctgtcgattatcagctgaagcagtggtgtgctcctgcgttgcttctgacctgggaaatgaagcagcaagactctttctgctgtgtctttgcccagaagggccatccccccagagcagagtacccaggccggcaggagcagtggtggaagcgtggaaaccacgtctcctacagcagagaccatcagaagcggagcctcgggtataagggaaacaacgcgttctccctaacctgggagtgacagacagcgtcattcctcacagtgataccctgtgttctagccatctggcccatgacagagccagcccagagccagcccagagccagcccctcaccaacctggagcctggccagctcgccaagctgcaccataggcctggaaggcgtggagacctgcggcagtgccctgtcctcccgtgaggcctgccatccctgccaggggtcgcctctggcttctcctccaggaccgcacggtccagaggctcagtgcctggagtaggtgttgcccccctgcttctaggcccagaccctcccttgttcctgaccccgggcctttccctctggcttggacatccagggccctgtctcagctggggagctgctcctgctcaaggactgtcttccgcgggatcgaaaggccgcgtcctgaacaatgcgtgggccacgtgagcggagcaggctctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgggagcggagcagactctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgtgagcggagcaggctctaaaggccgcgtcctaaacagtgcgtgggccacgggagcggagcagactctaaaggccgcgtcctaaacagtgtgtgggccacgtgagcggagcaggctctaaaggccgcgtcctaaacagtgcgtgggccacgggagcggagcagactctaaaggccgcgtcctaaacagtgtgtgggccacgtgagcggagcgccctctccactgccctcggggccgcagctcccagctcagctcccagccctgctcagggcagccaggccaggaggtaccatccaggctaagtgaccctcaggggggacaggtgccccaggagatgccagctgttgggagaggctgggggaccaactcgacctggcctgtgggccctgccctggccacccattgtaggatccagccgccacgcctgtgacactcgtgtgctttccctggtgtgtgcttgtggcaggtgggggcagagggtcctcaggccagagagccactcccccagcgccagaccaccctcttcctcactcccccacctcaccccctcacaggtgcctcccaggccatcagggcccaaccacccctaaacaaatgggttctcggcccctcgtggctggaggtgggttctctcaccattcccagcctaaggctccatccccatgctggcagctgttcaaccatgtctagagagatccactgtcccagacagcacctcagggtcccccgtcctgcctggaaccctgtaggaaactccacaaaccgccgccattctgtccacacccctacaggagccccaaccctctccccacatccaggcttccctcccagacccctcatccctgcccgcacggtgcctgagggggccttcttgggcagcgcctaagcaagcccccagcacccttcggccccttcaaggcacacaggccccctttccacccagcctcaggaaaccacctgtgtcctccaacgacaggtcccagcctcccagcctttgccttgcctgttcctctccctggaactctgccccgacacagaccctccccagcaagcccgcaggggcacctcccctgcccccagacaccctgtgcccgtcagttcatccccagcagaggccctcaccaggcacacccccatgctcacacctggccgcaggcctcagcctccctgagggccccacccagcccgcgtctggccagtggtgcgtgcaaagcccctcacccagactcggcggaaggcagccagtgcaggcctggggaggggctctccttagaccaccttgcaccttccctggcacccaccatgggaagagctgagactcactgaggaccagctgaggctcagagaagggacccagcactggtggacacgcagggagcccacgccagggcgccgtggtgagtgaggcccagtgccacccactgaggcctcccgttcagtgggacgacggtgaacaggtggaaccaaccaggcaacccccgccgggccccacagacgggatcagagcaggaaaggcttcctgcccctgcaggccagcgaggagccctggcgggggccatggccctccaggcgaggaggctcccctggccaccgccacccgggcctctctgctgctgggaaaacaagtcagaaagcaagtggatgagaggtggcgtgacagacccagcttcagatctgctctaatttacaaaagaaaaggaaaaacacacttggcagccttcagcactctaatgattcttaacagcagcaaattattggcacaagactccagagtgactggcagggttgagggctggggtctcccgcgtgttttggggctaacagcggaagggagagcactggcaaaggtgctgggggcccctggacccgacccgccctggagaccgcagccacatcagcccccagccccacaggccccctaccagccgcagggttttggctgagctgagaaccactgtgaggatattgtactggtggtgtatgctataccggtacaactggaacgaccacagtgattggcagctctacaaaaaccatgctcccccgggaccccgggctgtgggtttctgtagcccctggctcagggctgactcaccgtggctgaatacttccagcactggggccagggcaccctggtcaccgtctcctcaggtgagtctgctgtctggggatagcggggagccaggtgtactgggccaggcaagggctttggcttcagacttggggacaggtgctcagcaaaggaggtcggcaggagggcggagggtgtgtttttgtatgggagaagcaggagggcagaggctgtgctactggtacttcgatctctggggccgtggcaccctggtcactgtctcctcaggtgagtcccactgcagccccctcccagtcttctctgtccaggcaccaggccaggtatctggggtctgcagccggcctgggtctggcctgaggccacaccagctgccatccctggggtctccgccatgggctgcatgccagagccctgctgtcacttagccctggggccagctggagcccccaaggacaggcagggaccccgctgggcttcagccccgtcagggaccctccacaggtagcaagcaggccgagggcagggacgggaaggagaagttgtgggcagagcctgggctggggctgggcgctggctgttcatgtgccggggaccaggcctgcgctttagtgtggctacaagtgcttggagcactggggccagggcagcccggccaccgtctccctgggaacgtcacccctccctgcctgggtctcagcccgggggtctgtgtggctggggacagggacgccggctgcctctgctctgtgcttgggccatgtgacccattcgagtgtcctgcacgggcacaggtttatgtctgggcaggaacagggactgtgtccctgtgtgatgcttttgatatctggggccaagggacaatggtcaccgtctcttcaggtaagatggctttccttctgcctcctttctctgggcccagcgtcctctgtcctggagctgggagataatgtccgggggctccttggtctgcgctgggccatgtggggccctccggggctccttctccggctgtttgggaccacgttcagcagaaggcctttctttgggaactgggactctgctgctggggcaaagggtgggcagagtcatgcttgtgctggggacaaaatgaccttgggacacggggctggctgccacggccggcccgggacagtcggagagtcaggtttttgtgcaccccttaatggggcctcccacaatgtgactactttgactactggggccagggaaccctggtcaccgtctcctcaggtgagtcctcacaacctctctcctgctttaactctgaagggttttgctgcatttttggggggaaataagcgtgctgggtctcctgccaagagagccccggagcagcctggggggctcaggaggatgccctgaggcaacagcggccacacagacgaggggcaagggctccagatgctccttcctcctgagcccagcagcacgggtctctctgtggccagggccaccctgggcctctggggtccaatgtccaacaacccccgggccctccccgggctcagtctgagagggtcccagggacttagcggggtgccagttcttgcctggggtcctggcattgttgtcacaatgtgacaactggttcgacccctggggccagggaaccctggtcaccgtctcctcaggtgagtcctcaccaccccctctctgagtccacttagggagactcagcttgccagggtctcagggtcagagtcttggaggcattttggaggtcaggaaagaaagctggggagagggacccttcgaatgggaacccagcctgtcctccccaagtccggccacagatgtcggcagctggggggctccttcggctggtctggggtgacctctctccgcttcacctggagcattctcaggggctgtcgtgatgattgcgtggtgggactctgtcccgctccaaggcacccgctctctgggacgggtgccccccggggtttttggactcctgggggtgacttagcagccgtctgcttgcagttggacttcccaggccgacagtggtctggcttctgaggggtcaggccagaatgtggggtacgtgggaggccagcagagggttccatgagaagggcaggacagggccacggacagtcagcttccatgtgacgcccggagacagaaggtctctgggtggctgggtttttgtggggtgaggatggacattctgccattgtgattactactactactactacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgatagggacaaagagtggagtggggcactttctttagatttgtgaggaatgttccgcactagattgtttaaaacttcatttgttggaaggagagctgtcttagtgattgagtcaaggaagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactatgactttaaaatgtgagagggttttcaagtactcatttttttaaatgtccaaaatttttgtcaatcaatttgagtcatgtttgtgtagaactgatattactgaaagtttaaccaaggaataggaatgatgctctttcataccctattcagaactgacttttaacaataataaattaagtttaaatatttttaaatgaattgagcaatgttgagttggagtcaagatggccgatcagaaccagaacacctgcagcagctgtcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttagaattcattttcaaaattaggttatgtaagaaattgaaggactttagtgtctttaatttctaatatatttagaaaacttcttaaaattactctattattcttccctctgattattggtctccattcaattcttttccaatacccaaagcatttacagtgactttgttcatgatcttttttagttgtttgttttgtcttactattaagactttggacagcatttatacagtatccgatgcatagggacaaagagtggagcggccgcggcgcgccgcggccgc

SEQ ID NO: 2 - DNA, 48 bp, synthetic construct
  source 1..48: mol_type = other DNA
ggtacaactggaacgacaggatattgtactggtggtgtatgctatacc

SEQ ID NO: 3 - DNA, 62 bp, synthetic construct
  source 1..62: mol_type = other DNA
aggatattgtagtagtaccagctgctatgccgtattacgatttttggagtggttattatacc

SEQ ID NO: 4 - DNA, 52 bp, synthetic construct
  source 1..52: mol_type = other DNA
aggatattgtagtagtaccagctgctatgccgggtatagcagcagctggtac

SEQ ID NO: 5 - DNA, 31 bp, synthetic construct
  source 1..31: mol_type = other DNA
aggatattgtactggtggtgtatgctatacc

SEQ ID NO: 6 - DNA, 48 bp, synthetic construct
  source 1..48: mol_type = other DNA
aggatattgtactggtggtgtatgctataccggtacaactggaacgac

SEQ ID NO: 7 - DNA, 59 bp, synthetic construct
  source 1..59: mol_type = other DNA
aggatattgtagtggtggtagctgctactccagcatattgtggtggtgattgctattcc

SEQ ID NO: 8 - DNA, 62 bp, synthetic construct
  source 1..62: mol_type = other DNA
aggatattgtagtggtggtagctgctactccgtattactatgatagtagtggttattactac

SEQ ID NO: 9 - DNA, 47 bp, synthetic construct
  source 1..47: mol_type = other DNA
aggatattgtagtggtggtagctgctactcctgactacggtgactac

SEQ ID NO: 10 - DNA, 51 bp, synthetic construct
  source 1..51: mol_type = other DNA
aggatattgtagtggtggtagctgctactccgtggatacagctatggttac

SEQ ID NO: 11 - DNA, 51 bp, synthetic construct
  source 1..51: mol_type = other DNA
aggatattgtagtggtggtagctgctactccgtagagatggctacaattac

SEQ ID NO: 12 - DNA, 31 bp, synthetic construct
  source 1..31: mol_type = other DNA
gtattacgatttttggagtggttattatacc

SEQ ID NO: 13 - DNA, 62 bp, synthetic construct
  source 1..62: mol_type = other DNA
gtattacgatttttggagtggttattataccgtattactatggttcggggagttattataac

SEQ ID NO: 14 - DNA, 37 bp, synthetic construct
  source 1..37: mol_type = other DNA
gtattatgattacgtttgggggagttatcgttatacc

SEQ ID NO: 15 - DNA, 51 bp, synthetic construct
  source 1..51: mol_type = other DNA
gtggatacagctatggttacgtattactatggttcggggagttattataac

SEQ ID NO: 16 - DNA, 51 bp, synthetic construct
  source 1..51: mol_type = other DNA
gtggatacagctatggttacgtattactatgatagtagtggttattactac

SEQ ID NO: 17 - DNA, 41 bp, synthetic construct
  source 1..41: mol_type = other DNA
gtggatacagctatggttacgggtatagcagcagctggtac

SEQ ID NO: 18 - DNA, 54 bp, synthetic construct
  source 1..54: mol_type = other DNA
gtggatatagtggctacgattacaggatattgtagtggtggtagctgctactcc

SEQ ID NO: 19 - DNA, 54 bp, synthetic construct
  source 1..54: mol_type = other DNA
gtggatatagtggctacgattacgtattactatgatagtagtggttattactac

SEQ ID NO: 20 - DNA, 39 bp, synthetic construct
  source 1..39: mol_type = other DNA
gtggatatagtggctacgattactgactacggtgactac

SEQ ID NO: 21 - DNA, 43 bp, synthetic construct
  source 1..43: mol_type = other DNA
gtggatatagtggctacgattacgtggatacagctatggttac

SEQ ID NO: 22 - DNA, 44 bp, synthetic construct
  source 1..44: mol_type = other DNA
gtggatatagtggctacgattacgggtatagcagtggctggtac

SEQ ID NO: 23 - DNA, 49 bp, synthetic construct
  source 1..49: mol_type = other DNA
gagtatagcagctcgtccgtattactatggttcggggagttattataac

SEQ ID NO: 24 - DNA, 39 bp, synthetic construct
  source 1..39: mol_type = other DNA
gagtatagcagctcgtccgggtatagcagtggctggtac

SEQ ID NO: 25 - DNA, 49 bp, synthetic construct
  source 1..49: mol_type = other DNA
gggtatagcagcagctggtacagcatattgtggtggtgattgctattcc

SEQ ID NO: 26 - DNA, 42 bp, synthetic construct
  source 1..42: mol_type = other DNA
gggtatagcagcagctggtacgggtatagcagtggctggtac

SEQ ID NO: 27 - DNA, 41 bp, synthetic construct
  source 1..41: mol_type = other DNA
gggtatagcagtggctggtacggtatagtgggagctactac

SEQ ID NO: 28 - DNA, 52 bp, synthetic construct
  source 1..52: mol_type = other DNA
gggtatagcagtggctggtacgtattactatgatagtagtggttattactac

(19) United States
(12) Patent Application Publication
(10) Pub. No.: US 2024/0239919 A1
(43) Pub. Date: Jul. 18, 2024

(54) HUMAN IMMUNOGLOBULIN HEAVY CHAIN LONG CDR3 TRANSGENE CONSTRUCTS AND USES THEREOF

(71) Applicant: Gilead Sciences, Inc., Foster City, CA (US)

(72) Inventors: Daniel Rohrer, Los Gatos, CA (US); Peter Brams, Sacramento, CA (US)

(21) Appl. No.: 18/414,063

(22) Filed: Jan. 16, 2024

Related U.S. Application Data
(60) Provisional application No. 63/439,797, filed on Jan. 18, 2023.

(51) Int. Cl. C07K 16/46; A01K 67/0278; C12N 9/22; C12N 15/11

(52) U.S. Cl. CPC C07K 16/461; A01K 67/0278; C12N 9/22; C12N 15/11; A01K 2217/206; A01K 2227/105; A01K 2267/01; C12N 2310/20

(57) ABSTRACT

Human immunoglobulin heavy chain transgene constructs are provided that encode long CDR3 regions. The heavy chain transgenes comprise a plurality of longer than average VH regions operatively linked to a plurality of D-D fusion segments. Transgenic animals comprising the transgene are also provided. Methods of using the transgenic animals are also provided.

### CROSS-REFERENCE TO RELATED APPLICATIONS

This application claims the benefit under 35 U.S.C. § 119(c) of U.S. Provisional Application No. 63/439,797, filed on Jan. 18, 2023, which is hereby incorporated herein by reference in its entirety for all purposes.

### SEQUENCE LISTING

The instant application contains a Sequence Listing which has been submitted electronically in .XML file format and is hereby incorporated by reference in its entirety. Said .XML copy, created on created on Jan. 12, 2024, is named ZL8017-WO-PCT_SL.xml and is 128,639 bytes in size.

### BACKGROUND OF THE DISCLOSURE

Immunotherapy has revolutionized the treatment of a wide variety of diseases, including cancer and autoimmune disorders. While therapeutic antibodies have been raised successfully against large numbers of antigens, there still are certain types of targets that have proven challenging for antibody generation by standard methods in rodents. These include targets with epitopes that are inaccessible to or rarely bound by typical CDRs (e.g., targets with clefts or minimal surface access), such as multipass membrane proteins (e.g., G protein coupled receptors (GPCRs) and ion channels), as well as enzyme active sites and allosteric epitopes. Such targets also can be difficult to stabilize as immunogen preparations, adding to the challenge of their successful use as antigens.

Despite these hurdles, antibodies to intractable antigens such as transmembrane receptors have been described, including against the M2 ion channel of Influenza A (Wei et al. (2011) PLOS One 6:e28309), the formyl peptide receptor 1 (FPR1) GPCR (Douthwaite et al. (2015) MABS 7:152-66), the voltage gated potassium channel Kv1.3 (Wang et al. (2016) Proc. Natl. Acad. Sci. USA 113:11501-11506) and the 5-hydroxytryptamine 2B (5HT2B) GPCR (Ishchenko et al. (2017) Proc. Natl. Acad. Sci. USA 114:8223-8228). These antibodies were observed to have or were modified to have unusually long CDR H3 regions.

Some of these antibodies were raised in cows or camels, which are known to have alternative immunoglobulin locus scaffold structures that can give rise to CDR H3 regions that are longer than typical in rodents or humans (reviewed in de los Rios et al. (2015) Curr. Opin. Struct. Biol. 33:27-41; see also De Genst et al. (2006) Proc. Natl. Acad. Sci. USA 103:4586-4591; Wang et al. (2013) Cell 153:1379-1393; Sok et al. (2017) Nature 548:108-111). For example, the bovine Ig locus has limited combinatorial diversity potential because it has only 12 VH regions, but it can generate unusually long CDR H3 regions that can reach lengths of over 60 amino acids long, whereas human CDR H3 regions typically are only 8-16 amino acids in length. The camelid Ig locus can give rise to heavy chain-only antibodies having a dedicated variable domain (VHH) with long CDR H3 regions.

Approaches to take advantage of long CDR H3 regions for antibody generation have been described, including transgenic chickens with long CDR H3 regions (US Patent Publication No. 20210230253) and libraries of genetic packages with long CDR H3 regions (US Patent Publication No. 20200399785).

While some advances have been made, additional approaches and compositions are needed for designing, preparing and using heavy chain long CDR3 transgenes, particularly for use in raising antibodies to intractable antigens.

### SUMMARY OF THE DISCLOSURE

The disclosure provides human immunoglobulin heavy chain transgene constructs that encode a long CDR H3 region. The extended CDR H3 region results from the inclusion in the transgene of D-D fusion segments, which comprise two D regions joined together. The long CDR H3 heavy chain transgenes of the disclosure can be introduced into an animal host for raising antibodies, in particular to intractable antigens such as multipass transmembrane receptors (e.g., GPCRs and ion channels) that may be more readily bound by long CDR H3-containing antibodies.

Accordingly, in one aspect, the disclosure pertains to a transgene construct encoding an immunoglobulin heavy chain variable region comprising:
(a) a plurality of human unrearranged immunoglobulin heavy chain variable segments (VHs), wherein each VH is between 98 and 101 amino acids in length; operatively linked to
(b) a plurality of human D-D fusion segments; operatively linked to
(c) a plurality of human J segments.

In an embodiment, the plurality of human D-D fusion segments comprises at least one naturally-occurring human D-D fusion pair. In an embodiment, the plurality of human D-D fusion segments comprises at least one synthetic human D-D fusion pair. In an embodiment, the plurality of human D-D fusion segments comprises at least one synthetic human D-D fusion pair and at least one naturally-occurring human D-D fusion pair.

In an embodiment, the transgene construct encodes at least five, at least ten, at least fifteen, at least twenty or at least twenty-five VH regions. In an embodiment, the transgene construct encodes human VH regions in 5′ to 3′ orientation as follows: 3-73, 3-72, 2-70D, 1-69, 4-61, 5-51, 3-49, 3-43, 4-39, 4-32, 2-26, 1-24, 3-23, 3-15, 3-9, 1-8, 2-5, 7-4-1, 1-2 and 6-1.

In an embodiment, the transgene construct encodes a synthetic 1-1/2-8 D-D fusion or a synthetic 2-8/1-1 D-D fusion. In an embodiment, the transgene construct encodes a synthetic 1-1/2-8 D-D fusion and a synthetic 2-8/1-1 D-D fusion.

In an embodiment, the transgene construct encodes at least one naturally-occurring D-D fusion pair selected from the group consisting of 2-2/3-3; 5-12/4-17; 5-5/3-22; 6-6/3-10; 6-6/6-19; 5-12/5-5; 6-13/6-19; 2-2/6-13; 5-12/6-19; 6-13/2-21; 5-5/3-10; 2-15/3-22; 5-12/2-15; 2-15/5-24; 6/19-1/26; 2-15/5-5; 5-12/3-22; 2-15/4-17; 5-5/6-13; 6-19/3-22; 2-15/2-21 and 3-3/3-10.

In an embodiment, the transgene construct encodes naturally-occurring D-D fusion pairs 2-2/3-3; 5-12/4-17; 5-5/3-22; 6-6/3-10; 6-6/6-19; 5-12/5-5; 6-13/6-19; 2-2/6-13; 5-12/6-19; 6-13/2-21; 5-5/3-10; 2-15/3-22; 5-12/2-15; 2-15/5-24; 6/19-1/26; 2-15/5-5; 5-12/3-22; 2-15/4-17; 5-5/6-13; 6-19/3-22; 2-15/2-21 and 3-3/3-10.

In an embodiment, the transgene construct further encodes at least one natural D segment.

In an embodiment, the transgene construct encodes D segments and D-D fusion pairs in 5′ to 3′ orientation as follows: 1-1/2-8; 2-2/3-3; 3-3; 5-12/4-17; 5-5/3-22; 6-6/3-10; 6-6/6-19; 2-8; 5-12/5-5; 6-13/6-19; 2-2/6-13; 5-12/6-19; 6-13/2-21; 5-5/3-10; 2-15/3-22; 3-16; 5-12/2-15; 2-15/5-24; 6/19-1/26; 2-15/5-5; 5-12/3-22; 2-15/4-17; 5-5/6-13; 6-19/3-22; 2-15/2-21; 3-3/3-10 and 2-8/1-1.

In an embodiment, the transgene construct encodes J1-J6 segments.

In an embodiment, the transgene construct further encodes a constant region (e.g., a mouse or human Ig constant region) downstream (3′) of the plurality of J segments.

In an embodiment, the transgene construct further comprises lox sites to facilitate cre/lox mediated RMCE (Recombinase Mediated Cassette Exchange). In an embodiment, the transgene construct further comprises Guide Recombination Sequences (GRS) to facilitate CRISPR/CAS-mediated recombination.

In an embodiment, the transgene construct comprises the sequence shown in SEQ ID NO: 1.

In an embodiment, the transgene construct is carried on a bacterial artificial chromosome (BAC).

In another aspect, the disclosure pertains to a transgenic animal comprising a transgene construct of the disclosure. In an embodiment, the transgenic animal is a mouse. In an embodiment, the transgenic mouse further comprises a transgene construct encoding an immunoglobulin light chain such that the mouse expresses antibodies comprising the light chain paired with a heavy chain comprising a long CDR3 region.

In another aspect, the disclosure pertains to a method of generating antibodies to an antigen of interest, the method comprising administering the antigen of interest to the transgenic animal (e.g., mouse) of the disclosure, such that antibodies that bind to the antigen of interest are generated. In an embodiment, the method further comprises isolating an antibody of interest from the animal and determining the heavy chain CDR3 sequence thereof.

### BRIEF DESCRIPTION OF THE DRAWINGS

FIG. 1A-FIG. 1C provide a summary of the sequences of the indicated natural D, synthetic D-D fusions and naturally-occurring D-D fusions, in their “sense” orientation. SEQ ID NOs: 2-109. Polynucleotide sequences for the D segments are in Reading Frame 1; because of the joining mechanism for antibodies, it is possible that Reading Frame 2 or Reading Frame 3 could be utilized. The amino acid sequences include all 3 of the possible frames. Yellow highlighted text=in-frame stop codon; green=native; pink=D-D fusion synthetic; orange=D-D fusion natural.

FIG. 2A-FIG. 2B provide an alignment of the open reading frames of the indicated D segments and D-D fusions; this does not account for additional sequences that could result from junctional variation, or from inversion of the D or D-D segment. FIG. 2A. SEQ ID NOs: 110-156. FIG. 2B. SEQ ID NOs: 157-228.

FIG. 3 is a summary of the transgenic locations of the indicated D segments and D-D fusions; these locations correspond to their naturally occurring positions within the human IGH locus.

FIG. 4 is a schematic diagram illustrating a representative example of a long CDR3 heavy chain construct of the disclosure.

FIG. 5 is a schematic diagram of a representative vector construct of a long CDR3 heavy chain transgene of the disclosure.

FIG. 6 is a schematic diagram of the knock-in scheme used to deliver the long CDR3 heavy chain transgene donor site-specifically to the mouse Ig heavy chain locus.

### DETAILED DESCRIPTION

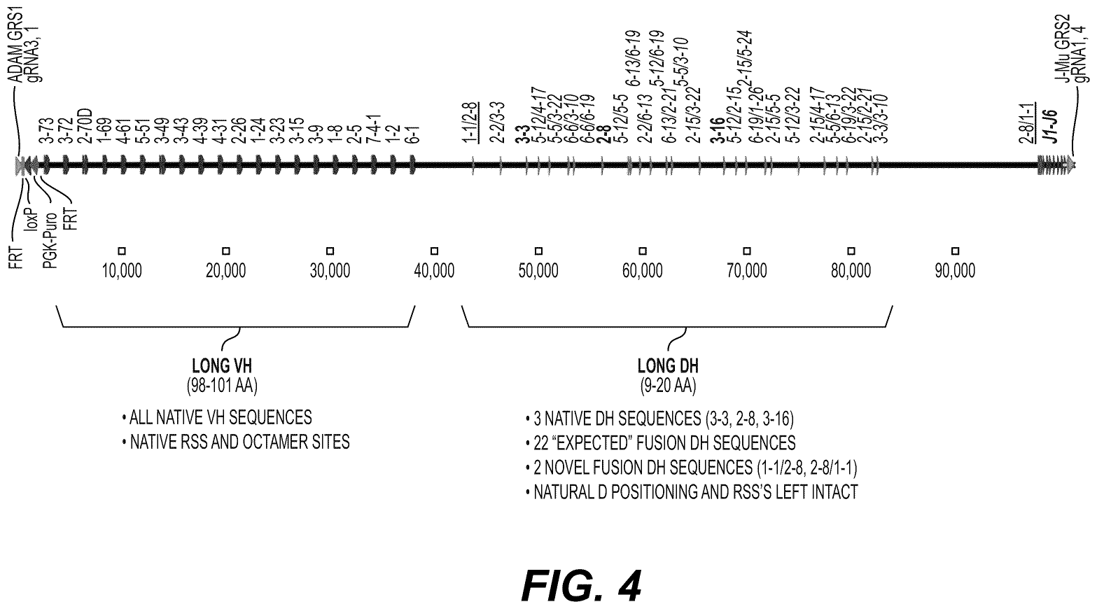

The long CDR3 heavy chain transgene constructs of the disclosure encode a combination of longer than average VH regions and D-D fusions, as illustrated schematically in FIG. 4. Various aspects of the disclosure are described in further detail below.

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook el al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer defined protocols and/or parameters unless otherwise noted.

### I. Construct Design

The design and construction of a long CDR3 human heavy chain transgene is described in detail in Example 1. As illustrated in FIG. 4, the construct encodes a combination of longer than average VH regions and D-D fusions. Accordingly, in one aspect, the disclosure pertains to a transgene construct encoding an immunoglobulin heavy chain variable region comprising:
(a) a plurality of human unrearranged immunoglobulin heavy chain variable segments (VHs), wherein each VH in its unrearranged form is between 98 and 101 amino acids in length; operatively linked to
(b) a plurality of human D-D fusion segments; operatively linked to
(c) a plurality of human J segments.

Non-limiting examples of human VH regions between 98 and 101 amino acids in length include regions: 6-1, 2-5, 2-26, 2-70, 2-70D, 3-15, 3-49, 3-72, 3-73, 3-9, 3-23, 3-43, 4-30-2, 4-30-4, 4-31, 4-32, 4-39, 4-61, 5-51, 7-4-1, 1-2, 1-3, 1-8, 1-18, 1-24, 1-38-4, 1-45, 1-46, 1-58 and 1-69. In an embodiment, the transgene construct encodes at least five, at least ten, at least fifteen, at least twenty or at least twenty-five VH regions, e.g., selected from the aforementioned list.

In an embodiment, the transgene construct encodes human VH regions in 5′ to 3′ orientation as follows: 3-73, 3-72, 2-70D, 1-69, 4-61, 5-51, 3-49, 3-43, 4-39, 4-32, 2-26, 1-24, 3-23, 3-15, 3-9, 1-8, 2-5, 7-4-1, 1-2 and 6-1.

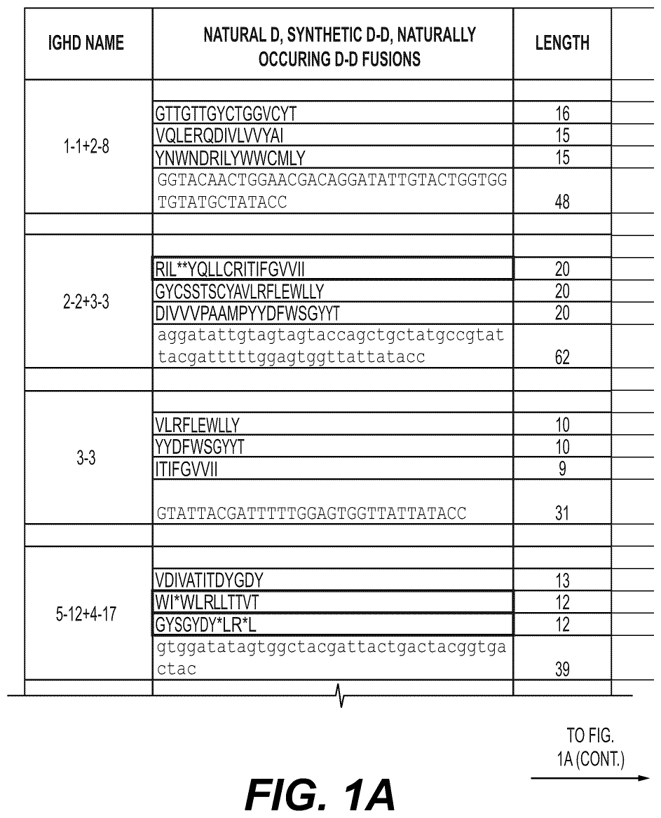

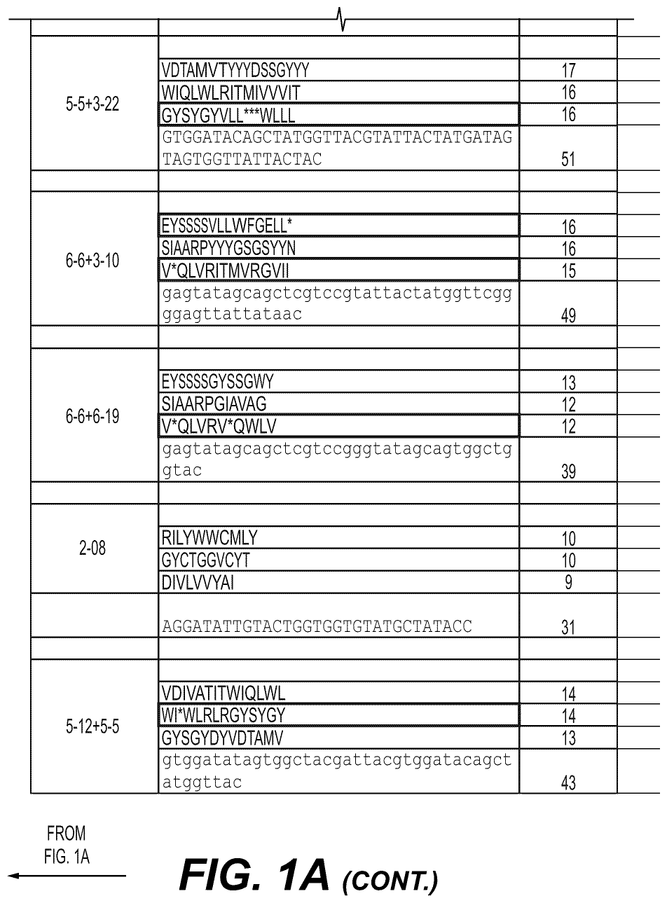

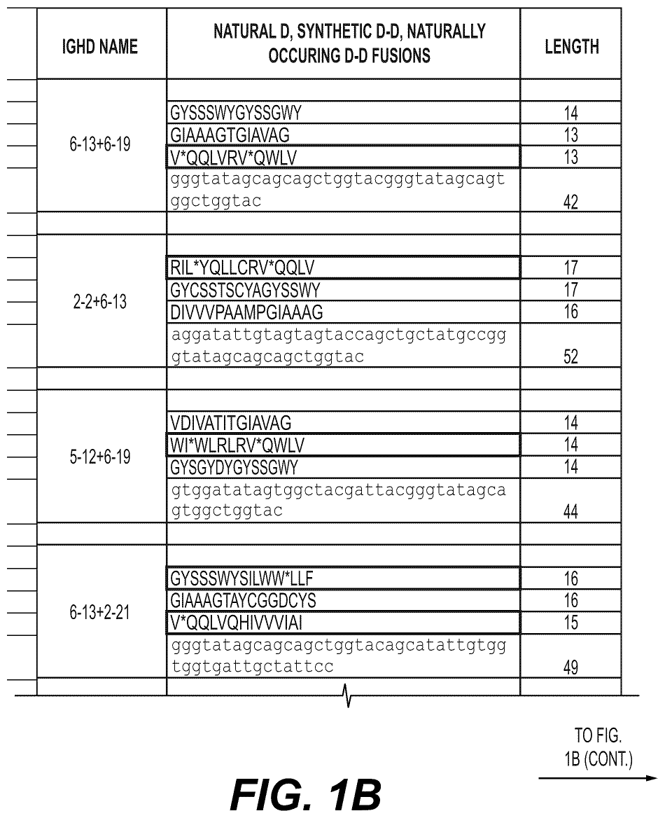

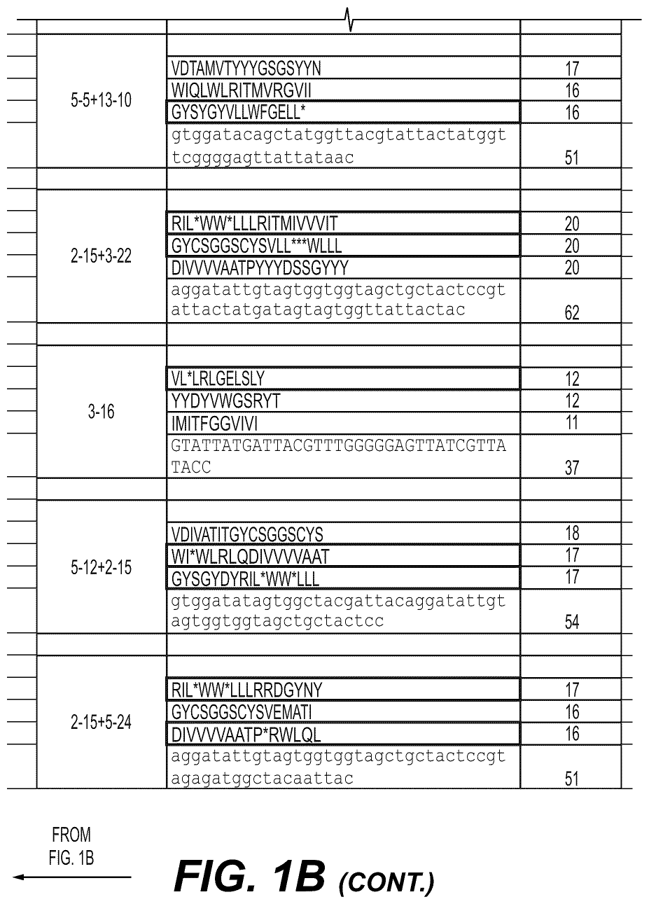

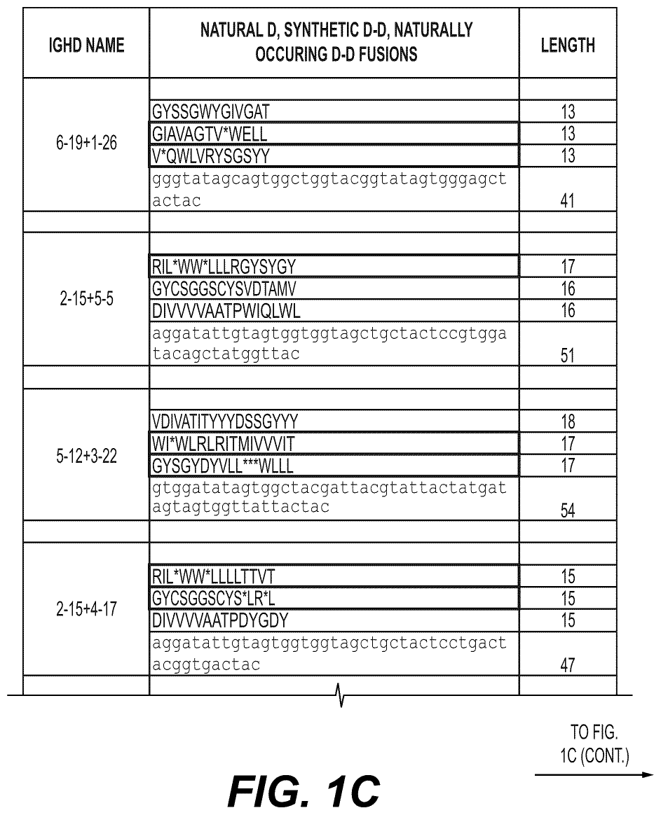

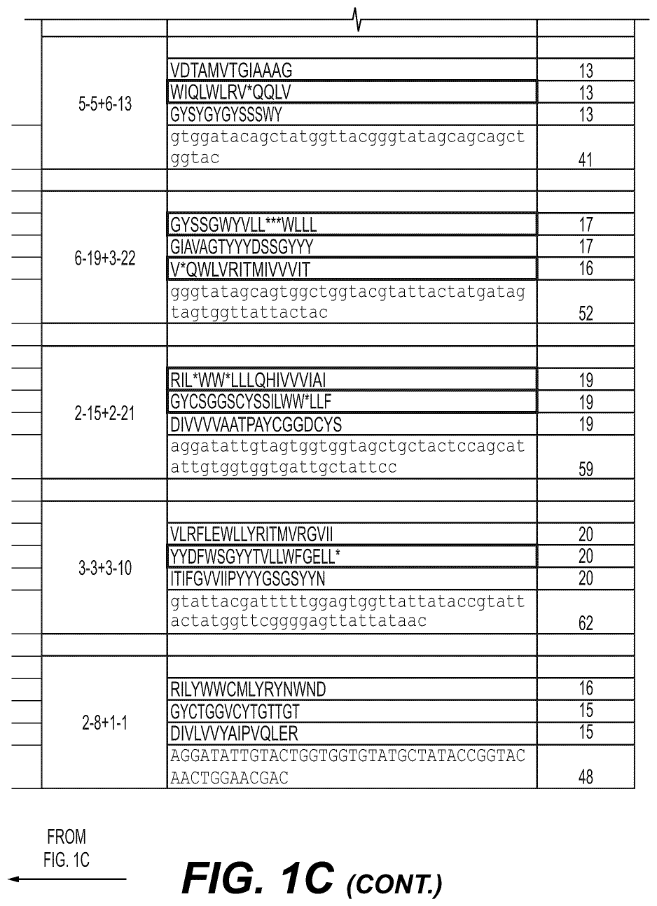

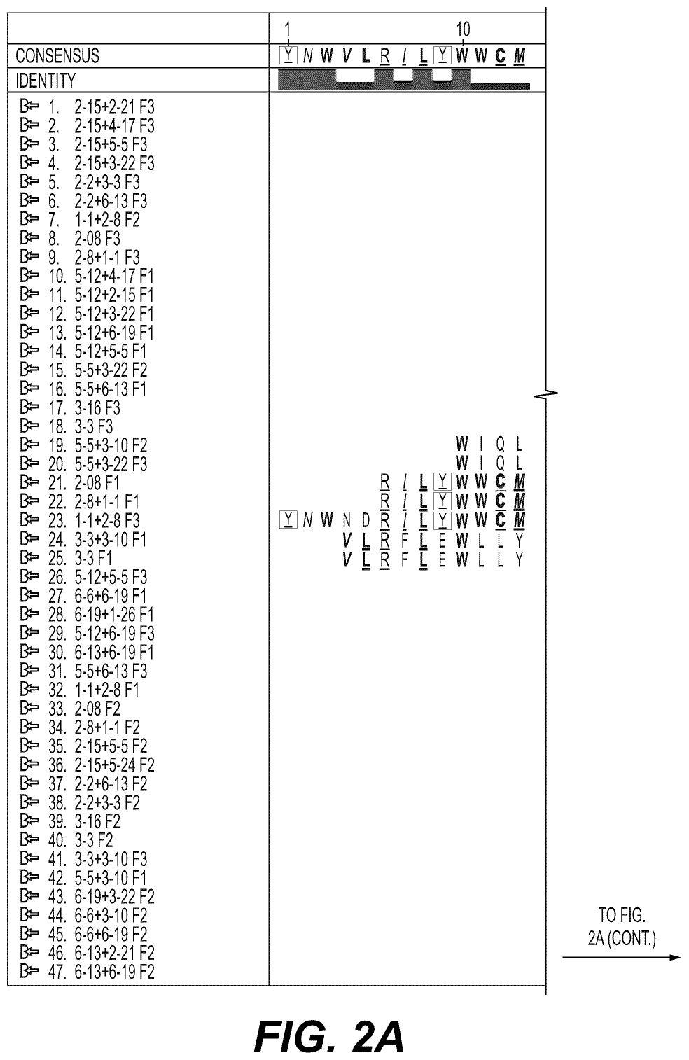

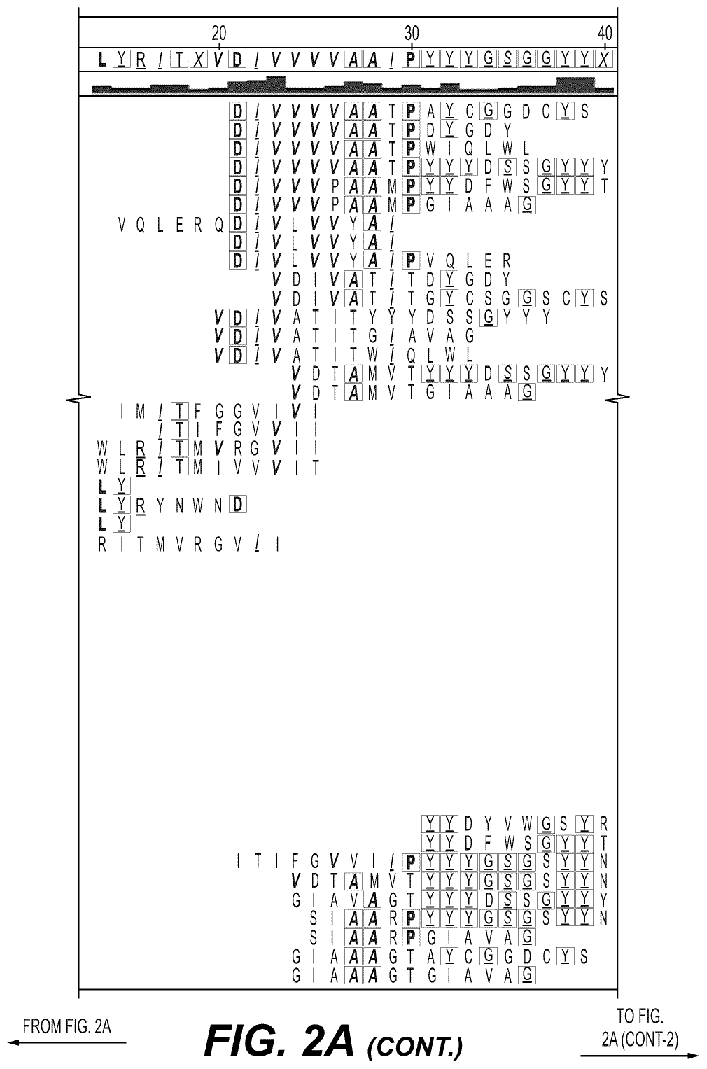

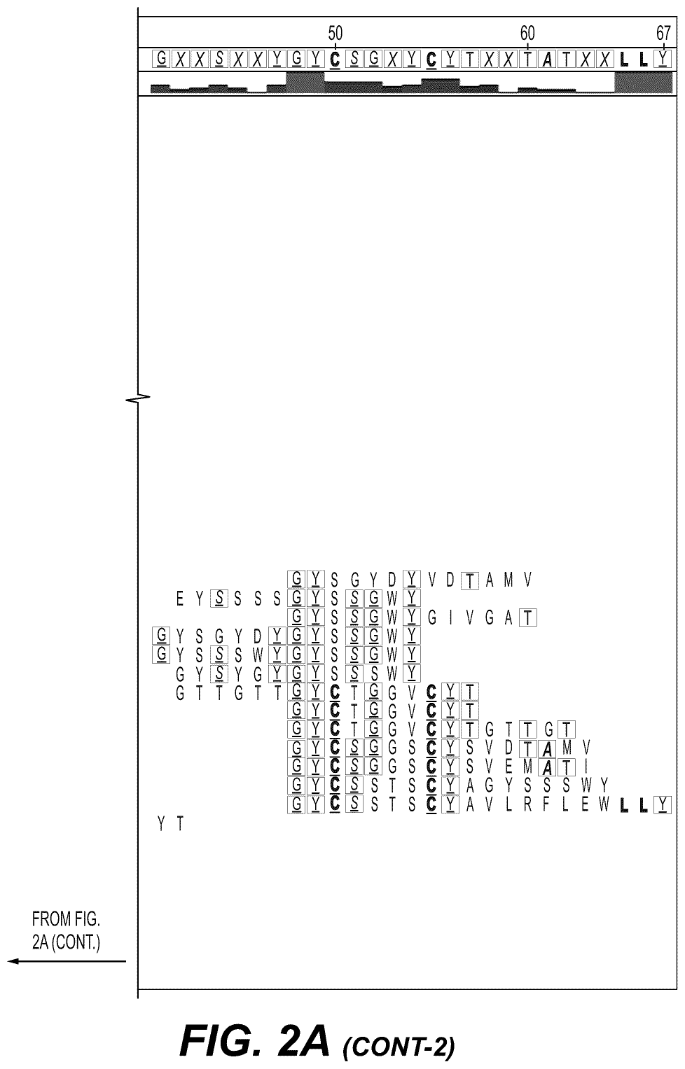

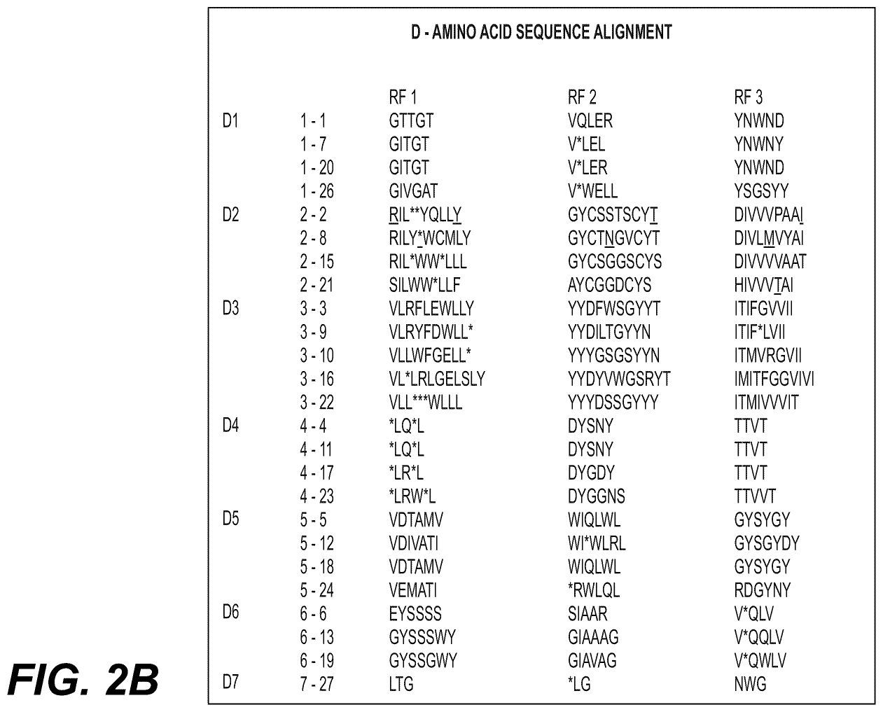

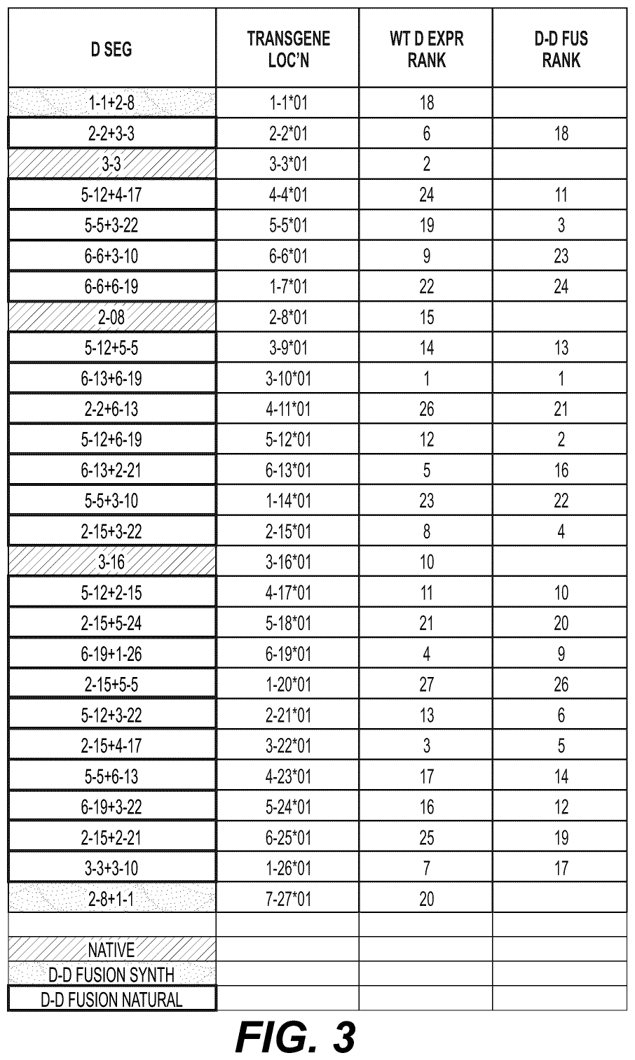

In an embodiment, the plurality of human D-D fusion segments comprises at least one naturally-occurring human D-D fusion pair. In an embodiment, the plurality of human D-D fusion segments comprises at least one synthetic human D-D fusion pair. In an embodiment, the plurality of human D-D fusion segments comprises at least one synthetic human D-D fusion pair and at least one naturally-occurring human D-D fusion pair. Non-limiting examples of naturally-occurring and synthetic human D-D fusion pairs are shown in FIG. 1, FIG. 2 and FIG. 3 (described in further detail in Example 1). Naturally-occurring D-D fusion pairs have also been described in the art, such as in Larimore et al. (2012) J. Immunol. 189:3221-3230; Briney et al. (2012) Immunol. 137:56-64; Yu and Guan (2014) Front. Immunol. 5:250; Safonova and Pevzner (2019) Front. Immunol. 10:987; and Safonova and Pevzner (2020) Genome Res. 30:1547-1558, the entire contents of each of which is specifically incorporated by reference.

In an embodiment, the transgene construct encodes a synthetic 1-1/2-8 D-D fusion or a synthetic 2-8/1-1 D-D fusion. In an embodiment, the transgene construct encodes a synthetic 1-1/2-8 D-D fusion and a synthetic 2-8/1-1 D-D fusion.

In an embodiment, the transgene construct encodes at least one, at least two, at least three, at least four, or at least five, at least six, at least seven, at least eight, at least nine, at least ten or more naturally-occurring D-D fusion pairs selected from the group consisting of 2-2/3-3; 5-12/4-17; 5-5/3-22; 6-6/3-10; 6-6/6-19; 5-12/5-5; 6-13/6-19; 2-2/6-13; 5-12/6-19; 6-13/2-21; 5-5/3-10; 2-15/3-22; 5-12/2-15; 2-15/5-24; 6/19-1/26; 2-15/5-5; 5-12/3-22; 2-15/4-17; 5-5/6-13; 6-19/3-22; 2-15/2-21 and 3-3/3-10.

In an embodiment, the transgene construct encodes naturally-occurring D-D fusion pairs 2-2/3-3; 5-12/4-17; 5-5/3-22; 6-6/3-10; 6-6/6-19; 5-12/5-5; 6-13/6-19; 2-2/6-13; 5-12/6-19; 6-13/2-21; 5-5/3-10; 2-15/3-22; 5-12/2-15; 2-15/5-24; 6/19-1/26; 2-15/5-5; 5-12/3-22; 2-15/4-17; 5-5/6-13; 6-19/3-22; 2-15/2-21 and 3-3/3-10.

In an embodiment, the transgene construct further encodes at least one human natural D segment (i.e., a D segment that is not a D-D fusion). In embodiments, the transgene construct further encodes at least two, at least three, at least four, at least five, at least six, at least seven, at least eight, at least nine, at least ten or more natural D segments. In an embodiment, the natural D segment(s) is longer than average, e.g., encodes a translated sequence of ≥9 a.a., ≥10 a.a., ≥11 a.a., or is 9-12 amino acids in length. Non-limiting examples of human natural D segments that are 9-12 amino acids in length include segments 2-2, 2-8, 2-15, 2-21, 3-3, 3-9, 3-10, 3-16 and 3-22. In an embodiment, the transgene construct comprises natural human D segments 3-3, 2-8 and 3-16.

In an embodiment, the transgene construct encodes D segments and D-D fusion pairs in 5′ to 3′ orientation as follows: 1-1/2-8; 2-2/3-3; 3-3; 5-12/4-17; 5-5/3-22; 6-6/3-10; 6-6/6-19; 2-8; 5-12/5-5; 6-13/6-19; 2-2/6-13; 5-12/6-19; 6-13/2-21; 5-5/3-10; 2-15/3-22; 3-16; 5-12/2-15; 2-15/5-24; 6/19-1/26; 2-15/5-5; 5-12/3-22; 2-15/4-17; 5-5/6-13; 6-19/3-22; 2-15/2-21; 3-3/3-10 and 2-8/1-1.

In an embodiment, the transgene construct encodes human J1-J6 segments.

In an embodiment, the transgene construct further encodes a constant region (e.g., a mouse or human Ig constant region) downstream (3′) of the plurality of J segments.

In an embodiment, the transgene construct further comprises lox sites to facilitate cre/lox mediated RMCE (Recombinase Mediated Cassette Exchange). In an embodiment, the transgene construct further comprises Guide Recombination Sequences (GRS) to facilitate CRISPR/CAS-mediated recombination.

The nucleotide sequence of the transgene construct can be further optimized for intended purposes. For example, the construct can be altered for codon optimization (e.g., to increase expression of the encoded regions). Additionally or alternatively, the construct can be altered to avoid excessive somatic hypermutation (SHM), for example by analyzing hypermutable regions in CDR1, CDR2 and or CDR3 of the heavy chain variable regions and eliminating sequence(s) that could enhance SHM. Approaches for codon optimization and SHM reduction are well established in the art.

The transgene construct can further comprise sequences that allow for targeted insertion of the transgene into a specific locus, e.g., an endogenous mouse heavy chain locus. Knock-in technology for replacing an endogenous locus with a targeted transgene is well established in the art. In a preferred embodiment, the transgene construct comprises recombination sequences (Guide Recombination Sequences, or GRS) allowing for the transgene to be knocked-in to the endogenous mouse heavy chain locus.

In an embodiment, the long CDR3 heavy chain construct comprises the nucleotide sequence shown in SEQ ID NO: 1.

### II. Construct Preparation

The transgene constructs of the disclosure can be prepared using standard recombinant DNA techniques. Cloning vectors containing polylinkers are useful as starting vectors for insertion of DNA fragments of interest. Suitable cloning vectors are well established in the art. Moreover, plasmids or other vectors (e.g., YACs) carrying human unrearranged light chain immunoglobulin sequences have been described in the art (see e.g., U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; and U.S. Pat. Nos. 5,939,598; 6,075,181; 6,114,598; 6,150,584 and 6,162,963, all to Kucherlapati et al.) and can be used as a source of heavy chain V. D and J region sequences. Alternatively, desired sequences can be synthesized by standard methods. The appropriate DNA fragments are then operatively linked through ligation into a cloning vector, followed by characterization of the vector (e.g., by restriction fragment analysis or sequencing or the like) to ensure proper arrangement of the fragments.

In an embodiment, the transgene construct is carried on a bacterial artificial chromosome (BAC). BAC technology for carrying Ig transgenes is well-established in the art.

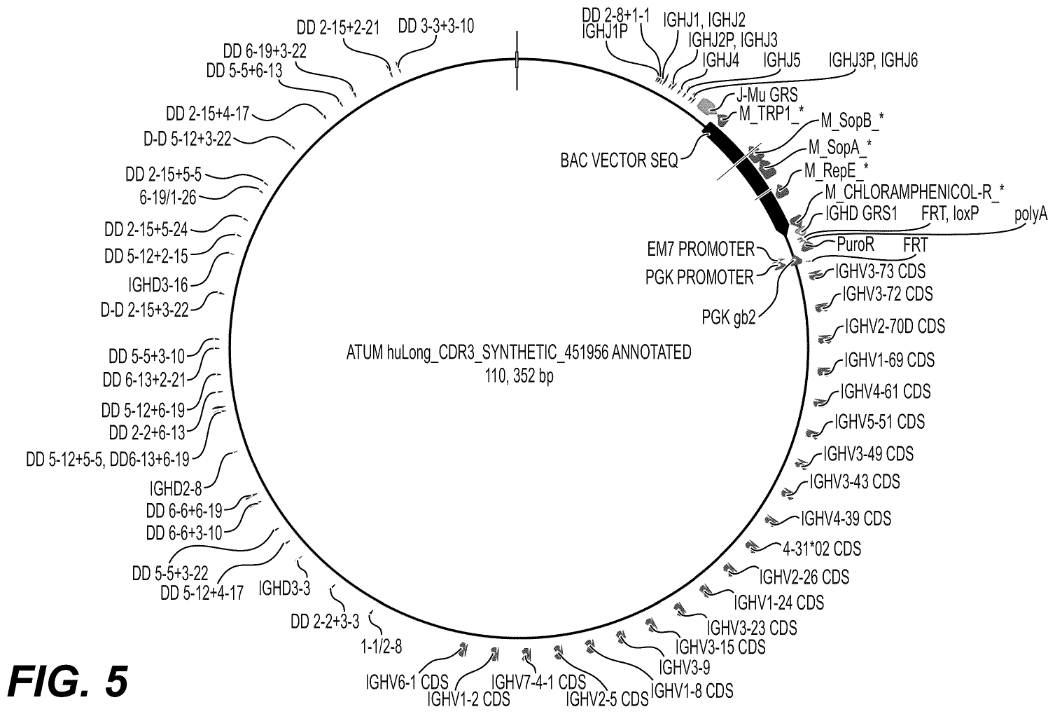

A non-limiting example of a long CDR3 heavy chain vector of the disclosure is illustrated schematically in FIG. 5.

To prepare the transgene construct for microinjection or other technique for transgenesis, the transgene construct can be isolated from the vector in which it is carried by cleavage with appropriate restriction enzymes to release the transgene construct fragment. The fragment can be isolated using standard techniques, such as by pulse field gel electrophoresis on an agarose gel, followed by isolation of the fragment from the agarose gel, such as by [beta]-agarase digestion or by electroelution. For example, the agarose gel slice containing the transgene construct fragment can be excised from the gel and the agarose can be digested with [beta]-agarase (e.g., from Takara), using standard methodology. Alternatively, preparation of the transgene for knock-in purposes may be carried out by standard BAC or plasmid purification techniques, isolating the closed circular form for direct transfection or introduction into recipient mouse cells or embryos.

### III. Preparation of Transgenic Animals

Another aspect of the disclosure pertains to a transgenic non-human host animal that comprises a transgene construct of the disclosure (i.e., the transgene construct is integrated into the genome of the host animal), such that the animal expresses an immune repertoire that comprises antibodies using heavy chains comprising long CDR3 regions. The transgenic non-human host animals of the disclosure are prepared using standard methods known in the art for introducing exogenous nucleic acid into the genome of a non-human animal. In a preferred embodiment, the transgene construct is inserted into the genome of the host animal using knock-in technology to replace all or a part of an endogenous heavy chain locus (e.g., an endogenous mouse heavy chain locus) with the transgene (e.g., a human heavy chain transgene). Alternatively, the transgene construct can be introduced into the genome of the host animal by, for example, pronuclear microinjection for random genomic insertion, or transfection into mouse embryonic stem (mES) cells.

For knock-in approaches, typically loxP flanking sites are included in the construct such that these sites facilitate recombination between host loxP flanking sites and loxP flanking sites in the transgene donor upon expression of Cre recombinase. Recombination is performed in embryonic stem cells (e.g., mouse embryonic stem cells) and then embryonic stem cells with the modification of interest are implanted into a viable blastocyst, which then grows into a mature chimeric animal (e.g., mouse) with some cells having the original blastocyst cell genetic information and other cells having the modifications introduced to the embryonic stem cells. Subsequent offspring of the chimeric animal will then have the gene knock-in. Knock-in technology is summarized in, for example, Manis (2007) New Engl. J. Med. 357:2426-2429.

As an alternative knock-in approach, the construct can include flanking Guide Recombination Sequences (GRS) to facilitate CRISPR/CAS-mediated recombination. These are 500-1500 bp sequences which flank the transgene insert, and have specific homology to endogenous mouse sequences that adjoin specific CRISPR/CAS cleavage sites in the mouse genome. Appending the same CRISPR/CAS cleavage sites to the ends of the GRS flanking sequences allows for CRISPR/CAS mediated digestion to simultaneously cleave the endogenous mouse genome as well as the circular BAC transgene donor. In this manner, the cleaved ends of the mouse CRISPR/CAS sites are available for homologous recombination mediated repair via the similarly cleaved and linearized transgene donor insert, resulting in a site-specific knock-in.

In a preferred embodiment, the transgene construct is inserted into the genome of a mouse using knock-in technology to replace all or part of the endogenous heavy chain locus. In a preferred embodiment, the transgene construct is a human heavy chain construct that is inserted into the endogenous mouse heavy chain locus by homologous recombination, thereby deleting at least some portion of the mouse VH, DH, and JH and CH sequences. In another embodiment, a heavy chain transgene that lacks a constant region is inserted into the endogenous heavy chain locus such that at least some portion of the VH. DH and JH sequences is deleted but the CH sequences are left intact and operatively linked to the functional heavy chain variable region of the transgene, thereby producing chimeric antibodies in the mice (which can be reverse engineered to be fully human).

Another method for preparing a transgenic non-human animal, in particular a transgenic mouse, is that of pronuclear microinjection. This technology is well established in the art (see e.g., Wagner. T. E. et al. (1981) Proc. Natl. Acad. Sci. USA 78:6376-6380; U.S. Pat. No. 4,873,191 by Wagner and Hoppe). In general, the method involves introducing exogenous genetic material into the pronucleus of a mammalian zygote (e.g., mouse zygote) by microinjection to obtain a genetically transformed zygote and then transplanting the genetically transformed zygote into a pseudopregnant female animal. The embryo is then allowed to develop to term and the genome of the resultant offspring is analyzed for the presence of the transgenic material.

Southern blot analysis. PCR, or other such technique for analyzing genomic DNA is used to detect the presence of a unique nucleic acid fragment that would not be present in the non-transgenic animal but would be present in the transgenic animal. Selective breeding of transgenic offspring allows for homozygosity of the transgene to be achieved.

If a long CDR3 heavy chain transgene is inserted into the genome randomly, it is preferable to also disable the endogenous heavy chain locus to achieve a limited repertoire of heavy chain options in the animal (e.g., mouse). The endogenous heavy chain locus can be engineered to render it non-operational by standard knock-out technology, for example by deleting all or a portion of the endogenous heavy chain V, D, J and C regions such that they are non-functional.

Although the preferred embodiment of the disclosure comprises transgenic mice, the invention encompasses other non-human host animals, including but not limited to rats, rabbits, pigs, goats, sheep, cows, and chickens. Techniques for creating transgenic animals of each of these species have been described in the art. For example, preparation of transgenic rats is described in Tesson, L. et al. (2005) Transgenic Res. 14:531-546, including by techniques such as DNA microinjection, lentiviral vector mediated DNA transfer into early embryos and sperm-mediated transgenesis. Methods of transgenesis in rats are also described in Mullin, L. J. et al. (2002) Methods Mol. Biol. 180:255-270. Preparation of transgenic rabbits is described in, for example, Fan, J. et al. (1999) Pathol. Int. 49:583-594; Fan, J. and Watanabe, T. (2000) J. Atheroscler. Thromb. 7:26-32; Bosze, Z. et al (2003) Transgenic Res. 12:541-553. Preparation of transgenic pigs is described in, for example. Zhou. CY. et al. (2002) Xenotransplantation 9:183-190; Vodicka, P. et al. (2005) Ann. N. Y. Acad. Sci. 1049:161-171.

Alternative transgenesis techniques to pronuclear microinjection in pigs include adenovirus mediated introduction of DNA into pig sperm (see e.g., Farre, L. et al. (1999) Mol. Reprod. Dev. 53:149-158) and linker-based sperm-mediated gene transfer (Chang, K. et al. (2002) BMC Biotechnol. 2:5). Preparation of transgenic goats is described in, for example. Ebert, K. M. et al. (1991) Biotechnology (NY) 9:835-838; Baldassarre. H. et al. (2004) Reprod. Fertil. Dev. 16:465-470. Somatic cell nuclear transfer in goats is described in, for example. Behboodi. E. et al. (2004) Transgenic Res. 11:215-224. Preparation of transgenic sheep is described in, for example. Ward, K. A. and Brown. B. W. (1998) Reprod. Fertil. Dev. 10:659-665. Preparation of transgenic cows is described in, for example, Donovan, D. M. et al. (2005) Transgenic Res. 14:563-567. Gene transfection of donor cells for nuclear transfer of bovine embryos is described in, for example, Lee S. L. et al. (2005) Mol. Reprod. Dev. 72:191-200. Preparation of transgenic domestic farm animals is also reviewed in Niemann, H. et al. (2005) Rev. Sci. Tech. 24:285-298. Preparation of transgenic chickens is described in, for example, Pain, B. et al. (1999) Cells Tissues Organs 165:212-219; Lillico, S. G. et al. (2005) Drug Discov. Today 10:191-196; and Ishii, Y. et al. (2004) Dev. Dyn. 229:630-642.

An animal of the disclosure (e.g., mouse) carrying a long CDR3 heavy chain transgene construct can be cross-bred with an animal (e.g., mouse) that carries an immunoglobulin light chain transgene to thereby produce an animal (e.g., mouse) that expresses antibodies comprising the light chain paired with a heavy chain comprising long CDR3 regions. Immunoglobulin light chain transgenic animals (e.g., mice) are well-established in the art.

### IV. Use of Transgenic Animals

The transgenic animals of the disclosure are useful for generating antibodies to a wide variety of antigens of interest. For animals carrying only the long CDR3 heavy chain transgene and an endogenous light chain locus, the animal will produce chimeric light chain/heavy chain antibodies that, if desired, can be reverse engineered to pair the long CDR3 heavy chain with a light chain of the same species. Alternatively, for animals (e.g., mice) carrying both a long CDR3 heavy chain Ig transgene (e.g., human) and a light chain Ig transgene (e.g., human), fully heterologous antibodies (e.g., fully human antibodies) can be prepared in the host transgenic animal. For animals carrying chimeric Ig transgene loci (e.g., human variable regions appended to mouse constant regions, of the heavy and/or light chains), combinations of the humanized loci may functionally pair with other humanized loci, or alternatively with their wild-type mouse counterparts.

Accordingly, in another aspect, the disclosure pertains to a method of generating antibodies to an antigen of interest, the method comprising administering the antigen of interest to a transgenic animal of the disclosure. In an embodiment, the animal is a transgenic mouse and the antigen is administered to the mouse such that antibodies that bind to the antigen of interest are generated in the mouse. In an embodiment, the animal is a transgenic mouse carrying both a human Ig long CDR3 heavy chain transgene and a human Ig light chain transgene and the antigen is administered to the mouse such that human or human-mouse chimeric antibodies that bind to the antigen of interest are generated in the mouse. In an embodiment, the antigen is a GPCR or an ion channel protein. In an embodiment, the method can further comprise isolating an antibody of interest from the host animal (e.g., mouse) and determining the heavy chain CDR3 sequence of the antibody.

Transgenic animals can be immunized with an antigen(s) of interest by standard methodologies known in the art and antibodies generated in the animals can be isolated and characterized also be standard established methods. Polyclonal antibodies can be directly isolated form the host animal and monoclonal antibodies can be prepared by standard methods, such as hybridoma technology. Procedures for making monoclonal antibodies using hybridomas are well established in the art (see, e.g., U.S. Pat. No. 4,977,081, PCT Publication WO 97/16537, and European Patent No. 491057B1, the disclosures of which are incorporated herein by reference). Alternatively, in vitro production of monoclonal antibodies from cloned cDNA molecules is also established in the art (see e.g., Andris-Widhopf et al. (2000) J. Immunol. Methods 242:159; and Burton (1995) Immunotechnology 1:87, the disclosures of which are incorporated herein by reference). B cell clones from the immunized transgenic animals can be isolated and cDNAs encoding the antibodies can be isolated and cloned by standard molecular biology techniques into expression vectors. Further recombinant engineering of the cloned Ig cDNAs is also possible and well established in the art.

### V. Definitions

As used herein, the term “D-D fusion segment” is intended to refer to the direct joining of two different immunoglobulin heavy chain D region nucleic acid sequences. For example, a D-D fusion segment of a 5-12 D segment and a 4-17 D segment is referred to herein as a 5-12+4-17 D-D fusion segment (or simply “D-D fusion”). A “naturally-occurring D-D fusion pair” refers to a D-D fusion whose two constituent D segments have been observed in nature arising from a V(D-D)J recombination event. Such naturally-occurring D-D fusion pairs have been described in the art, such as in Larimore et al. (2012) J. Immunol. 189:3221-3230; Briney et al. (2012) Immunol. 137:56-64; Yu and Guan (2014) Front. Immunol. 5:250; Safonova and Pevzner (2019) Front. Immunol. 10:987; and Safonova and Pevzner (2020) Genome Res. 30:1547-1558. A “synthetic D-D fusion pair” refers to a D-D fusion that has not been observed in nature arising from a V(D-D)J recombination event. Regardless of whether a D-D fusion pair is “naturally-occurring” (i.e., has been observed in nature) or “synthetic” (i.e., has not been observed in nature), the D-D fusion segment(s) used in the transgenes of the disclosure can be engineered ex vivo by genetically linking the sequences of the two D segments by standard methods.

As used herein, “long CDR3”, “long H CDR3”, “long CDR H3” or “long HCDR3” refers to a heavy chain CDR3 region that is longer than the typical or average length of human heavy chain CDR3 regions, e.g., typically longer than 8-16 amino acids in length. A transgene of the disclosure, after VDJ recombination, generates heavy chain variable regions having CDR3s of various lengths and is considered to be a transgene encoding long CDR3s when a significant portion, e.g., at least 25%, at least 30%, at least 35%, at least 40%, at least 45% or at least 50% or more of the generated HCDR3s are longer than the typical or average length of HCDR3s.

As used herein, the term “operatively linked” is intended to describe the configuration of a nucleic acid sequence that is placed into a functional relationship with another nucleic acid sequence. For example, a promoter or enhancer is operatively linked to a coding sequence if it affects the transcription of the sequence. With respect to the joining of two protein coding regions, operatively linked means that the nucleic acid sequences being linked are contiguous and in reading frame. For splice donor/acceptor and RSS sequences, operatively linked means that the sequences are capable of effecting their functional purposes.

As used herein, the term “transgene” refers to a gene that is introduced as an exogenous source to a site within a host genome (e.g., mouse heavy chain Ig locus).

As used herein, the term “transgene construct” refers to a nucleic acid preparation suitable for introduction into the genome of a host animal.

As used herein, the term “transgenic mouse” refers to a mouse comprising cells having a transgene, as defined herein. The transgene may be present in all or some cells of the mouse.

As used herein, the term “unrearranged” with respect to an immunoglobulin V segment refers to an immunoglobulin V segment that is in its germline configuration wherein the V segment is not recombined so as to be immediately adjacent to a D or J segment.

The present invention is further illustrated by the following examples, which should not be construed as further limiting. The contents of figures and all references, patents and published patent applications cited throughout this application are expressly incorporated herein by reference.

### EXAMPLES

### Example 1: Preparation of Human Long CDR3 Heavy Chain Constructs

In this example, the preparation of a human immunoglobulin heavy chain transgene construct is described, wherein the CDR3 domain of the construct is on average larger (longer) than that found in normal naïve human B cell heavy chain CDR3s. The rationale for this approach is that certain antigens can have functionally important epitopes that are inaccessible or rare to find amongst normal-length heavy chain CDR3 domain antibodies. The likelihood of an antibody interacting with such epitopes is increased by the use of a set of CDR3 domains that are longer (larger) than average.

### Conceptual Framework

Long CDR3s have been reported in the art to have potential for difficult membrane targets. In particular, GPCRs, ion channels and other membrane targets with clefts or limited surface access may benefit from long CDR3 mAbs to better access functional sites (see e.g., Douthwaite et al. (2015) MAbs 7:152-166; Corti et al. (2013) Annu. Rev. Immunol. 31:705-742; Wei et al. (2011) PLOS One 6:e28309; Wang et al. (2016) Proc. Natl. Acad. Sci. USA 113:11501-11506; Ishchenko et al. (2017) Proc. Natl. Acad. Sci. USA 114:8223-8228). Camelid and cow antibodies with long CDR3 domains have been exploited for this purpose (see e.g., Wang et al. (2013) Cell 153:1379-1393; de los Rios et al. (2015) Curr. Opin. Struct. Biol. 33:27-41; Sok et al. (2017) Nature 548:108-111).

### Construct Design and Transgene Preparation

Using mechanisms for CDR3 lengthening, transgenes were designed that skew expression to longer domains. First, the longest VH and D segments were identified and transgenes were designed to selectively use some of the longer VH segments and longer D segments. Additionally, D-D fusions using some of the shorter D segments were included so as to retain some of the naturally occurring D amino acid sequences while also keeping to the longer-length CDR3s.

The choices for VH segments were based on length and uniqueness. VH segments of 98-101 amino acids in length were selected for possible inclusion in the constructs.

Natural long D segments to be used in wild-type configuration were also selected for possible inclusion in the constructs, examples of which include: 2-02, 2-08, 2-15, 2-21, 3-03, 3-09, 3-10, 3-16 and 3-22.

Regarding D-D fusions, the natural formation of D-D fusions during recombination can be explained by the presence of so-called “cryptic nonamers”, which are nonamer sequences (normally part of the RSS motifs enabling VDJ recombination) that are out of their normal context but still able to support recombination (Safonova and Pevzner (2020) Genome Res. 30:1547-1558). Some D segments have a higher probability of the non-canonical nonamers in spacing that would allow for recombination, examples of which include: 2-02, 2-15, 3-03, 3-09, 3-10, 3-16, 3-22, 6-06, 6-19 and 6-25. Also, empirically it seems that the appearance of D-D fusions generally follows their germline order (5′ D fusing to a 3′ D), such that 3′ D segments less commonly fuse to 5′ segments.

The sequences of exemplary natural D segments, synthetic D-D fusions and naturally-occurring D-D fusions are shown in FIG. 1. In all, the 27 different D or D-D components shown in FIG. 1 comprise 47 different ORFs, which are aligned in FIG. 2, wherein F1, F2 and F3 correspond to the different reading frames. These illustrate the overall structure and sequence of the elements that can be included in the transgene. It should also be noted, however, that recombined D segments as part of a V-D-J recombinational sequence, can be found in an inverted orientation (for example, see Meck, et al (1989) J Exp Med. 170: 39-57). Thus, for the purposes of the disclosure, all of the D and D-D segments included herein are intended to be encompassed both in their forward orientation as well as their inverse orientation.

The strategy for placement of the various D and D-D fusions into the construct was (in order of priority): (i) place a D-D fusion in a position where one of the D-D pair normally resides within the D domain, if possible; (ii) if its own locus is not possible, try to match expression of the naturally-occurring D segment there to the expression level of one of the D segments present in the D-D fusion; and (iii) find remaining slots where possible. The D segments and D-D fusions (and their associated nucleotide and amino acid sequences) were placed genomically into the transgene construct in the endogenous D locations shown in FIG. 3. The last two columns of FIG. 3 compare the endogenous D segment expression rank (as reported in the IMGT database; https://www.imgt.org/genefrequency/query) to the expression level of D-D fusion pairs as reported in Safonova and Pevzner (2019) Front. Immunol. 10:987. While there is some correlation between the two, the primary objective of putting the D-D fusion cassettes into their “native” locations means that rank order cannot be matched for both parameters.

A schematic diagram of a representative long CDR3 construct is shown in FIG. 4 and a representative nucleotide sequence of the construct is shown in SEQ ID NO: 1.

This construct comprises the following native VH segments in 5′ to 3′ order: 3-73, 3-72, 2-70D, 1-69, 4-61, 5-51, 3-49, 3-43, 4-39, 4-32, 2-26, 1-24, 3-23, 3-15, 3-9, 1-8, 2-5, 7-4-1, 1-2 and 6-1. The VH segments are each 98-101 amino acids in length and use native RSS and octamer sites.

This construct also comprises three native DH sequences (3-3, 2-8 and 3-16), two novel synthetic D-D fusions (1-1/2-8 and 2-8/1-1) and 22 naturally-occurring D-D fusion pairs (2-2/3-3; 5-12/4-17; 5-5/3-22; 6-6/3-10; 6-6/6-19; 5-12/5-5; 6-13/6-19; 2-2/6-13; 5-12/6-19; 6-13/2-21; 5-5/3-10; 2-15/3-22; 5-12/2-15; 2-15/5-24; 6/19-1/26; 2-15/5-5; 5-12/3-22; 2-15/4-17; 5-5/6-13; 6-19/3-22; 2-15/2-21 and 3-3/3-10). The long D regions are 9-20 amino acids in length, they retain natural positioning as much as possible and the RSS sites are left intact. The 5′ to 3′ order of the D segments and D-D fusions in the construct is as follows: 1-1/2-8; 2-2/3-3; 3-3; 5-12/4-17; 5-5/3-22; 6-6/3-10; 6-6/6-19; 2-8; 5-12/5-5; 6-13/6-19; 2-2/6-13; 5-12/6-19; 6-13/2-21; 5-5/3-10; 2-15/3-22; 3-16; 5-12/2-15; 2-15/5-24; 6/19-1/26; 2-15/5-5; 5-12/3-22; 2-15/4-17; 5-5/6-13; 6-19/3-22; 2-15/2-21; 3-3/3-10 and 2-8/1-1.

The D-D fusion segments inserted into the construct used the DNA sequences shown in FIG. 1. These sequences are pasted into and replaced the native D segment coding sequence, the amino acid sequence of which is shown in FIG. 2. For this replacement exercise, the coding sequences were placed directly between the flanking RSS sites on either side of the CDS for that segment. No changes in RSS sequences were made.

LoxP flanks the insert at the 5′ end. There is also an ADAM/IGHD GRS1 site at the 5′ end and a J-Mu GRS site at the 3′ end to facilitate donor transgene and mouse host genome recombination. The success of GRS donor delivery for knockins has been validated to a level where this is the preferred mechanism for site-specific donor transgene delivery. The PGK-Puro cassette is flanked by FRT sites, which allows the cassette to be removed after GRS donor gene delivery.

The segment from the end of the “Long DH” as shown in FIG. 4 through the J segments (J1-J6) is germline configuration, unaltered with respect to human GenBank reference sequences, except for the last D-D fusion (2-8/1-1), which is placed in the region where the endogenous 7-27 D allele normally resides, within J sequences.

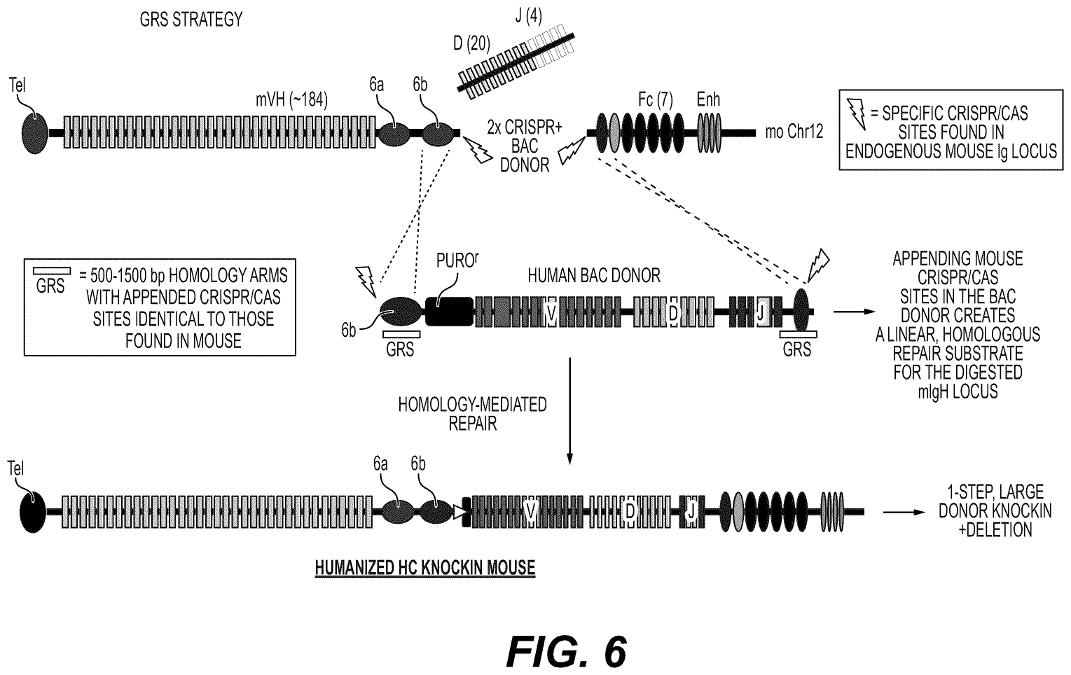

The long CDR3 bacterial artificial chromosome (BAC) transgene donor, as illustrated in FIG. 4 and FIG. 5, is used for CRISPR/Cas-mediated one-step deletion/delivery of the transgene via the GRS sequences. FIG. 6 provides a graphical illustration of the GRS knockin strategy and the resultant knock-in allele when the BAC donor is used in concert with mouse Ig-specific CRISPR/CAS reagents in mouse ES cells capable of mediating a homologous recombination event. Mice containing the long CDR3 heavy chain transgene can be bred to homozygosity. Mice can be cross-bred with a light chain transgenic mouse to create a HC/LC transgenic mouse expressing the long CDR3 transgene repertoire.

### Summary of Sequence Listing

| # | SEQUENCE |
|---|---|
| 1 | SEQ ID NO: 1 |
| | GCGGCCGCACCATCATGATATCCCACAAGTATGGAAGATACATAAGTAGATGGCCAGACTA |
| | TACAGGAAAGAAGCAAAACTTGGGCTAACAGAAGAGCAAACACAGAGCTCAGACAGAACTA |
| | CCTGGCAATGCGACTGGGCACACTGAAAGCACTGGGCATCAGCACTGAGCCCCAAATATGC |
| | ACTCAGGATCCTCTGCATAATAATGTGACATAACAGGAAGGTTAGAACAGGCCAAAAGAGG |
| | AAACAGAACAAATGCCCCCAACCAAAGAAGTATAAACAAATTGGGAAGAGTAAAGAAGGAT |
| | TGTAAGGATTGAGTACCACACAGAACATGCTCTTAATGGCCTCAATGCTGAAGCTAGGAAG |
| | AACTAAGTTAAAAGAAACATGTTCAACGGGATTCCCTGTCACTGGACTTCACAACAAGCAA |
| | AATTCAATCTTTCTGTTAAGGAGATGAGAAGAGAATATCTGAACCTTGTGTTGACAGTGCC |
| | CCACCCCGACTGTCAGGCTGTGGGAAATGCCAGAGCAATCACTAGGAACACACAAGGATGA |
| | GGGAGACGAGGGTTAGGACACAATTCTCGAGAAGTTCCTATTCTCTAGAAAGTATAGGAAC |
| | TTCCTGATCACTCGATCTCGTGCTACGATAACTTCGTATAGCATACATTATACGAAGTTAT |
| | CGCGCCGCACACAAAAACCAACACACAGATCATGAAAATAAAGCTCTTTTATTGGTACCGA |
| | ATTCGCCAGGGAGCTCTCAGACGTCGCTTGGTCGGTCTTTATTCGAACCCCAGAGTCCCGC |
| | TCAGGCACCGGGCTTGCGGGTCATGCACCAGGTGCGCGGTCCTTCGGGCACCTCGACGTCG |
| | GCGGTGACGGTGAAGCCGAGCCGCTCGTAGAAGGGGAGGTTGCGGGGCGCGGAGGTCTCCA |
| | GGAAGGCGGGCACCCCGGCGCGCTCGGCCGCCTCCACTCCGGGGAGCACGACGGCGCTGCC |
| | CAGACCCTTGCCCTGGTGGTCGGGCGAGACGCCGACGGTGGCCAGGAACCACGCGGGCTCC |
| | TTGGGCCGGTGCGGCGCCAGGAGGCCTTCCATCTGTTGCTGCGCGGCCAGCCGGGAACCGC |
| | TCAACTCGGCCATGCGCGGGCCGATCTCGGCGAACACCGCCCCCGCTTCGACGCTCTCCGG |
| | CGTGGTCCAGACCGCCACCGCGGCGCCGTCGTCCGCGACCCACACCTTGCCGATGTCGAGC |
| | CCGACGCGCGTGAGGAAGAGTTCTTGCAGCTCGGTGACCCGCTCGATGTGGCGGTCCGGGT |
| | CGACGGTGTGGCGCGTGGCGGGGTAGTCGGCGAACGCGGCGGCGAGGGTGCGTACGGCCCG |
| | GGGGACGTCGTCGCGGGTGGCGAGGCGCACCGTGGGCTTGTACTCGGTCATGGTTTAGTTC |
| | CTCACCTTGTCGTATTATACTATGCCGATATACTATGCCGATGATTAATTGTCAACACGTG |
| | CTGCTGCAGGTCGAAAGGCCCGGAGATGAGGAAGAGGAGAACAGCGCGGCAGACGTGCGCT |
| | TTTGAAGCGTGCAGAATGCCGGGCCTCCGGAGGACCTTCGGGCGCCCGCCCCGCCCCTGAG |
| | CCCGCCCCTGAGCCCGCCCCCGGACCCACCCCTTCCCAGCCTCTGAGCCCAGAAAGCGAAG |
| | GAGCAAAGCTGCTATTGGCCGCTGCCCCAAAGGCCTACCCGCTTCCATTGCTCAGCGGTGC |
| | TGTCCATCTGCACGAGACTAGTGAGACGTGCTACTTCCATTTGTCACGTCCTGCACGACGC |
| | GAGCTGCGGGGGGGGGGGGAACTTCCTGACTAGGGGAGGAGTGGAAGGTGGCGCGAAGGGG |
| | CCACCAAAGAACGGAGCCGGTTGGCGCCTACCGGTGGATGTGGAATGTGTGCGAGCCAGAG |
| | GCCACTTGTGTAGCGCCAAGTGCCCAGCGGGGCTGCTAAAGCGCATGCTCCAGACTGCCTT |
| | GGGAAAAGCGCCTCCCCTACCCGGTAGGAAGTTCCTATTCTCTAGAAAGTATAGGAACTTC |
| | AATTTCTTGAAAAAAATCAGTGCTATTTTGGGGTCAGAACAATCCTCAAAAATGGTCCCCG |
| | GAAAGAAGCCCAAATTTAATTGCACCAGATTGTTGAACGATTTATGCTACAAAACAGTGTC |
| | ACATGTCAGAAATGAGCACAGTGTAACATCTACATGGTTTGTTAAGAGACACAAATGGTCA |
| | AGTAGAACAATCAGGTAATTAGGCTGTCGAAGGCAACACTGCCAAATGACAGCATTTCCGT |
| | GGAAACTGCGTGTACATCCAGGACTGCACCTGTGAACGATGACATCATACCCTTCACAGTG |
| | TCGAGGAAAGAGACATCACTCAAACAGACAAGCCAAGGGACTTCAGAAAATATAAGGGGAA |
| | ATACAGTGTGCAAATATGTAAAAAATGCAATAAGATGATTACTCCTAAATGAATATCAAGA |
| | CACAATCACATAATATGAAATTAAATTTTCCTGAATGATAGGATTACTACCAATCACCCCC |
| | CAGGACACCCTCATCTACTCTGTGCACAGCCTTCTCGTCAGGCGTCCCAGCCCAGACCTTG |
| | CTATGTAGCAGAAGACATGCAAATAAGACCCCCCTTTTTGCTGATGAAAAGCAGCCCAGCC |
| | CTGACCCTGCAGCTCTGGGAGAGGAGCTCCAGCCTTGGGATTCCCAGCTGTCTCCACTCGG |
| | TGATCGGCACTGAATACAGGAGACTCACCATGGAGTTTGGGCTGAGCTGGGTTTTCCTTGT |
| | TGCTATTTTAAAAGGTGATTCATGGGGAACTAGAGATACTGAGTGTGAGTGGACATGAGTG |
| | AGAGAAACAGTGGACGTGTGTGGCACTTTCTGACCAGGGTGTCTCTGTGTTTGCAGGTGTC |
| | CAGTGTGAGGTGCAGCTGGTGGAGTCTGGGGGAGGCTTGGTCCAGCCTGGGGGGTCCCTGA |
| | AACTCTCCTGTGCAGCCTCTGGGTTCACCTTCAGTGGCTCTGCTATGCACTGGGTCCGCCA |
| | GGCTTCCGGGAAAGGGCTGGAGTGGGTTGGCCGTATTAGAAGCAAAGCTAACAGTTACGCG |
| | ACAGCATATGCTGCGTCGGTGAAAGGCAGGTTCACCATCTCCAGAGATGATTCAAAGAACA |
| | CGGCGTATCTGCAAATGAACAGCCTGAAAACCGAGGACACGGCCGTGTATTACTGTACTAG |
| | ACACACAGTGAGGGGAGGTCAGTGTGAGCCCGGACACAAACCTCCCTGCAGGGGCGCGCGG |
| | GGCTACCAGGGGGCGCTCGGGACTCACTGAGGGCGGGACAGGTCCCAGGAACAGGTGCAGC |
| | GGTAGGTTTTCTTTCTCCTCAGCTGGAGAAGTCAGGTTTGTGTTTTCAGAACTCTGGAGTC |
| | TTACAGGTTGCTACATTTTCATACAGTTATTAGTATGTATTTATTATCATTGGTATTTAAG |
| | TTTTAATAATTTTAAACCTTTTATGTAGTGTTATTTTTTAAAACTGTTTACTTTCATTTGC |
| | AGTTATTCTTCCAGAGTTTCATTAACATCTATTGCTATGAGCAACTACATAGCTATGAGAG |
| | CATAAATTTACACCTGTAGACGTAGGTCTAAATGCCACAAACCTGTGCATAAATGTATAGT |
| | GACTTATATTTAACATTATAATAAGATAATTTTTAAAATATATCCTAAACGATCAAACTTA |
| | CTGATGAACTAAATATAAATTATCAGAGTAATGCATAATTGATTTCAATAATTTTATATTG |
| | TTTATATTAATTAATATCTATTTCTTTACTGAAACATAATATATTGGTCATTTCAAAATAG |
| | CTACGATACATTTCAAATGGCCTTGATGCTAATAATGAAAAGATTTTGAGGTGATTAATAT |
| | GCTAATTAGTTATATTTAATTATTCCATATTGGGTCCCACACACTGTTAAACAACCAAATC |
| | TCATGAGAACTCATTTACTATCATAGCAAGGGGACCGTGCTAACCATTATTCATAAATCCG |
| | GACCCACGATACAACCACCGGGCCCCGCCTCAAACATTAGGGAAGCAAAATCACATACAGT |
| | TGTTATGACAGGTGGGACATCCTGAAAACCTCTCTAGGCATGTCCCACACGGCCCTGGAGC |
| | TGTCTCAGGGGAGCAGTCTCCTCCAGTGTTTAGACGCACAGGCACGGATAATAGGGCTAAG |
| | TCTGGCCAGATGTGTGATATTCAACACATTGCACAACTGCTCTGTTCTGTATGTAATTTAT |
| | CTTCTCTACAAATGTAACATTGATGGTTGCATTAAATATATTCTGCAAATATGTAAAAATA |
| | AAATAAGATGATGATTGCTAAATGATTATCAAGGCACAATCACATAATCCGAAGTTACAAT |
| | TTCCTGAGAGATAGGATTACTGCCCATGCTTACCAGGACACTCACATCTGCTCTGGGCACT |
| | GCCTTCTCCTCAGGCGTCCCACCCCAGAGCTTGCTATATAGTAGGAGACATGCAAATAGGG |
| | TCCTCCCTCTGCTGATGAAAACCAGCCCAGCCCTGACCCTGCAGCTCTGAGAGCGGAGCCC |
| | CAGCCCCAGAATTCCCAGGTGTTTTCATTTGGTGATCAGCACTGAACACAGAGGACTCACC |
| | ATGGAGTTTGGGCTGAGCTGGGTTTTCCTTGTTGTTATTTTACAAGGTGATTTATGGAGAA |
| | CTAGAGATGTTAAGTGTGAGTGGACGTGAGTGAGAGAAACAGTGGATTTGTGTGACAGTTT |
| | CTGACCAGGGTGTCTCTGTGTTTGCAGGTGTCCAGTGTGAGGTGCAGCTGGTGGAGTCTGG |
| | GGGAGGCTTGGTCCAGCCTGGAGGGTCCCTGAGACTCTCCTGTGCAGCCTCTGGATTCACC |
| | TTCAGTGACCACTACATGGACTGGGTCCGCCAGGCTCCAGGGAAGGGGCTGGAGTGGGTTG |
| | GCCGTACTAGAAACAAAGCTAACAGTTACACCACAGAATACGCCGCGTCTGTGAAAGGCAG |
| | ATTCACCATCTCAAGAGATGATTCAAAGAACTCACTGTATCTGCAAATGAACAGCCTGAAA |
| | ACCGAGGACACGGCCGTGTATTACTGTGCTAGAGACACAGCGAGGGGAGGTCAGTGTGAGC |
| | CCGGACACAAACCTCCCTGCAGGGGCGCGTGGGGCCACCAGGGGGCGGTCGGGAACCACTG |
| | AGGACGGGACAGGTCCCAGGAGCAGGTGCAGGGGGCGGTTTCCTGTCAGCTGCAGGAGGCG |
| | GGTTTGTTTTTGCAGGACTCTGGAGCCTTATGAGGTTGCACTATTTTATTATGGTTATTTA |
| | TCATGTGTTTTTTATTGATATTTGTGTTTTAGTAATTTTTACATTTATATGTAGGGGTATT |
| | TTTTAAAAATACATAACTTCAAGAAATAATTCTTCCTAGTAATTTACACCTATTCCTCTAA |
| | AGTTTTATTAACACCTGTTGATATCAGCAACTACATAGCTATGCAACATTAATTTACATCT |
| | ATAAACATATGTGTGAATACATGAAACTATGCATACATGTGTAGTCATTTATATTTAACAT |
| | TATAATAAAATAATAAATTAAATAAAATAATTGAATTAAATTAAAAGAATTAAACTTAATG |
| | ATGAACTAAATATAAATTAGAGTAATCCATAATTGATTTCAATCCTTCTCTATGGTTTACA |
| | TAAATCGATGTCTACTTGTAAGCTTAAGTATAGTGAATTGGTCATTTTAAAATAGCCAAGA |
| | ACAAATTTCAAATGTTCTTGTCACCAAAAAATGATAAGGATTTGAAGATTTGAGGTGATAT |
| | ATAAGGAGTCTTCCAATTCCCTTGTCCAAAGCATCCGCTCATTCATGGGCTGAGAAAAGGG |
| | AAGTCATTCATGCAGTCTACTCTCTCCACAGAACTGACGGGGCACAAGGACAACATCGATT |
| | TTTCATGGAACATGCCTCTAGGAATGCAGCTGTGTGCACACACACTGCTAAGCACACACTT |
| | CTTTACATAATTACTTGTAACTGTATTTTCTTATTTATTCTCTCCAATTTTTTTACACAAA |
| | TTCATCACTTTTCCCCATAATCAAAGAGGATTTTGATCAGAATGCTTGTGGGGAGCCCCTT |
| | GCCTGCCAGATGCCCAACATCACTACTCTTGAAGGGAGGAGGAACGGCAGCTCTCTTGATT |
| | TCTACTCTAATCCTCTAGGACTAAAACCAGAAGGTTGCATGTCCAGTGCGGGAGCATCGAA |
| | GAAGATCCTGTCTGTAGAAGCAGGAGCGTCAAGACTTGACTGAGAGCCATGGTGCTGAAAT |
| | GAGATAGATTCCCTGATGGAGAGCACACGTGGACCCCCACACCTGAGGGCTCACTGCTCCT |
| | CACCACAGATGCACTCCCCTACTGAGTCCTGAGACCTGAGTGCACCCCATAGAGTAGGGCT |
| | CAGATGAGGGGATGCAAATCTCCACCAGCTCCACCCTCCCCTGGGTTCAAAAGACGAGGAC |
| | AGGGCCTCGCTCAGTGAATCCTGCTCTCCACCATGGACATACTTTGTTCCACGCTCCTGCT |
| | ACTGACTGTCCCGTCCTGTGAGTGCTGTGGTCAGGTAGTACTTCAGAAGCAAAAAATCTAT |
| | TCTCTCCTTTGTGGGCTTCATCTTCTTATGTCTTCTCCACAGGGGTCTTATCCCAGGTCAC |
| | CTTGAAGGAGTCTGGTCCTGCGCTGGTGAAACCCACACAGACCCTCACACTGACCTGCACC |
| | TTCTCTGGGTTCTCACTCAGCACTAGTGGAATGCGTGTGAGCTGGATCCGTCAGCCCCCAG |
| | GGAAGGCCCTGGAGTGGCTTGCACGCATTGATTGGGATGATGATAAATTCTACAGCACATC |
| | TCTGAAGACCAGGCTCACCATCTCCAAGGACACCTCCAAAAACCAGGTGGTCCTTACAATG |
| | ACCAACATGGACCCTGTGGACACGGCCGTGTATTACTGTGCACGGATACCACAGAGACACA |
| | GCCCAGGGCGCCTCCTGTACAAGAACCCAGGCTGCTTCTCAGTGGTGCTCCCTCCCCACCT |
| | CTGCAGAACAGGATAGTGTGGCTGAGATGCCATTTCCTGCCAGGGCCTGCGTTTCCCATCC |
| | CCATCTGACTCAGAGCCTTGTTTTCCTCCCTCTTCTTTACTAATAAATGGCATGTCCCCTG |
| | TTAGTGGTTCGTGCAAGCAGAAGCTGTATCCTGTTTGACAAAGATTCAGCATGAAAGGTTC |
| | CTGTTACCTAAAAAAAAATAGACAGATGAGACTTAATTAACCTAAATAATTTTTTTCACAA |
| | CAACAGAGTGAATACACAATTTGCAGAATGACAGAAAACTTTTGCACACTTTGTCTGTGAC |
| | AGGGAACTAATATGAAGAATTTGCAAGGAACTCAAACAACTCTACAACAACAACAGCAACG |
| | AGAACCAAATAACCCCATTAAAATGAGCAAAGAACATGAGTAGACATTTTCAAAAGAACAC |
| | ATAGAAATGGATAATAAATATATAAACAATGCTCAACATCACTAACCATCAGGGAAATGCA |
| | AATTAAAACCACAATAAGATATCATCTTCCACCAGTCACAATGACTGTTACTAAAAACTCA |
| | AATAATATCAGATGTTGCTGAGGATGGGAAATAAAGGCAACTCTTAGACATTGTTGATGAG |
| | GATGTAGACGAGTACAAACTCAAATTCCTATTTCTATTTCAACCCCTGATTCCTACTGTCA |
| | ATGGGAGGGAAGTCTCAGAACCAATCACACATCAGACGGCAAATCTGTCAACCAAGAGTCT |
| | TTCCACTGAAGGACCTGGGAGGTCAGGACCCTCAGGAAAGTGCTGGGGACCCTGTCTTGGG |
| | AGTGCCCAGCAGATCTCAGAACTCTCCATGGGTCCTGCTGGACACTCATGTAGGGTAACGA |
| | GTGGCCACCTTTTCAGTGTTACCAGTGAGCTCTGAGTGTTCCTAATGGGACCAGGATGGGT |
| | CTAGGTGCCTGCTCAATGTCAGAGACAGCAATGGTCCCACAAAAAACCCAGGTAATCTTTA |
| | GGCCAATAAAATGTGGGTTCACAGTGAGGAGTGCATCCTGGGGTTGGGGTTTGTTCTGCAG |
| | CGGGAAGAGCGCTGTGCACAGAAAGCTTAGAAATGGGGCAAGAGATGCTTTTCCTCAGGCA |
| | GGATTTAGGGCTTGGTCTCTCAGCATCCCACACTTGTACAGCTGATGTGGCATCTGTGTTT |
| | TCTTTCTCATCCTAGATCAGGCTTTGAGCTGTGAAATACCCTGCCTCATGCATATGCAAAT |
| | AACCTGAGGTCTTCTGAGATAAATATAGATATATTGGTGCCCTGAGAGCATCACATAACAA |
| | CCACATTCCTCCTCTGAAGAAGCCCCTGGGAGCACAGCTCATCACCATGGACTGGACCTGG |
| | AGGTTCCTCTTTGTGGTGGCAGCAGCTACAGGTAAGGGGCTTCCTAGTCCTAAGGCTGAGG |
| | AAGGGATCCTGGTTTAGTTAAAGAGGATTTTATTCACCCCTGTGTCCTCTCCACAGGTGTC |
| | CAGTCCCAGGTGCAGCTGGTGCAGTCTGGGGCTGAGGTGAAGAAGCCTGGGTCCTCGGTGA |
| | AGGTCTCCTGCAAGGCTTCTGGAGGCACCTTCAGCAGCTATGCTATCAGCTGGGTGCGACA |
| | GGCCCCTGGACAAGGGCTTGAGTGGATGGGAGGGATCATCCCTATCTTTGGTACAGCAAAC |
| | TACGCACAGAAGTTCCAGGGCAGAGTCACGATTACCGCGGACGAATCCACGAGCACAGCCT |
| | ACATGGAGCTGAGCAGCCTGAGATCTGAGGACACGGCCGTGTATTACTGTGCGAGAGACAC |
| | AGTGTGAAAACCCACATCCTGAGAGTGTCAGAAACCCTGAGGGAGAAGGCAGCTGTGCCGG |
| | GCTGAGGAGATGACAGGGGTTATTAGGTTTAAGGCTGTTTACAAAATGGGTTATATATTTG |
| | AGAAAAAAAGAACAGTAGAAACAAGTACATACTCTAATTTTAAGATAAATATTCCATTCAA |
| | GAGTCGTAATATAAGCCAAATTCACAGAGTGGAAAAGGCCACACTCTATAACGTTGATACA |
| | AACATTCCATGAAGGTGCTACTGTGAACAAGTTTTCAAATTGGATGAATACATGATTTGGA |
| | GCAAGGTTATTTGATCATGTGGTGAGACTAAGAATGATTCTTAAAAAGTGCCAAAAGTTTC |
| | CTTCAAATGTTTCTGTCACTCCTTATCATAAAGTTTATTTTACAGCAGTTTTAGGATTACA |
| | AAGAAATTGCACAGGAGGCGTGAGAATTCCCATGACTCCCTGCCCTACACAGGCACAGCCT |
| | CCTCCACTACGACCATCCTGCACCGCAGTCACAAATCAGTTACAATGGAGGAATCTCCAAG |
| | GACGCTTGGTTCTTTCTTTTTCTGGTGATCTCCTAATATAACAAGCCTAAGTATCTCAAGA |
| | TTCCACGGTTTTTTCAGTGTTTTCTAGAACTGATATTAGTCAGAGGGAAAGTGGGTAAGGC |
| | TATTACTATTTGAACTCTTTCTTCCATACATATTTTCAATCAGAAGTGTTTAGAGGCCAGG |
| | ACATATCTTCACGGTCACACATTGAGAAGGATGTAGATATGTCCCACTACCTTCTCCTGAG |
| | ATCTCAGACAGAATCCCAGATTTCAAAAGGACACAGAAGGACAGCTCTCAGGTGCTTTTAA |
| | AAAATGACCCACTTCCAGGGACAGGGAGCTTCCCTATAACCATGGTGGATGTTCTGAACTA |
| | CAATAAACATTGGATGGATCCAGGATTGTTTGAAGTCACTGTCATTATTACATTCAGCTGC |
| | TGTTTCAATGTGTCTGAAGTAGTAAATGACAATTTAGATGACAATTTATATGAATCTTCAA |
| | GGGTAGAACAATATTGACCATATTCCAAAATCTGTCCTTGATCCATGATCACACTCATCTC |
| | CCAGACCAGGTCCTTCAGCACGTCTCTTTACCTGAAAGAAGAGGACTCTGGGCTTGGAGAG |
| | GGGAGACCCCAAGAAGACAACTGAGTTCTCAAAGGGCACAGCCAGCATCCTACTCCCAGGG |
| | CGAGCCCAAAAGACTGGGGCCTCCCTCCTCCTTTTTCACCTCTCCGTACAAAGGCACCACC |
| | CACATGCAAATCCTTACTTAAGCACCCACAGGAAACCACCACACATTTCCTTAAATTCAGG |
| | TTCCAGCTCACATGGGAAATACTTTCTGAGAGTCCTGGACCTCCTGTGCAAGAACATGAAA |
| | CACCTGTGGTTCTTCCTCCTCCTGGTGGCAGCCCCGAGATGTGAGTGTCTCAGGGATCCAG |
| | ACATGGGGGTATGGGAGGTGCCTCTGATCCCAGGGCTCACTGTGGGTCTCTCTGTTCACAG |
| | GGGTCCTGTCCCAGGTGCAGCTGCAGGAGTCGGGCCCAGGACTGGTGAAGCCTTCGGAGAC |
| | CCTGTCCCTCACCTGCACTGTCTCTGGTGGCTCCGTCAGCAGTGGTAGTTACTACTGGAGC |
| | TGGATCCGGCAGCCCCCAGGGAAGGGACTGGAGTGGATTGGGTATATCTATTACAGTGGGA |
| | GCACCAACTACAACCCCTCCCTCAAGAGTCGAGTCACCATATCAGTAGACACGTCCAAGAA |
| | CCAGTTCTCCCTGAAGCTGAGCTCTGTGACCGCTGCGGACACGGCCGTGTATTACTGTGCG |
| | AGAGACACAGTGAGGGGAGGTGAGTGTGAGCCCAGACACAAACCTCCCTGCATGGACGCGG |
| | AGGGGACCGGCGCAGGTGCTGCTCAGGACCAGCAGGTGGCGCGCGGGGCCCCCAGAGCATG |
| | AGGCCGGGTCAGGAGCAGGTGCAGGGAGGGGGGGGCTTCCTCATCTGCTCAGTGGTCTCCG |
| | TCCTCGCCAGCACCTCGCTGTCACCAGGGCTCCTCTTTCTTTATTATCTGTGGTTCTGCTT |
| | CCTCACATTCTTGTGCCAGGAAAGAAACGAGGAAGACAAATTTTCGTCTATAGTTGAAGCT |
| | TCACCAATTACTAGGAACTTGCCTACAAGTTCCTGCATGACCCATTATAACTTATCGATTA |
| | AAAAATATATATTCTAATGCTTCTCACCATCTCTTGATTTGTATCATCAACTGAATTGTAC |
| | CCTCTTTGAAATTCATATGATGAAACCTTAAATTCAATGGATCTATATTGGAATTTTAATG |
| | AAATAATTAAGGTTAAATGTGGTCATAATTGTAAGACCCTAATGCAATAGACGTGTTGTCT |
| | TTATAAGAAGAGGAAGAGACACCAGAGACCTCTCACTTTTCACGTGCAGGCAGAGAAGAGG |
| | CCATGTGGAGACATAGTGCACTAGAAGGTGGCCCAGTGCAAGCCAGGAAGAAGCCGCGCCA |
| | AGAACCAGCCCTGCCAGCACACTAATCTTCAACATTGGGTGCAGCAATGGGCATCCCTTCA |
| | GGTCCATCAGCAAGTCAGAGACCTAAACAAATATTCCCTATTCATGTTGAATTGGCAGAGT |
| | GTAAAAATCTAGAAGACAAAATGGCCAGGAAAAATGTGCGATTTTGCATGGGTGAAGGTCT |
| | GGCAAGTTAATCATCTTGAGGACTGTTGAGGTCCTGAGGTGGTCCTGCCCTTGCTTGATGC |
| | CCAGGCCGTAGTCCAACTCACACGAAATTGCCAGGGAATAGACAGTAGTTTTTTTAGTAGT |
| | TCTTGTTATCAGGCATAAGTGCATTTGAATTTTCTCTTCATGGCCTTTCCTGGCACTATTT |
| | CTCATTTTTTTTAACACACATAGTTTCAACTAGATTTATCACCTTCACAGGGTCACAGAGA |
| | AGGGTGGAAGAAGGGAGGCCCTGTATGGGTCTCGAAGAAACATGGAAAAGAGTGGAGAGGG |
| | ACAATAGCAGGGTGTAAGGAATTATTGAGACCTTACTCTGCCCCTCCCAGGAGGCTCAGGC |
| | CAGCCTTTTTCTGCATTTGAGGTTCTGGGTTATAAACGCTGTAGACTCCTCCCTTCAGGGC |
| | AGGGTGACAACTATGCAAATGCAAGTGGGGGCCTCCCCACTTAAACCCAGGGCTCCCCTCC |
| | ACAGTGAGTCTCCCTCACTGCCCAGCTGGGATCTCAGGGCTTCATTTTCTGTCCTCCACCA |
| | TCATGGGGTCAACCGCCATCCTCGCCCTCCTCCTGGCTGTTCTCCAAGGTCAGTCCTGCCG |
| | AGGGCTTGAGGTCACAGAGGAGAACGGGTGGAAAGGAGCCCCTGATTCAAATTTTGTGTCT |
| | CCCCCACAGGAGTCTGTTCCGAGGTGCAGCTGGTGCAGTCTGGAGCAGAGGTGAAAAAGCC |
| | CGGGGAGTCTCTGAAGATCTCCTGTAAGGGTTCTGGATACAGCTTTACCAGCTACTGGATC |
| | GGCTGGGTGCGCCAGATGCCCGGGAAAGGCCTGGAGTGGATGGGGATCATCTATCCTGGTG |
| | ACTCTGATACCAGATACAGCCCGTCCTTCCAAGGCCAGGTCACCATCTCAGCCGACAAGTC |
| | CATCAGCACCGCCTACCTGCAGTGGAGCAGCCTGAAGGCCTCGGACACCGCCATGTATTAC |
| | TGTGCGAGACACACAGTGAGAGAAACCAGCCCCGAGCCCGTCTAAAACCCTCCACACCGCA |
| | GGTGCAGAATGAGCTGCTAGAGACTCACTCCCCAGGGGCCTCTCTATTCATCCGGGGAGGA |
| | AACACTGGCTGTTTGTGTCCTCAGGAGCAAGAACCAGAGAACAATGTGGGAGGGTTCCCAG |
| | CCCCTAAGGCAACTGTATAGGGGACCTGACCATGGGAGGTGGATTCTCTGACGGGGCTCTT |
| | GTGTGTTCTACAAGGTTGTTCATGGTGTATATTAGATGGTTAACATCAAAAGGCTGCCTAA |
| | CAGGCACCTCTCCAATATGACAGTATTTTAATTAGTGAAAATTTTACACAGTTCATCATTG |
| | CTTGCTTGCCTTCCTCCCTCCTGTCCACTCTCACTCACTCCTTCTTTTATTTTCTACTTAA |
| | TTTTACAAAATCATTTAACCCCTTTTTGAACTATTAATAGGTTATCTTTGTTTGGTGATTG |
| | TTTTCCTTTCAATAATATGTACTGAATAATTCATCTTTGTGCCAATTCATAAGTATTCTGG |
| | TGTAATAAAGACTTCTTTCATAAAAATTGGATAAATTAAAATAAAGATAAATTTTTAAAAA |
| | CATACGATCTATCAAAACTGAACCATAAAGAAATAAAAACTCTGGGTTGGGTGTGTTTGCT |
| | CATTCCTGTAATCCCAGCACTTTGGGAGGCCATGGCCGGTTTACAAGCAAAAATTGAAAGT |
| | TAGACAAGAAGGAGTGATAGAAAGAGAAAATGTATATTAAATTTCAGAAATATTTAAGAAT |
| | GTATCTGCCTGAACCCTAGTTCTCACCATATCTTTAGGTGAATGCTAAAATGCAGCAAAAT |
| | CACGCATGTTCTCACTACAGAAAGTGGGTTCTACAAACCACACTCGGCACATTTAGCTTTG |
| | TCCTGGAGTTGGTTCAGGGAGTTATTGGGGCCAGTGATGAGGAGCACAGGCCAAGATACCA |
| | GCGATCACTTATCCCAAACATGAGCTCTAACATACACACTTAGTCCCTTTTCCGTGTGTGG |
| | TTTACTTCCACATCTGTACATGGAGAGACCACTGACTGACAAAATATAATTTATACAAATA |
| | TGTAAAATTAAATAGGGTGATCAGTTCAAGGTGTTTATCACAGCATAATTTTACAATAAGA |
| | CAGCATATTTCCCAAATACCATCATTGTCACCAAACTCCTTCAAGGCACAGTCATCTTATC |
| | TGGGCCCCGTCCTCTCCTCAGGTGTCCCACCCCAGAGCTTGGTATATAGTAGGAGACATGC |
| | AAATAAGGCCCTCCCTCTGCTGATGAAAATGAGCCCAGCCCTGACCCTGCAGCTCTGGGAG |
| | AGGAGCCCCAGCCGTGAGATTCCCAGGAGTTTCCACTTGGTGATCAGCACTGAACACAGAC |
| | CACCAACCATGGAGTTTGGGCTTAGCTGGGTTTTCCTTGTTGCTATTTTAAAAGGTAATTC |
| | ATGGTGTACTAGAGATACTGAGTGTGAGGGGACATGAGTGGTAGAAACAGTGGATATGTGT |
| | GGCAGTTTCTGACCTTGGTGTTTCTGTGTTTGCAGGTGTCCAATGTGAGGTGCAGCTGGTG |
| | GAGTCTGGGGGAGGCTTGGTACAGCCAGGGCGGTCCCTGAGACTCTCCTGTACAGCTTCTG |
| | GATTCACCTTTGGTGATTATGCTATGAGCTGGTTCCGCCAGGCTCCAGGGAAGGGGCTGGA |
| | GTGGGTAGGTTTCATTAGAAGCAAAGCTTATGGTGGGACAACAGAATACACCGCGTCTGTG |
| | AAAGGCAGATTCACCATCTCAAGAGATGGTTCCAAAAGCATCGCCTATCTGCAAATGAACA |
| | GCCTGAAAACCGAGGACACAGCCGTGTATTACTGTACTAGAGACACAGTGAGGGGAGGTCA |
| | GTGTGAGCCCAGACACAAACCTCCCTGCAGGGGCGCACAGAGCCACCAGGGGGCGCTAGGG |
| | ACCGACTGAGTACGGGACAGGTCCCAGGAGCAGGTGCAGGGGGAGGTTTCCTTTTTCCTTG |
| | GCTGGAAAAGTCACCTTTATCTTCCCAGGACTCGAGCCTTCTAGGCTGTGATATTTTATTA |
| | CTTGTATTTACTGTTCATTTATTATCATTAGTTTTTAAATTTTGGTAATTTTTACAACTCT |
| | ATGGATATATTTTTAAGTGTATACTTTCAAGAAATAAACATTCCTAATTATTTGCACTGAT |
| | TCTCCCAGAGTTTTATTAACATTTGTTGACATCAGCAACTACATAGCTATAGGGACAAACA |
| | CTTTTAACGATAGACAGTTGTTTAGGCCTGAAACCCCGTTTATACTAAAAATTTACAAAAA |
| | TTAGCCTGGCGTGGTGAGGGGCGCCTGTAATCCCAGCTACTCGGGAGGCTGAGCAAGGAGA |
| | ATTGCTTGAATCCGGGAAGCGGAAGTCACAGTGAGTGGAGTGGACTGCCACTACACTCCAG |
| | CCTGGCGACAGAGCGAGACTTTGTATGAAAGAAAGAAAAGAAAAGGAAGAAAGGAAGAAAG |
| | GAAGGAAGGAAGGAAGGAAGGAAGGAAGGAAGGAAGGAAGGAAGGAAGGAAAAGAGAGAAA |
| | GAAAGAGAGAGAATGTTTGTCAAATGGTGAACATATTGTGTGAAGATTATATGTCTGTATT |
| | ACTTCATTAAAGCTATTATAAATAAAAGTCTAATGTGGTAGAAAAAGATGAAGAGAGAAAT |
| | AAAAATAATACAAGAAAAGTCATGAACTCCTGAATGAATTAACCCTTAGTTTTTCTCTATT |
| | ACTTATAAAAACACCAAGATACAGCCAAATAATATCACGATATCATTATAAGAAGAGTGTT |
| | TTGTAAACCTCACTGGGAATTTATAGCTCTTTCCTAGAGTTAATTTTGGGAACAGTTGGAT |
| | CCAATTGTGAGAAATGCAGGCTGGACACTGAGACTGGCTCTTATGAGATGTGAGCTCTTGT |
| | CTATGTCACATGGTCCTTCCATACTTGGGGGTTTACATTCACATCTGTAAATGAAGGAAAC |
| | ATTGACTCTCAAAGAACATATTTCATGTGCATGTAAAAGTATGAATGCTAGTGAGAATTAA |
| | TTACTTATGAAGTATAATCACCCACATCCACTCTTGGACACAGCCCACTCTGAGGCATCTG |
| | TTACAGAACTCATTATATAGTAGGAGACATGCAAATAGGGTCCTCCCTCTGCTGATGAAAA |
| | CCAGCCCAGCCCTGACCCTGCAGCTCTGGGAGAGGAGCCCCAGCCCTGAGATTCCCAGGTG |
| | TTTCCATTCGGTGATCAGCACTGAACACAGAGAACGCACCATGGAGTTTGGACTGAGCTGG |
| | GTTTTCCTTGTTGCTATTTTAAAAGGTGATTCATGGATAAATAGAGATGTTGAGTGTGAGT |
| | GAACATGAGTGAGAGAAACAGTGGATATGTGTGGCAGTGTCTGACCAGGGTGTCTCTGTGT |
| | TTGCAGGTGTCCAGTGTGAAGTGCAGCTGGTGGAGTCTGGGGGAGTCGTGGTACAGCCTGG |
| | GGGGTCCCTGAGACTCTCCTGTGCAGCCTCTGGATTCACCTTTGATGATTATACCATGCAC |
| | TGGGTCCGTCAAGCTCCGGGGAAGGGTCTGGAGTGGGTCTCTCTTATTAGTTGGGATGGTG |
| | GTAGCACATACTATGCAGACTCTGTGAAGGGCCGATTCACCATCTCCAGAGACAACAGCAA |
| | AAACTCCCTGTATCTGCAAATGAACAGTCTGAGAACTGAGGACACCGCCTTGTATTACTGT |
| | GCAAAAGATACACAGTGAGGGGAAGTCAGCGAGAGCCCAGACAAAAACCTTGCTGCAGGAA |
| | GACAGGAGGGGCCTGGGCTGCAGAGGCCACTCAAGACACACTGAGCATAGGGTTAACTCTG |
| | GGACAAGTTGCTCAGGAAGGTTAAGAGCTGGTTTCCTTTCAGAGTCTTCACAATTTCTCCA |
| | TCTAACAGTTTCCCCAGGAACCCTGTCTAGATCTGTGATCTGGATCTGCTGAAACTGCCTG |
| | TGTCACCTTCCTCACCTGTGACTTTGGGGGAGCTGATTGTGGACACTCCAGTGTGTGGGAT |
| | TTCTTGGTGACAGCAATTGTGTCTTCTGTCTAGGCATGTCTAGGGCTGGCCATCAGGAAGG |
| | GCAGGCTGGAATTTTTGGAAAGAGGCGCACCTGCCATCCACCAGGAAATTTTGTTGTCTTT |
| | TGTTCTGCTAGAATTAAATCAGACACACCAAGGTTAACTAGCACTATCTTCCTAGCTTGAG |
| | AAACTTGATGGCAGGCTTGAATAACACCTGTATGAAGCCATCAGAGCAACAACTAGATTAA |
| | TGTCTGCTAGAATTAAATCAGGCACACCAAGGTTAACTAGCACTATCTTCCTAGCTCGAGA |
| | AACTTGATAGCAGGCTTGAATAACACTTGTATGAAACCATCAGAGCAACACCTAGAATAGT |
| | GTCTGATTTAATAATTGAGACTATGGTCTAGCCAAGGAGCAGAAGTGTTTAGAGGCCAGGA |
| | CACATCTTCAAGGTCACACATTGAGAAGGATGGAGATATGTCCCACTACCTTCTCCTACGA |
| | TCTCAGACAGAATCCCAAATTTCAAAAGGACACAGAAGGACAGCTCTCAGGTGCTTTTAAA |
| | AAATGACCCACTTCCAGGGACAGTGAGCTTCCCTGTAACCATGGTGGATGTTCTGAACTAC |
| | AATAAACATTGGATGGATCCAGTATTGTTTGAAGTCACTGTCATTATTACATTCAGCTGTT |
| | GTTTCAATGTGTCTGAAAGGGTAAATGACTATTTAGATGGCCTGGGTGTGTGGTTGGTTTT |
| | ATATGAATCTTTAAGGGTTGAACAGTACTGACCCTATTCCAAAATCTGTCCTTGATCCAGG |
| | ATCACACTCATCTCTCAGACCAGCTCCTTCAGCACATCTCTTTACCTGGAAGAAGAGGACT |
| | CTGGGCTTGGAGAGGGGAGGCCCCAAGAAGAGAACTGAGTTCTCAAAGGGCACAGCCAGCA |
| | TTCTCCTCCCAGGGTGAGCTCAAAAGACTGGCGCCTCTCTCATCCCTTTTCACTGCTCCGT |
| | ACAAACGCACCACCCCCATGCAAATCCTCACTTAGGCGCCCACAGGAAGCCACCACACATT |
| | TCCTTAAATTCAGGTCCAACTCATAAGGGAAATGCTTTCTGAGAGTCATGGATCTCATGTG |
| | CAAGAAAATGAAGCACCTGTGGTTCTTCCTCCTGCTGGTGGCGGCTCCCAGATGTGAGTGT |
| | TTCTAGGATGCAGACATGGAGATATGGGAGGCTGCCTCTGATCCCAGGGCTCACTGTGGGT |
| | TTTTCTGTTCACAGGGGTCCTGTCCCAGCTGCAGCTGCAGGAGTCGGGCCCAGGACTGGTG |
| | AAGCCTTCGGAGACCCTGTCCCTCACCTGCACTGTCTCTGGTGGCTCCATCAGCAGTAGTA |
| | GTTACTACTGGGGCTGGATCCGCCAGCCCCCAGGGAAGGGGCTGGAGTGGATTGGGAGTAT |
| | CTATTATAGTGGGAGCACCTACTACAACCCGTCCCTCAAGAGTCGAGTCACCATATCCGTA |
| | GACACGTCCAAGAACCAGTTCTCCCTGAAGCTGAGCTCTGTGACCGCCGCAGACACGGCTG |
| | TGTATTACTGTGCGAGACACACAGTGAGGGGAGGTGAGTGTGAGCCCAGACAAAAACCTCC |
| | CTGCAGGGAGGCTGAGGGGGCGGGCGCAGGTGCAGCTCAGGGCCAGCAGGGGGCGTGCGGA |
| | GCTCACGGAATACAAGGCCGGGTCAGGAGCAGGTGCAGGGTGAGCGGGGCTTGCTCATCTT |
| | CTCAGAGATCATCATCTCCCTCCTCGCCAGCACCTCAGCTTTCCGTAGAGGTCCTCTTTCT |
| | TTATTGTGTGTGGTTCTACTTCCTCACATCCTTGTGCCAGGAAAGAAAGGAGTAAGGCAAA |
| | TTTTCCTGTTACAATTGAAGTTTCACCAATTACTAAGAACTTTCCTGCAAGTACCTGCACA |
| | GCCCATTATACCTTATTTATATATGTATATATTCTAATGCTTCTCACCATCTCTTGATTTG |
| | TGTCATCAATTTAATTGTGCCCTTTTTGAAATTCATATGCTGAAACTTTAAATCCAATGGA |
| | TCTATATCGGAATTTTAATGGTATAATTAATGTTAAATGTGGTCATAAATGAGACCCTAAT |
| | GCAATAGAGCTGTTGTCTTTATAAGAAGAGGAAGAGACACCAGATACCTCTCACTTCTCAC |
| | ATGCACTCAGAGAAGAGGCCACGTGGAGACATAGTGCACTAGAAGGTGGGCCTCTGCAAGC |
| | CAGGAAGAAGCCGCACCAAGAACCAACCCTGCCAGCACCTTGATCTTCTTTCATTACAGGA |
| | AGAAAGTTCTGAAAATGTCACTGGGGTGAACCACATTGTGCTGGGCTTGGTTCAGGGAGCA |
| | GTCAGGCCCAGTGTTGTGACCTTCAACCACAGAATCCTGAGGCTGCCTCAAAGTCCCCATC |
| | AGTTCCCGGACTCGCTATGTTTCTCGATCGTATCAGTGCATCCGGAGCTCCCTGGTGGCTT |
| | TAGTGATTCCTTGCTTGCCATGCTGAGGTCTCGCTGTAGATTATGTTGGGTTTTCTAAGAC |
| | CGTTTTGCTATTGTAAGATCCACTCCCTGGGACAGAGCGATTCCCTCTAAACCTGATGGAG |
| | GTTCTGAACTACAAATAACGTTAAGTGAATCCTGGTGTGTCTGAACTCAAGTGATTGTTAC |
| | ATTAAGCTGCTGTTGCAATCTGTTTCCTCACCTGGGAAAAGAGGAGCCAGGACATAGTGAG |
| | TTGAGGCCCCAGGAAGATAACTGAATTCTCAGAGGGCACAGCCAGCATCCTCTTCCCAGGG |
| | AGAGTCTAAAAGACTGGGGCCTCCCTCATCCCTTTTCACCTGTCCATACAGAGGCACCACC |
| | CACATGCAAATCTCACTTAGGCACCCACAGAAAACCACCACACATTTCCTTAAATTCAGGG |
| | TCCTGCTCACATGGGAAATACTTTCTGAGAGTCCTGGACCTCCTGTGCAAGAACATGAAAC |
| | ACCTGTGGTTCTTCCTCCTGCTGGTGGCAGCTCCCAGATGTGAGTGTCTCAAGGCTGCAGA |
| | CATGGAGATATGGGAGGTGCCTCTGATCCCAGGGCTCACTGTGTGTCTCTCTGTTCACAGG |
| | GGTCCTGCCCCAGGTGCAGCTGCAGGAGTCGGGCCCAGGACTGGTGAAGCCTTCACAGACC |
| | CTGTCCCTCACCTGTACTGTCTCTGGTGGCTCCATCAGCAGTGGTGGTTACTACTGGAGCT |
| | GGATCCGCCAGCACCCAGGGAAGGGCCTGGAGTGGATTGGGTACATCTATTACAGTGGGAG |
| | CACCTACTACAACCCGTCCCTCAAGAGTCGAGTTACCATATCAGTAGACACGTCTAAGAAC |
| | CAGTTCTCCCTGAAGCTGAGCTCTGTGACTGCCGCGGACACGGCCGTGTATTACTGTGCGA |
| | GAGACACAATGAGGGGAGGTGAGTGTGAGCCCAGACACAAACCTCCCTGCAGGGAGGCGGA |
| | GGGGACCGGCGCAGTTGCTGCTCAGGACCAGCAGGGGGTGCGCGGGGCCCACAGAGCATGA |
| | GGCCGGGTCAGGAGCAGGTGCAGGGAGGGCGGGGCTTCCTCATCTGCTCAGTGCTCTCCCT |
| | CCTCGCCAGCACCTCAGCTGTCCCCAGGGCTCCTCTTTCTTTATTATCTGTGGTTCTGCTT |
| | CCTCACATCCTTGTGGTAGGAAAGGAAAGAGGAAGGCAAATTTTCCTCTTAGAGTCAAAGT |
| | GTCACTAATTACTAGGAACTTTCCTACAAGTTCCTGAATGTCCCATTTTTCCTTCTTAATT |
| | AAAAAAAATATATATTCTAATACTTCTCACCATCTCTTGATTTGTGTCATCAGTTGAATTG |
| | TGCTGTCTTTGAAATTCAAATGCTGAAACCTTAAATCCAATTGATCTATATTGGAATTTTA |
| | AGGATGGAATTAAGGTTAAATGTGATCATAAGTCTGAGATTCTAATGCAATAGATCTGTTG |
| | TCTTTATAAGAAGTGGAAGAGTCACCAGAGACCTCTCACTTTTCCCGTGCACGCAGAGAAG |
| | AGGCCATGTGGAGACATAGTGGACTAGAAGGTGCAAGCCAAGAAGAAGCCGCACCAAGAAC |
| | CAACCCTGCCAGCACCTGGACCTTGGACATTCATGCTCACTCATGCGCTGGAAGAGGGATG |
| | TCAGAGATGCAGTCTACTCTTCCTAAAGGGCTGACAGGGCATAAAGACAAGACTGATTATC |
| | CATGGAACATTCCTATAGGAATGCACCTATGTGCAGACACACAAATAAGCTGTGTGTCTTG |
| | CAGTGTGTAAATGGCTGAGATGCCATTTACACTTCTTTACACAATACATTTTTAAATGTTT |
| | TGTTGATGTATTCTCTATCATTCAAAAAAATCATCACTTTCCCCCTAAACAAAGAGGATTT |
| | TTATCAGAACACTTATGGGAAGCCCCTTGCTCTCCCAGATTCCCACCCTCATTTCTCCTGA |
| | AGGAAGAAAGAAAACCATCTCTATTGATTTCCACTCTCATCCTCTAGGACTAAAACCAGAA |
| | AGCTGCATGCTCCCTGGGTGAGCCTTAGAGAAGACCCTGTCTGTAGGAGCAGGAATCTCAG |
| | AGCCTTGGCTGAGAGGCATGGTCCTGAAATGAGATGGAGTCCCCCATGGAGATCCCACGTG |
| | GACGCCCACACCTGAGGGCTCACTGCTCCTCACCACAGATGCACTCCCCTACTGAGTCCTG |
| | AGACCTGAGTGCACCCCATAGAGTAGGACTCAGATGAGGGAATGCAAATCTCCACCAGCTC |
| | CACCCTCCTCTGGGTTCAAAAGCTGAGCACGGGGCCTCGCTCAGTGACTCCTGTGCCCCAC |
| | CATGGACACACTTTGCTACACACTCCTGCTGCTGACCACCCCTTCCTGTGAGTGCTGTGGT |
| | CAGGGACTTCCTCAGAAGTGAAACATCAGTTGTCTCCTTTGTGGGCTTCATCTTCTTATGT |
| | CTTCTCCACAGGGGTCTTGTCCCAGGTCACCTTGAAGGAGTCTGGTCCTGTGCTGGTGAAA |
| | CCCACAGAGACCCTCACGCTGACCTGCACCGTCTCTGGGTTCTCACTCAGCAATGCTAGAA |
| | TGGGTGTGAGCTGGATCCGTCAGCCCCCAGGGAAGGCCCTGGAGTGGCTTGCACACATTTT |
| | TTCGAATGACGAAAAATCCTACAGCACATCTCTGAAGAGCAGGCTCACCATCTCCAAGGAC |
| | ACCTCCAAAAGCCAGGTGGTCCTTACCATGACCAACATGGACCCTGTGGACACAGCCACAT |
| | ATTACTGTGCACGGATACCACAGAGACACAGCCCAGGATGCCTCCTGTACAAGAACCTAGC |
| | TGCATCTCAGTGGTGCTCCCTCCCTACCTCTGCAGAACAGGAAAGTGTGACTGAGATGCCA |
| | TTTCCTGCCAGGGCTTGTGTTTCCTATACCAACCAGACTCAGAGCCCTGTCTGTTTTCCTA |
| | TTCTGTACTATTAAATGGCATGTTCCCTGTTAGTGATTCACGCAAGCAGAGGCTGTATCCT |
| | GTTTGACAAAGATTGAGCATGAAAGATTCCCATTACCTGGGCCACATGCATCACTGATATG |
| | TGGCCACTTATTTCTTGAGCTCATATCCTTCCAAGGGGCTGAATACAAATATCTATAACAT |
| | AGGCCATCTTAAACTGCAGGAAGTAATGTTGGAGAAGAATGTGGAGATAAAAGAGCAGGAT |
| | GATTAATTAAAATCCAGATACCATCTTTTTTTGCAGAGAAAAATTTACACTAATAAAGTAA |
| | TTTTATGAAACAACAAGAAAGATGGAATGTGTCCTCATCATGGAGTGTCTCACTTGCAGTG |
| | CACAAATAAATTTCTAAAAATTTTATAGGGAAGGTGTGATAGATGAGGCTGATTTCCACAC |
| | AGGGAGTGATTAAATACCCAGGTAAGTATAAAATCCAACACTTGGAAAGGAAAATGCCTCA |
| | GCTTGACATCAGAAACCCAGCTCAGTAAGTCTGAGGAGCAATGCGGACAGGGAGCACCCAG |
| | CACAGGACCCAGCCCTGGGGCAGGTGCACAGGAGGCTGGGGCAGGGTTTTCTCTCAGGAAT |
| | TGAATGTTCCTTATTTCAAAGCAATAATGACCTAACATTTAAATAAGAATTTAGCAAGTAC |
| | TGATGTGCCTGTAAGTATTTTATTGTATACGCAGCCTATACCTAACCCAATAATTTAATGC |
| | AAACCGGATTTAAAAGGAGAAATGTCTAGGTCTTTCAAATGTATTTCTAGTTAGGAATTGA |
| | GGGGTGGTTTTATTAATTCAATGGGTGTTACTGTCCGGAGACACACTCATCCCAGAAGTTA |
| | GATGTGCAGAGGGCAAGGCTCAGGAAAAGTTCAGGTAGACAGTGAGCCATATGAACAGGGA |
| | ATCACATTGAGGACCATGTCCTGTGAGATTCTGGGTTTCTGTGAGAGGAGTTCTGTCTTCA |
| | TGGACTTCTGGGCATGTCAGAGGACAAATATTAAACAAAGTTCAGGGCAGGGAGCTCAGCA |
| | TCCCACTGTGGCGTGGTCCACATGTCACCTATCTTCTTCCTCAGGGTGGCGTGGCCTGAGC |
| | TATGAAAAACCTGCCTCATGAATATGCAAATGCACTGCTGTCTACCGAGGTAAATACAGAT |
| | CTTTCCTTGCCCAGAGACCATCACACAACAGCCACATCCCTCCCCTACAGAAGCCCCCAGA |
| | GCGCAGCACCTCACCATGGACTGCACCTGGAGGATCCTCTTCTTGGTGGCAGCAGCTACAG |
| | GCAAGAGAATCCTTAGTTCCAGGGCTGATGAGGGGACTGGGTCCAGTTAAGTGGTGTCTCA |
| | TCCACTCCTCTGTCCTCTCCACAGGCACCCACGCCCAGGTCCAGCTGGTACAGTCTGGGGC |
| | TGAGGTGAAGAAGCCTGGGGCCTCAGTGAAGGTCTCCTGCAAGGTTTCCGGATACACCCTC |
| | ACTGAATTATCCATGCACTGGGTGCGACAGGCTCCTGGAAAAGGGCTTGAGTGGATGGGAG |
| | GTTTTGATCCTGAAGATGGTGAAACAATCTACGCACAGAAGTTCCAGGGCAGAGTCACCAT |
| | GACCGAGGACACATCTACAGACACAGCCTACATGGAGCTGAGCAGCCTGAGATCTGAGGAC |
| | ACGGCCGTGTATTACTGTGCAACAGACACAGTGTGAAAACCCACATCCTGAGAGCGTCAGA |
| | AACCCTGAGGAATGAGGCAGCTGTGCTGAGGCTGAGGAGATGACAGGATTTATGAAGTTTA |
| | AATCTGTTTAGAAAATGGGTTGAGTAATTGAGTAAAACAGCAATGGAAAGATGTGTACACT |
| | GTCATTATGTAGGAAATAGTATTTTCAACTGTCACCCTATAAGTAACACTCACAGAGTGGG |
| | AAAGGCAGCCATCAATCAAGCTGATGCAAACATTCCCATGGGGGCTTTGTGGGGACATACA |
| | TTTTATAATTGGATCTATAAATCATTAGAAACAGGATTGCTTGATCTCATGGTAAGACTAA |
| | ATATAATTTCCAAGAAGTGGCCGAAATTTCTTCCAAAATGTCTTTGCCCTTCCTTTTACAC |
| | TGAATTTATTTTAAAGGAGTTTTAGGATCACAACAAGTTTGAGTAGAAGAAACAGAGTTCC |
| | CATGCACTCCTGCCCCCCGACGCTCAGCCTTCTCTGCTATCAACACCCTGCACCAGTGTCA |
| | TAAATCGGTTACAGAGGATGAATCTCCACAGACACATTGGTTGTTTCCTTTTCTGGTGGTC |
| | CCTGGTATAACAAGCCTCAACTATCTTGAAGCACCCCAGGTTCAAGTGGATTCTTCAAGTG |
| | GGTTACTGGGAATGATGCCAGGTAGAAGGAAAGTGGGTGGGACCATTCCTCTTTGTGCTTT |
| | TCTTTATATTTGTTAGGTAATCACCACAGTGTGTACACATCACACCATGTTCCCATTACAG |
| | AGAAAAGGTTCTGCGAACCTCACGAGCTGTGACCCCTGTGTGCTGGGCTTGGTTCAGGGAG |
| | AAGTCAGGTCCAGTGGTGAGAAGCACAGGCCCAGATGCCCAGGCTCACTCTGACCAAATGT |
| | GAGCACTGGGGACATTGTAAAACCCACCTGTGCTTTTGCTGATAATTTTTCATCTTTAACA |
| | TGGAAATAATATTGATACTATATACCATGGTTTCTCTGCGTATGTAAAAATAAAAGATGAT |
| | TGGTGCTAACTTTAAAAATATGCAGTTTATGTAGATCTATGGTACCTCAATAAAACTGTTT |
| | TAAAATAAAAATTACAAAATTATAAGATTTTTAGGTTTTAAGGTTTAAGTTTATCACAAAA |
| | CAAACTGACAATAGGAAAGCACAATTTCCCAATGCTTTCAATATCACAGATCTCCCCGAGG |
| | ACATTCTGACATGCTCTGAGCCCCACTATCTCCAAAGGCCTCTCACCCCAGAGCTTACTAT |
| | ATAGTAGGAGATATGCAAATAGAGCCCTCCGTCTGCTGATGAAAACCAGCCCAGCCCTGAC |
| | CCTGCAGCTCTGAGAGAGGAGCCCAGCCCTGGGATTTTCAGGTGTTTTCATTTGGTGATCA |
| | GGACTGAACAGAGAGAACTCACCATGGAGTTTGGGCTGAGCTGGCTTTTTCTTGTGGCTAA |
| | AATAAAAGGTAATTCATGGAGAAATAGAAAAATTGAGTGTGAATGGATAAGAGTGAGAGAA |
| | ACAGTGGATACGTGTGGCAGTTTCTGACCAGGGTTTCTTTTTGTTTGCAGGTGTCCAGTGT |
| | GAGGTGCAGCTGTTGGAGTCTGGGGGAGGCTTGGTACAGCCTGGGGGGTCCCTGAGACTCT |
| | CCTGTGCAGCCTCTGGATTCACCTTTAGCAGCTATGCCATGAGCTGGGTCCGCCAGGCTCC |
| | AGGGAAGGGGCTGGAGTGGGTCTCAGCTATTAGTGGTAGTGGTGGTAGCACATACTACGCA |
| | GACTCCGTGAAGGGCCGGTTCACCATCTCCAGAGACAATTCCAAGAACACGCTGTATCTGC |
| | AAATGAACAGCCTGAGAGCCGAGGACACGGCCGTATATTACTGTGCGAAAGACACAGTGAG |
| | GGGAAGTCATTGTGAGCCCAGACACAAACCTCCCTGCAGGAACGATGGGGGGGAAATCAGC |
| | GGCAGGGGGCGCTCAGGACCCGCTGATCAGAGTCATCCCCAGAGGCAGGTGCAGATGGAGG |
| | CTGTTTCCTGTCAGGGTGTGGGACTTCATCTTCTTCTGACAGTTTCTCTAGTGAACCTCTC |
| | TAACCTCAGAATTCTGTGCTTACTAATGTCATCTCTACGTATTTTTTAAAAGATCATTTTA |
| | ATATGAGCACCTATTCTCACACGCACCAAATGCAGATTGACGCTTACAGAGATGAAAAGTT |
| | CTCAACCATGGTCACCAGGATGAGAGTCCTGAGGAAACTCAGGGGTGCCTGGTGACTCTTC |
| | CGCAATCAGTCCTGGGACAGAAACCTCAGGGACAGCCTTTATGAGTTTTGATCACATCTAA |
| | CAGAGAGGATGGGCCAGGGCCAGAGTCATGTAGAACCTCACAGGTTCCACGTCTGACCCTT |
| | CTCCTGACACTAAAGCCAATCAGCATCAGCACTGACCTGGTGCTCCTTTTGCTCCCAATCC |
| | ATGTTCTTTCTTTGGAGAGTTTCTTCTCCCTTTTTTATTTGCTTTTCCTGTTTCCTGAAAA |
| | AGAAACAGATGGTCCCCTTGGTCCACATTCCAGGGCTCAAGGCATTTTCTTGGAGCTCAGG |
| | TGGGGCTCAGGCTGTGGCTCAGCAAACAAATGAAAACTTGCACAAGAAATAAGTGACATAA |
| | AGATAGAAAAAATATTAAATTTCAGAAACACCTAATAATTTATCTTCGTGAACCCTAGTTC |
| | TCACCATATTTTTAGGTGAATGCTAGAATGCAGCAAAATTACACATGCTCTCAATACAGAA |
| | AGTGGGTTTCACAAACCACACTAGGCATGCTCAGCTCTGTCCTGGAGTTGGGTTAGGGAGT |
| | AATATAGGGCCAGTGGATGAGGAGCACAGGCCTAGATACTGGGGCTCACTAACCTCAGGTA |
| | TGAGCTCTTAGATACATACAAAGCCCCTCCACGCATGGGTTTACTTCCCCATCTGTAAATT |
| | GAGAAACCATTGACCCCTAAAAATATGATTTACACAAATATGTAAAAATGTAAGAGAGTGA |
| | TTAGTGCAAAGTGTTTATCACAGCACAATTTCATAACAAGACAGCAAGTTTTCCAAACAGC |
| | ATCATTGTCATTAGATTCCTGCAGGGCATCATTACCTTATCTGGGCCCTGCCCTCTGCTCA |
| | GGCATCCCACCCCAGAGCTTGCTATATAGTAGGTGACATGCAAATAGGGCCCTCCCTCTCC |
| | TGATGAAAACCAGCCCAGTCCTGACCCTGCAGCTCTGGGAGAGGAGCCCCAGCCTTGGGAT |
| | TCCCAAGTGTTTTCATTCAGTGATCAGGACTGAACACAGAGGACTCACCATGGAGTTTGGG |
| | CTGAGCTGGATTTTCCTTGCTGCTATTTTAAAAGGTGATTTATGGAGAACTAGAGAGATTA |
| | AGTGTGAGTGGACGTGAGTGAGAGAAACAGTGGATATGTGTGGCAGTTTCTGATCTTAGTG |
| | TCTCTGTGTTTGCAGGTGTCCAGTGTGAGGTGCAGCTGGTGGAGTCTGGGGGAGGCTTGGT |
| | AAAGCCTGGGGGGTCCCTTAGACTCTCCTGTGCAGCCTCTGGATTCACTTTCAGTAACGCC |
| | TGGATGAGCTGGGTCCGCCAGGCTCCAGGGAAGGGGCTGGAGTGGGTTGGCCGTATTAAAA |
| | GCAAAACTGATGGTGGGACAACAGACTACGCTGCACCCGTGAAAGGCAGATTCACCATCTC |
| | AAGAGATGATTCAAAAAACACGCTGTATCTGCAAATGAACAGCCTGAAAACCGAGGACACA |
| | GCCGTGTATTACTGTACCACAGACACAGTGAGGGGAGGTCAGTGTGAGCCCGGACACAAAC |
| | CTCCCTGCAGGGGCGCGCGGGGCCACCAGGGGGCGCTCGTGACCCACTGAGGGCGGGACAG |
| | GTCCCAGGAGCAGGTGCCGGGAGAGGTTTCCTTTCTCCTCAGCTGGAAAAGTCAGGTTTAT |
| | CTTCGCAGGACTCTGGAGTCTTCTAGGCTGTGATATTTTGTTACTTATATTTATTATGAAT |
| | TTTATCATTAATACTTAAATTTTAGTAATTATTAACATTCTACATATTATTATATTTTTAA |
| | GTATATACTTTCAAGAAATAAACATTCCTAATTGCATAATAATTATTGTAACATAATTATT |
| | GTAACATATAATTATATCCTAAATGCATAATAATTATTGTAACAATCACATTCTCATTTTG |
| | TATTTGAATTTTCATATTTATTTTACTAAGATTTTAATTTTAATATAAAATATTTTTCTAC |
| | AATTGTCTGTTTTCCATTTTTGTGAGATAAAATTAGCATATACAAAAATGCACAGATTCTC |
| | CAGGTACAGTTAGAGGGTTTCAGGCAAATGTGCACATACTTGTCTACAGCAGCTAAGTGAG |
| | GGTGAGGAACAGGTGAGGTCCATCTCCCCACAAAGTGTCCTCTTAGTGCTTCCAGTTAGTT |
| | CTCACATAAGGATTTTTTTTTATTTCAACTTTATAATTTAGATACAGAGGGTTCAAATTTT |
| | GAATGTATTGTTTGAAGATTGTTATTTGTCAATATTACCTCATTAAAACTATTAAAAATGG |
| | AAGTGTATAATTTGGTAGAAAAGGATTAAAAGAAAGATAAAAATAATACAAGAAAAGTCAC |
| | AGACTCCTGAATCCACATGTGAATTAGCACTCAGTTTTTCTATATTATTTATAAAAACACT |
| | AAGATACAGCCAAATAATATCATGATATCCTTATAAGAAGAGTGTTTCGTAAACCTCACTG |
| | GGAATTTCTAGCTCTGTCCTAGAGTTAATTTAGGGAGCAGTCGGATCCAGTCCTGAGCAAC |
| | ACAGGCCAGACACTGAGACTTACCACATCTTACCAGATGTGAGCTCTTAGACACGTCACAT |
| | GGCCTCTCCATGCTTGGGGGTTTACCTTCACATCTGTAAATGAAGGAAACATTGACTGCCA |
| | CAGAACATACTTCATGTGCATCTATACAGTAGAAATGCTAATAAGAATTAATTGTTTATGA |
| | AGTGTAATCACTCTGGGACACAGCCCACTCAGAGGCATCCCTTCCAGAACCCGCTATATAG |
| | TAGGAGACATGCAAATAGGGCCCTCCCTCTGCTGATGAAAACCAGCCCAGCCCTGACCCTG |
| | CAGCTCTGGGAGAGGAGCCCCAGCCCTGAGATTCCCAGGTGTTTCCATTCAGTGATCAGCA |
| | CTGAACACAGAGGACTCACCATGGAGTTGGGACTGAGCTGGATTTTCCTTTTGGCTATTTT |
| | AAAAGGTGATTCATGGAGAAATAGAGAGATTGAGTGTGAGTGGACATGAGTGGATTTGTGT |
| | GGCAGTTTCTGACCTTGGTGTCTCTGTGTTTGCAGGTGTCCAGTGTGAAGTGCAGCTGGTG |
| | GAGTCTGGGGGAGGCTTGGTACAGCCTGGCAGGTCCCTGAGACTCTCCTGTGCAGCCTCTG |
| | GATTCACCTCTGATGATTATGCCATGCACTGGGTCCGGCAAGCTCCAGGGAAGGGCCTGGA |
| | GTGGGTCTCAGGTATTAGTTGGAATAGTGGTAGCATAGGCTATGCGGACTCTGTGAAGGGC |
| | CGATTCACCATCTCCAGAGACAACGCCAAGAACTCCCTGTATCTGCAAATGAACAGTCTGA |
| | GAGCTGAGGACACGGCCTTGTATTACTGTGCAAAAGATACACAGTGAGGGGAAGTCAGCGA |
| | GAGCCCAGACAAAAACCTCCTGCAGGAAGACAGGAGGGGCCTGGGCTGCAGAGGGCACTCA |
| | AGACACACTGAAAACACGGTTAACACTGGGACAAGTTGCTCAGGAAGGTTAAGGGCTGGTT |
| | TCCTTTCAGAGTCTTTGCTGTTTCTGTATCTAACAGTTTCCCCAGGAACCCTGTCTAGATT |
| | TGCGATCTTGATCTGCTTAAACTGTCTCTGTGTCATTTTTCTTACCTGTGACTTTAGGGGA |
| | GCTGATCGTGGACACTCCAGTGTGTGGAGATTTCCTGGTGACAGCAATTGTGTGTTCTGTC |
| | TAAGCATGTCTAGGGCCGGTCATCAGGAAGGACAGGAAGGACAGGCTGGAATTCTTGGAAA |
| | CAGCTGCACCTGCCATCCACCAGGGAACTTTATTGTCTTCTGTTCTGCTAAAATTAAATCA |
| | GGCACACCAAGTTTAACTAGCACTATCTTCCTAGCTTAAGAAAATTGATGGCAGGTTTCAA |
| | TAACACCTATATTAAACCTTCAGAGCAACACCTACATTAGTGTCTGATTTGATAATTGAGA |
| | CTATGTTCTAGCCAAGCAGACACATAAAACTGGTGATTGCCATGCTTAGTTCATATTTCAT |
| | ATTTATCAATAGAATCTGGCTGGTGTTATAAACAGATTTGCCAACACATGTGTGTTAGTGT |
| | TTGTCTTCAAATTCCTATTTCTATTTAAACTCCCGATTCCTACTCTCAGTGGGAGGGAAAA |
| | CTCACAGCCAATCACACATCACAGGACAAATCTGTAAACGAAGAGTCATTCCTCTGAAGGT |
| | CCTGGGTGTTCAGGACTCTCAGGCAGGTGCTGAGGACCCTGTCTTGGGAGTGCCCAGCAGA |
| | TCTCAGAACCCTACATGGGGCCTGCTGGACACTCATGTGGGATAACTAGTCGCCACTTATT |
| | CAGAGTTACCAGTGAGCTTTGACTGTTCCGAATGGGACCAGCATGGAGTCAAGGTGCCTGC |
| | TCAATGTCAGAGACAGCGATGGTCTCAGAAACAATCCAGGTAATCTCTAGGCCAATAAAAT |
| | GTGGATTCACAGTGAGAAGTACATCCTGGAGGTGGAGCTTGTTCTTCAGTGGGAAGAGTGC |
| | TGTGCACAGAAAGCTTAGAAATGGGGAAGGGGGTGCGTTTCCTCAGGCAGGATTAGGGCTT |
| | CGTCCCTCAGCGTCCCACTCTTGTATGGCTGATGTGGCATCTGTGTTTTCTTTCTCATACT |
| | AGATAAGGCTTTGAGCTGTGAAATACCCTGCCTCATGAATATGCAAATAACCTGAGCTCTT |
| | CTGAGGTAAATATAGGTATATTGGTGCCCTGAGAGCATCACTCAACAACCACATCTGTCCT |
| | CTAGAGAAAACCCTGTGAGCACAGCTCCTCACCATGGACTGGACCTGGAGGATCCTCTTCT |
| | TGGTGGCAGCAGCTACAAGTAAGGGGCTTCCTAGTCTCAAAGCTGAGGAACGGATCCTGGT |
| | TCAGTCAAAGAGGATTTTATTCTCTCCTGTGTTCTCTCCACAGGTGCCCACTCCCAGGTGC |
| | AGCTGGTGCAGTCTGGGGCTGAGGTGAAGAAGCCTGGGGCCTCAGTGAAGGTCTCCTGCAA |
| | GGCTTCTGGATACACCTTCACCAGTTATGATATCAACTGGGTGCGACAGGCCACTGGACAA |
| | GGGCTTGAGTGGATGGGATGGATGAACCCTAACAGTGGTAACACAGGCTATGCACAGAAGT |
| | TCCAGGGCAGAGTCACCATGACCAGGAACACCTCCATAAGCACAGCCTACATGGAGCTGAG |
| | CAGCCTGAGATCTGAGGACACGGCCGTGTATTACTGTGCGAGAGGCACAGTGTGAAAAACC |
| | ACATCCTCAGAGAGTCAGAAACCCTAGGGGAGAAGGCAGCTGTGCTGGGCTGAGGAGATGA |
| | CAGGGGTTATCAGGTTTAAGGCTTTTTTGAAAATGGGTTATATATTTGAGAAAAAAATAAC |
| | AATAGAAACAAGTACACACTCTAATTTTAAGAGATATATTCAATTCAAGAATTGTAGAAGC |
| | CGAATTCACAGTGGGAAAGGCCACACTCAATAAAGTTGATAAAAACATTCCAGGAAGGTGC |
| | TACTGTGAACAAGTTTTCAAATTAGATGAATAAATAATTTGGAGCAATGTTATTTGATCAT |
| | GAGGTGAGACAGTATGATTCTTAAAAAGTGGAAAAAATTCTTTCAAATGTTTCTGCCACTC |
| | TTTATCATAAAATTAATTTTAGAGCAGTTTTAGGATTACAATGAAATTGCACAGAAGGCAT |
| | GAGAATTCCCATGACTCCCTGCCCTACACAGGCAGAGCCTCCTCCACTATGACCATCCAGC |
| | ACCACAGTCACAAATCAGTTACAATGGAGGAATCTCCAAGGACATTTGATTCTTTCTTTTT |
| | TGGTGATGCCCTAATATAACAAGCCTAATTTATCTCGGAATACCACAAGTTTTGTTCAGTG |
| | GATTTCTAGGAATCATATTAGTTAGAGGGAAAGTTGGTGAGGCTCTTACTATTTGAACTCA |
| | TTCTTCCAAAATCCACAATGGAAAAGGTAAATGTGGTCTCCGTGACCTCAATTCAAGGGCT |
| | GAAGCCCTTTCCCTGTAGCTCAGCTGGGGCTCAGGCTGTGGCTACTGCAGCCATGTGGAAG |
| | AGGCTGAAGGGACTTTCTTCACTCTCCTTGCTCAGGACCATCCACTGTATTGTGTATAGGC |
| | TTCTCTGGAAATGCAAGTGGCCATTTGTAGTGAAAGAAATATGTTTGTCTGGTTAAAATGG |
| | GAGGTGGATGTAGAGTTAATTGGCTGCTACATAAACTGTCCTTCTCCACCAGTGCTTTTAG |
| | GATGAGATTGTGAAATTTGTAAGAATCAAAATGGAGTCACATATGTTAAAACCCTGACAAA |
| | TGGATTCAGGAAGTGTAGGGAGAATTCTTACACACATATCCCTGACAACAAGAACTATCAT |
| | AAAATAGTTCTTGCAAAAAGACCAACATGACCTCATAATCATGACTTCTGCAAAGACTTCT |
| | ACTCAGAATCTACTTGCCCAGCCTTAGATTAATGCCATCTGAATTACACTGATCATGTTAC |
| | TATCACTGCTCCTCACCACAGATGCAACACCCTCCTGAGTCCTGAAACCTGACTCCATCCC |
| | ATAGAGTAGGGCACAGATGAGGGGAATGCAAATCTCCACCAGCTCCACCCTCCTCTGGGTT |
| | GAAAAAGCCGAGCACAGGTCCCAGCTCAGTGACTCCTGTGCCCCACCATGGACACACTTTG |
| | CTCCACGCTCCTGCTGCTGACCATCCCTTCATGTGAGTGCTGTGGTCAGGGACTCCTTCAC |
| | GGGTGAAACATCAGTTTTCTTGTTTGTGGGCTTCATCTTCTTATGCTTTCTCCACAGGGGT |
| | CTTGTCCCAGATCACCTTGAAGGAGTCTGGTCCTACGCTGGTGAAACCCACACAGACCCTC |
| | ACGCTGACCTGCACCTTCTCTGGGTTCTCACTCAGCACTAGTGGAGTGGGTGTGGGCTGGA |
| | TCCGTCAGCCCCCAGGAAAGGCCCTGGAGTGGCTTGCACTCATTTATTGGGATGATGATAA |
| | GCGCTACAGCCCATCTCTGAAGAGCAGGCTCACCATCACCAAGGACACCTCCAAAAACCAG |
| | GTGGTCCTTACAATGACCAACATGGACCCTGTGGACACAGCCACATATTACTGTGCACACA |
| | GACCACAAAGACACAGCCCAGGGCACCTCCTGTACAAAAACCCAGGCTGCTTCTCATTGGT |
| | GCTCCCTCCCCACCTCTGCAGAACAGGAAAGTGCAGCTGAGATACGTTTTCCTGCCAGGGC |
| | CTGCATTTCCCATCCCCATTAGACTCAGAGCCCTGTCTTCCTCCTTCTTCTTTAATAATAA |
| | ATGGCATGACTCCTGTTAATAGTTCATAGAAGCAGAAGCTGAGTCCTGTTTGTCAAACATT |
| | CAGCATGAAATGTTCATGTTACCTGGGCCAGATGCATCACTGGTATGTGGCCGCCAGTTTA |
| | ATGGGCTCATACTACTCCAAGCGGCCGAATACAAAACAGCAGAACATATGCCTTTATTAAA |
| | CTGCAGGAAATGACACTGGAGAAAAATGTGGGGATAAGGGAGCAGGAGGAATTGTGAAAAT |
| | TCAGATAATAGCTTTTCTCGTAGAGAGAAAGGTGCGTTAATAAAATAATTTTATAAAACAA |
| | TAAAACAGATGGAATGTGTCCCCATCACGGAGTGTCTCACAGTAACAGTGCAGAAATAAAT |
| | TTCTAAAAATGTCGTAGGGTAGGTGTGATAGACAAGGCTTATTTCCACACAGGGAGCCCAT |
| | AAATACCCATGTAATTAAAAATCCAGCTCTTAGAAAGGGAGTGCCTCAACTTGACATCAGA |
| | AACCCACGTCAGTGAGTCTGAGGAGCAATGTGCTCTGCAGTGCAAGGGCTGGGCTGCAGGG |
| | GACGCTCAAAGCCCACCCAGTACAAGTTTCAGCCCCAGAGCAGGTGCACAGGAGGCTGGGG |
| | AGGGATTCCTCTCGAGATCTGTGTCATTCTTTAAAAAATCTAAAATACATATTTGACAACT |
| | GAACATTCTATAAATATCCTATTCAATTGCGAGCATTTATCAAACTTGATGTTGTAATGAG |
| | AACCACTTTTACACTGGAGATTTCTAACCCTTCTAGATATCTTAATAGTATGCAGCTGGAG |
| | GTTAAGGAAACTACTTTTCTTTTCTATAAATAAGTGAAACTTTTGGAGAAACACACTCATC |
| | CCCCAAATAATACATTCATGTATTAAAGCCTAGAAATGCTTATATTACCTCTGAGCCATTT |
| | GAGTGTGGGTTCCCAGTGAAGTCCTGTTCTAGGGAAAATTGTTAGCCAATGGGAGAAGCTC |
| | TGTCAACATAGAGCTTAGGGATATGGCAGGGCTCACATGGCCTCTAAGGGGATTACAGCTT |
| | GAACCTTGAGCATCCTCCGTGTGTCATCTCTCTGCTCTTTCTCATGCAATATCAGGTATGA |
| | AATAGGATCACTCATGAATATGCAAATAACTGAGGTAAATATAGCTATCTTTGGGCCCTGA |
| | GAGCATCACCCAACAACCACACCCCTCCTAAGAAGAAGCCCCTAGACCACAGCTCCACACC |
| | ATGGACTGGACCTGGAGGATCCTCTTCTTGGTGGCAGCAGCAACAGGTAAGGGGCTCCCCA |
| | GTCACTGGGCTGAGGGAGAAACCAGCACAGTCAAGTGAGACTTCATGCACTCCCATCTCCT |
| | CTCCACAGGTGCCCACTCCCAGGTGCAGCTGGTGCAATCTGGGTCTGAGTTGAAGAAGCCT |
| | GGGGCCTCAGTGAAGGTTTCCTGCAAGGCTTCTGGATACACCTTCACTAGCTATGCTATGA |
| | ATTGGGTGCGACAGGCCCCTGGACAAGGGCTTGAGTGGATGGGATGGATCAACACCAACAC |
| | TGGGAACCCAACGTATGCCCAGGGCTTCACAGGACGGTTTGTCTTCTCCTTGGACACCTCT |
| | GTCAGCACGGCATATCTGCAGATCAGCAGCCTAAAGGCTGAGGACACTGCCGTGTATTACT |
| | GTGCGAGAGACACAGTGTGGAAACCCACATCCTGAGAGTGTCAAAAACCCTGTGGGAGGAG |
| | GCAGCTGTGCTGAGCTGAGGCAGTGACAGGGACAACGTGGCTGCACCCTTTGTAGGGTTAT |
| | AAACATACATATATATGCACCCAACAGTGGAGTACCCAGATATATCAAGCAAACATTATTA |
| | GAGCTAAACAGAGAGATAGACCCAATACAGTAAAAGCTGGAGACTTTAACACCCCACCCTC |
| | AGCATTGGACAGATCTTCCAGACAGAAAATTAACAATGAAATCTTAGACTAAATGTACATG |
| | ACAGACCAAATGGACATAATGGATATTGACAGAACACTTCATCCAATGGCTGTAGAGTGCA |
| | CAGTCTTTTCCTAAACATATGGATCAGTCTCAAGGATAGACCATATAGTAGACCAGAAAAT |
| | TAGTCATTTTTTAATTGAGAAGATATCAAGTACCTTCTCTGATCATAATGGAAGAAAACTA |
| | AATTAATAAGAGGAATCTTGGAAAACTATGCAAACACATGGAAATTAAACAATATGCTCCC |
| | TAATTAAACAATGTGCTCCCAAATTGAACCACATGCTCCAAAATGGGTTAATAAAGACATA |
| | AAAAATTTTAAAATTTATTGAAAGAAATGATAAAGAAGACAAAGCATATGAAAACCTATGA |
| | AATACAGTGAAAGCAGTATTAAGAGGAAAGTTTATAGCTCACAGGCTGCAGCCCTGGAGCA |
| | GGTGCAAGGGAGGCTGGGGAGGGGTTCCTCCCAGGGTCTGATGTCTTCCTTTTCTCGGACA |
| | AACATGCTTTAATAAGTTAAACAAGACTTTAGTAAAGACTATTGATGTGTCTTTGTGTCTT |
| | TCAGTATACAGTTCTATTTGTAGGATTTATCTAACCTAACAAGTCAATGAGAATCACATGT |
| | AAAAGGAGAAATTTCTAGGATTTTCAGATATCTTAATAGGTAGGAGATGGAGAAAAGGGAT |
| | GGTTTTATTAATTCAGTGCTTGCCAATCTTAACAGAGACAGTAGTAAGACATGCAGAAAGC |
| | AAAGCCCAGAAAAGTATGAAGGTGTCAAAGTGCCATTTAAGTATGGGTTCACTTGGAGGAC |
| | CATGTTCTGCGGGAACTTGTTTTCAGCAGACAATCTATTTTAGCAGAGTTCTGGGCATACA |
| | AGGGGACACACATCATTAAACAAGGATTGGGACAGGGACTTCAGCGTCCCACTGTTGCATG |
| | GCCCATAAATTATGTGTGTTCTCTTTCTCATCTTGGATCAAGTCTAGAGCTATGAAATAGT |
| | ATCCCTCATGAATATGCAAATAACCTGAGATTTACTGAAGTAAATACAGATCTGTCCTGTG |
| | CCCTGAGAGCATCACCCAGCAACCACATCTGTCCTCTAGAGAATCCCCTGAGAGCTCCGTT |
| | CCTCACCATGGACTGGACCTGGAGGATCCTCTTCTTGGTGGCAGCAGCCACAGGTAAGAGG |
| | CTCCCTAGTCCCAGTGATGAGAAAGAGATTGAGTCCAGTCCAGGGAGATCTCATCCACTTC |
| | TGTGTTCTCTCCACAGGAGCCCACTCCCAGGTGCAGCTGGTGCAGTCTGGGGCTGAGGTGA |
| | AGAAGCCTGGGGCCTCAGTGAAGGTCTCCTGCAAGGCTTCTGGATACACCTTCACCGGCTA |
| | CTATATGCACTGGGTGCGACAGGCCCCTGGACAAGGGCTTGAGTGGATGGGACGGATCAAC |
| | CCTAACAGTGGTGGCACAAACTATGCACAGAAGTTTCAGGGCAGGGTCACCAGTACCAGGG |
| | ACACGTCCATCAGCACAGCCTACATGGAGCTGAGCAGGCTGAGATCTGACGACACGGTCGT |
| | GTATTACTGTGCGAGAGACACAGTGTGAAAACCCACATCCTGAGGGTGTCAGAAACCCCAG |
| | GGAGGAGGCAGCTGTGCTGGGGCTGAGAAATGAAAGGGATTACTATTTTTAATGTTGTTTA |
| | CAGTATGTCATTAATAAATTGAAAAAAAGTAACAATAGAAGTATATACTCTAATTATATGG |
| | GAACTTTGTTTTTTCAGTTTTTTCATTTTTTTTTTTTTTTTTGGTTTGTTTGTGACAGAGT |
| | CTCACTCTGCCACCCAGGCTGGAGTGTAACGGCACAATCTCAGCTCACTACAACCTCCACC |
| | TCCCAGGTTCAAGCAATTCTCCTGCCTCGGCCTCCAGAGTAGTTGGGATTACAGGCACCCG |
| | CCACCATGCCCGGTGAATTTTTGTATTTTTAGTAGAGACGGGGTTTCACCATGTTAGCTAG |
| | GCTGGTCTCAAACTGCTGATCTCAGGTGATCTACCCTCCTCAGCCTCCCAAAGTCCTGGGA |
| | TTACAGGCGTGAGCCACTGCGCCTGGCCCAATTATATGGGAATTGTTTATATAATTATCAC |
| | CCTATAAGCAAAATTCATGGAGGAGGAAAAGCTCTACTGAAGAAAGCTGATACCGGCATTC |
| | CCATGAAAGTATCTGTGTAGAAGTAAGTATTAAAATCAGTTGAATAGGCAAGGCATGGTGG |
| | CTCACGCCTATAATCCCAGCACTTTGGGAGACCGAGGCAGGTGGATCCCCTTGGGGTTTGG |
| | GATGAGCTGCCCTTGCCCACTGTGCTCTGTGGACCTCCCTTTAGAAGCTCACAGCTCCCTG |
| | CACTCGGCTCCATCCTGCCCCACCACACAGAAGCAAAACCCCTCTCCTTTCCACTGCAGGC |
| | TTTTCCTGGACCAGAATGCTGACCTGCTGCCCTTCACTCCCGAAGTGGTGGGACTGCCTGG |
| | GGTGGTGTGGGTGTTGAGCCTTCTTACTCTAGGGACCTGGCACCTGGCCCCAGGGGCACAG |
| | GGATGGTGCATCTGCCTAGGGATGCCTCCTCATGCCAGGGGGTGGGGGTTAGTACCATCGG |
| | CCCTCAGGATTTGTTGCATGAATGAGTGAATGGGTGAATAAATGAAGGGGATCTGATCTAT |
| | GAATAAGGGTATATAGACTTTGGTTGATGTAGGACGCCAAATGCTGGAATTTCAGAGTCAT |
| | CACACCCAGGGGCCCTGCCTCTGAGCTCCTCTTTGCATCCAATCTGCTGAAGAACATGGCT |
| | CTAGGGAAACCCAGTTGTAGACCTGAGGGCCCCGGCTCTTCAATGAGCCATCTCCGTCCCG |
| | GGGCCTTATATCAGCAAGTGACGCACACATGCAAATGCCAGGGTGTGGTTTCCTGTTTAAA |
| | TGTAGCCTCCCCCGCTGCAGAACTGCAGAGCCTGCTGAATTCTGGCTGACCAGGGCAGTCA |
| | CCAGAGCTCCAGACAATGTCTGTCTCCTTCCTCATCTTCCTGCCCGTGCTGGGCCTCCCAT |
| | GGGGTCAGTGTCAGGGAGATGCCGTATTCACAGCAGCATTCACAGACTGAGGGGTGTTTCA |
| | CTTTGCTGTTTCCTTTTGTCTCCAGGTGTCCTGTCACAGGTACAGCTGCAGCAGTCAGGTC |
| | CAGGACTGGTGAAGCCCTCGCAGACCCTCTCACTCACCTGTGCCATCTCCGGGGACAGTGT |
| | CTCTAGCAACAGTGCTGCTTGGAACTGGATCAGGCAGTCCCCATCGAGAGGCCTTGAGTGG |
| | CTGGGAAGGACATACTACAGGTCCAAGTGGTATAATGATTATGCAGTATCTGTGAAAAGTC |
| | GAATAACCATCAACCCAGACACATCCAAGAACCAGTTCTCCCTGCAGCTGAACTCTGTGAC |
| | TCCCGAGGACACGGCTGTGTATTACTGTGCAAGAGACACAGTGAGGGGAAGTCAGTGTGAG |
| | CCCAGACACAAACCTCCCTGCAGGGATGCTCAGGACCCCAGAAGGCACCCAGCACTACCAG |
| | CGCAGGGCCCAGACCAGGAGCAGGTGTGGAGTTAAGCAAAAATGGAACTTCTTGCTGTGTC |
| | TTAAACTGTTGTTGTTTTTTTTTTTTTTTTGGCTCAGCAACAGAGATCATAGAAAACCCTT |
| | TTTCATATTTTTGAAATCTGTTCTTAGTCTAATGGAGATTCTCTAATATGTGACAATGTTT |
| | TTCTCTTGCTGTTTTTGGAATTCTTTGTCTTTGACTTTTGACAACTTGACTTTTGACAGTG |
| | TGCCTCAAAGAAGTTCTATTTTGGGTTCTGTGAACCTCCTGGATCTGGGAAGTTTTCAGCT |
| | ATGATTTCATTAAACGTGTTTTCTACACCATTTCCCTACTCTTTTGGAATACCCATAATGC |
| | AAATATTTGTTCACTTAATTGTGTCCCATAAATGCTGGGGATTTTCTTCATTCCTTTTTAC |
| | TCTTTTTTTCTTTTTATTCATCTGCCTGAATTATTTCAAAAGATCTGTCTTCAACTTCAGA |
| | AACTCTTTTGCTTGGCCTAGTCTAATCTTGAAGGTCTCAATTGTACTTTTAATTTCATTCA |
| | TTGAATTCTTCAACTCTGGAATTTCTGTTGGTTCTTTTTTATGATACTTATCTCTTTGTTG |
| | AATTCGAACACAAAATAGTGACAGCAGAGATGACGGCAAGTTGGCATTTTTCTTTTCTAGC |
| | AATAAAACTTAAAGCTGACTCAAGGAGAAATGGAAATCATAATTGGAACAGTAATCCTCAA |
| | GAAAGCATTAAGATTATTAAATAATTGCCCTCACAGATGACTTCAGGCCAAGATGGCTTTA |
| | TGGGTGAAGTTTAGACTTTCACAAAACTAATCAGTTCCCATAAGAACTGCTCCAGGATTTG |
| | GAGGAACATGGGAAAGTCTATTAAAGGGATCACAATTCACAGTCCCCAGAGTAAAACATGG |
| | GCTAACTTGCATTTTGGCAAAGAGCCAAATGTTATAAATGACATCCTAGAAGGCCAAATTC |
| | TGTCCATCTCGTTGAACAAGGACTTACACCAGGAATTTAGAACTATTTATAGCTCATCCCA |
| | CCACTCAGGCCAATGATGACCCATGATCATCTCACCAGAAATGGAAAGACTCAGATGATTA |
| | ATAGAGTCTCAATTTCTCTGAGACATCTAAGAGCCCAGCCCAAGCCCAGACCCAGGAGGGC |
| | ACCCAGGCCTGGACAGAGAACACTGATATCACACCAGCCCTCCAGAGGGAAGCAGAGACTC |
| | CTTCAAGCTCTGGAAACACAGGCCCAGACAGCTGCCTAAAGTTGGGCAGGCTTCACTGCAA |
| | ACCCAAATCATGAAGCTAGGTAACACCTTTACAGATTCTTTACATTTAAAAATCATCAAAA |
| | CAAGAGTAAATAATAAACTCAAATAATATTAATCTAATATGTAAAGGTCTTGTACCATTAT |
| | TATGCAAACAACATACATAAGCTAATAAGAAAAAGAACAAATCCCTTAAGAAATCGGCAAA |
| | AAGGATATAACACAATTTCTAAAAGAAAACAAATGGCTAGCACACATAAGGAAAACACTTT |
| | GTGAACAGACATTCTTCAGAACATTATTTATAATTATAAAATAGTTGAAAGCAAGATAGTG |
| | CCTGAAGAAATTATGGTGCATACATTAGTGGGACTATTCTGCAAACATTCCCAATTATACT |
| | TGTCACATATCTGTGATAACGTGACAGCCAGCATTCATGGGGTGACCTCATTTGGTAAAAG |
| | GGTGCAAAGCTCAACACGCATTGTGAGATGACTGTGGTGTAAAATTAGTGGGATTATTCCG |
| | CAAACATTCCCAATTATACTTACCGCATATCTGTGATAACATGACAGCATTCATGGGGTGA |
| | CCTCATTTGGTAAAAGGGTGCAAAGCTCAACACGCATTGTGAGATGACTGGTGTAAATACA |
| | AAGACCAAACTGTGAAAAGGAGTCCATCAATTAATCGATGCTTACCTTCAGTTTTGGGCTA |
| | ATTTTTAAAGTATGCTATAAGCATATGCTCCTGTTATAACAGAATGGAGGGATTATGAGAG |
| | ATGATGCAGGTGTGTCCTGGGCCTCCCCTGGCCCACTGGGCCCTAGAGATGCCTTCCCAGG |
| | CATCGCTGTCAGGGCTTCCCTCAGAGGGAGTCCTGTATTGACCTCACCACCAAGGTCTGGA |
| | GCAGGGGATCCTTAGATATTGGTTGGGGTTATCTCACCTTAGGTCTGAATTTGGGGTTGTC |
| | TTAGACTGTTTTGTGCTGTTAGAATAGAATACCCAAGACTGGGAAATTTATACTGAACGGA |
| | AATTTATTTCTCACAGTTCTAGAGGCTGTGAAGTCCAAGAGCACAGGTGCCAGAGCAAGTC |
| | CAAGAGCAAGGGAAAGTCCAAAGCAAGTCCAGGAGCATCTGGCGAGGACCTTCTTGCTGTG |
| | TCATCACATGGCGGAAGGCAAGAAAGAGAGCAAGAGGGGGCCGAACTCACCCTTTTATAAC |
| | AGCACCAATCCCACCCATGAGGTGGGGACCTTATGACCTAATCACTCTTCATACTGTTACA |
| | ATGGCAATGAAATTTCAACATGAGTTTTGGAGGAGAGAAGCATTCAAACCACAGCAAGGGT |
| | GCTCCTACCTCCTCTCTCAGGGCATCTGCAGAAAGAGCTGCAACTGCACGTCCTTCCTCCG |
| | TCCATCCTCCATCCCTTCCCAATGTCCGTGCATATCCTGTGACCCAGGAGGTCTGGCATAG |
| | GGGGTGCTCCTGCCTTAGGTCTGAGGCCCTGTCTGAAGAGGGGTAGGTGAGGAGGCCATCT |
| | GATGGTCTGGGCCAAGACAGTCACAGGACGCATCATTTATCATCAAGGAGGCTGAGGGTTG |
| | AGTCTCCAGGTCCAGGGAACTCCCCACAAAGTGGGAACCCTGCCCAGCTCCACACAGCCTC |
| | TGCTGGGGGACCCTGCTCTGGTGCAGAGCCTGGGGACAGGTCTTGAGCTCAGCCAGAGTCT |
| | GCCTCCCTGTCATTTAGGAACTAAACCAAGCGGCAGGATGCTGGAGCCCAGCCCCCATCTG |
| | ACCTTACAGGGCCAAGGCTGGGGCCCTGGGTTCCCCTCAAGGCGCAGCAGGACTGGAGCCC |
| | CAGGCAGTGCAGGAGTGGCCAAAGCTGGGGCTTCCTCCAGAGCCCCCAAGCATCACGGCAC |
| | CAAGAAGGGTAGGACCCTGGCCTGAGGAATTGGCACCAAAGCCCCAGAAACTACCCTGGAC |
| | ACCATGGAGAGAGGCCTGGAGGGGAAGCACCAGGCACTGCCTCCCCTTCTGATCCCACCTG |
| | AGGTGGCTGCCAAGCCCAGAGAGCCGCTCTGATGTCCCCCAGCCCTGCAGCCCAGGGATAC |
| | CTGTACTGTGCCCCTGGGGGACCCCTGGCCAGTCTGTGCAAAGAAGTCACCACCCTACACT |
| | CAGAGACAGTGGGGGTCCTCGTCCCACATCCTCAGAGCATGGCCCGGCTGCTGCAGGGATG |
| | GTCTCCTGGTCCTCAGAGCATGGCCCGGCTGCTGCAGGGATGGTCTCCTGGTCCTCAGAGC |
| | ATGGCCCAGCTGCTGCAGGGATGGTCTCCTGGAGGCCCCCCAGTGCTCTATTGTCAGGGCT |
| | CCCTCCACCCCCCGCACCAAGAGAGAGCCAGACCCCAGCAAGGCTTCCAGTGGCTTCAGGT |
| | CACACCCCTAGGCTGACCCCAGCCCCATTAACACCTGCCTGAGAAAGCTCCACGCACCAGA |
| | ACTGACCGTCTGCTCCAACTCTTGACCTCCCGTTCTCAGGGCGTCTGCTGAAAAGGCTGCA |
| | ACTGCACATCCTTCCTCCGTCCGTTCCCGATGTCCGTGTGTCTCCTGTGGCCAGGAAGGTC |
| | TTTCTCGGGACCTGAGAGCCGCTCCCTGAAGTGTCCCCATTGGGAAGGATGGGGCCTGTGT |
| | CTCCAGGCTCTGGGAGGACAGAATCCTGACCTCAACAGTGGCCGGCACGGACACAACTGGC |
| | CCCATCCCGGGGACGCTGACCAGCGCTGGGCAACTTTTCCCTTCCCCGACGACTGAGCCCC |
| | GAGCACCCTCCCTGCTCCCCTACCACCTCCCTTTACAAGGCTGTGGCCTCTGCACAGATGA |
| | TAATGGAGCTTGGCTCATTCCCCTAGAGTCGGTAGGGAGTTAAGGACAAAACTCAGTTTCC |
| | TCCACCTGAACTCAAGTCTGCCTATGTTTACCTAATCACACCTGGTGGACAGTTTGGACAA |
| | ACTTGCACACTCAGAGACACAGACACTTCTAGAAATCATTATCTCCCTGCCCCGGGGACCC |
| | CACTCCAGCAGAAGTCTGCTAGGCACTGGCCTGGGCCCTCCTGCTGTCCTAGGAGGCTGCT |
| | GACCTCCTGCCTGGCTCCTGTCCCCAGGTCCAGAGTCAGAGCAGACTCCAGGGACGCTGCA |
| | GGCTAGGAAGCCGCCCCCTCCAGGCGAGGGTCTAGTGCAGGTGCCCAGGACAAGAAAGATT |
| | GTGAATGCAGGAATGACTGGGCCACACCCCTCCCGTGCACGCCCCCTCCTGCCCTGCACCC |
| | CACAGCCCAGCCCCCCGTGCTGGATGCCCCCCCACAGCAGAGGTGCTGTTCTGTGATCCCC |
| | TGGGAAAGACGCCCTCAACCTCCACCCTGTCCCACGGCCCAAGGAAGACAAGACACAGGCC |
| | CTCTCCTCACAGTCTCCCCACCTGGCTCCTGCTGGGACCCTCAAGGTGTGAACAGGGAGGA |
| | TGGTTGTCTGGGTGGCCCCTAGGAGCCCAGATCTTCACTCCACAGACCCCAACCCAAGCAC |
| | CCCCTTCTGCAGGGCCCAGCTCATCCCCCTCCTCCTCCCTCTGCTCTCCTCTCGTCGCCTC |
| | TACGGGAAATCCGGGACTCAGCAGTAACCCTCAGGAAGCAGGGCCCAGGCGCCGTTTAATA |
| | GGAGGCTTCCTCACAATGAAACTTTTAGAAAGCCTTGACTACAATGATGACCTTGGTGTGG |
| | CTGTGAACACTGTCAGCTCCCACAGCTGCTGCAGCAAAAAATGTCCATAGACAGGGTGGGG |
| | GCCCGGGGTCGTCTGCTGTCCTGCTCAGCCCACAGCACGCATGGAGGATCTGAGGTGCCAC |
| | ACCTGACGCCCAGGCCAGAACATGCCTCCCTCCAGGGTGACCTGCCATGTCCTGCATTGCT |
| | GGAGGGACAGGGGCAGCCTATGAGGATCTGGGGCCAGGAGATGAATCCTATTAACCCAGAG |
| | GAAAACTAACAGGACCCAAGCACCCTCCCCGTTGAAGCTGACCTGCCCAGAGGGGCCTGGG |
| | CCCACCCCACACACCGGGGCGGAATGTGTACAGGCCCCGGTCTCTGTGGGTGTTCCGCTAA |
| | CTGGGGCTCCCAGTGCTCACCCCACAACTAAAGCGAGCCCCAGCCTCCAGAGCCCCCGAAG |
| | GAGATGCCGCCCACAAGCCCAGCCCCCATCCAGGAGGCCCCAGAGCTCAGGGCGCCGGGGC |
| | AGATTCTGAACAGCCCCGAGTCACGGTGGGTACAACTGGAACGACAGGATATTGTACTGGT |
| | GGTGTATGCTATACCCACCGTGAGAAAAACTGTGTCCAAAACTCTCTCCTGGCCCCTGCTG |
| | GAGGCCGCGCCAGAGAGGGGAGCAGCCGCCCCGAACCTAGGTCCTGCTCAGCTCACACGAC |
| | CCCCAGCAGCCAGAGCACAGTGGAGTCCCCACTGAACCCCACTGAATGGTGAGGACGGGGA |
| | CCAGGGCTCCAGGGGGTCATGGAAGGGGCTGGACCCCATCCTACTGCTATGGTCCCAGTGC |
| | TCCTGGCCAGAACTGACCCTACCACCGACAAGAGTCCCTCAGGGAAACGGGGGTCACTGGC |
| | ACCTCCCAGCATCAACCCCAGGCAGCACAGGCATAAACCCCACATCCAGAGCCGACTCCAG |
| | GAGCAGAGACACCCCAGTACCCTGGGGGACACCGACCCTGATGACTCCCCACTGGAATCCA |
| | CCCCAGAGTCCACCAGGACCAAAGACCCCGCCCCGGTCTCTGTCCCTCACTCAGGACCTGC |
| | TGCGGGGGGGGCCATGAGACCAGACTCGGGCTTAGGGAACACCACTGTGGCCCCAACCTCG |
| | ACCAGGCCACAGGCCCTTCCTTCCTGCCCTGCGGCAGCACAGACTTTGGGGTCTGTGCAGA |
| | GAGGAATCACAGAGGCCCCAGGCTGAGGTGGTGGGGGTGGAAGACCCCCAGGAGGTGGCCC |
| | ACTTCCCTTCCTCCCAGCTGGAACCCACCATGACCTTCTTAAGATAGGGGTGTCATCCGAG |
| | GCAGGTCCTCCATGGAGCTCCCTTCAGGCTCCTCCCTGGTCCTCACTAGGCCTCAGTCCCG |
| | GCTGTGGGAATGCAGCCACCACAGGCACACCAGGCAGCCCAGACCCAGCCAGCCTGCAGTG |
| | CCCAAGCCCACATTCTGGAGCAGAGCAGGCTGTGTCTGGGAGAGTCTGGGCTCCCCACCGC |
| | CCCCCGCACACCCCACCCACCCCTGTCCAGGCCCTATGCAGGAGGGTCAGAGCCCCCCATG |
| | GGGTATGGACTTAGGGTCTCACTCACGCGGCTCCCCTCCTGGGTGAAGGGGTCTCATGCCC |
| | AGATCCCCACAGCAGAGCTGGTCAAAGGTGGAGGCAGTGGCCCCAGGGCCACCCTGACCTG |
| | GACCCTCAGGCTCCTCTAGCCCTGGCTGCCCTGCTGTCCCTGGGAGGCCTGGACTCCACCA |
| | GACCACAGGTCCAGGGCACCGCCCATAGGTGCTGCCCACACTCAGTTCACAGGAAGAAGAT |
| | AAGCTCCAGACCCCCAAGACTGGGACCTGCCTTCCTGCCACCGCTTGTAGCTCCAGACCTC |
| | CGTGCCTCCCCCGACCACTTACACACGGGCCAGGGAGCTGTTCCACAAAGATCAACCCCAA |
| | ACCGGGACCGCCTGGCACTCGGGCCGCTGCCACTTCCCTCTCCATTTGCTCCCAGCACCTC |
| | TGTGCTCCCTCCCTCCTCCCTCCTTCAGGGGAACAGCCTGTGCAGCCCCTCCCTGCACCCC |
| | ACACCCTGGGGAGGCCCAACCCTGCCTCCAGCCCTTTCTCCCCCGCTGCTCTTCCTGCCCA |
| | TCCAGACAACCCTGGGGTCCCATCCCTGCAGCCTACACCCTGGTCTCCACCCAGACCCCTG |
| | TCTCTCCCTCCAGACACCCCTCCCAGGCCAACCCTGCACATGCAGGCCCTCCCCTTTTCTG |
| | CTGCCAGAGCCTCAGTTTCTACCCTCTGTGCCTACCCCCTGCCTCCTCCTGCCCACAACTC |
| | GAGCTCTTCCTCTCCTGGGGCCCCTGAGCCATGGCACTGACCGTGCACTCCCACCCCCACA |
| | CTGCCCATGCCCTCACCTTCCTCCTGGACACTCTGACCCTGCTCCCCTCTTGGACCCAGCC |
| | CTGGTATTTCCAGGACAAAGGCTCACCCAAGTCTTCCCCATGCAGGCCCTTGCCCTCACTG |
| | CCCGGTTACACGGCAGCCTCCTGTGCACAGAAGCAGGGAGCTCAGCCCTTCCACAGGCAGA |
| | AGGCACTGAAAGAAATCGGCCTCCAGCACCCTGATGCACGTCCGCCTGTGTCTCTCACTGC |
| | CCGCACCTGCAGGGAGGCTCGGCACTCCCTGTAAAGACGAGGGATCCAGGCAGCAACATCA |
| | TGGGAGAATGCAGGGCTCCCAGACAGCCCAGCCCTCTCGCAGGCCTCTCCTGGGAAGAGAC |
| | CTGCAGCCACCACTGAACAGCCACGGAGCCCGCTGGATAGTAACTGAGTCAGTGACCGACC |
| | TGGAGGGCAGGGGAGCAGTGAACCGGAGCCCAGACCATAGGGACAGAGACCAGCCGCTGAC |
| | ATCCCGAGCCCCTCACTGGCGGCCCCAGAACACCGCGTGGAAACAGAACAGACCCACATTC |
| | CCACCTGGAACAGGGCAGACACTGCTGAGCCCCCAGCACCAGCCCTGAGAAACACCAGGCA |
| | ACGGCATCAGAGGGGGCTCCTGAGAAAGAAAGGAGGGGAGGTCTCCTTCACCAGCAAGTAC |
| | TTCCCTTGACCAAAAACAGGGTCCACGCAACTCCCCCAGGACAAAGGAGGAGCCCCCTGTA |
| | CAGCACTGGGCTCAGAGTCCTCTCCAACACACCCTGAGTTTCAGACAAAAACCCCCTGGAA |
| | ATCATAGTATCAGCAGGAGAACTAGCCAGAGACAGCAAGAGGGGACTCAGTGACTCCCGCG |
| | GGGACAGGAGGATTTTGTGGGGGCTCGTGTCACTGTGAGGATATTGTAGTAGTACCAGCTG |
| | CTATGCCGTATTACGATTTTTGGAGTGGTTATTATACCCACAGTGACACAGCCCCATTCCC |
| | AAAGCCCTGCTGTAAACGCTTCCACTTCTGGAGCTGAGGGGCTGGGGGGAGCGTCTGGGAA |
| | GTAGGGCCTAGGGGTGGCCATCAATGCCCAAAACGCACCAGACTCCCCCCCAGACATCACC |
| | CCACTGGCCAGTGAGCAGAGTAAACAGAAAATGAGAAGCAGCTGGGAAGCTTGCACAGGCC |
| | CCAAGGAAAGAGCTTTGGCGGGTGTGCAAGAGGGGATGCGGGCAGAGCCTGAGCAGGGCCT |
| | TTTGCTGTTTCTGCTTTCCTGTGCAGATAGTTCCATAAACTGGTGTTCAAGATCGATGGCT |
| | GGGAGTGAGCCCAGGAGGACAGTGTGGGAAGGGCACAGGGAAGGAGAAGCAGCCGCTATCC |
| | TACACTGTCATCTTTCAAGAGTTTGCCCTGTGCCCACAATGCTGCATCATGGGATGCTTAA |
| | CAGCTGATGTAGACACAGCTAAAGAGAGAATCAGTGAAATGGATTTGCAGCACAGATCTGA |
| | ATAAATTCTCCAGAATGTGGAGCCACACAGAAGCAAGCACAAGGAAAGTGCCTGATGCAAG |
| | GGCAAAGTACAGTGTGTACCTTCAGGCTGGGCACAGACACTCTGAAAAGCCTTGGCAGGAA |
| | CTCCCTGCAACAAAGCAGAGCCCTGCAGGCAATGCCAGCTCCAGAGCCCTCCCTGAGAGCC |
| | TCATGGGCAAAGATGTGCAGAACATATGTTTGTCATAGCCCCAAACTGAGAATGAAGCAAA |
| | CAGCCATCTGAAGGAAAACAGGCAAATAAACGATGGCAGGTTCATGAAATGCAAACCCAGA |
| | CAGCCAGAAGGACAACAGTGAGGGTTACAGGTGACTCTGTGGTTGAGTTCATGACAATGCT |
| | GAGTAATTGGAGTAACAAAGGAAAGTCCAAAAAATACTTTCAATGTGATTTCTTCTAAATA |
| | AAATTTACAGCCGGCAAAATGAACTATCTTCTTAAGGGATAAACTTTCCACTAGGAAAACT |
| | ATAAGGAAAATCAAGAAAAGGATGATCACATAAACACAGTGGTCGTTACTTCTACTGGGGA |
| | AGGAAGAGGGTATGAACTGAGACACACAGGGTTGGCAAGTCTCCTAACAAGAAGAGAACAA |
| | ATACATTACAGTACCTTGAAAACAGCAGTTAAAATTCTAAATTGCAAGAAGAGGAAAATGC |
| | ACACAGCTGTGTTTAGAAAATTCTCAGTCCAGCACTGTTCATAATAGCAAAGACATTAACC |
| | CAGGTTGGATAAATAAACGATGACACAGGCAATTGCACAATGATACAGACATACATTCAGT |
| | ATATGAGACATTGATGATGTATCCCCAAAGAAATGACTTTAAAGAGAAAAGGCCTGATATG |
| | TGGTGGCACTCACCTCCCTGGGCATCCCCGGACAGGCTGCAGGCACACTGTGTGGCAGGGC |
| | AGGCTGGTACCTGCTGGCAGCTCCTGGGGCCTGATGTGGAGCAGGCACAGAGCCGTATCCC |
| | CCCGAGGACATATACCCCCAAGGACGGCACAGTTGGTACATTCCGGAGACAAGCAACTCAG |
| | CCACACTCCCAGGCCAGAGCCCGAGAGGGACGCCCATGCACAGGGAGGCAGAGCCCAGCTC |
| | CTCCACAGCCAGCAGCACCTGTGCAGGGGCCGCCATCTGGCAGGCACAGAGCATGGGCTGG |
| | GAGGAGGGGCAGGGACACCAGGCAGGGTTGGCACCAACTGAAAATTACAGAAGTCTCATAC |
| | ATCTACCTCAGCCTTGCCTGACCTGGGCCTCACCTGACCTGGACCTCACCTGGCCTGGACC |
| | TCACCTGGCCTAGACCTCACCTCTGGGCTTCACCTGAGCTCGGCCTCACCTGACTTGGACC |
| | TTGCCTGTCCTGAGCTCACATGATCTGGGCCTCACCTGACCTGGGTTTCACCTGACCTGGG |
| | CTTCACCTGACCTGGGCCTCATCTGACCTGGGCCTCACTGGCCTGGACCTCACCTGGCCTG |
| | GGCTTCACCTGGCCTCAGGCCTCATCTGCACCTGCTCCAGGTCTTGCTGGAACCTCAGTAG |
| | CACTGAGGCTGCAGGGGCTCATCCAGGGTTGCAGAATGACTCTAGAACCTCCCACATCTCA |
| | GCTTTCTGGGTGGAGGCACCTGGTGGCCCAGGGAATATAAAAAGCCTGAATGATGCCTGCG |
| | TGATTTGGGGGCAATTTATAAACCCAAAAGGACATGGCCATGCAGCGGGTAGGGACAATAC |
| | AGACAGATATCAGCCTGAAATGGAGCCTCAGGGCACAGGTGGGCACGGACACTGTCCACCT |
| | AAGCCAGGGGCAGACCCGAGTGTCCCCGCAGTAGACCTGAGAGCGCTGGGCCCACAGCCTC |
| | CCCTCGGTGCCCTGCTACCTCCTCAGGTCAGCCCTGGACATCCCGGGTTTCCCCAGGCCTG |
| | GCGGTAGGTTTGGGGTGAGGTCTGTGTCACTGTGGTATTACGATTTTTGGAGTGGTTATTA |
| | TACCCACAGTGTCACAGAGTCCATCAAAAACCCATCCCTGGGAACCTTCTGCCACAGCCCT |
| | CCCTGTGGGGCACCGCTGCGTGCCATGTTAGGATTTTGACTGAGGACACAGCACCATGGGT |
| | ATGGTGGCTACCGCAGCAGTGCAGCCTGTGACCCAAACACACAGGGCAGCAGGCACAACAG |
| | ACAAGCCCACAAGTGACCACCCTGAGCTCCTGCCTGCCAGCCCTGGAGACCATGAAACAGA |
| | TGGCCAGGATTATCCCATAGGTCAGCCAGACCTCAGTCCAACAGGTCTGCATCGCTGCTGC |
| | CCTCCAATACCAGTCCGGATGGGGACAGGGCCGGCCCACATTACCATTTGCTGCCATCCGG |
| | CCAACAGTCCCAGAAGCCCCTCCCTCAAGGCTGGGCCACATGTGTGGACCCTGAGAGCCCC |
| | CCATGTCTGAGTAGGGGCACCAGGAAGGTGGGGCTGGCCCTGTGCACTGTCACTGCCCCTG |
| | TGGTCCCTGGCCTGCCTGGCCCTGACACCTGGGCCTCTCCTGGGTCATTTCCAAGACAGAA |
| | GACATTCCCAGGACAGCTGGAGCTGGGAGTCCATCATCCTGCCTGGCCATCCTGAGTCCTG |
| | CGCCTTTCCAAACCTCACCCGGGAAGCCAACAGAGGAATCACCTCCCACAGGCAGAGACAA |
| | AGACCTTCCAGAAATCTCTGTCTCTCTCCCCAGTGGGCACCCTCTTCCAGGGCAGTCCTCA |
| | GTGATATCACAGTGGGAACCCACATCTGGATCGGGACTGCCCCCAGAACACAAGATGGCCC |
| | ACAGGGACAGCCCCACAGCCCAGCCCTTCCCAGACCCCTAAAAGGCGTCCCACCCCCTGCA |
| | TCTGCCCCAGGGCTCAAACTCCAGGAGGACTGACTCCTGCACACCCTCCTGCCAGACATCA |
| | CCTCAGCCCCTCCTGGAAGGGACAGGAGCGCGCAAGGGTGAGTCAGACCCTCCTGCCCTCG |
| | ATGGCAGGCGGAGAAGATTCAGAAAGGTCTGAGATCCCCAGGACGCAGCACCACTGTCAAT |
| | GGGGGCCCCAGACGCCTGGACCAGGGCCTGCGTGGGAAAGGCCTCTGGGCACACTCAGGGG |
| | CTTTTTGTGAAGGGTCCTCCTACTGTGGTGGATATAGTGGCTACGATTACTGACTACGGTG |
| | ACTACCACAGTGATGAACCCAGCAGCAAAAACTGACCGGACTCCCAAGGTTTATGCACACT |
| | TCTCCGCTCAGAGCTCTCCAGGATCAGAAGAGCCGGGCCCAAGGGTTTCTGCCCAGACCCT |
| | CGGCCTCTAGGGACATCTTGGCCATGACAGCCCATGGGCTGGTGCCCCACACATCGTCTGC |
| | CTTCAAACAAGGGCTTCAGAGGGCTCTGAGGTGACCTCACTGATGACCACAGGTGCCCTGG |
| | CCCCTTCCCCGCCAGCTGCACCAGACCCCGTCCTGACAGATGCCCCGATTCCAACAGCCAA |
| | TTCCTGGGGCCAGGAATCGCTGTAGACACCAGCCTCCTTCCAACACCTCTTGCCAATTGCC |
| | TGGATTCCCATCCCGGTTGGAATCAAGAGGACAGCATCCCCCAGGCTCCCAACAGGCAGGA |
| | CTCCCACACCCTCCTCTGAGAGGCCGCTGTGTTCCGTAGGGCCAGGCTGCAGACAGTCCCC |
| | CTCACCTGCCACTAGACAAATGCCTGCTGTAGATGTCCCCACCTGGAAAAGACCACTCATG |
| | GAGCCCCCAGCCCCAGGTACAGCCATAGAGAGAGTCTCTGAGGCCCCTAAGAAGTAGCCAT |
| | GCCCAGTTCTGCCGGGACCCTCGGCCAGGCTGACAGGAGTGGACGCTGGAGCTGGGCCCAC |
| | ACTGGGCCACATAGGAGCTCACCAGTGAGGGCAGGAGAGCACATGCCGGGGAGCACCCAGC |
| | CTCCTGCTGACCAGAGGCCCGTCCCAGAGCCCAGGAGGCTGCAGAGGCCTCTCCAGGGGGA |
| | CACTGTGCATGTCTGGTCCCTGAGCAGCCCCCCATGTCCCCAGTCCTGGGGGCCCCTGGCA |
| | CAGCTGTCTGGACCCTCTCTATTCCCTGGGAAGCTCCTCCTGACAGCCCCGCCTCCAGTTC |
| | CAGGTGTGGTTATTGTCAGGGGGTGTCAGACTGTGGTGGATACAGCTATGGTTACGTATTA |
| | CTATGATAGTAGTGGTTATTACTACCACAGTGGTGCTGCCCATAGCAGCAACCAGGCCAAG |
| | TAGACAGGCCCCTGCTGTGCAGCCCCAGGCCTCCAGCTCACCTGCTTCTCCTGGGGCTCTC |
| | AAGGCTGCTGTTTTCTGCACTCTCCCCTCTGTGGGGAGGGTTCCCTCAGTGGGAGATCTGT |
| | TCTCAACATCCCAGGGCCTCATTCCTGCAAGGAAGGCCAATGGATGGGCAACCTCACATGC |
| | CGCGGCTAAGATAGGGTGGGCAGCCTGGCGGGGACAGGACATCCTGCTGGGGTATCTGTCA |
| | CTGTGCCTAGTGGGGCACTGGCTCCCAAACAACGCAGTCCTCGCCAAAATCCCCACGGCCT |
| | CCCCCGCTAGGGGCTGGCCTGATCTCCTGCAGTCCTAGGAGGCTGCTGACCTCCAGAATGG |
| | CTCCGTCCCCAGTTCCAGGGCGAGAGCAGATCCCAGGCCGGCTGCAGACTGGGAGGCCACC |
| | CCCTCCTTCCCAGGGTTCACTGCAGGTGACCAGGGCAGGAAATGGCCTGAACACAGGGATA |
| | ACCGGGCCATCCCCCAACAGAGTCCACCCCCTCCTGCTCTGTACCCCGCACCCCCAAGGCC |
| | AGCCCATGACATCCGACAACCCCACACCAGAGTCACTGCCCGGTGCTGCCCTAGGGAGGAC |
| | CCCTCAGCCCCCACCCTGTCTAGAGGACTGGGGAGGACAGGACACGCCCTCTCCTTATGGT |
| | TCCCCCACCTGGCTCTGGCTGGGACCCTTGGGGTGTGGACAGAAAGGACGCTTGCCTGATT |
| | GGCCCCCAGGAGCCCAGAACTTCTCTCCAGGGACCCCAGCCCGAGCACCCCCTTACCCAGG |
| | ACCCAGCCCTGCCCCTCCTCCCATCTGCTCTCCTCTCATCACCCCATGGGAATCCAGAATC |
| | CCCAGGAAGCCATCAGGAAGGGCTGAGGGAGGAAGTGGGGCCACTGCACCACCAGGCAGGA |
| | GGCTCCGTCTTTGTGAACCCAGGGAGGTGCCAGCCTCCTAGAGGGTATGGTCCACCCTGCC |
| | TATGGCTCCCACAGTGGCAGGCTGCAGGGAAGGACCAGGGACGGTGTGGGGGAGGGCTCAG |
| | GGCCCCGCGGGTGCTCCATCTTGGATGAGCCCATCTCTCTCACCCACGGACTCACCCACCT |
| | CCTCTCCACCCTGGCCACACGTCGTCCACACCATCCTAAGTCCCACCTACACCAGAGCCGG |
| | CACAGCCAGTGCAGACAGAGGCTGGGGTGCAGGGGGGCCGCCAGGGCAGCTTTGGGGAGGG |
| | AAGGATGGAGGAAGGGGAGTTCAGTGAAGAGGCCCCCCTCCCCTGGGTCCAGGATCCTCCT |
| | CTGGGACCCCCGGATCCCATCCCCTCCAGGCTCTGGGAGGAGAAGCAGGATGGGAGAATCT |
| | GTGCGGGACCCTCTCACAGTGGAATACCTCCACAGCGGCTCAGGCAAGACCCAAAAGCCCC |
| | TCAGTGAGCCCTCCACTGCAGTCCTGGGCCTGGGTAGCAGCCCCTCCCACAGAGGATGAAC |
| | CCAGCACCCCGAGGATGTCCTGCCAGGGGGAGCTCAGAGCCATGAAGGAGCAGGATATGGG |
| | ACCCCCGATACAGGCACAGACCTCAGCTCCATTCAGGACTGCCACGTCCTGCCCTGGGAGG |
| | AACCCCTTTCTCTAGTCCCTGCAGGCCAGGAGGCAGCTGACTCCTGACTTGGACGCCTATT |
| | CCAGACACCAGACAGAGGGGCAGGCCCCCCAGAACCAGGGATGAGGACGCCCCGTCAAGGC |
| | CAGAAAAGACCAAGTTGTGCTGAGCCCAGCAAGGGAAGGTCCCCAAACAAACCAGGAAGTT |
| | TCTGAAGGTGTCTGTGTCACAGTGGAGTATAGCAGCTCGTCCGTATTACTATGGTTCGGGG |
| | AGTTATTATAACCACAGTGACACTCGCCAGGCCAGAAACCCCATCCCAAGTCAGCGGAATG |
| | CAGAGAGAGCAGGGAGGACATGTTTAGGATCTGAGGCCGCACCTGACACCCAGGCCAGCAG |
| | ACGTCTCCTGTCCATGGCACCCTGCCATGTCCTGCATTTCTGGAAGAACAAGGGCAGGCTG |
| | AAGGGGGTCCAGGACCAGGAGATGGGTCCCCTCTACCCAGAGAAGGAGCCAGGCAGGACAC |
| | AAGCCCCCTCCCCATTGAGGCTGACCTGCCCAGAGGGTCCTGGGCCCACCCCACACACCGG |
| | GGCGGAATGTGTGCAGGCCTCGGTCTCTGTGGGTGTTCCGCTAGCTGGGGCTCACAGTGCT |
| | CACCCCACACCTAAAACGAGCCACAGCCTCAGAGCCCCTGAAGGAGACCCCGCCCACAAGC |
| | CCAGCCCCCACCCAGGAGGCCCCAGAGCACAGGGCGCCCCGTCGGATTCTGAACAGCCCCG |
| | AGTCACAGTGGAGTATAGCAGCTCGTCCGGGTATAGCAGTGGCTGGTACCACTGTGAGAAA |
| | AGCTTCGTCCAAAACGGTCTCCTGGCCACAGTCGGAGGCCCCGCCAGAGAGGGGAGCAGCC |
| | ACCCCAAACCCATGTTCTGCCGGCTCCCATGACCCCGTGCACCTGGAGCCCCACAGTGTCC |
| | CCACTGGATGGGAGGACAAGGGCCGGGGGCTCCGGCGGGTCGGGGCAGGGGCTTGATGGCT |
| | TCCTTCTGCCGTGGCTCCAGTGCCCCTGGCTGGAGTTGACCCTTCTGACAAGTGTCCTCAG |
| | AGAGTCAGGGATCAGTGGCACCTCCCAACATCAACCCCACGCAGCCCAGGCACAAACCCCA |
| | CATCCAGGGCCAACTCCAGGAACAGAGACACCCCAATACCCTGGGGGACCCCAACCCTGAT |
| | GACTCCCGTCCCATCTCTGTCCCTCACTTGGGGCCTGCTGCGGGGCGAGCACTTGGGAGCA |
| | AACTCAGGCTTAGGGGACACCACTGTGGGCCTGACCTCGAGCAGGCCACAGACCCTTCCCT |
| | CCTGCCCTGGTGCAGCACAGACTTTGGGGTCTGGGCAGGGAGGAACTTCTGGCAGGTCACC |
| | AAGCACAGAGCCCCCAGGCTGAGGTGGCCCCAGGGGGAACCCCAGCAGGTGGCCCACTACC |
| | CTTCCTCCCAGCTGGACCCCATGTCTTCCCCAAGATAGGGGTGCCATCCAAGGCAGGTCCT |
| | CCATGGAGCCCCCTTCAGGCTCCTCTCCAGACCCCACTGGGCCTCAGTCCCCACTCTAGGA |
| | ATGCAGCCACCACGGGCACACCAGGCAGCCCAGGCCCAGCCACCCTGCAGTGCCCAAGCCC |
| | ACACCCTGGAGGAGAGCAGGGTGCGTCTGGGAGGGGCTGGGCTCCCCACCCCCACCCCCAC |
| | CTGCACACCCCACCCACCCTTGCCCGGGCCCCCTGCAGGAGGGTCAGAGCCCCCATGGGAT |
| | ATGGACTTAGGGTCTCACTCACGCACCTCCCCTCCTGGGAGAAGGGGTCTCATGCCCAGAT |
| | CCCCCCAGCAGCGCTGGTCACAGGTAGAGGCAGTGGCCCCAGGGCCACCCTGACCTGGCCC |
| | CTCAGGCTCCTCTAGCCCTGGCTGCCCTGCTGTCCCTGGGAGGCCTGGGCTCCACCAGACC |
| | ACAGGTCTAGGGCACCGCCCACACTGGGGCCGCCCACACACAGCTCACAGGAAGAAGATAA |
| | GCTCCAGACCCCCAGGCCCGGGACCTGCCTTGCTGCTACGACTTCCTGCCCCAGACCTCGT |
| | TGCCCTCCCCCGTCCACTTACACACAGGCCAGGAAGCTGTTCCCACACAGACCAACCCCAG |
| | ACGGGGACCACCTGGCACTCAGGTCACTGCCATTTCCTTCTCCATTCACTTCCAATGCCTC |
| | TGTGCTTCCTCCCTCCTCCTTCCTTCGGGGGAGCACCCTGTGCAGCTCCTCCCTGCAGTCC |
| | ACACCCTGGGGAGACCCGACCCTGCAGCCCACACCCTGGGGAGACCTGACCCTCCTCCAGC |
| | CCTTTCTCCCCCGCTGCTCTTGCCACCCACCAAGACAGCCCTGGGGTCCTGTCCCTACAGC |
| | CCCCACCCAGTTCTCTACCTAGACCCGTCTTCCTCCCTCTAAACACCTCTCCCAGGCCAAC |
| | CCTACACCTGCAGGCCCTCCCCTCCACTGCCAAAGACCCTCAGTTTCTCCTGCCTGTGCCC |
| | ACCCCCGTGCTCCTCCTGCCCACAGCTCGAGCTCTTCCTCTCCTAGGGCCCCTGAGGGATG |
| | GCATTGACCGTGCCCTCGCACCCACACACTGCCCATGCCCTCACATTCCTCCTGGCCACTC |
| | CAGCCCCACTCCCCTCTCAGGCCTGGCTCTGGTATTTCTGGGACAAAGCCTTACCCAAGTC |
| | TTTCCCATGCAGGCCTGGGCCCTTACCCTCACTGCCCGGTTACAGGGCAGCCTCCTGTGCA |
| | CAGAAGCAGGGAGCTCAGCCCTTCCACAGGCAGAAGGCACTGAAAGAAATCGGCCTCCAGC |
| | GCCTTGACACACGTCTGCCTGTGTCTCTCACTGCCCGCACCTGCAGGGAGGCTCGGCACTC |
| | CCTCTAAAGACGAGGGATCCAGGCAGCAGCATCACAGGAGAATGCAGGGCTACCAGACATC |
| | CCAGTCCTCTCACAGGCCTCTCCTGGGAAGAGACCTGAAGACGCCCAGTCAACGGAGTCTA |
| | ACACCAAACCTCCCTGGAGGCCGATGGGTAGTAACGGAGTCATTGCCAGACCTGGAGGCAG |
| | GGGAGCAGTGAGCCCGAGCCCACACCATAGGGCCAGAGGACAGCCACTGACATCCCAAGCC |
| | ACTCACTGGTGGTCCCACAACACCCCATGGAAAGAGGACAGACCCACAGTCCCACCTGGAC |
| | CAGGGCAGAGACTGCTGAGACCCAGCACCAGAACCAACCAAGAAACACCAGGCAACAGCAT |
| | CAGAGGGGGCTCTGGCAGAACAGAGGAGGGGAGGTCTCCTTCACCAGCAGGCGCTTCCCTT |
| | GACCGAAGACAGGATCCATGCAACTCCCCCAGGACAAAGGAGGAGCCCCTTGTTCAGCACT |
| | GGGCTCAGAGTCCTCTCCAAGACACCCAGAGTTTCAGACAAAAACCCCCTGGAATGCACAG |
| | TCTCAGCAGGAGAGCCAGCCAGAGCCAGCAAGATGGGGCTCAGTGACACCCGCAGGGACAG |
| | GAGGATTTTGTGGGGGCTCGTGTCACTGTGAGGATATTGTACTAATGGTGTATGCTATACC |
| | CACAGTGACACAGCCCCATTCCCAAAGCCCTACTGCAAACGCATTCCACTTCTGGGGCTGA |
| | GGGGCTGGGGGAGCGTCTGGGAAATAGGGCTCAGGGGTGTCCATCAATGCCCAAAACGCAC |
| | CAGACTCCCCTCCATACATCACACCCACCAGCCAGCGAGCAGAGTAAACAGAAAATGAGAA |
| | GCAAGCTGGGGAAGCTTGCACAGGCCCCAAGGAAAGAGCTTTGGCGGGTGTGTAAGAGGGG |
| | ATGCGGGCAGAGCCTGAGCAGGGCCTTTTGCTGTTTCTGCTTTCCTGTGCAGAGAGTTCCA |
| | TAAACTGGTGTTCGAGATCAATGGCTGGGAGTGAGCCCAGGAGGACAGCGTGGGAAGAGCA |
| | CAGGGAAGGAGGAGCAGCCGCTATCCTACACTGTCATCTTTCGAAAGTTTGCCTTGTGCCC |
| | ACACTGCTGCATCATGGGATGCTTAACAGCTGATGTAGACACAGCTAAAGAGAGAATCAGT |
| | GAGATGGATTTGCAGCACAGATCTGAATAAATTCTCCAGAATGTGGAGCAGCACAGAAGCA |
| | AGCACACAGAAAGTGCCTGATGCAAGGACAAAGTTCAGTGGGCACCTTCAGGCATTGCTGC |
| | TGGGCACAGACACTCTGAAAAGCCCTGGCAGGAACTCCCTGTGACAAAGCAGAACCCTCAG |
| | GCAATGCCAGCCCCAGAGCCCTCCCTGAGAGCCTCATGGGCAAAGATGTGCACAACAGGTG |
| | TTTCTCATAGCCCCAAACTGAGAGCAAAGCAAACGTCCATCTGAAGGAGAACAGGCAAATA |
| | AACGATGGCAGGTTCATGAAATGCAAACCCAGACAGCCACAAGCACAAAAGTACAGGGTTA |
| | TAAGCGACTCTGGTTGAGTTCATGACAATGCTGAGTAATTGGAGTAACAAAGTAAACTCCA |
| | AAAAATACTTTCAATGTGATTTCTTCTAAATAAAATTTACACCCTGCAAAATGAACTGTCT |
| | TCTTAAGGGATACATTTCCCAGTTAGAAAACCATAAAGAAAACCAAGAAAAGGATGATCAC |
| | ATAAACACAGTGGTGGTTACTTCTGCTGGGGAAGGAAGAGGGTATGAACTGAGATACACAG |
| | GGTGGGCAAGTCTCCTAACAAGAACAGAACGAATACATTACAGTACCTTGAAAACAGCAGT |
| | TAAACTTCTAAATTGCAAGAAGAGGAAAATGGACACAGTTGTGTTTAGAAAATTCTCAGTC |
| | CAGCACTGTTCATAATAGCAAAGACATTAACCCAGGTCGGATAAATAAGCGATGACACAGG |
| | CAATTGCACAATGATACAGACATATATTTAGTATATGAGACATCGATGATGTATCCCCAAA |
| | TAAACGACTTTAAAGAGATAAAGGGCTGATGTGTGGTGGCATTCACCTCCCTGGGATCCCC |
| | GGACAGGTTGCAGGCTCACTGTGCAGCAGGGCAGGCGGGTACCTGCTGGCAGTTCCTGGGG |
| | CCTGATGTGGAGCAAGCGCAGGGCCATATATCCCGGAGGACGGCACAGTCAGTGAATTCCA |
| | GAGAGAAGCAACTCAGCCACACTCCCCAGGCAGAGCCCGAGAGGGACGCCCACGCACAGGG |
| | AGGCAGAGCCCAGCACCTCCGCAGCCAGCACCACCTGTGCACGGGCCACCACCTTGCAGGC |
| | ACAGAGTGGGTGCTGAGAGGAGGGGCAGGGACACCAGGCAGGGTGAGCACCCAGAGAAAAC |
| | TGCAGACGCCTCACACATCCACCTCAGCCTCCCCTGACCTGGACCTCACTGGCCTGGGCCT |
| | CACTTAACCTGGGCTTCACCTGACCTTGGCCTCACCTGACTTGGACCTCGCCTGTCCCAAG |
| | CTTTACCTGACCTGGGCCTCAACTCACCTGAACGTCTCCTGACCTGGGTTTAACCTGTCCT |
| | GGAACTCACCTGGCCTTGGCTTCCCCTGACCTGGACCTCATCTGGCCTGGGCTTCACCTGG |
| | CCTGGGCCTCACCTGACCTGGACCTCATCTGGCCTGGACCTCACCTGGCCTGGACTTCACC |
| | TGGCCTGGGCTTCACCTGACCTGGACCTCACCTGGCCTCGGGCCTCACCTGCACCTGCTCC |
| | AGGTCTTGCTGGAGCCTGAGTAGCACTGAGGGTGCAGAAGCTCATCCAGGGTTGGGGAATG |
| | ACTCTAGAAGTCTCCCACATCTGACCTTTCTGGGTGGAGGCAGCTGGTGGCCCTGGGAATA |
| | TAAAAATCTCCAGAATGATGACTCTGTGATTTGTGGGCAACTTATGAACCCGAAAGGACAT |
| | GGCCATGGGGTGGGTAGGGACATAGGGACAGATGCCAGCCTGAGGTGGAGCCTCAGGACAC |
| | AGGTGGGCACGGACACTATCCACATAAGCGAGGGATAGACCCGAGTGTCCCCACAGCAGAC |
| | CTGAGAGCGCTGGGCCCACAGCCTCCCCTCAGAGCCCTGCTGCCTCCTCCGGTCAGCCCTG |
| | GACATCCCAGGTTTCCCCAGGCCTGCCGGTAGGTTTAGAATGAGGTCTGTGTCACTGTGGT |
| | GGATATAGTGGCTACGATTACGTGGATACAGCTATGGTTACCACAGTGTCACAGAGTCCAT |
| | CAAAAACCCATGCCTGGAAGCTTCCCGCCACAGCCCTCCCCATGGGGCCCTGCTGCCTCCT |
| | CAGGTCAGCCCCGGACATCCCGGGTTTCCCCAGGCTGGGCGGTAGGTTTGGGGTGAGGTCT |
| | GTGTCACTGTGGGGTATAGCAGCAGCTGGTACGGGTATAGCAGTGGCTGGTACCACAGTGT |
| | CACAGAGTCCATCAAAAACCCATCCCTGGGAGCCTCCCGCCACAGCCCTCCCTGCAGGGGA |
| | CCGGTACGTGCCATGTTAGGATTTTGATCGAGGAGACAGCACCATGGGTATGGTGGCTACC |
| | ACAGCAGTGCAGCCTGTGACCCAAACCCGCAGGGCAGCAGGCACGATGGACAGGCCCGTGA |
| | CTGACCACGCTGGGCTCCAGCCTGCCAGCCCTGGAGATCATGAAACAGATGGCCAAGGTCA |
| | CCCTACAGGTCATCCAGATCTGGCTCCGAGGGGTCTGCATCGCTGCTGCCCTCCCAACGCC |
| | AGTCCAAATGGGACAGGGACGGCCTCACAGCACCATCTGCTGCCATCAGGCCAGCGATCCC |
| | AGAAGCCCCTCCCTCAAGGCTGGGCCACATGTGTGGACACTGAGAGCCCTCATGTCTGAGT |
| | AGGGGCACCAGGAGGGAGGGGCTGGCCCTGTGCACTGTCCCTGCCCCTGTGGTCCCTGGCC |
| | TGCCTGGCCCTGACACCTGAGCCTCTCCTGGGTCATTTCCAAGACAGAAGACATTCCTGGG |
| | GACAGCCGGAGCTGGGCGTCGCTCATCCTGCCCGGCCGTCCTGAGTCCTGCTCATTTCCAG |
| | ACCTCACCGGGGAAGCCAACAGAGGACTCGCCTCCCACATTCAGAGACAAAGAACCTTCCA |
| | GAAATCCCTGCCTCTCTCCCCAGTGGACACCCTCTTCCAGGACAGTCCTCAGTGGCATCAC |
| | AGCGGCCTGAGATCCCCAGGACGCAGCACCGCTGTCAATAGGGGCCCCAAATGCCTGGACC |
| | AGGGCCTGCGTGGGAAAGGTCTCTGGCCACACTCGGGCTTTTTGTGAAGGGCCCTCCTGCT |
| | GTGAGGATATTGTAGTAGTACCAGCTGCTATGCCGGGTATAGCAGCAGCTGGTACCATAGT |
| | GATGAACCCAGTGGCAAAAACTGGCTGGAAACCCAGGGGCTGTGTGCACGCCTCAGCTTGG |
| | AGCTCTCCAGGAGCACAAGAGCCGGGCCCAAGGATTTGTGCCCAGACCCTCAGCCTCTAGG |
| | GACACCTGGGCCATCTCAGCCTGGGCTGGTGCCCTGCACACCATCTTCCTCCAAATAGGGG |
| | CTTCAGAGGGCTCTGAGGTGACCTCACTCATGACCACAGGTGACCTGGCCCTTCCCTGCCA |
| | GCTATACCAGACCCTGTCTTGACAGATGCCCCGATTCCAACAGCCAATTCCTGGGACCCTG |
| | AATAGCTGTAGACACCAGCCTCATTCCAGTACCTCCTGCCAATTGCCTGGATTCCCATCCT |
| | GGCTGGAATCAAGAAGGCAGCATCCGCCAGGCTCCCAACAGGCAGGACTCCCGCACACCCT |
| | CCTCTGAGAGGCCGCTGTGTTCCGCAGGGCCAGGCCCTGGACAGTTCCCCTCACCTGCCAC |
| | TAGAGAAACACCTGCCATTGTCGTCCCCACCTGGAAAAGACCACTCGTGGAGCCCCCAGCC |
| | CCAGGTACAGCTGTAGAGAGAGTCCTCGAGGCCCCTAAGAAGGAGCCATGCCCAGTTCTGC |
| | CGGGACCCTCGGCCAGGCCGACAGGAGTGGACGCTGGAGCTGGGCCCACACTGGGCCACAT |
| | AGGAGCTCACCAGTGAGGGCAGGAGAGCACATGCCGGGGAGCACCCAGCCTCCTGCTGACC |
| | AGAGGCCTGCCCCAGAGCCCAGGAGGCTGCAGAGGCCTCTCCAGGGAGACACTGTGCATGT |
| | CTGGTACCTAAGCAGCCCCCCACGTCCCCAGTCCTGGGGGCCCCTGGCTCAGCTGTCTGGA |
| | CCCTCCCTGTTCCCTGGGAAGCTCCTCCTGACAGCCCCGCCTCCAGTTCCAGGTGTGGTTA |
| | TTGTCAGGCGATGTCAGACTGTGGTGGATATAGTGGCTACGATTACGGGTATAGCAGTGGC |
| | TGGTACCACAGTGGTGCCGCCCATAGCAGCAACCAGGCCAAGTAGACAGGCCCCTGCTGCG |
| | CAGCCCCAGGCATCCACTTCACCTGCTTCTCCTGGGGCTCTCAAGGCTGCTGTCTGTCCTC |
| | TGGCCCTCTGTGGGGAGGGTTCCCTCAGTGGGAGGTCTGTGCTCCAGGGCAGGGATGATTG |
| | AGATAGAAATCAAAGGCTGGCAGGGAAAGGCAGCTTCCCGCCCTGAGAGGTGCAGGCAGCA |
| | CCACGGAGCCACGGAGTCACAGAGCCACGGAGCCCCCATTGTGGGCATTTGAGAGTGCTGT |
| | GCCCCCGGCAGGCCCAGCCCTGATGGGGAAGCCTGTCCCATCCCACAGCCCGGGTCCCACG |
| | GGCAGCGGGCACAGAAGCTGCCAGGTTGTCCTCTATGATCCTCATCCCTCCAGCAGCATCC |
| | CCTCCACAGTGGGGAAACTGAGGCTTGGAGCACCACCCGGCCCCCTGGAAATGAGGCTGTG |
| | AGCCCAGACAGTGGGCCCAGAGCACTGTGAGTACCCCGGCAGTACCTGGCTGCAGGGATCA |
| | GCCAGAGATGCCAAACCCTGAGTGACCAGCCTACAGGAGGATCCGGCCCCACCCAGGCCAC |
| | TCGATTAATGCTCAACCCCCTGCCCTGGAGACCTCTTCCAGTACCACCAGCAGCTCAGCTT |
| | CTCAGGGCCTCATCCCTGCAAGGAAGGTCAAGGGCTGGGCCTGCCAGAAACACAGCACCCT |
| | CCCTAGCCCTGGCTAAGACAGGGTGGGCAGACGGCTGTGGACGGGACATATTGCTGGGGCA |
| | TTTCTCACTGTCACTTCTGGGTGGTAGCTCTGACAAAAACGCAGACCCTGCCAAAATCCCC |
| | ACTGCCTCCCGCTAGGGGCTGGCCTGGAATCCTGCTGTCCTAGGAGGCTGCTGACCTCCAG |
| | GATGGCTCCGTCCCCAGTTCCAGGGCGAGAGCAGATCCCAGGCAGGCTGTAGGCTGGGAGG |
| | CCACCCCTGCCCTTGCCGGGGTTGAATGCAGGTGCCCAAGGCAGGAAATGGCATGAGCACA |
| | GGGATGACCGGGACATGCCCCACCAGAGTGCGCCCCTTCCTGCTCTGCACCCTGCACCCCC |
| | CAGGCCAGCCCACGACGTCCAACAACTGGGCCTGGGTGGCAGCCCCACCCAGACAGGACAG |
| | ACCCAGCACCCTGAGGAGGTCCTGCCAGGGGGAGCTAAGAGCCATGAAGGAGCAAGATATG |
| | GGGCCCCCGATACAGGCACAGATGTCAGCTCCATCCAGGACCACCCAGCCCACACCCTGAG |
| | AGGAACGTCTGTCTCCAGCCTCTGCAGGTCGGGAGGCAGCTGACCCCTGACTTGGACCCCT |
| | ATTCCAGACACCAGACAGAGGCGCAGGCCCCCCAGAACCAGGGTTGAGGGACGCCCCGTCA |
| | AAGCCAGACAAAACCAAGGGGTGTTGAGCCCAGCAAGGGAAGGCCCCCAAACAGACCAGGA |
| | GGTTTCTGAAGGTGTCTGTGTCACAGTGGGGTATAGCAGCAGCTGGTACAGCATATTGTGG |
| | TGGTGATTGCTATTCCCACAGTGACACTCACCCAGCCAGAAACCCCATTCCAAGTCAGCGG |
| | AAGCAGAGAGAGCAGGGAGGACACGTTTAGGATCTGAGACTGCACCTGACACCCAGGCCAG |
| | CAGACGTCTCCCCTCCAGGGCACCCCACCCTGTCCTGCATTTCTGCAAGATCAGGGGCGGC |
| | CTGAGGGGGGGTCTAGGGTGAGGAGATGGGTCCCCTGTACACCAAGGAGGAGTTAGGCAGG |
| | TCCCGAGCACTCTCCCCATTGAGGCTGACCTGCCCAGAGAGTCCTGGGCCCACCCCACACA |
| | CCGGGGCGGAATGTGTGCAGGCCTCGGTCTCTGTGGGTGTTCCGCTAGCTGGGGCTCACAG |
| | TGCTCACCCCACACCTAAAATGAGCCACAGCCTCCGGAGCCCCCGCAGGAGACCCCGCCCA |
| | CAAGCCCAGCCCCCACCCAGGAGGCCCCAGAGCTCAGGGCGCCCCGTCGGATTCCGAACAG |
| | CCCCGAGTCACAGCGGTGGATACAGCTATGGTTACGTATTACTATGGTTCGGGGAGTTATT |
| | ATAACCACTGTCAGAATAGCTACGTCAAAAACTGTCCAGTGGCCACTGCCGGAGGCCCCGC |
| | CAGAGAGGGCAGCAGCCACTCTGATCCCATGTCCTGCCGGCTCCCATGACCCCCAGCACGC |
| | GGAGCCCCACAGTGTCCCCACTGGATGGGAGGACAAGAGCTGGGGATTCCGGCGGGTCGGG |
| | GCAGGGGCTTGATCGCATCCTTCTGCCGTGGCTCCAGTGCCCCTGGCTGGAGTTGACCCTT |
| | CTGACAAGTGTCCTCAGAGAGACAGGCATCACCGGCGCCTCCCAACATCAACCCCAGGCAG |
| | CACAGGCACAAACCCCACATCCAGAGCCAACTCCAGGAGCAGAGACACCCCAATACCCTGG |
| | GGGACCCCGACCCTGATGACTTCCCACTGGAATTCGCCGTAGAGTCCACCAGGACCAAAGA |
| | CCCTGCCTCTGCCTCTGTCCCTCACTCAGGACCTGCTGCCGGGCGAGGCCTTGGGAGCAGA |
| | CTTGGGCTTAGGGGACACCAGTGTGACCCCGACCTTGACCAGGACGCAGACCTTTCCTTCC |
| | TTTCCTGGGGCAGCACAGACTTTGGGGTCTGGGCCAGGAGGAACTTCTGGCAGGTCGCCAA |
| | GCACAGAGGCCACAGGCTGAGGTGGCCCTGGAAAGACCTCCAGGAGGTGGCCACTCCCCTT |
| | CCTCCCAGCTGGACCCCATGTCCTCCCCAAGATAAGGGTGCCATCCAAGGCAGGTGCTCCT |
| | TGGAGCCCCATTCAGACTCCTCCCTGGACCCCACTGGGCCTCAGTCCCAGCTCTGGGGATG |
| | AAGCCACCACAAGCACACCAGGCAGCCCAGGCCCAGCCACCCTGCAGTGCCCAAGCACACA |
| | CTCTGGAGCAGAGCAGGGTGCCTCTGGGAGGGGCTGAGCTCCCCACCCCACCCCCACCTGC |
| | ACACCCCACCCACCCCTGCCCAGCGGCTCTGCAGGAGGGTCAGAGCCCCACATGGGGTATG |
| | GACTTAGGGTCTCACTCACGTGGCTCCCATCATGAGTGAAGGGGCCTCAAGCCCAGGTTCC |
| | CACAGCAGCGCCTGTCGCAAGTGGAGGCAGAGGCCCGAGGGCCACCCTGACCTGGTCCCTG |
| | AGGTTCCTGCAGCCCAGGCTGCCCTGCTGTCCCTGGGAGGCCTGGGCTCCACCAGACCACA |
| | GGTCCAGGGCACCGGGTGCAGGAGCCACCCACACACAGCTCACAGGAAGAAGATAAGCTCC |
| | AGACCCCCAGGGCCAGAACCTGCCTTCCTGCTACTGCTTCCTGCCCCAGACCTGGGCGCCC |
| | TCCCCCGTCCACTTACACACAGGCCAGGAAGCTGTTCCCACACAGAACAACCCCAAACCAG |
| | GACCGCCTGGCACTCAGGTGGCTGCCATTTCCTTCTCCATTTGCTCCCAGCGCCTCTGTCC |
| | TCCCTGGTTCCTCCTTCGGGGGAACAGCCTGTGCAGCCAGTCCCTGCAGCCCACACCCTGG |
| | GGAGACCCAACCCTGCCTGGGGCCCTTCCAACCCTGCTGCTCTTACTGCCCACCCAGAAAA |
| | CTCTGGGGTCCTGTCCCTGCAGTCCCTACCCTGGTCTCCACCCAGACCCCTGTGTATCACT |
| | CCAGACACCCCTCCCAGGCCAACCCTGCACCTGCAGGCCCTGTCCTCTTCTGTCGCTAGAG |
| | CCTCAGTTTCTCCCCCCTGTGCCCACACCCTACCTCCTCCTGCCCACAACTCTAACTCTTC |
| | TTCTCCTGGAGCCCCTGAGCCATGGCATTGACCCTGCCCTCCCACCACCCACAGCCCATGC |
| | CCTCACCTTCCTCCTGGCCACTCCGACCCCGCCCCCTCTCAGGCCAAGCCCTGGTATTTCC |
| | AGGACAAAGGCTCACCCAAGTCTTTCCCAGGCAGGCCTGGGCTCTTGCCCTCACTTCCCGG |
| | TTACACGGGAGCCTCCTGTGCACAGAAGCAGGGAGCTCAGCCCTTCCACAGGCAGAAGGCA |
| | CTGAAAGAAATCGGCCTCCAGCACCTTGACACACGTCCCCCCGTGTCTCTCACTGCCCGCA |
| | CCTGCAGGGAGGCTCCGCACTCCCTCTAAAGACAAGGGATCCAGGCAGCAGCATCACGGGA |
| | GAATGCAGGGCTCCCAGACATCCCAGTCCTCTCACAGGCCTCTCCTGGGAAGAGACCTGCA |
| | GCCACCACCAAACAGCCACAGAGGCTGCTGGATAGTAACTGAGTCAATGACCGACCTGGAG |
| | GGCAGGGGAGCAGTGAGCCGGAGCCCATACCATAGGGACAGAGACCAGCCGCTGACATCCC |
| | GAGCTCCTCAATGGTGGCCCCATAACACACCTAGGAAACATAACACACCCACAGCCCCACC |
| | TGGAACAGGGCAGAGACTGCTGAGCCCCCAGCACCAGCCCCAAGAAACACCAGGCAACAGT |
| | ATCAGAGGGGGCTCCCGAGAAAGAGAGGAGGGGAGATCTCCTTCACCATCAAATGCTTCCC |
| | TTGACCAAAAACAGGGTCCACGCAACTCCCCCAGGACAAAGGAGGAGCCCCCTATACAGCA |
| | CTGGGCTCAGAGTCCTCTCTGAGACACCCTGAGTTTCAGACAACAACCCGCTGGAATGCAC |
| | AGTCTCAGCAGGAGAACAGACCAAAGCCAGCAAAAGGGACCTCGGTGACACCAGTAGGGAC |
| | AGGAGGATTTTGTGGGGGCTCGTGTCACTGTGAGGATATTGTAGTGGTGGTAGCTGCTACT |
| | CCGTATTACTATGATAGTAGTGGTTATTACTACCACAGTGACACAGACCCATTCCCAAAGC |
| | CCTACTGCAAACACACCCACTCCTGGGGCTGAGGGGCTGGGGGAGCGTCTGGGAAGTAGGG |
| | TCCAGGGGTGTCTATCAATGTCCAAAATGCACCAGACTGCCCGCCAAACACCACCCCACCA |
| | GCCAGCGAGCAGGGTAAACAGAAAATGAGAGGCTCTGGGAAGCTTGCACAGGCCCCAAGGA |
| | AAGAGCTTTGGCGGGTGTGCAAGAGGGGATGCAGGCAGAGCCTGAGCAGGGCCTTTTGCTG |
| | TTTCTGCTTTCCTGTGCAGAGAGTTCCATAAACTGGTGTTCAAGATCAGTGGCTGGGAATG |
| | AGCCCAGGAGGGCAGTCTGTGGGAAGAGCACAGGGAAGGAGGAGCAGCCGCTATCCTACAC |
| | TGTCATCTTTCAAAAGTTTGCCTTGTGACCACACTATTGCATCATGGGATGCTTAAGAGCT |
| | GATGTAGACACAGCTAAAGAGAGAATCAGTGAGATGAATTTGCAGCATAGATCTGAATAAA |
| | CTCTCCAGAATGTGGAGCAGTACAGAAGCAAACACACAGAAAGTGCCTGATGCAAGGACAA |
| | AGTTCAGTGGGCACCTTCAGGCATTGCTGCTGGGCACAGACACTCTGAAAAGCCCTGGCAG |
| | GATCTCCCTGCGACAAAGCAGAACCCTCAGGCAATGCCAGCCCCAGAGCCCTCCCTGAGAG |
| | CGTCATGGGGAAAGATGTGCAGAACAGCTGATTATCATAGACTCAAACTGAGAACAGAGCA |
| | AACGTCCATCTGAAGAACAGTCAAATAAGCAATGGTAGGTTCATGCAATGCAAACCCAGAC |
| | AGCCAGGGGACAACAGTAGAGGGCTACAGGCGGCTTTGCGGTTGAGTTCATGACAATGCTG |
| | AGTAATTGGAGTAACAGAGGAAAGCCCAAAAAATACTTTTAATGTGATTTCTTCTAAATAA |
| | AATTTACACCAGGCAAAATGAACTGTCTTCTTAAGGGATAAACTTTCCCCTGGAAAAACTA |
| | CAAGGAAAATTAAGAAAACGATGATCACATAAACACAGTTGTGGTTACTTCTACTGGGGAA |
| | GGAAGAGGGTATGAGCTGAGACACACAGAGTCGGCAAGTCTCCAAGCAAGCACAGAACGAA |
| | TACATTACAGTACCTTGAATACAGCAGTTAAACTTCTAAATCGCAAGAAGAGGAAAATGCA |
| | CACAGCTGTGTTTAGAAAATTCTCAGTCCAGCACTATTCATAATAGCAAAGACATTAACCC |
| | AGGTTGGATAAATAAATGATGACACAGGCAATTGCACAATGATACAGACATACATTTAGTA |
| | CATGAGACATCGATGATGTATCCCCAAAGAAATGACTTTAAAGAGAAAAGGCCTGATGTGT |
| | GGTGGCACTCACCTCCCTGGGATCCCCGGACAGGTTGCAGGCACACTGTGTGGCAGGGCAG |
| | GCTGGTACATGCTGGCAGCTCCTGGGGCCTGATGTGGAGCAAGCGCAGGGCTGTATACCCC |
| | CAAGGATGGCACAGTCAGTGAATTCCAGAGAGAAGCAGCTCAGCCACACTGCCCAGGCAGA |
| | GCCCGAGAGGGACGCCCACGCACAGGGAGGCAGAGCCCAGCTCCTCCACAGCCACCACCAC |
| | CTGTGCACGGGCCACCACCTTGCAGGCACAGAGTGGGTGCTGAGAGGAGGGGCAGGGACAC |
| | CAGGCAGGGTGAGCACCCAGAGAAAACTGCAGAAGCCTCACACATCCACCTCAGCCTCCCC |
| | TGACCTGGACCTCACCTGGTCTGGACCTCACCTGGCCTGGGCCTCACCTGACCTGGACCTC |
| | ACCTGGCCTGGGCTTCACCTGACCTGGACCTCACCTGGCCTCCGGCCTCACCTGCACCTGC |
| | TCCAGGTCTTGCTGGAACCTGAGTAGCACTGAGGCTGCAGAAGCTCATCCAGGGTTGGGGA |
| | ATGACTCTGGAACTCTCCCACATCTGACCTTTCTGGGTGGAGGCATCTGGTGGCCCTGGGA |
| | ATATAAAAAGCCCCAGAATGGTGCCTGCGTGATTTGGGGGCAATTTATGAACCCGAAAGGA |
| | CATGGCCATGGGGTGGGTAGGGACATAGGGACAGATGCCAGCCTGAGGTGGAGCCTCAGGA |
| | CACAGTTGGACGCGGACACTATCCACATAAGCGAGGGACAGACCCGAGTGTTCCTGCAGTA |
| | GACCTGAGAGCGCTGGGCCCACAGCCTCCCCTCGGTGCCCTGCTGCCTCCTCAGGTCAGCC |
| | CTGGACATCCCGGGTTTCCCCAGGCCAGATGGTAGGTTTGAAGTGAGGTCTGTGTCACTGT |
| | GGTATTATGATTACGTTTGGGGGAGTTATCGTTATACCCACAGCATCACACGGTCCATCAG |
| | AAACCCATGCCACAGCCCTCCCCGCAGGGGACCGCCGCGTGCCATGTTACGATTTTGATCG |
| | AGGACACAGCGCCATGGGTATGGTGGCTACCACAGCAGTGCAGCCCATGACCCAAACACAC |
| | AGGGCAGCAGGCACAATGGACAGGCCTGTGAGTGACCATGCTGGGCTCCAGCCCGCCAGCC |
| | CCGGAGACCATGAAACAGATGGCCAAGGTCACCCCACAGTTCAGCCAGACATGGCTCCGTG |
| | GGGTCTGCATCGCTGCTGCCCTCTAACACCAGCCCAGATGGGGACAAGGCCAACCCCACAT |
| | TACCATCTCCTGCTGTCCACCCAGTGGTCCCAGAAGCCCCTCCCTCATGGCTGAGCCACAT |
| | GTGTGAACCCTGAGAGCACCCCATGTCAGAGTAGGGGCAGCAGAAGGGGGGGGCTGGCCCT |
| | GTGCACTGTCCCTGCACCCATGGTCCCTCGCCTGCCTGGCCCTGACACCTGAGCCTCTTCT |
| | GAGTCATTTCTAAGATAGAAGACATTCCCGGGGACAGCCGGAGCTGGGCGTCGCTCATCCC |
| | GCCCGGCCGTCCTGAGTCCTGCTTGTTTCCAGACCTCACCAGGGAAGCCAACAGAGGACTC |
| | ACCTCACACAGTCAGAGACAAAGAACCTTCCAGAAATCCCTGTCTCACTCCCCAGTGGGCA |
| | CCTTCTTCCAGGACATTCCTCGGTCGCATCACAGCAGGCACCCACATCTGGATCAGGACGG |
| | CCCCCAGAACACAAGATGGCCCATGGGGACAGCCCCACAACCCAGGCCTTCCCAGACCCCT |
| | AAAAGGCGTCCCACCCCCTGCACCTGCCCCAGGGCTAAAAATCCAGGAGGCTTGACTCCCG |
| | CATACCCTCCAGCCAGACATCACCTCAGCCCCCTCCTGGAGGGGACAGGAGCCCGGGAGGG |
| | TGAGTCAGACCCACCTGCCCTCGATGGCAGGCGGGGAAGATTCAGAAAGGCCTGAGATCCC |
| | CAGGACGCAGCACCACTGTCAATGGGGGCCCCAGACGCCTGGACCAGGGCCTGCGTGGGAA |
| | AGGCCGCTGGGCACACTCAGGGGCTTTTTGTGAAGGCCCCTCCTACTGTGGTGGATATAGT |
| | GGCTACGATTACAGGATATTGTAGTGGTGGTAGCTGCTACTCCCACAGTGATGAAACTAGC |
| | AGCAAAAACTGGCCGGACACCCAGGGACCATGCACACTTCTCAGCTTGGAGCTCTCCAGGA |
| | CCAGAAGAGTCAGGTCTGAGGGTTTGTAGCCAGACCCTCGGCCTCTAGGGACACCCTGGCC |
| | ATCACAGCGGATGGGCTGGTGCCCCACATGCCATCTGCTCCAAACAGGGGCTTCAGAGGGC |
| | TCTGAGGTGACTTCACTCATGACCACAGGTGCCCTGGCCCCTTCCCCGCCAGCTACACCGA |
| | ACCCTGTCCCAACAGCTGCCCCAGTTCCAACAGCCAATTCCTGGGGCCCAGAATTGCTGTA |
| | GACACCAGCCTCGTTCCAGCACCTCCTGCCAATTGCCTGGATTCACATCCTGGCTGGAATC |
| | AAGAGGGCAGCATCCGCCAGGCTCCCAACAGGCAGGACTCCCGCACACCCTCCTCTGAGAG |
| | GCCGCTGTGTTCCGCAGGGCCAGGCCCTGGACAGTTCCCCTCACCTGCCACTAGAGAAACA |
| | CCTGCCATTGTCGTCCCCACCTGGAAAAGACCACTCGTGGAGCCCCCAGCCCCAGGTACAG |
| | CTGTAGAGAGACTCCCCGAGGGATCTAAGAAGGAGCCATGCGCAGTTCTGCCGGGACCCTC |
| | GGCCAGGCCGACAGGAGTGGACACTGGAGCTGGGCCCACACTGGGCCACATAGGAGCTCAC |
| | CAGTGAGGGCAGGAGAGCACATGCCGGGGAGCACCCAGCCTCCTGCTGACCAGAGGCCCGT |
| | CCCAGAGCCCAGGAGGCTGCAGAGGCCTCTCCAGGGGGACACTGTGCATGTCTGGTCCCTG |
| | AGCAGCCCCCCACGTCCCCAGTCCTGGGGGCCCCTGGCACAGCTGTCTGGACCCTCCCTCT |
| | TCCCTGGGAAGCTCCTCCTGACAGCCCCGCCTCCAGTTCCAGGTGTGGTTATTGTCAGGGG |
| | GTGTCAGACTGTGAGGATATTGTAGTGGTGGTAGCTGCTACTCCGTAGAGATGGCTACAAT |
| | TACCACAGTGGTGCTGCCCATAGCAGCAACCAGGCCAAGTAGACAGGCCCCTGCTGTGCAG |
| | CCCCAGGCCTCCAGCTCACCTGCTTCTCCTGGGGCTCTCAAGGTCACTGTTGTCTGTACTC |
| | TGCCCTCTGTGGGGAGGGTTCCCTCAGTGGGAGGTCTGTTCTCAACATCCCAGGGCCTCAT |
| | GTCTGCACGGAAGGCCAATGGATGGGCAACCTCACATGCCGCGGCTAAGATAGGGTGGGCA |
| | GCCTGGCGGGGGACAGTACATACTGCTGGGGTGTCTGTCACTGTGCCTAGTGGGGCACTGG |
| | CTCCCAAACAACGCAGTCCTCGCCAAAATCCCCACAGCCTCCCCTGCTAGGGGCTGGCCTG |
| | ATCTCCTGCAGTCCTAGGAGGCTGCTGACCTCCAGAATGTCTCCGTCCCCAGTTCCAGGGC |
| | GAGAGCAGATCCCAGGCCGGCTGCAGACTGGGAGGCCACCCCCTCCTTCCCAGGGTTCACT |
| | GGAGGTGACCAAGGTAGGAAATGGCCTTAACACAGGGATGACTGCGCCATCCCCCAACAGA |
| | GTCAGCCCCCTCCTGCTCTGTACCCCGCACCCCCCAGGCCAGTCCACGAAAACCAGGGCCC |
| | CACATCAGAGTCACTGCCTGGCCCGGCCCTGGGGCGGACCCCTCAGCCCCCACCCTGTCTA |
| | GAGGACTTGGGGGGACAGGACACAGGCCCTCTCCTTATGGTTCCCCCACCTGCCTCCGGCC |
| | GGGACCCTTGGGGTGTGGACAGAAAGGACACCTGCCTAATTGGCCCCCAGGAACCCAGAAC |
| | TTCTCTCCAGGGACCCCAGCCCGAGCACCCCCTTACCCAGGACCCAGCCCTGCCCCTCCTC |
| | CCCTCTGCTCTCCTCTCATCACCCCATGGGAATCCGGTATCCCCAGGAAGCCATCAGGAAG |
| | GGCTGAAGGAGGAAGCGGGGCCGTGCACCACCGGGCAGGAGGCTCCGTCTTCGTGAACCCA |
| | GGGAAGTGCCAGCCTCCTAGAGGGTATGGTCCACCCTGCCTGGGGCTCCCACCGTGGCAGG |
| | CTGCGGGGAAGGACCAGGGACGGTGTGGGGGAGGGCTCAGGGCCCTGCGGGTGCTCCTCCA |
| | TCTTCGGTGAGCCTCCCCCTTCACCCACCGTCCCGCCCACCTCCTCTCCACCCTGGCTGCA |
| | CGTCTTCCACACCATCCTGAGTCCTACCTACACCAGAGCCAGCAAAGCCAGTGCAGACAAA |
| | GGCTGGGGTGCAGGGGGGCTGCCAGGGCAGCTTCGGGGAGGGAAGGATGGAGGGAGGGGAG |
| | GTCAGTGAAGAGGCCCCCTTCCCCTGGGTCCAGGATCCTCCTCTGGGACCCCCGGATCCCA |
| | TCCCCTCCTGGCTCTGGGAGGAGAAGCAGGATGGGAGAATCTGTGCGGGACCCTCTCACAG |
| | TGGAATATCCCCACAGCGGCTCAGGCCAGACCCAAAAGCCCCTCAGTGAGCCCTCCACTGC |
| | AGTCCTGGGCCTGGGTAGCAGCCCCTCCCACAGAGGACAGACCCAGCACCCCGAAGAAGTC |
| | CTGCCAGGGGGAGCTCAGAGCCATGAAAGAGCAGGATATGGGGTCCCCGATACAGGCACAG |
| | ACCTCAGCTCCATCCAGGCCCACCGGGACCCACCATGGGAGGAACACCTGTCTCCGGGTTG |
| | TGAGGTAGCTGGCCTCTGTCTCGGACCCCACTCCAGACACCAGACAGAGGGGCAGGCCCCC |
| | CAAAACCAGGGTTGAGGGATGATCCGTCAAGGCAGACAAGACCAAGGGGCACTGACCCCAG |
| | CAAGGGAAGGCTCCCAAACAGACGAGGAGGTTTCTGAAGCTGTCTGTATCACAGTGGGGTA |
| | TAGCAGTGGCTGGTACGGTATAGTGGGAGCTACTACCACAGTGACACTCGCCAGGCCAGAA |
| | ACCCCGTCCCAAGTCAGCGGAAGCAGAGAGAGCAGGGAGGACACGTTTAGGATCTGAGGCC |
| | GCACCTGACACCCAGGGCAGCAGACGTCTCCCCTCCAGGGCACCCTCCACCGTCCTGCGTT |
| | TCTTCAAGAATAGGGGCGGCCTGAGGGGGTCCAGGGCCAGGCGATAGGTCCCCTCTACCCC |
| | AAGGAGGAGCCAGGCAGGACCCGAGCACCGTCCCCATTGAGGCTGACCTGCCCAGACGGGC |
| | CTGGGCCCACCCCACACACCGGGGCGGAATGTGTGCAGGCCCCAGTCTCTGTGGGTGTTCC |
| | GCTAGCTGGGGCCCCCAGTGCTCACCCCACACCTAAAGCGAGCCCCAGCCTCCAGAGCCCC |
| | CTAAGCATTCCCCGCCCAGCAGCCCAGCCCCTGCCCCCACCCAGGAGGCCCCAGAGCTCAG |
| | GGCGCCTGGTCGGATTCTGAACAGCCCCGAGTCACAGTGAGGATATTGTAGTGGTGGTAGC |
| | TGCTACTCCGTGGATACAGCTATGGTTACCACCGTGAGAAAAACTGTGTCCAAAACTGACT |
| | CCTGGCAGCAGTCGGAGGCCCCGCCAGAGAGGGGAGCAGCCGGCCTGAACCCATGTCCTGC |
| | CGGTTCCCATGACCCCCAGCACCCAGAGCCCCACGGTGTCCCCGTTGGATAATGAGGACAA |
| | GGGCTGGGGGCTCCGGTGGTTTGCGGCAGGGACTTGATCACATCCTTCTGCTGTGGCCCCA |
| | TTGCCTCTGGCTGGAGTTGACCCTTCTGACAAGTGTCCTCAGAAAGACAGGGATCACCGGC |
| | ACCTCCCAATATCAACCCCAGGCAGCACAGACACAAACCCCACATCCAGAGCCAACTCCAG |
| | GAGCAGAGACACCCCAACACTCTGGGGGACCCCAACCGTGATAACTCCCCACTGGAATCCG |
| | CCCCAGAGTCTACCAGGACCAAAGGCCCTGCCCTGTCTCTGTCCCTCACTCAGGGCCTCCT |
| | GCAGGGCGAGCGCTTGGGAGCAGACTCGGTCTTAGGGGACACCACTGTGGGCCCCAACTTT |
| | GATGAGGCCACTGACCCTTCCTTCCTTTCCTGGGGCAGCACAGACTTTGGGGTCTGGGCAG |
| | GGAAGAACTACTGGCTGGTGGCCAATCACAGAGCCCCCAGGCCGAGGTGGCCCCAAGAAGG |
| | CCCTCAGGAGGTGGCCACTCCACTTCCTCCCAGCTGGACCCCAGGTCCTCCCCAAGATAGG |
| | GGTGCCATCCAAGGCAGGTCCTCCATGGAGCCCCCTTCAGACTCCTCCCGGGACCCCACTG |
| | GACCTCAGTCCCTGCTCTGGGAATGCAGCCACCACAAGCACACCAGGAAGCCCAGGCCCAG |
| | CCACCCTGCAGTGGGCAAGCCCACACTCTGGAGCAGAGCAGGGTGCGTCTGGGAGGGGCTA |
| | ACCTCCCCACCCCCCACCCCCCATCTGCACACAGCCACCTACCACTGCCCAGACCCTCTGC |
| | AGGAGGGCCAAGCCACCATGGGGTATGGACTTAGGGTCTCACTCACGTGCCTCCCCTCCTG |
| | GGAGAAGGGGCCTCATGCCCAGATCCCTGCAGCACTAGACACAGCTGGAGGCAGTGGCCCC |
| | AGGGCCACCCTGACCTGGCATCTAAGGCTGCTCCAGCCCAGACAGCACTGCCGTTCCTGGG |
| | AAGCCTGGGCTCCACCAGACCACAGGTCCAGGGCACAGCCCACAGGAGCCACCCACACACA |
| | GCTCACAGGAAGAAGATAAGCTCCAGACCCCAGGGCGGGACCTGCCTTCCTGCCACCACTT |
| | ACACACAGGCCAGGGAGCTGTTCCCACACAGATCAACCCCAAACCGGGACTGCCTGGCACT |
| | AGGGTCACTGCCATTTCCCTCTCCATTCCCTCCCAGTGCCTCTGTGCTCCCTCCTTCTGGG |
| | GAACACCCTGTGCAGCCCCTCCCTGCAGCCCACACGCTGGGGAGACCCCACCCTGCCTCGG |
| | GCCTTTTCTACCTGCTGCACTTGCCGCCCACCCAAACAACCCTGGGTACGTGACCCTGCAG |
| | TCCTCACCCTGATCTGCAACCAGACCCCTGTCCCTCCCTCTAAACACCCCTCCCAGGCCAA |
| | CTCTGCACCTGCAGGCCCTCCGCTCTTCTGCCACAAGAGCCTCAGGTTTTCCTACCTGTGC |
| | CCACCCCCTAACCCCTCCTGCCCACAACTTGAGTTCTTCCTCTCCTGGAGCCCTTGAGCCA |
| | TGGCACTGACCCTACACTCCCACCCACACACTGCCCATGCCATCACCTTCCTCCTGGACAC |
| | TCTGACCCCGCTCCCCTCCCTCTCAGACCCGGCCCTGGTATTTCCAGGACAAAGGCTCACC |
| | CAAGTCTTCCCCATGCAGGCCCTTGCCCTCACTGCCTGGTTACACGGGAGCCTCCTGTGCG |
| | CAGAAGCAGGGAGCTCAGCTCTTCCACAGGCAGAAGGCACTGAAAGAAATCGGCCTCCAGT |
| | GCCTTGACACACGTCCGCCTGTGTCTCTCACTGCCTGCACCTGCAGGGAGGCTCCGCACTC |
| | CCTCTAAAGATGAGGGATCCAGGCAGCAACATCACGGGAGAATGCAGGGCTCCCAGACAGC |
| | CCAGCCCTCTCGCAGGCCTCTCCTGGGAAGAGACCTGCAGCCACCACTGAACAGCCACGGA |
| | GGTCGCTGGATAGTAACCGAGTCAGTGACCGACCTGGAGGGCAGGGGAGCAGTGAACCGGA |
| | GCCCATACCATAGGGACAGAGACCAGCCGCTAACATCCCGAGCCCCTCACTGGCGGCCCCA |
| | GAACACCCCGTGGAAAGAGAACAGACCCACAGTCCCACCTGGAACAGGGCAGACACTGCTG |
| | AGCCCCCAGCACCAGCCCCAAGAAACACTAGGCAACAGCATCAGAGGGGGCTCCTGAGAAA |
| | GAGAGGAGGGGAGGTCTCCTTCACCATCAAATGCTTCCCTTGACCAAAAACAGGGTCCACG |
| | CAACTCCCCCAGGACAAAGGAGGAGCCCCCTGTACAGCACTGGGCTCAGAGTCCTCTCTGA |
| | GACAGGCTCAGTTTCAGACAACAACCCGCTGGAATGCACAGTCTCAGCAGGAGAGCCAGGC |
| | CAGAGCCAGCAAGAGGAGACTCGGTGACACCAGTCTCCTGTAGGGACAGGAGGATTTTGTG |
| | GGGGTTCGTGTCACTGTGGTGGATATAGTGGCTACGATTACGTATTACTATGATAGTAGTG |
| | GTTATTACTACCACAGTGACACAACCCCATTCCTAAAGCCCTACTGCAAACGCACCCACTC |
| | CTGGGACTGAGGGGCTGGGGGAGCATCTGGGAAGTATGGCCTAGGGGTGTCCATCAATGCC |
| | CAAAATGCACCAGACTCTCCCCAAGACATCACCCCACCAGCCAGTGAGCAGAGTAAACAGA |
| | AAATGAGAAGCAGCTGGGAAGCTTGCACAGGCCCCAAGGAAAGAGCTTTGGCAGGTGTGCA |
| | AGAGGGGATGTGGGCAGAGCCTCAGCAGGGCCTTTTGCTGTTTCTGCTTTCCTGTGCAGAG |
| | AGTTCCATAAACTGGTATTCAAGATCAATGGCTGGGAGTGAGCCCAGGAGGACAGTGTGGG |
| | AAGAGCACAGGGAAGGAGGAGCAGCCGCTATCCTACACTGTCATCTTTTGAAAGTTTGCCC |
| | TGTGCCCACAATGCTGCATCATGGGATGCTTAACAGCTGATGTAGACACAGCTAAAGAGAG |
| | AATCAGTGAAATGCATTTGCAGCACAGATCTGAATAAATCCTCCAGAATGTGGAGCAGCAC |
| | AGAAGCAAGCACACAGAAAGTGCCTGATGCCAAGGCAAAGTTCAGTGGGCACCTTCAGGCA |
| | TTGCTGCTGGGCACAGACACTCTGAAAAGCACTGGCAGGAACTGCCTGTGACAAAGCAGAA |
| | CCCTCAGGCAATGCCAGCCCTAGAGCCCTTCCTGAGAACCTCATGGGCAAAGATGTGCAGA |
| | ACAGCTGTTTGTCATAGCCCCAAACTATGGGGCTGGACAAAGCAAACGTCCATCTGAAGGA |
| | GAACAGACAAATAAACGATGGCAGGTTCATGAAATACAAACTAGGACAGCCAGAGGACAAC |
| | AGTAGAGAGCTACAGGCGGCTTTGCGGTTGAGTTCATGACAATGCTGAGTAATTGGAGTAA |
| | CAGAGGAAAGCCCAAAAAATACTTTTAATGTGATTTCTTCTAAATAAAATTTACACCCGGC |
| | AAAATGAACTATCTTCTTAAGGGATAAACTTTCCCCTGGAAAAACTATAAGGAAAATCAAG |
| | AAAACGATGATCACATAAACACAGTGGTGGTTACTTCTACTGGGGAAGGAAGAGGGTATGA |
| | GCTGAGACACACAGAGTCGGCAAGTCTCCTAACAAGAACAGAACAAATACATTACAGTACC |
| | TTGAAAACAGCAGTTAAACTTCTAAATCGCAAGAAGAGGAAAATGCACACACCTGTGTTTA |
| | GAAAATTCTCAGTCCAGCACTGTTCATAATAGCAAAGACATTAACCCAGGTTGGATAAATA |
| | AGCGATGACACAGGCAATTGCACAATGATACAGACATACATTCAGTATATGAGACATCGAT |
| | GATGTATCCCCAAAGAAATGACTTTAAAGAGAAAAGGCCTGATGTGTGGTGGCAATCACCT |
| | CCCTGGGCATCCCCGGACAGGCTGCAGGCTCACTGTGTGGCAGGGCAGGCAGGCACCTGCT |
| | GGCAGCTCCTGGGGCCTGATGTGGAGCAGGCACAGAGCTGTATATCCCCAAGGAAGGTACA |
| | GTCAGTGCATTCCAGAGAGAAGCAACTCAGCCACACTCCCTGGCCAGAACCCAAGATGCAC |
| | ACCCATGCACAGGGAGGCAGAGCCCAGCACCTCCGCAGCCACCACCACCTGCGCACGGGCC |
| | ACCACCTTGCAGGCACAGAGTGGGTGCTGAGAGGAGGGGCAGGGACACCAGGCAGGGTGAG |
| | CACCCAGAGAAAACTGCAGAAGCCTCACACATCCACCTCAGCCTCCCCTGACCTGGACCTC |
| | ACCTGGCCTGGGCCTCACCTGACCTGGACCTCACCTGGCCTGGGCTTCACCTGGCCTGGGC |
| | TTCACCTGACCTGGACCTCACCTGGCCTCGGGCCTCACCTGGCCTGGGCTTCACCTGGCCT |
| | GGGCTTCACCTGACCTGGACCTCACCTGGCCTGGGCCTCACCTGACCTGGACCTCACCTGG |
| | CCTGGGCTTCACCTGGCCTGGGCTTCACCTGGCCTGGGCTTCACCTGACCTGGACCTCACC |
| | TGGCCTGGGCTTCACCTGACCTGGACCTCACCTGGCCTCGGGCCTCACCTGCACCTGCTCC |
| | AGGTCTTGCTGGAGCCTGAGTAGCACTGAGGCTGTAGGGACTCATCCAGGGTTGGGGAATG |
| | ACTCTGCAACTCTCCCACATCTGACCTTTCTGGGTGGAGGCACCTGGTGGCCCAGGGAATA |
| | TAAAAAGCCCCAGAATGATGCCTGTGTGATTTGGGGGCAATTTATGAACCCGAAAGGACAT |
| | GGCCATGGGGTGGGTAGGGACAGTAGGGACAGATGTCAGCCTGAGGTGAAGCCTCAGGACA |
| | CAGGTGGGCATGGACAGTGTCCACCTAAGCGAGGGACAGACCCGAGTGTCCCTGCAGTAGA |
| | CCTGAGAGCGCTGGGCCCACAGCCTCCCCTCGGGGCCCTGCTGCCTCCTCAGGTCAGCCCT |
| | GGACATCCCGGGTTTCCCCAGGCCTGGCGGTAGGTTTGAAGTGAGGTCTGTGTCACTGTGA |
| | GGATATTGTAGTGGTGGTAGCTGCTACTCCTGACTACGGTGACTACCACAGTGTCACAGAG |
| | TCCATCAAAAACTCATGCCTGGGAGCCTCCCACCACAGCCCTCCCTGCGGGGGACCGCTGC |
| | ATGCCGTGTTAGGATTTTGATCGAGGACACGGCGCCATGGGTATGGTGGCTACCACAGCAG |
| | TGCAGCCCATGACCCAAACACACGGGGCAGCAGAAACAATGGACAGGCCCACAAGTGACCA |
| | TGATGGGCTCCAGCCCACCAGCCCCAGAGACCATGAAACAGATGGCCAAGGTCACCCTACA |
| | GGTCATCCAGATCTGGCTCCAAGGGGTCTGCATCGCTGCTGCCCTCCCAACGCCAAACCAG |
| | ATGGAGACAGGGCCGGCCCCATAGCACCATCTGCTGCCGTCCACCCAGCAGTCCCGGAAGC |
| | CCCTCCCTGAACGCTGGGCCACGTGTGTGAACCCTGCGAGCCCCCCATGTCAGAGTAGGGG |
| | CAGCAGGAGGGCGGGGCTGGCCCTGTGCACTGTCACTGCCCCTGTGGTCCCTGGCCTGCCT |
| | GGCCCTGACACCTGAGCCTCTCCTGGGTCATTTCCAAGACATTCCCAGGGACAGCCGGAGC |
| | TGGGAGTCGCTCATCCTGCCTGGCTGTCCTGAGTCCTGCTCATTTCCAGACCTCACCAGGG |
| | AAGCCAACAGAGGACTCACCTCACACAGTCAGAGACAATGAACCTTCCAGAAATCCCTGTT |
| | TCTCTCCCCAGTGAGAGAAACCCTCTTCCAGGGTTTCTCTTCTCTCCCACCCTCTTCCAGG |
| | ACAGTCCTCAGCAGCATCACAGCGGGAACGCACATCTGGATCAGGACGGCCCCCAGAACAC |
| | GCGATGGCCCATGGGGACAGCCCAGCCCTTCCCAGACCCCTAAAAGGTATCCCCACCTTGC |
| | ACCTGCCCCAGGGCTCAAACTCCAGGAGGCCTGACTCCTGCACACCCTCCTGCCAGATATC |
| | ACCTCAGCCCCCTCCTGGAGGGGACAGGAGCCCGGGAGGGTGAGTCAGACCCACCTGCCCT |
| | CAATGGCAGGCGGGGAAGATTCAGAAAGGCCTGAGATCCCCAGGACGCAGCACCACTGTCA |
| | ATGGGGGCCCCAGACGCCTGGACCAGGGCCTGTGTGGGAAAGGCCTCTGGCCACACTCAGG |
| | GGCTTTTTGTGAAGGGCCCTCCTGCTGTGGTGGATACAGCTATGGTTACGGGTATAGCAGC |
| | AGCTGGTACCACAGTGATGAAACCAGCAGCAAAAACTGACCGGACTCGCAGGGTTTATGCA |
| | CACTTCTCGGCTCGGAGCTCTCCAGGAGCACAAGAGCCAGGCCCGAGGGTTTGTGCCCAGA |
| | CCCTCGGCCTCTAGGGACACCCGGGCCATCTTAGCCGATGGGCTGATGCCCTGCACACCGT |
| | GTGCTGCCAAACAGGGGCTTCAGAGGGCTCTGAGGTGACTTCACTCATGACCACAGGTGCC |
| | CTGGTCCCTTCACTGCCAGCTGCACCAGACCCTGTTCCGAGAGATGCCCCAGTTCCAAAAG |
| | CCAATTCCTGGGGCCGGGAATTACTGTAGACACCAGCCTCATTCCAGTACCTCCTGCCAAT |
| | TGCCTGGATTCCCATCCTGGCTGGAATCAAGAGGGCAGCATCCGCCAGGCTCCCAACAGGC |
| | AGGACTCCCACACACCCTCTTCTGAGAGGCCGCTGTGTTCCGCAGGGCCAGGCCGCAGACA |
| | GTTCCCCTCACCTGCCCATGTAGAAACACCTGCCATTGTCGTCCCCACCTGGCAAAGACCA |
| | CTTGTGGAGCCCCCAGCCCCAGGTACAGCTGTAGAGAGAGTCCTCGAGGCCCCTAAGAAGG |
| | AGCCATGCCCAGTTCTGCTGGGACCCTCGGCCAGGCCGACAGGAGTGGACGCTGGAGCTGG |
| | GCCCACACTGGGCCACATAGGAGCTCACCAGTGAGGGCAGGAGAGCACATGCCGGGGAGCA |
| | CCCAGCCTCCTGCTGACCAGAGACCCGTCCCAGAGCCCAGGAGGCTGCAGAGGCCTCTCCA |
| | GGGGGACACAGGGCATGTCTGGTCCCTGAGCAGCCCCCAGGCTCTCTAGCACTGGGGGCCC |
| | CTGGCACAGCTGTCTGGACCCTCCCTGTTCCCTGGGAAGCTCCTCCTGACAGCCCCGCCTC |
| | CAGTTCCAGGTGTGGTTATTGTCAGGGGGTGCCAGGCCGTGGGGTATAGCAGTGGCTGGTA |
| | CGTATTACTATGATAGTAGTGGTTATTACTACCACAGTGGTGCCGCCCATAGCAGCAACCA |
| | GGCCAAGTAGACAGACCCCTGCCACGCAGCCCCAGGCCTCCAGCTCACCTGCTTCTCCTGG |
| | GGCTCTCAAGGCTGCTGTCTGCCCTCTGGCCCTCTGTGGGGAGGGTTCCCTCAGTGGGAGG |
| | TCTGTGCTCCAGGGCAGGGATGACTGAGATAGAAATCAAAGGCTGGCAGGGAAAGGCAGCT |
| | TCCCGCCCTGAGAGGTGCAGGCAGCACCACAGAGCCATGGAGTCACAGAGCCACGGAGCCC |
| | CCAGTGTGGGCGTGTGAGGGTGCTGGGCTCCCGGCAGGCCCAGCCCTGATGGGGAAGCCTG |
| | CCCCGTCCCACAGCCCAGGTCCCCAGGGGCAGCAGGCACAGAAGCTGCCAAGCTGTGCTCT |
| | ACGATCCTCATCCCTCCAGCAGCATCCACTCCACAGTGGGGAAACTGAGCCTTGGAGAACC |
| | ACCCAGCCCCCTGGAAACAAGGCGGGGAGCCCAGACAGTGGGCCCAGAGCACTGTGTGTAT |
| | CCTGGCACTAGGTGCAGGGACCACCCGGAGATCCCCATCACTGAGTGGCCAGCCTGCAGAA |
| | GGACCCAACCCCAACCAGGCCGCTTGATTAAGCTCCATCCCCCTGTCCTGGGAACCTCTTC |
| | CCAGCGCCACCAACAGCTCGGCTTCCCAGGCCCTCATCCCTCCAAGGAAGGCCAAAGGCTG |
| | GGCCTGCCAGGGGCACAGTACCCTCCCTTGCCCTGGCTAAGACAGGGTGGGCAGACGGCTG |
| | CAGATAGGACATATTGCTGGGGCATCTTGCTCTGTGACTACTGGGTACTGGCTCTCAACGC |
| | AGACCCTACCAAAATCCCCACTGCCTCCCCTGCTAGGGGCTGGCCTGGTCTCCTCCTGCTG |
| | TCCTAGGAGGCTGCTGACCTCCAGGATGGCTTCTGTCCCCAGTTCTAGGGCCAGAGCAGAT |
| | CCCAGGCAGGCTGTAGGCTGGGAGGCCACCCCTGTCCTTGCCGAGGTTCAGTGCAGGCACC |
| | CAGGACAGGAAATGGCCTGAACACAGGGATGACTGTGCCATGCCCTACCTAAGTCCGCCCC |
| | TTTCTACTCTGCAACCCCCACTCCCCAGGTCAGCCCATGACGACCAACAACCCAACACCAG |
| | AGTCACTGCCTGGCCCTGCCCTGGGGAGGACCCCTCAGCCCCCACCCTGTCTAGAGGACTT |
| | GGGGGGACAGGACACAGGCCCTCTCCTTATGGTTCCCCCACCTGGCTCCTGCCGGGACCCT |
| | TGGGGTGTGGACAGAAAGGACGCCTGCCTAATTGGCCCCCAGGAACCCAGAACTTCTCTCC |
| | AGGGACCCCAGCCCGAGCACCCCCTTACCCAGGACCCAGCCCTGCCCCTCCTCCCCTCTGC |
| | TCTCCTCTCATCACTCCATGGGAATCCAGAATCCCCAGGAAGCCATCAGGAAGGGCTGAAG |
| | GAGGAAGCGGGGCCGCTGCACCACCGGGCAGGAGGCTCCGTCTTCGTGAACCCAGGGAAGT |
| | GCCAGCCTCCTAGAGGGTATGGTCCACCCTGCCTGGGGCTCCCACCGTGGCAGGCTGCGGG |
| | GAAGGACCAGGGACGGTGTGGGGGAGGGCTCAGGTCCCTGCAGGTGCTCCATCTTGGATGA |
| | GCCCATCCCTCTCACCCACCGACCCGCCCACCTCCTCTCCACCCTGGCCACACGTCGTCCA |
| | CACCATCCTGAGTCCCACCTACACCAGAGCCGGCAGAGCCAGTGCAGACAGAGGCTGGGGT |
| | GCAGGGGGGCCGCCAGGGCAGCTTTGGGGAGGGAGGAATGGAGGAAGGGGAGGTCAGTGAA |
| | GAGGCCCCCCTCCCCTGGGTCTAGGATCCACCTTTGGGACCCCCGGATCCCATCCCCTCCA |
| | GGCTCTGGGAGGAGAAGCAGGATGGGAGAATCTGTGCGGGACCCTCTCACAGTGGAATACC |
| | TCCACAGCGGCTCAGGCCAGATACAAAAGCCCCTCAGTGAGCCCTCCACTGCAGTGCTGGG |
| | CCTGGGGGCAGCCCCTCCCACAGAGGACAGACCCAGCACCCCGAAGAAGTCCTGCCAGGGG |
| | GAGCTCAGAGCCATGAAGGAGCAAGATATGGGGACCCCAATACTGGCACAGACCTCAGCTC |
| | CATCCAGGCCCACCAGGACCCACCATGGGTGGAACACCTGTCTCCGGCCCCTGCTGGCTGT |
| | GAGGCAGCTGGCCTCTGTCTCGGACCCCCATTCCAGACACCAGACAGAGGGACAGGCCCCC |
| | CAGAACCAGTGTTGAGGGACACCCCTGTCCAGGGCAGCCAAGTCCAAGAGGCGCGCTGAGC |
| | CCAGCAAGGGAAGGCCCCCAAACAAACCAGGAGGTTTCTGAAGCTGTCTGTGTCACAGTCA |
| | GGATATTGTAGTGGTGGTAGCTGCTACTCCAGCATATTGTGGTGGTGATTGCTATTCCCAC |
| | AATGACACTGGGCAGGACAGAAACCCCATCCCAAGTCAGCCGAAGGCAGAGAGAGCAGGCA |
| | GGACACATTTAGGATCTGAGGCCACACCTGACACTCAAGCCAACAGATGTCTCCCCTCCAG |
| | GGCGCCCTGCCCTGTTCAGTGTTCCTGAGAAAACAGGGGCAGCCTGAGGGGATCCAGGGCC |
| | AGGAGATGGGTCCCCTCTACCCCGAGGAGGAGCCAGGCGGGAATCCCAGCCCCCTCCCCAT |
| | TGAGGCCATCCTGCCCAGAGGGGCCCGGACCCACCCCACACACCCAGGCAGAATGTGTGCA |
| | GGCCTCAGGCTCTGTGGGTGCCGCTAGCTGGGGCTGCCAGTCCTCACCCCACACCTAAGGT |
| | GAGCCACAGCCGCCAGAGCCTCCACAGGAGACCCCACGCAGCAGCCCAGCCCCTACCCAGG |
| | AGGCCCCAGAGCTCAGGGCGCCTGGGTGGATTCTGAACAGCCCCGAGTCACGGTGGTATTA |
| | CGATTTTTGGAGTGGTTATTATACCGTATTACTATGGTTCGGGGAGTTATTATAACCACTG |
| | TGAGAAAAGCTATGTCCAAAACTGTCTCCCGGCCACTGCTGGAGGCCCAGCCAGAGAAGGG |
| | ACCAGCCGCCCGAACATACGACCTTCCCAGCCCTCATGACCCCCAGCACTTGGAGCTCCAC |
| | AGTGTCCCCATTGGATGGTGAGGACGGGGGCCGGGGCCATCTGCACCTCCCAACATCACCC |
| | CCAGGCAGCACAGGCACAAACCCCAAATCCAGAGCCGACACCAGGAACACAGACACCCCAA |
| | TACCCTGGGGGACCCTGGCCCTGGTGACTTCCCACTGGGATCCACCCCCGTGTCCACCTGG |
| | ATCAAAGACCCCACCGCTGTCTCTGTCCCTCACTCAGGGCCTGCTGAGGGGCGGGTGCTTT |
| | GGAGCAGACTCAGGTTTAGGGGCCACCATTGTGGGGCCCAACCTCGACCAGGACACAGATT |
| | TTTCTTTCCTGCCCTGGGGCAACACAGACTTTGGGGTCTGTGCAGGGAGGACCTTCTGGAA |
| | AGTCACCAAGCACAGAGCCCTGACTGAGGTGGTCTCAGGAAGACCCCCAGGAGGGGGTTTG |
| | TGCCCCTTCCTCTCATGTGGACCCCATGCCCCCCAAGATAGGGGCATCATGCAGGGCAGGT |
| | CCTCCATGCAGCCACCACTAGGCAACTCCCTGGCGCCGGTCCCCACTGCGCCTCCATCCCG |
| | GCTCTGGGGATGCAGCCACCATGGCCACACCAGGCAGCCCGGGTCCAGCAACCCTGCAGTG |
| | CCCAAGCCCTTGGCAGGATTCCCAGAGGCTGGAGCCCACCCCTCCTCATCCCCCCACACCT |
| | GCACACACACACCTACCCCCTGCCCAGTCCCCCTCCAGGAGGGTTGGAGCCGCCCATAGGG |
| | TGGGCGCTCCAGGTCTCACTCACTCGCTTCCCTTCCTGGGCAAAGGAGCCTCGTGCCCCGG |
| | TCCCCCCTGACGGCGCTGGGCACAGGTGTGGGTACTGGGCCCCAGGGCTCCTCCAGCCCCA |
| | GCTGCCCTGCTCTCCCTGGGAGGCCTGGGCACCACCAGACCACCAGTCCAGGGCACAGCCC |
| | CAGGGAGCCGCCCACTGCCAGCTCACAGGAAGAAGATAAGCTTCAGACCCTCAGGGCCGGG |
| | AGCTGCCTTCCTGCCACCCCTTCCTGCCCCAGACCTCCATGCCCTCCCCCAACCACTTACA |
| | CACAAGCCAGGGAGCTGTTTCCACACAGTTCAACCCCAAACCAGGACGGCCTGGCACTCGG |
| | GTCACTGCCATTTCTGTCTGCATTCGCTCCCAGCGCCCCTGTGTTCCCTCCCTCCTCCCTC |
| | CTTCCTTTCTTCCTGCATTGGGTTCATGCCGCAGAGTGCCAGGTGCAGGTCAGCCCTGAGC |
| | TTGGGGTCACCTCCTCACTGAAGGCAGCCTCAGGGTGCCCAGGGGCAGGCAGGGTGGGGGT |
| | GAGGCTTCCAGCTCCAACCGCTCCACTAGCCGAGACTAAGGAAGTGAGAGGCAGCCAGAAA |
| | TCCAGACCATTCCATAGCAAATGGATTTCATTAAAGTTACCAGACTTCAGTGTAAGTAACA |
| | TGAGCCCCATGCACAACAATCCCTTATGAAGGGGAAGTCAGTGTCGCCTCGGATTTCTTGA |
| | AAAACACAAAAACTTATCAATGCCTGTAAAAGTCTGTTGGAAAGAAAATATGATTCAAGAA |
| | TGTTATGCCCAACAAAGCTGGCATATTTTCTACCCGGACACACTCAGGGAATGTGGTCCCT |
| | TGAGTGCTTCTCTCACTGCGTAAATCCTACGTGGTGTTTAAGCATATTCATAAATGTGTAT |
| | GTCTATTTTTATGTGTAAGATGGTTCATTTTTATTTTATTTATTCAATATGTACAATAAAG |
| | AATATTGACAAATAGGCTGGACATGGTGGCTCCCACCTGTAATCCCAGCCCTTTGGGAGGC |
| | CGAGGCGGGCAGATCACCTGAGGTCTGGAGTTCGAGACCAGCCTGGCCAACATGATGAAAA |
| | CCCATCTCTACTAAAAATACAAAGATTAGCCAGGCATGGTGGTGCATGCCTGTAATCCCAG |
| | CCACTCAGGAGGCTGAGACAGGAGAAATGCGTGAACCCGGAAGGCGGAGGTTGCAGTGAGC |
| | CGAGATCACACCACTGCACTCCAGCCTGGCGACAGAGCAAGATTCCATCTCAAAAAAAAAA |
| | AGACAAAGAAATTTGTTTTTTTGAATAAAGACAAATTTCATCACACGAAGATAAAGATGCA |
| | AAGCTCCAGACAGGAAGGCACGGACAGCACAGTGAAGCCCGGAGCGGGCGCTGGGGGGCCA |
| | GGGGCATGGCGGGGGTGCCAGCGTCTCTCGGTGCCTACCATGGCCACTCCAGCCTGTGTTC |
| | TCACGAGGATGGCTGTGCAATGCTAGGAGCGTGTTCGAAGCTCTAGGGCAACCACTGGAAG |
| | TGAGGCTGAGGAGCAGAGCCCAGAGGCCCGTGGAGCTGATGAAAAGAAAGCTGGAGAAAGT |
| | GTTTGCTGCCTCCCAACATGGTAAGAAAAGATAGAAAGAGAGAGCACACGGCAAAGGGAGC |
| | TTGCTGAGGGACTCTTTACAATGGCTTGCACAGAGCTCAGGGGGTCTGGGAGGCTAGGGCC |
| | CTGCGCAGGGCAGTCACCCCAGCCTGCTGACCAAGGTTTGCTGCAGGCAGCTCTGGGGGTG |
| | GTTGAGGCGCGGTCCCTGGAGCCACCCCTCAAGGGAACGAGGCAGCAGAGTGGGCCAAGGC |
| | CCAGGTCGGCTGCAAGGCTGCCCAGGACTTGGGGTCCTTACATCAGCAGCCACTGATGCAG |
| | CTGGCCCAGAGAGAGGCGCCGAGCAGGTTGCCTCCAGGGGACAAACCAGGTCGGAGAGGGT |
| | GAGGCAGTGGATGGAGCCACAACAACCCCGGGCACGGGTGACACGCACGTTCATGCACATC |
| | TGACCCTTCCTCCCTCACCAAACAGGTCCCCCTGCCTTCCCCATGGTTGCGAAAAAGCAAA |
| | ATGTAGACGTTTTTTCTTTTTTAATTCATGTTTTAATTGACAAATGAAGCCGTATATATTT |
| | ATTGTGTACAACATGATGCTTTAAAATATGTATACATCGTGGAACAGCAACGTTGAGCTAA |
| | TTTAACACGCATTACTTCACATACTTGTCATCTTTTGTGGCGAGAATGCTTAAAATCCACT |
| | CTCTTAGTATTTTTTAAGAATGCAATACATTGTTGTCAACTGTGGTCACCGTCATGCATAG |
| | CCAAGCTCCCGACCTCACCCTCCTGCCAGCTCAGGCTGTGCATCCTTTCACCAGCATCCCC |
| | CACCCCGGCCCCTGGCCCTGGTAACTACCACTCTATACTCTACGTATGAGTTCAGCTTTTT |
| | AAGATTCCACAGATGAATGAGATCATACAGTATTTGCTTTCTATGCCTGGCTTATTTTAGT |
| | TAACACACTGTCCTCCAGATCCATCCGTTGTTGCAAATGACAGGGTTTCATTCTTTTTAAA |
| | GTCTAAAGAGTATTCCATTGTGTCAATGGACCTCATTTGCTTTATCCATGCATCAACTATG |
| | GACATTTAGGTTGATTCCATTTCTTAGCTGTTGTGGATGGTGCTGCAGTAAACATGGGGCT |
| | GCAGATGTCTCTTCAACATACTGACATCATGTCCTTTGGATAAATACCCAGTAGTGGGATC |
| | GCTGGATCACAATGTACAGTTTTTTTTTTAATGGAAACTTTCATTTTTTGGTGAAATTAGG |
| | AAAACAGATAAAACCCACAGAATCCAAAATATATGTGAAGATGCCAAAAACAGTTGACATT |
| | GGGCAGAGGTCACATGGAAGGAAGTGAATACATGACGGGGTGTGAGGGCCCAGAGGCAGCT |
| | GAAATACGCTTTCTAAACACAAGGACCTCTTCTGAGAGGGCAGAAGTTTTATCCTGCACAT |
| | GCAATGACCAGCACAGCTAAAATACACTTTCTAAACATGAGGACCTCTTCTGAGAGGGCAG |
| | CTTTATCCTGCAAATGCAATGACCAGCACAGGACCCAGAATAAAGAGAGTTGCCAGCGGAC |
| | GCCTGGTGTCCATGTGTCCAGGTGAGTTCGAGATGCGGACGGCGCTGGCCAGCCAGTCACA |
| | CCCTAAGTCAATCTGCTGCATGCATTTGTCCTTGCCACAGCAGAAAACGAGAAAGCCTTTG |
| | GGCTGCAAAGCTTCACAGGCTCCTCTTCTCCCGACTCCATGGAAACAGCTACAAAGAGCAG |
| | GCCCAGTAGAGCTTAATTCATGAAAATGAGTAATAAACTTGAACTGGAACAGTATCGACTT |
| | TTTAGAAACGGCAGCAAAGTGTATAAAAAATATTCACCAGAACAATATTTCCAAACGATGA |
| | GATGAGAATTTCAGCCAAGTAATCCTCCATGGATAGAAAATAATGAAGGGATTGGATTTAT |
| | GAAGGAAAATCATGGAGCTCAAATACAAGAGAAGAGAATCAAAAATGAACAGGAGGAGATA |
| | AAATATGGTTTGGCCAAAGTTACAAAATAAATTTTTTAAAAACCCTTCATCATGGCAAGTA |
| | GAAAGAGCGAGAGGAAAAACAGATCCCGTGGAAGACACAAATAGGACATGGGGAGAAAAAT |
| | GAATGAGATGAAACAGAGCAGAAATAAAATTTTACGGAACTAAAGACAAGTGATCTGAACC |
| | TGCCTGGGGCCTGGGGGACCTCGCCACCCTGAAGGGAAAGAACATGCCTGGCTGGCTTTGC |
| | CACCTGCTCATTGCAGAGCCCCACAGCTTGCAACAAACATAGGCGGTAGCCAGGGAGTGGT |
| | TACAGCAGGCCTTGAGCAAGACCCAGTGTTGTGCTGACTTCAGGTCTGACCCAGCACTGTC |
| | ATAGTGGTGGTGTCCATAGTGGTAGTGGGGGTGCTTGTGTCACTCCACCCCCATCTCCAGG |
| | AGGCTCAGAACAGACAGAGAGAGACTCCATTTGTTTGGGAGAAAGTAAGGGATGAGAACAA |
| | GAGTCTCTGCCTGGTAATCCAGAGAATTATTCTAGATCTTGGCCAAGATTATCAAAGCAGT |
| | ACCTCTATGAGTCTTTTGGGCTTGGAGTCCCCCTAAAGCAGATATAGCTAAGATCACAACA |
| | CCCAAGTCCTTTTGAATATGTGGGAAGACTTCCCAAGGACAGGAGCAAACAAACAAGCCCA |
| | GACTGCAAAAAAACAAGCCCAGACTGCAATAAACACCTCACTCTTCAATGCCCAGGCACTG |
| | AAGAACATCTCCTAGCAGCAACACCATCCAGGAAAACATGGCCTCAACCAGTGAACTAAAT |
| | AAGGCACCAGGGACCAGTCTCGGAGAAATAGAGGTATGTTATCTTTCAGAGAATTCAAAGT |
| | AGCTTTGTTGAGGAAACTCAAAGAAATTCAAGATAACACAGTGAAGGAATTCAGAATCCTA |
| | TCCGATAAATTTAACAGAGATTGAAGCAATTAAAAAGAATTAAGCAGAAATTATGGAGCTG |
| | AAAAATGCAATTGGCATACTGAAAAATGCATCAGAGTATTTTCATAGCCTCTTATATCAAG |
| | TAGAAGAAAGAATTAGTGAGCTTGAAAACAGGCTATTTGGAAAAGCACGATAAAAGGAGAC |
| | AAAAGAGAAAAGAATAAATAACAATGAAGCATATCTACAGGATCTAGAAAATAGCCTCAAA |
| | AGGCCAAATCTAAGAATTATTAGCCTTAAAGAGGAGGTAGAGAAAGAGGGATGGAGAGTTT |
| | ATTCAAAGGGATAATAACAGAAAACTTCCCAAACCTAGAGAAAGATATCAATATCCAAATG |
| | CAAGAAGGATGTAGTACACCAAGGAGATTTAATGCAAAGAAGACTACCTCAAGGCATTCAA |
| | TACTCAAACTCCCATATGACAAGGACTTTAAAAAGATCCTAAAAGCAGCAAAAGAAAAGAA |
| | ATGAATAAAATACTATGGAGCTCCAATATGTCTGGCAGCAGACTTTTCAGTGAAGACTTTA |
| | TATGCCAGGAGAGAGTGTCATAATGGATTTAAAGTGCTGAAGGAAAAAACTTTTACCCTCG |
| | AACAGTATAGCTGGTGAAATTATCCTTCAAACATGAAGGAGAAATAATTTGTTTCCAGACA |
| | AATGTTGAGGGATTTCATGAACACCAGACCTGTCTTTTAAGAAATGCTAAAGGGAGTACTT |
| | CAATCAGAAAGAAACACGTTAGTGAACAATAAGAAATCATCTGAAGGCACAAAACTCACCG |
| | GTAATAGTAAGTACACAGAAAAACACAGAATATTATAACACTGTAACTGTGGTGTGTAAAC |
| | TCCTTTTGTTTGTTTGTTTGTTTGTTTGTTTGTTTGTTTGTTTTTGTTTTTAGACGGAGTT |
| | TTGCTCCAGCCCAGGCTGGAGTGCAATGGCACAATCTCAGCTCACTGCAACTTCCACCTCC |
| | CGGGTTCAAGCAATTCTCCTGCCTCAGCCTCCCAAGTAGCTGGGATTACAGGCATGTGCTA |
| | CCATGTCCAGCTAATTTTGTATTTTAGTAGAGACGGTGTTTCACCATGTTGGTCAGGCTAG |
| | CCTTATCTTGAGTAGAAAAACTAAATGATGAAGCAATGAAAAATAATAACTACAACTTTTC |
| | AAGACATAGTACAATAAGATATAAATCATAACAAAAAGTTAAAAGGTGGAGGGATGAAGTT |
| | AAGGCAAAGAGTCTTTATTAGTTTTCTTTTTACTTGTCTGTTTATGCAAACAGTGTTAAGT |
| | TGTCATCAGTTTAAAATAATGGGTCATAAGATACTATTTGCAAGCCTCATGGTAACGTCAA |
| | ACCAAAAGCAATACAACAGATACACAAAAAACAAAAAGCAAGAAGCTAAATTACGTCATCA |
| | GAGAAAATCACCTTCACTAAAAGGAAGACGGAGAAAAGAATGAAGAGAGAGAAGACCAAAA |
| | GCAAATAGCAATATGGCAGGAGTAAGTCCTTACTTATCAATAATACCATTGAATGTAAATG |
| | GACTAAACTCTCCAATCAAAAGACATAGAGTGGCTGAATCAATTAAAGAAAAAACAAGACC |
| | CATTGATCTGTTGTCCACAAGAAACACACTTTATCTATAAAGACACACATAGACTGAAAAC |
| | AAAGGGATGGAAAAAGATACTCCACGCCAATGGAAACCAAAGAAAGAGCAGGAGTAGCTAC |
| | ACTTATATCAGGCAAAATAGATTTCAAGACAAAAACTATAAGAAGAGACAAGGTCACTAAT |
| | GATAAACAGGTCAATTCAGCAAGAGGATATAACAATTGTAAATATATATGCACCCAATGCT |
| | GGAGCACCCAGATATATAAAGCAAGTATTTACTAGAGCTAAAGAGAGAAATAGACTCCAAT |
| | GCAATAATAGCTGGAGATTTCAACATCCCACTTTCAACATTGAACAGATCCTCCAGATAGA |
| | AAATCAACAAAGAAATATTGGACTTAATCTGCACTATCGACCAAATGGATCTAACAGATAT |
| | TTACAGAACATTTCATCCAACAGCTGCAGAACACACATTCTTTTCCTCAGCACATAGATCA |
| | TTCTCAAGGATAGACCATATGTTGGGTCACAAAACAAGTTTTAAAATATTCAAATACATTG |
| | AAATAATATCAAGCATCTTCTGTGACCACAATGGACTAAAACTAGAAATCAATAACAAGAG |
| | GAATTTTGGAAACTATATAAATATATGGAAATTAATGAATGCTGAGTGGGTCAATGAAGCA |
| | ATTAAGAAGGAAACTGAAATTTTTCTTGGAACGAATGATCATGGAAACAGAAAATACCAAA |
| | ACCTATGGGATACAGCAAAAGCAGTACTAAGAGGGAAGTTTACAGCTACAAATGCTTACAT |
| | TAAAAAAGAAGAAAAACTTCAATAAAAAAACCTAACAATGCATCTTAAAGAACTAGAAAAG |
| | CAAGAGGAAATCAAATCCAAAATTAGTAGAAGAAAACAGTAAAGGTCAGAGCAGAAATAAG |
| | TAAAATTGAAATGAAGAAAACAATACAAAAGATCAATAAAACAACAGGTTGTTTTCTTGAA |
| | AAGTTAAACAAAATTGACAAACCTTTAGCCAGACTAAGAAAAAAAGACAGAAGATCCAAAT |
| | AAATAAAATCAGAGATGAAAAAGGTGACATTACAACTTACACCACAGAAATTCAAAGGATC |
| | ATTAGTGGCTACTATAAGCAACTATATGCCAATAAATTGGAAAATCTAGAAGAAATGCAGA |
| | AATTCCTAGACACATACAACCTCCCAAGATTAAACCAAGAAGAAATTCAAAACCTGAACAG |
| | ACTGATAACAAGTAATGAGATCAAAGCCGTAATAAAAAGCCTCCCAGTAAAGAGAAGCCCA |
| | GGACCCGACGGCTTCACTGCTGAATTCTACCAAACATTTAAAGTAGAACTAATACCAATCC |
| | TACTCAAACTATTCCAAAAAATAGAGGTGGAAGGAATACTTCAAAACTCATTATACGAGGC |
| | CAGTATTAACCTGACACCAAAACTAGACAAAGACACATGAAAAAAAGAAAACTACAGGCCA |
| | ATATGTCTGATGAATATTGACACAAAAATCCTCAACAAAATACTAGCAAACCAAATTCAAC |
| | TACACATTAGAAAGTTCACTCATCATGACCAAGTGGAATTTATCTAACTTGGGATGCAAAG |
| | ATGGTTCAACATATGCAAATCAATCAATGTGATACATCATATCAACAGAATGAACAACAAA |
| | AACCATTTGATCATTTAATTGATACTGAAAAAGCATTTGATAAAATTCAACATTCCTTCAT |
| | AATAAAAATTCTCTTCTATACTAGGTACAAAAGAAACTTACCTCAACATAATAAAGCCATA |
| | TATGACAGTCCCACAGTATGATACTAAATGAGGAAAAACTGAGAGCCTTTCCTCTACGATC |
| | TGGAACATGACAAAGATGCCCACTTTCATCACTGTTATTCAACATAGTACTGGAAGTCCTA |
| | GCTGGAGCGATCAGACAAGAGAAAGATATAAAAGACATCCAAATTGGAAAGGAATAAGTCA |
| | AATTATCCTCATTTGCATATGGTATGATCTTCTATTTAGAGCTAACTAAAGACTCCACCAA |
| | AAAAAGTTATTAGAACTGACGAACAAATTCAGTAAAGCTGCAGGATACAAAATCAACATAC |
| | AAAAATCAGTAGCATTTCTATATGCCAACAATGACCAATGTGAAAAAGAAATTAAAAAGTA |
| | ACCCTATTTACAATAACCACAAATAAACACCTAGGAATTAACCAAAGAGGTAAAAGATTTC |
| | TGTAATGAAAACTATAAAAACTGATGAAAGAAATTGAAGAGTACACCAAAAAATGGAAAGC |
| | AATTGCATGTTCATGGATTAGAAGAATCAGTGTTGTTATAATGTCCATACTATCCAAAGCA |
| | ATCTACAGATTCAATGCAATCCTTATCAAAATACCAATGACATCATTCACAGAAATAGAAA |
| | AAAAAAATCCTAAAATTTACGTGGAACCACAAAGACCCAGAATAGCCAAAGCTCTCCTAAG |
| | CAAAAAGAACGAAACTGTAGGAATGACATTGCCTGTCTTCAAATTCTACTACAGAGCTATA |
| | GATAGTAACCAAAACAGCGTGGTACTAGCATAAAAACAGACACAGAGACAAACAGAACAAA |
| | ATTTAAAAACCCAGAAATAAATCCACACACCTACAGCAAATTCATTTTTGACAAAGTTGCC |
| | AAGAACATACTCTGGGGAATAGATAATGATATCTCTTCAATAAATAATGTGGGGAAAACTG |
| | GATATCCATATACATAACAGTGAAACTAGACCCCTCTCTCTCTCACTATATACAAAAATCA |
| | AATCAAAATTGTTTAAGGACTTAAATCTAAGACCTCATACTATGAAACCACTGCAAGACAA |
| | CCTTGGCGGAAACTCTCCAAGACATCAGTCCAGGCAAAGATTTCTTGAGTAATATCCCACA |
| | AGCACAGACAACCAAAGCAAAAATGGACAAATGGGATCACATCAAGTTAAAAAGCTTCTGC |
| | ACAGTAAGGGAAACAACCAACAAAATGAAGAGACAACCCACAGAATGGGAGAAAATATTTG |
| | AAAAATACCCATCTGGCAAGGGATTAAAAACCAGAATATATGCAGAATATATAAGGAGCTC |
| | AAACAGTGCTATAGAAAAAAAAATCTAATAATCTGATTTAAAAATGGGAAAAATGTTAGAA |
| | TAGACATTTCTTAAAATAAGACATACAGATGGCAAACCGACATGGAACGGTGCTCAACATC |
| | ATGGATTATCACAGAAACACAATCAATCAAAACTAAAACTAAAATGTGCTATCATCTCACC |
| | CCAGTTAAAATGGCTGATATCCAGAAGACAGGCAATAACAAATGCTGGCAAGGATGTGGGG |
| | AAAAGGGAGCCCCCATACACTGTTGCTGGGATTGTAAATTAGTACAACCACTGTGGAGAGC |
| | AGCATGAAAGTTCCTCAAAAAACTGAAAGAAAGCTACCATAGGATCCAGCAATCCCACTGC |
| | TGTGTATATACTACAAAAGAAAGGAAGTCAGTATATGAAGAGGTATCTGCACTCCCATGTT |
| | TGTTGCAGCCCTGTTCACAACAGCCAAGATTTGGAAGCAACCTAAGTGTCCATCAGCAGTT |
| | GAATGTATAAAGAAAATGTGGTGCATATACACAATGGAGTATTATTCAATAATAAAAAGGA |
| | ATGAGATTGAGTCATTTGCAACAACATGGATGGAACTGGAGATCATTATGTGAAGTGAAAT |
| | AAGCCAGGCACAGAAAGACAAACATTACAATGTTCTTACTTATTAATGAGATCTAAAAATC |
| | AAAACAATTGCACCCATGTTCATAAAGAGTAAAAGGATGGTTACCAGATGCTGAGAACGGT |
| | GGTGGGGGGATAGGGAAAGGTGGCAGTGGTTAACGGGTACAAAAAAATAGAAAGAATGAAT |
| | AAGACTTACTACTTGATAGCACAGCAAGGTGGCTATAGTCAGTAATTTAGTTGTATATTTT |
| | TAATAATGAAAGGTGTATAATTGGATTGTTTCTAACACAAAGGATAATGCTTAAGAGGATG |
| | GATACCCCATTTTCCATGATGTGATTATTTCACATTGCACGCCTAGATCAAAACATCCAAT |
| | GTACCCCATAAATATATACATCTTCTATGTACCCATAAAAATTCTGTAAAATAAAATATAT |
| | AAAAAGAGGTGACAGATATGGAAGACAGGCAAAGAAGAGACGACATCCACATAATCCGAGT |
| | ACCTAAGAAAGAATGGAGTCCAGTGCATCTCAGGAGCCACCATTCTAAGCCAATTTTCTCT |
| | GGTTCTCTCAGTCACCCTACTAATACGTGGGCAATCTTGTTTTATTTCAGGATAGAGTTTT |
| | TGAAATTATAGATTTAAGTATGCTTTCTGTTCTATTACTTTTGGTAATTAATTTTAGAAAG |
| | AACTAATTTGGGCACAAATTTGAAAAAATTCTAAATCCAAAAAAAAAAAGAAAAAAACACA |
| | CACACAATCATCTATAAGGGGGATGATGACCAGTCCTAGATTTCTCACCAGCCACATTCAA |
| | GATCAGTAAATGGTAGGACAAAACCTGTAGGGTCCTTAAGGGGGAAAGAAGTAGTGGATAG |
| | TCCAGAGTCTATATACAGCCAACTGTTCTTGAAGAAAAAAGGCTGCTGAAAAGGAGTTCCA |
| | AACATTCTATAATCCATAATCTCATGATGAAACTACTAGAGGAAGACCACCAGCCATCAAA |
| | AGGTGCTTGGAGAACCCAGGGCCAAGAACCAAAAGTAAATATTAAGTGTCCTTAACTGCGA |
| | GACTAAGATAGAAATGACTGTGGGGGACCATGTGGCCTCAACAGAGGTGAAATGGTGTCTG |
| | CCTGACAAAGTGGACATTTTACAATGATCAAAACACAGAATATGAGATAGAGAGCACTTCT |
| | GAATTACTGCCTCACTCCAAATAACTCTCAGCCAAAGGACTTCAGTAAAACCAAATTGGGC |
| | ATATTAGACAGTACAAACAAATTCTAAGAAAATAATATTACTGATTACAATCACATGATGC |
| | TAGAGATGGAGGGGAAAAGGAAGAGGAAACCAGGTAATTTCATACTCGTATATAGTAAAGA |
| | ACTAAAGTACATTGTCCAAAGAAGAACAAAGAATATTTTGGAAAGTTATAAAGGTAGCCAC |
| | TACACATAGAAGATAGCAAAGAACAAGAAAACTTAAGATGGAAAACTTTTTGGAAGCATAA |
| | AAATAGAAAATATAAACTACTAAGATAAGATTGAAGCCAAACAGATCTATGAAAACAACAA |
| | ACATCAATGGCCTTAACTTGCCTATTAAAAGGAAGAGACTTTCAAATTGGACCACAAGATA |
| | AAACCCAACTCTATATAGCATATGAGTATTACACACAAAATGGGAAAAGCTGAAAAAACTT |
| | GGGCAAAATTCACCCCAAGCAAATTCCACTGTTTCCTTTGGGACAAAATGCCAAGCTCCAT |
| | GCCAGGGAAGATGATTCTCCTCAGACCTTCTCCTCACTCTCCCAGTCCTCTTAGGGAAGGA |
| | ATTGGGTGTTAGAGGAGGGAGACTCTGTCGATTATCAGCTGAAGCAGTGGTGTGCTCCTGC |
| | GTTGCTTCTGACCTGGGAAATGAAGCAGCAAGACTCTTTCTGCTGTGTCTTTGCCCAGAAG |
| | GGCCATCCCCCCAGAGCAGAGTACCCAGGCCGGCAGGAGCAGTGGTGGAAGCGTGGAAACC |
| | ACGTCTCCTACAGCAGAGACCATCAGAAGCGGAGCCTCGGGTATAAGGGAAACAACGCGTT |
| | CTCCCTAACCTGGGAGTGACAGACAGCGTCATTCCTCACAGTGATACCCTGTGTTCTAGCC |
| | ATCTGGCCCATGACAGAGCCAGCCCAGAGCCAGCCCAGAGCCAGCCCCTCACCAACCTGGA |
| | GCCTGGCCAGCTCGCCAAGCTGCACCATAGGCCTGGAAGGCGTGGAGACCTGCGGCAGTGC |
| | CCTGTCCTCCCGTGAGGCCTGCCATCCCTGCCAGGGGTCGCCTCTGGCTTCTCCTCCAGGA |
| | CCGCACGGTCCAGAGGCTCAGTGCCTGGAGTAGGTGTTGCCCCCCTGCTTCTAGGCCCAGA |
| | CCCTCCCTTGTTCCTGACCCCGGGCCTTTCCCTCTGGCTTGGACATCCAGGGCCCTGTCTC |
| | AGCTGGGGAGCTGCTCCTGCTCAAGGACTGTCTTCCGCGGGATCGAAAGGCCGCGTCCTGA |
| | ACAATGCGTGGGCCACGTGAGCGGAGCAGGCTCTAAAGGCCGCGTCCTAAACAGTGCGTGG |
| | GCCACGTGAGCGGAGCAGGCTCTAAAGGCCGCGTCCTAAACAGTGCGTGGGCCACGTGAGC |
| | GGAGCAGGCTCTAAAGGCCGCGTCCTAAACAGTGCGTGGGCCACGTGAGCGGAGCAGGCTC |
| | TAAAGGCCGCGTCCTAAACAGTGCGTGGGCCACGTGAGCGGAGCAGGCTCTAAAGGCCGCG |
| | TCCTAAACAGTGCGTGGGCCACGTGAGCGGAGCAGGCTCTAAAGGCCGCGTCCTAAACAGT |
| | GCGTGGGCCACGGGAGCGGAGCAGACTCTAAAGGCCGCGTCCTAAACAGTGCGTGGGCCAC |
| | GTGAGCGGAGCAGGCTCTAAAGGCCGCGTCCTAAACAGTGCGTGGGCCACGTGAGCGGAGC |
| | AGGCTCTAAAGGCCGCGTCCTAAACAGTGCGTGGGCCACGTGAGCGGAGCAGGCTCTAAAG |
| | GCCGCGTCCTAAACAGTGCGTGGGCCACGTGAGCGGAGCAGGCTCTAAAGGCCGCGTCCTA |
| | AACAGTGCGTGGGCCACGGGAGCGGAGCAGACTCTAAAGGCCGCGTCCTAAACAGTGTGTG |
| | GGCCACGTGAGCGGAGCAGGCTCTAAAGGCCGCGTCCTAAACAGTGCGTGGGCCACGGGAG |
| | CGGAGCAGACTCTAAAGGCCGCGTCCTAAACAGTGTGTGGGCCACGTGAGCGGAGCGCCCT |
| | CTCCACTGCCCTCGGGGCCGCAGCTCCCAGCTCAGCTCCCAGCCCTGCTCAGGGCAGCCAG |
| | GCCAGGAGGTACCATCCAGGCTAAGTGACCCTCAGGGGGGACAGGTGCCCCAGGAGATGCC |
| | AGCTGTTGGGAGAGGCTGGGGGACCAACTCGACCTGGCCTGTGGGCCCTGCCCTGGCCACC |
| | CATTGTAGGATCCAGCCGCCACGCCTGTGACACTCGTGTGCTTTCCCTGGTGTGTGCTTGT |
| | GGCAGGTGGGGGCAGAGGGTCCTCAGGCCAGAGAGCCACTCCCCCAGCGCCAGACCACCCT |
| | CTTCCTCACTCCCCCACCTCACCCCCTCACAGGTGCCTCCCAGGCCATCAGGGCCCAACCA |
| | CCCCTAAACAAATGGGTTCTCGGCCCCTCGTGGCTGGAGGTGGGTTCTCTCACCATTCCCA |
| | GCCTAAGGCTCCATCCCCATGCTGGCAGCTGTTCAACCATGTCTAGAGAGATCCACTGTCC |
| | CAGACAGCACCTCAGGGTCCCCCGTCCTGCCTGGAACCCTGTAGGAAACTCCACAAACCGC |
| | CGCCATTCTGTCCACACCCCTACAGGAGCCCCAACCCTCTCCCCACATCCAGGCTTCCCTC |
| | CCAGACCCCTCATCCCTGCCCGCACGGTGCCTGAGGGGGCCTTCTTGGGCAGCGCCTAAGC |
| | AAGCCCCCAGCACCCTTCGGCCCCTTCAAGGCACACAGGCCCCCTTTCCACCCAGCCTCAG |
| | GAAACCACCTGTGTCCTCCAACGACAGGTCCCAGCCTCCCAGCCTTTGCCTTGCCTGTTCC |
| | TCTCCCTGGAACTCTGCCCCGACACAGACCCTCCCCAGCAAGCCCGCAGGGGCACCTCCCC |
| | TGCCCCCAGACACCCTGTGCCCGTCAGTTCATCCCCAGCAGAGGCCCTCACCAGGCACACC |
| | CCCATGCTCACACCTGGCCGCAGGCCTCAGCCTCCCTGAGGGCCCCACCCAGCCCGCGTCT |
| | GGCCAGTGGTGCGTGCAAAGCCCCTCACCCAGACTCGGCGGAAGGCAGCCAGTGCAGGCCT |
| | GGGGAGGGGCTCTCCTTAGACCACCTTGCACCTTCCCTGGCACCCACCATGGGAAGAGCTG |
| | AGACTCACTGAGGACCAGCTGAGGCTCAGAGAAGGGACCCAGCACTGGTGGACACGCAGGG |
| | AGCCCACGCCAGGGCGCCGTGGTGAGTGAGGCCCAGTGCCACCCACTGAGGCCTCCCGTTC |
| | AGTGGGACGACGGTGAACAGGTGGAACCAACCAGGCAACCCCCGCCGGGCCCCACAGACGG |
| | GATCAGAGCAGGAAAGGCTTCCTGCCCCTGCAGGCCAGCGAGGAGCCCTGGCGGGGGCCAT |
| | GGCCCTCCAGGCGAGGAGGCTCCCCTGGCCACCGCCACCCGGGCCTCTCTGCTGCTGGGAA |
| | AACAAGTCAGAAAGCAAGTGGATGAGAGGTGGCGTGACAGACCCAGCTTCAGATCTGCTCT |
| | AATTTACAAAAGAAAAGGAAAAACACACTTGGCAGCCTTCAGCACTCTAATGATTCTTAAC |
| | AGCAGCAAATTATTGGCACAAGACTCCAGAGTGACTGGCAGGGTTGAGGGCTGGGGTCTCC |
| | CGCGTGTTTTGGGGCTAACAGCGGAAGGGAGAGCACTGGCAAAGGTGCTGGGGGCCCCTGG |
| | ACCCGACCCGCCCTGGAGACCGCAGCCACATCAGCCCCCAGCCCCACAGGCCCCCTACCAG |
| | CCGCAGGGTTTTGGCTGAGCTGAGAACCACTGTGAGGATATTGTACTGGTGGTGTATGCTA |
| | TACCGGTACAACTGGAACGACCACAGTGATTGGCAGCTCTACAAAAACCATGCTCCCCCGG |
| | GACCCCGGGCTGTGGGTTTCTGTAGCCCCTGGCTCAGGGCTGACTCACCGTGGCTGAATAC |
| | TTCCAGCACTGGGGCCAGGGCACCCTGGTCACCGTCTCCTCAGGTGAGTCTGCTGTCTGGG |
| | GATAGCGGGGAGCCAGGTGTACTGGGCCAGGCAAGGGCTTTGGCTTCAGACTTGGGGACAG |
| | GTGCTCAGCAAAGGAGGTCGGCAGGAGGGCGGAGGGTGTGTTTTTGTATGGGAGAAGCAGG |
| | AGGGCAGAGGCTGTGCTACTGGTACTTCGATCTCTGGGGCCGTGGCACCCTGGTCACTGTC |
| | TCCTCAGGTGAGTCCCACTGCAGCCCCCTCCCAGTCTTCTCTGTCCAGGCACCAGGCCAGG |
| | TATCTGGGGTCTGCAGCCGGCCTGGGTCTGGCCTGAGGCCACACCAGCTGCCATCCCTGGG |
| | GTCTCCGCCATGGGCTGCATGCCAGAGCCCTGCTGTCACTTAGCCCTGGGGCCAGCTGGAG |
| | CCCCCAAGGACAGGCAGGGACCCCGCTGGGCTTCAGCCCCGTCAGGGACCCTCCACAGGTA |
| | GCAAGCAGGCCGAGGGCAGGGACGGGAAGGAGAAGTTGTGGGCAGAGCCTGGGCTGGGGCT |
| | GGGCGCTGGCTGTTCATGTGCCGGGGACCAGGCCTGCGCTTTAGTGTGGCTACAAGTGCTT |
| | GGAGCACTGGGGCCAGGGCAGCCCGGCCACCGTCTCCCTGGGAACGTCACCCCTCCCTGCC |
| | TGGGTCTCAGCCCGGGGGTCTGTGTGGCTGGGGACAGGGACGCCGGCTGCCTCTGCTCTGT |
| | GCTTGGGCCATGTGACCCATTCGAGTGTCCTGCACGGGCACAGGTTTATGTCTGGGCAGGA |
| | ACAGGGACTGTGTCCCTGTGTGATGCTTTTGATATCTGGGGCCAAGGGACAATGGTCACCG |
| | TCTCTTCAGGTAAGATGGCTTTCCTTCTGCCTCCTTTCTCTGGGCCCAGCGTCCTCTGTCC |
| | TGGAGCTGGGAGATAATGTCCGGGGGCTCCTTGGTCTGCGCTGGGCCATGTGGGGCCCTCC |
| | GGGGCTCCTTCTCCGGCTGTTTGGGACCACGTTCAGCAGAAGGCCTTTCTTTGGGAACTGG |
| | GACTCTGCTGCTGGGGCAAAGGGTGGGCAGAGTCATGCTTGTGCTGGGGACAAAATGACCT |
| | TGGGACACGGGGCTGGCTGCCACGGCCGGCCCGGGACAGTCGGAGAGTCAGGTTTTTGTGC |
| | ACCCCTTAATGGGGCCTCCCACAATGTGACTACTTTGACTACTGGGGCCAGGGAACCCTGG |
| | TCACCGTCTCCTCAGGTGAGTCCTCACAACCTCTCTCCTGCTTTAACTCTGAAGGGTTTTG |
| | CTGCATTTTTGGGGGGAAATAAGCGTGCTGGGTCTCCTGCCAAGAGAGCCCCGGAGCAGCC |
| | TGGGGGGCTCAGGAGGATGCCCTGAGGCAACAGCGGCCACACAGACGAGGGGCAAGGGCTC |
| | CAGATGCTCCTTCCTCCTGAGCCCAGCAGCACGGGTCTCTCTGTGGCCAGGGCCACCCTGG |
| | GCCTCTGGGGTCCAATGTCCAACAACCCCCGGGCCCTCCCCGGGCTCAGTCTGAGAGGGTC |
| | CCAGGGACTTAGCGGGGTGCCAGTTCTTGCCTGGGGTCCTGGCATTGTTGTCACAATGTGA |
| | CAACTGGTTCGACCCCTGGGGCCAGGGAACCCTGGTCACCGTCTCCTCAGGTGAGTCCTCA |
| | CCACCCCCTCTCTGAGTCCACTTAGGGAGACTCAGCTTGCCAGGGTCTCAGGGTCAGAGTC |
| | TTGGAGGCATTTTGGAGGTCAGGAAAGAAAGCTGGGGAGAGGGACCCTTCGAATGGGAACC |
| | CAGCCTGTCCTCCCCAAGTCCGGCCACAGATGTCGGCAGCTGGGGGGCTCCTTCGGCTGGT |
| | CTGGGGTGACCTCTCTCCGCTTCACCTGGAGCATTCTCAGGGGCTGTCGTGATGATTGCGT |
| | GGTGGGACTCTGTCCCGCTCCAAGGCACCCGCTCTCTGGGACGGGTGCCCCCCGGGGTTTT |
| | TGGACTCCTGGGGGTGACTTAGCAGCCGTCTGCTTGCAGTTGGACTTCCCAGGCCGACAGT |
| | GGTCTGGCTTCTGAGGGGTCAGGCCAGAATGTGGGGTACGTGGGAGGCCAGCAGAGGGTTC |
| | CATGAGAAGGGCAGGACAGGGCCACGGACAGTCAGCTTCCATGTGACGCCCGGAGACAGAA |
| | GGTCTCTGGGTGGCTGGGTTTTTGTGGGGTGAGGATGGACATTCTGCCATTGTGATTACTA |
| | CTACTACTACTACATGGACGTCTGGGGCAAAGGGACCACGGTCACCGTCTCCTCAGGTAAG |
| | AATGGCCACTCTAGGGCCTTTGTTTTCTGCTACTGCCTGTGGGGTTTCCTGAGCATTGCAG |
| | GTTGGTCCTCGGGGCATGTTCCGAGGGGACCTGGGCGGACTGGCCAGGAGGGGATGGGCAC |
| | TGGGGTGCCTTGAGGATCTGGGAGCCTCTGTGGATTTTCCGATGCCTTTGGAAAATGGGAC |
| | TCAGGTTGGGTGCGTCTGATGGAGTAACTGAGCCTGGGGGCTTGGGGAGCCACATTTGGAC |
| | GAGATGCCTGAACAAACCAGGGGTCTTAGTGATGGCTGAGGAATGTGTCTCAGGAGCGGTG |
| | TCTGTAGGACTGCAAGATCGCTGCACAGCAGCGAATCGTGAAATATTTTCTTTAGAATTAT |
| | GAGGTGCGCTGTGTGATAGGGACAAAGAGTGGAGTGGGGCACTTTCTTTAGATTTGTGAGG |
| | AATGTTCCGCACTAGATTGTTTAAAACTTCATTTGTTGGAAGGAGAGCTGTCTTAGTGATT |
| | GAGTCAAGGAAGAAAGGCATCTAGCCTCGGTCTCAAAAGGGTAGTTGCTGTCTAGAGAGGT |
| | CTGGTGGAGCCTGCAAAAGTCCAGCTTTCAAAGGAACACAGAAGTATGTGTATGGAATATT |
| | AGAAGATGTTGCTTTTACTCTTAAGTTGGTTCCTAGGAAAAATAGTTAAATACTATGACTT |
| | TAAAATGTGAGAGGGTTTTCAAGTACTCATTTTTTTAAATGTCCAAAATTTTTGTCAATCA |
| | ATTTGAGTCATGTTTGTGTAGAACTGATATTACTGAAAGTTTAACCAAGGAATAGGAATGA |
| | TGCTCTTTCATACCCTATTCAGAACTGACTTTTAACAATAATAAATTAAGTTTAAATATTT |
| | TTAAATGAATTGAGCAATGTTGAGTTGGAGTCAAGATGGCCGATCAGAACCAGAACACCTG |
| | CAGCAGCTGTCAGGAAGCAGGTCATGTGGCAAGGCTATTTGGGGAAGGGAAAATAAAACCA |
| | CTAGGTAAACTTGTAGCTGTGGTTTGAAGAAGTGGTTTTGAAACACTCTGTCCAGCCCCAC |
| | CAAACCGAAAGTCCAGGCTGAGCAAAACACCACCTGGGTAATTTGCATTTCTAAAATAAGT |
| | TGAGGATTCAGCCGAAACTGGAGAGGTCCTCTTTTAACTTATTGAGTTCAACCTTTTAATT |
| | TTAGCTTGAGTAGTTCTAGTTTCCCCAAACTTAAGTTTATCGACTTCTAAAATGTATTTAG |
| | AATTCATTTTCAAAATTAGGTTATGTAAGAAATTGAAGGACTTTAGTGTCTTTAATTTCTA |
| | ATATATTTAGAAAACTTCTTAAAATTACTCTATTATTCTTCCCTCTGATTATTGGTCTCCA |
| | TTCAATTCTTTTCCAATACCCAAAGCATTTACAGTGACTTTGTTCATGATCTTTTTTAGTT |
| | GTTTGTTTTGTCTTACTATTAAGACTTTGGACAGCATTTATACAGTATCCGATGCATAGGG |
| | ACAAAGAGTGGAGCGGCCGCGGCGCGCCGCGGCCGC |
| | >1-1_+_2-8 hu D-D Fusion (SEQ ID NO: 2) |
| | GGTACAACTGGAACGACAGGATATTGTACTGGTGGTGTATGCTATACC |
| | >2-2_+_3-3 hu D-D Fusion (SEQ ID NO: 3) |
| | AGGATATTGTAGTAGTACCAGCTGCTATGCCGTATTACGATTTTTGGAGTGGTTATTATAC |
| | C |
| | >2-2_+_6-13 hu D-D Fusion (SEQ ID NO: 4) |
| | AGGATATTGTAGTAGTACCAGCTGCTATGCCGGGTATAGCAGCAGCTGGTAC |
| | >2-08 hu long D (SEQ ID NO: 5) |
| | AGGATATTGTACTGGTGGTGTATGCTATACC |
| | >2-8_+_1-1 hu D-D Fusion (SEQ ID NO: 6) |
| | AGGATATTGTACTGGTGGTGTATGCTATACCGGTACAACTGGAACGAC |
| | >2-15_+_2-21 hu D-D Fusion (SEQ ID NO: 7) |
| | AGGATATTGTAGTGGTGGTAGCTGCTACTCCAGCATATTGIGGIGGTGATTGCTATTCC |
| | >2-15_+_3-22 hu D-D Fusion (SEQ ID NO: 8) |
| | AGGATATTGTAGTGGTGGTAGCTGCTACTCCGTATTACTATGATAGTAGIGGITATTACTA |
| | C |
| | >2-15_+_4-17 hu D-D Fusion (SEQ ID NO: 9) |
| | AGGATATTGTAGTGGGGTAGCTGCTACTCCTGACTACGGIGACTAC |
| | >2-15_+_5-5 hu D-D Fusion (SEQ ID NO: 10) |
| | AGGATATTGTAGTGGTGGTAGCTGCTACTCCGTGGATACAGCTATGGTTAC |
| | >2-15_+_5-24 hu D-D Fusion (SEQ ID NO: 11) |
| | AGGATATTGTAGTGGTGGTAGCTGCTACTCCGTAGAGATGGCTACAATTAC |
| | >3-3 hu long D (SEQ ID NO: 12) |
| | GTATTACGATTTTTGGAGTGGTTATTATACC |
| | >3-3_+_3-10 hu D-D Fusion (SEQ ID NO: 13) |
| | GTATTACGATTTTTGGAGTGGTTATTATACCGTATTACTATGGTTCGGGGAGTTATTATAA |
| | C |
| | >3-16 hu long D (SEQ ID NO: 14) |
| | GTATTATGATTACGTTTGGGGGAGTTATCGTTATACC |
| | >5-5_+_3-10 hu D-D Fusion (SEQ ID NO: 15) |
| | GTGGATACAGCTATGGTTACGTATTACTATGGTTCGGGGAGTTATTATAAC |
| | >5-5_+_3-22 hu D-D Fusion (SEQ ID NO: 16) |
| | GTGGATACAGCTATGGTTACGTATTACTATGATAGTAGTGGTTATTACTAC |
| | >5-5_+_6-13 hu D-D Fusion (SEQ ID NO: 17) |
| | GTGGATACAGCTATGGTTACGGGTATAGCAGCAGCTGGTAC |
| | >5-12 + 2-15 hu D-D Fusion (SEQ ID NO: 18) |
| | GTGGATATAGTGGCTACGATTACAGGATATTGTAGTGGTGGTAGCTGCTACTCC |
| | >5-12_+_3-22 hu D-D Fusion (SEQ ID NO: 19) |
| | GTGGATATAGTGGCTACGATTACGTATTACTATGATAGTAGTGGTTATTACTAC |
| | >5-12_+_4-17 hu D-D Fusion (SEQ ID NO: 20) |
| | GTGGATATAGTGGCTACGATTACTGACTACGGTGACTAC |
| | >5-12_+_5-5 hu D-D Fusion (SEQ ID NO: 21) |
| | GTGGATATAGTGGCTACGATTACGTGGATACAGCTATGGTTAC |
| | >5-12_+_6-19 hu D-D Fusion (SEQ ID NO: 22) |
| | GTGGATATAGTGGCTACGATTACGGGTATAGCAGTGGCTGGTAC |
| | >6-6_+_3-10 hu D-D Fusion (SEQ ID NO: 23) |
| | GAGTATAGCAGCTCGTCCGTATTACTATGGTTCGGGGAGTTATTATAAC |
| | >6-6_+_6-19 hu D-D Fusion (SEQ ID NO: 24) |
| | GAGTATAGCAGCTCGTCCGGGTATAGCAGTGGCTGGTAC |
| | >6-13_+_2-21 hu D-D Fusion (SEQ ID NO: 25) |
| | GGGTATAGCAGCAGCTGGTACAGCATATTGTGGTGGTGATTGCTATTCC |
| | >6-13_+_6-19 hu D-D Fusion (SEQ ID NO: 26) |
| | GGGTATAGCAGCAGCTGGTACGGGTATAGCAGTGGCTGGTAC |
| | >6-19_+_1-26 hu D-D Fusion (SEQ ID NO: 27) |
| | GGGTATAGCAGTGGCTGGTACGGTATAGTGGGAGCTACTAC |
| | >6-19_+_3-22 hu D-D Fusion (SEQ ID NO: 28) |
| | GGGTATAGCAGTGGCTGGTACGTATTACTATGATAGTAGTGGTTATTACTAC |
| | GTTGTTGYCTGGVCYT (SEQ ID NO: 29) |
| | VQLERQDIVLVVYAI (SEQ ID NO: 30) |
| | YNWNDRILYWWCMLY (SEQ ID NO: 31) |
| | RIL**YQLLCRITIFGVVII (SEQ ID NO: 32) |
| | GYCSSTSCYAVLRFLEWLLY (SEQ ID NO: 33) |
| | DIVVVPAAMPYYDFWSGYYT (SEQ ID NO: 34) |
| | VLRFLEWLLY (SEQ ID NO: 35) |
| | YYDFWSGYYT (SEQ ID NO: 36) |
| | ITIFGVVII (SEQ ID NO: 37) |
| | VDIVATITDYGDY (SEQ ID NO: 38) |
| | WI*WLRLLTTVT (SEQ ID NO: 39) |
| | GYSGYDY*LR*L (SEQ ID NO: 40) |
| | VDTAMVTYYYDSSGYYY (SEQ ID NO: 41) |
| | WIQLWLRITMIVVVIT (SEQ ID NO: 42) |
| | GYSYGYVLL***WLLL (SEQ ID NO: 43) |
| | EYSSSSVLLWFGELL* (SEQ ID NO: 44) |
| | SIAARPYYYGSGSYYN (SEQ ID NO: 45) |
| | V*QLVRITMVRGVII (SEQ ID NO: 46) |
| | EYSSSSGYSSGWY (SEQ ID NO: 47) |
| | SIAARPGIAVAG (SEQ ID NO: 48) |
| | V*QLVRV*QWLV (SEQ ID NO: 49) |
| | RILYWWCMLY (SEQ ID NO: 50) |
| | GYCTGGVCYT (SEQ ID NO: 51) |
| | DIVLVVYAI (SEQ ID NO: 52) |
| | VDIVATI TWI QLWL (SEQ ID NO: 53) |
| | WI*WLRLRGYSYGY (SEQ ID NO: 54) |
| | GYSGYDYVDTAMV (SEQ ID NO: 55) |
| | GYSSSWYGYSSGWY (SEQ ID NO: 56) |
| | GIAAAGTGIAVAG (SEQ ID NO: 57) |
| | V*QQLVRV*QWLV (SEQ ID NO: 58) |
| | RIL**YQLLCRV*QQLV (SEQ ID NO: 59) |
| | GYCSSTSCYAGYSSSWY (SEQ ID NO: 60) |
| | DIVVVPAAMPGIAAAG (SEQ ID NO: 61) |
| | VDIVATITGIAVAG (SEQ ID NO: 62) |
| | WI*WLRLRV*QWLV (SEQ ID NO: 63) |
| | GYSGYDYGYSSGWY (SEQ ID NO: 64) |
| | GYSSSWYSILWW*LLF (SEQ ID NO: 65) |
| | GIAAAGTAYCGGDCYS (SEQ ID NO: 66) |
| | V*QQLVQHIVVVIAI (SEQ ID NO: 67) |
| | VDTAMVTYYYGSGSYYN (SEQ ID NO: 68) |
| | WIQLWLRITMVRGVII (SEQ ID NO: 69) |
| | GYSYGYVLLWFGELL* (SEQ ID NO: 70) |
| | RIL*WW*LLLRITMIVVVIT (SEQ ID NO: 71) |
| | GYCSGGSCYSVLL***WLLL (SEQ ID NO: 72) |
| | DIVVVVAATPYYYDSSGYYY (SEQ ID NO: 73) |
| | VL*LRLGELSLY (SEQ ID NO: 74) |
| | YYDYVWGSYRYT (SEQ ID NO: 75) |
| | IMITFGGVIVI (SEQ ID NO: 76) |
| | VDIVATITGYCSGGSCYS (SEQ ID NO: 77) |
| | WI*WLRLQDIVVVVAAT (SEQ ID NO: 78) |
| | GYSGYDYRIL*WW*LLL (SEQ ID NO: 79) |
| | RIL*WW*LLLRRDGYNY (SEQ ID NO: 80) |
| | GYCSGGSCYSVEMATI (SEQ ID NO: 81) |
| | DIVVVVAATP*RWLQL (SEQ ID NO: 82) |
| | GYSSGWYGIVGAT (SEQ ID NO: 83) |
| | GIAVAGTV*WELL (SEQ ID NO: 84) |
| | V*QWLVRYSGSYY (SEQ ID NO: 85) |
| | RIL*WW*LLLRGYSYGY (SEQ ID NO: 86) |
| | GYCSGGSCYSVDTAMV (SEQ ID NO: 87) |
| | DIVVVVAATPWIQLWL (SEQ ID NO: 88) |
| | VDIVATITYYYDSSGYYY (SEQ ID NO: 89) |
| | WI*WLRLRITMIVVVIT (SEQ ID NO: 90) |
| | GYSGYDYVLL***WLLL (SEQ ID NO: 91) |
| | RIL*WW*LLLLTTVT (SEQ ID NO: 92) |
| | GYCSGGSCYS*LR*L (SEQ ID NO: 93) |
| | DIVVVVAATPDYGDY (SEQ ID NO: 94) |
| | VDTAMVTGIAAAG (SEQ ID NO: 95) |
| | WIQLWLRV*QQLV (SEQ ID NO: 96) |
| | GYSYGYGYSSSWY (SEQ ID NO: 97) |
| | GYSSGWYVLL***WILL (SEQ ID NO: 98) |
| | GIAVAGTYYYDSSGYYY (SEQ ID NO: 99) |
| | V*QWLVRITMIVVVIT (SEQ ID NO: 100) |
| | RIL*WW*LLLQHIVVVIAI (SEQ ID NO: 101) |
| | GYCSGGSCYSSILWW*LLF (SEQ ID NO: 102) |
| | DIVVVVAATPAYCGGDCYS (SEQ ID NO: 103) |
| | VLRFLEWLLYRITMVRGVII (SEQ ID NO: 104) |
| | YYDFWSGYYTVLLWFGELL* (SEQ ID NO: 105) |
| | ITIFGVVIIPYYYGSGSYYN (SEQ ID NO: 106) |
| | RILYWWCMLYRYNWND (SEQ ID NO: 107) |
| | GYCTGGVCYTGTTGT (SEQ ID NO: 108 |
| | DIVLVVYAIPVQLER (SEQ ID NO: 109) |
| | >2-15 + 2-21_F3 |
| | DIVVVVAATPAYCGGDCYS (SEQ ID NO: 110) |
| | >2-15_+_4-17_F3 |
| | DIVVVVAATPDYGDY (SEQ ID NO: 111) |
| | >2-15_+_5-5_F3 |
| | DIVVVVAATPWIQLWL (SEQ ID NO: 112) |
| | >2-15_+_3-22_F3 |
| | DIVVVVAATPYYYDSSGYYY (SEQ ID NO: 113) |
| | >2-2_+_3-3_F3 |
| | DIVVVPAAMPYYDFWSGYYT (SEQ ID NO: 114) |
| | >2-2_+_6-13_F3 |
| | DIVVVPAAMPGIAAAG (SEQ ID NO: 115) |
| | >1-1+2-8 F2 |
| | VQLERQDIVLVVYAI (SEQ ID NO: 116) |
| | >2-08 F3 |
| | DIVLVVYAI (SEQ ID NO: 117) |
| | >2-8+1-1_F3 |
| | DIVLVVYAIPVQLER (SEQ ID NO: 118) |
| | >5-12_+_4-17_F1 |
| | VDIVATITDYGDY (SEQ ID NO: 119) |
| | >5-12 + 2-15 F1 |
| | VDIVATITGYCSGGSCYS (SEQ ID NO: 120) |
| | >5-12_+_3-22_F1 |
| | VDIVATITYYYDSSGYYY (SEQ ID NO: 121) |
| | >5-12_+_6-19_F1 |
| | VDIVATITGIAVAG (SEQ ID NO: 122) |
| | >5-12_+_5-5 F1 |
| | VDIVATITWIQLWL (SEQ ID NO: 123) |
| | >5-5_+_3-22_F2 |
| | VDTAMVTYYYDSSGYYY (SEQ ID NO: 124) |
| | >5-5_+_6-13_F1 |
| | VDTAMVTGIAAAG (SEQ ID NO: 125) |
| | >3-16_F3 |
| | IMITFGGVIVI (SEQ ID NO: 126) |
| | >3-3_F3 |
| | ITIFGVVII (SEQ ID NO: 127) |
| | >5-5_+_3-10_F2 |
| | WIQLWLRITMVRGVII (SEQ ID NO: 128) |
| | >5-5_+_3-22_F3 |
| | WIQLWLRITMIVVVIT (SEQ ID NO: 129) |
| | >2-08_F1 |
| | RILYWWCMLY (SEQ ID NO: 130) |
| | >2-8+1-1_F1 |
| | RILYWWCMLYRYNWND (SEQ ID NO: 131) |
| | >1-1+2-8_F3 |
| | YNWNDRILYWWCMLY ((SEQ ID NO: 132) |
| | >3-3_+_3-10_F1 |
| | VLRFLEWLLYRITMVRGVII (SEQ ID NO: 133) |
| | >3-3_F1 |
| | VLRFLEWLLY (SEQ ID NO: 134) |
| | >5-12_+_5-5_F3 |
| | GYSGYDYVDTAMV (SEQ ID NO: 135) |
| | >6-6_+_6-19_F1 |
| | EYSSSSGYSSGWY (SEQ ID NO: 136) |
| | >6-19_+_1-26_F1 |
| | GYSSGWYGIVGAT (SEQ ID NO: 137) |
| | >5-12_+_6-19_F3 |
| | GYSGYDYGYSSGWY (SEQ ID NO: 138) |
| | >6-13_+_6-19_F1 |
| | GYSSSWYGYSSGWY (SEQ ID NO: 139) |
| | >5-5_+_6-13_F3 |
| | GYSYGYGYSSSWY (SEQ ID NO: 140) |
| | >1-1_+_2-8_F1 hu D-D Fusion |
| | GTTGTTGYCTGGVCYT (SEQ ID NO: 141) |
| | >2-08_F2 |
| | GYCTGGVCYT (SEQ ID NO: 142) |
| | >2-8+1-1 F2 |
| | GYCTGGVCYTGTTGT (SEQ ID NO: 143) |
| | >2-15_+_5-5_F2 |
| | GYCSGGSCYSVDTAMV (SEQ ID NO: 144) |
| | >2-15_+_5-24_F2 |
| | GYCSGGSCYSVEMATI (SEQ ID NO: 145) |
| | >2-2_+_6-13_F2 |
| | GYCSSTSCYAGYSSSWY (SEQ ID NO: 146) |
| | >2-2_+_3-3_F2 |
| | GYCSSTSCYAVLRFLEWLLY (SEQ ID NO: 147) |
| | >3-16_F2 |
| | YYDYVWGSYRYT (SEQ ID NO: 148) |
| | >3-3_F2 |
| | YYDFWSGYYT (SEQ ID NO: 149) |
| | >3-3_+_3-10_F3 |
| | ITIFGVVIIPYYYGSGSYYN (SEQ ID NO: 150) |
| | >5-5_+_3-10_F1 |
| | VDTAMVTYYYGSGSYYN (SEQ ID NO: 151) |
| | >6-19_+_3-22_F2 |
| | GIAVAGTYYYDSSGYYY (SEQ ID NO: 152) |
| | >6-6_+_3-10_F2 |
| | SIAARPYYYGSGSYYN (SEQ ID NO: 153) |
| | >6-6_+_6-19_F2 |
| | SIAARPGIAVAG (SEQ ID NO: 154) |
| | >6-13_+_2-21_F2 |
| | GIAAAGTAYCGGDCYS (SEQ ID NO: 155) |
| | >6-13_+_6-19_F2 |
| | GIAAAGTGIAVAG (SEQ ID NO: 156) |
| | GTTGT (SEQ ID NO: 157) |
| | VQLER (SEQ ID NO: 158) |
| | YNWND (SEQ ID NO: 159) |
| | GITGT (SEQ ID NO: 160) |
| | V*LEL (SEQ ID NO: 161) |
| | YNWNY (SEQ ID NO: 162) |
| | GITGT (SEQ ID NO: 163) |
| | V*LER (SEQ ID NO: 164) |
| | YNWND (SEQ ID NO: 165) |
| | GIVGAT (SEQ ID NO: 166) |
| | V*WELL (SEQ ID NO: 167) |
| | YSGSYY (SEQ ID NO: 168) |
| | RIL**YOLLY (SEQ ID NO: 169) |
| | GYCSSTSCYT (SEQ ID NO: 170) |
| | DIVVVPAAI (SEQ ID NO: 171) |
| | RILY*WCMLY (SEQ ID NO: 172) |
| | GYCTNGVCYT (SEQ ID NO: 173) |
| | DIVLMVYAI (SEQ ID NO: 174) |
| | RIL*WW*LLL (SEQ ID NO: 175) |
| | GYCSGGSCYS (SEQ ID NO: 176) |
| | DIVVVVAAT (SEQ ID NO: 177) |
| | SILWW*LLF (SEQ ID NO: 178) |
| | AYCGGDCYS (SEQ ID NO: 179) |
| | HIVVVTAI (SEQ ID NO: 180) |
| | VLRFLEWLLY (SEQ ID NO: 181) |
| | YYDFWSGYYT (SEQ ID NO: 182) |
| | ITIFGVVII (SEQ ID NO: 183) |
| | VLRYFDWLL* (SEQ ID NO: 184) |
| | YYDILTGYYN (SEQ ID NO: 185) |
| | ITIF*LVII (SEQ ID NO: 186) |
| | VLLWFGELL* (SEQ ID NO: 187) |
| | YYYGSGSYYN (SEQ ID NO: 188) |
| | ITMVRGVII (SEQ ID NO: 189) |
| | VL*LRLGELSLY (SEQ ID NO: 190) |
| | YYDYVWGSYRYT (SEQ ID NO: 191) |
| | IMITFGGVIVI (SEQ ID NO: 192) |
| | VLL***WLLL (SEQ ID NO: 193) |
| | YYYDSSGYYY (SEQ ID NO: 194) |
| | ITMIVVVIT (SEQ ID NO: 195) |
| | *LQ*L (SEQ ID NO: 196) |
| | DYSNY (SEQ ID NO: 197) |
| | TTVT (SEQ ID NO: 198) |
| | *LR*L (SEQ ID NO: 199) |
| | DYGDY (SEQ ID NO: 200) |
| | TTVT (SEQ ID NO: 201) |
| | *LRW*L (SEQ ID NO: 202) |
| | DYGGNS (SEQ ID NO: 203) |
| | TTVVT (SEQ ID NO: 204) |
| | VDTAMV (SEQ ID NO: 205) |
| | WIQLWL (SEQ ID NO: 206) |
| | GYSYGY (SEQ ID NO: 207) |
| | VDIVATI (SEQ ID NO: 208) |
| | WI*WLRL (SEQ ID NO: 209) |
| | GYSGYDY (SEQ ID NO: 210) |
| | VDTAMV (SEQ ID NO: 211) |
| | WIQLWL (SEQ ID NO: 212) |
| | GYSYGY (SEQ ID NO: 213) |
| | VEMATI (SEQ ID NO: 214) |
| | *RWLQL (SEQ ID NO: 215) |
| | RDGYNY (SEQ ID NO: 216) |
| | EYSSSS (SEQ ID NO: 217) |
| | SIAAR (SEQ ID NO: 218) |
| | V*QLV (SEQ ID NO: 219) |
| | GYSSSWY (SEQ ID NO: 220) |
| | GIAAAG (SEQ ID NO: 221) |
| | V*QQLV (SEQ ID NO: 222) |
| | GYSSGWY (SEQ ID NO: 223) |
| | GIAVAG (SEQ ID NO: 224) |
| | V*QWLV (SEQ ID NO: 225) |
| | LTG (SEQ ID NO: 226) |
| | *LG (SEQ ID NO: 227) |
| | NWG (SEQ ID NO: 228) |

1. A transgene construct encoding an immunoglobulin heavy chain variable region comprising:
(a) a plurality of human unrearranged immunoglobulin heavy chain variable segments (VHs), wherein each VH is between 98 and 101 amino acids in length; operatively linked to
(b) a plurality of human D-D fusion segments; operatively linked to
(c) a plurality of human J segments.

2. The transgene construct of claim 1, wherein the plurality of human D-D fusion segments comprises at least one naturally-occurring human D-D fusion pair.

3. The transgene construct of claim 1, wherein the plurality of human D-D fusion segments comprises at least one synthetic human D-D fusion pair.

4. The transgene construct of claim 1, wherein the plurality of human D-D fusion segments comprises at least one synthetic human D-D fusion pair and at least one naturally-occurring human D-D fusion pair.

5. The transgene construct of claim 1, which encodes at least twenty VH regions.

6. The transgene construct of claim 1, which encodes human VH regions in 5′ to 3′ orientation as follows: 3-73, 3-72, 2-70D, 1-69, 4-61, 5-51, 3-49, 3-43, 4-39, 4-32, 2-26, 1-24, 3-23, 3-15, 3-9, 1-8, 2-5, 7-4-1, 1-2 and 6-1.

7. The transgene construct of claim 1, which encodes a synthetic 1-1/2-8 D-D fusion or a synthetic 2-8/1-1 D-D fusion.

8. The transgene construct of claim 1, which encodes a synthetic 1-1/2-8 D-D fusion and a synthetic 2-8/1-1 D-D fusion.

9. The transgene construct of claim 1, which encodes at least one naturally-occurring D-D fusion pair selected from the group consisting of 2-2/3-3; 5-12/4-17; 5-5/3-22; 6-6/3-10; 6-6/6-19; 5-12/5-5; 6-13/6-19; 2-2/6-13; 5-12/6-19; 6-13/2-21; 5-5/3-10; 2-15/3-22; 5-12/2-15; 2-15/5-24; 6/19-1/26; 2-15/5-5; 5-12/3-22; 2-15/4-17; 5-5/6-13; 6-19/3-22; 2-15/2-21 and 3-3/3-10.

10. The transgene construct of claim 1, which encodes naturally-occurring D-D fusion pairs 2-2/3-3; 5-12/4-17; 5-5/3-22; 6-6/3-10; 6-6/6-19; 5-12/5-5; 6-13/6-19; 2-2/6-13; 5-12/6-19; 6-13/2-21; 5-5/3-10; 2-15/3-22; 5-12/2-15; 2-15/5-24; 6/19-1/26; 2-15/5-5; 5-12/3-22; 2-15/4-17; 5-5/6-13; 6-19/3-22; 2-15/2-21 and 3-3/3-10.

11. The transgene construct of claim 1, which further encodes at least one natural D segment.

12. The transgene construct of claim 11, which encodes D segments and D-D fusions in 5′ to 3′ orientation as follows: 1-1/2-8; 2-2/3-3; 3-3; 5-12/4-17; 5-5/3-22; 6-6/3-10; 6-6/6-19; 2-8; 5-12/5-5; 6-13/6-19; 2-2/6-13; 5-12/6-19; 6-13/2-21; 5-5/3-10; 2-15/3-22; 3-16; 5-12/2-15; 2-15/5-24; 6/19-1/26; 2-15/5-5; 5-12/3-22; 2-15/4-17; 5-5/6-13; 6-19/3-22; 2-15/2-21; 3-3/3-10 and 2-8/1-1.

13. The transgene construct of claim 1, which encodes J1-J6 segments.

14. The transgene construct of claim 1, which further encodes an immunoglobulin (Ig) constant region downstream of the plurality of J segments.

15. The transgene construct of claim 14, wherein the Ig constant region is a mouse Ig constant region.

16. The transgene construct of claim 14, wherein the Ig constant region is a human Ig constant region.

17. The transgene construct of claim 1, which further comprises lox sites to facilitate cre/lox mediated RMCE (Recombinase Mediated Cassette Exchange).

18. The transgene construct of claim 1, which further comprises Guide Recombination Sequences (GRS) to facilitate CRISPR/CAS-mediated recombination.

19. The transgene construct of claim 1, which comprises the sequence shown in SEQ ID NO: 1.

20. The transgene construct of claim 1, which is carried on a bacterial artificial chromosome (BAC).

21. A transgenic mouse comprising the transgene construct of claim 1.

22. The transgenic mouse of claim 21, which further comprises a transgene construct encoding an immunoglobulin light chain such that the mouse expresses antibodies comprising light and heavy chains.

23. A method of generating antibodies to an antigen of interest, the method comprising administering the antigen of interest to the transgenic mouse of claim 22, such that antibodies that bind to the antigen of interest are generated.

24. The method of claim 23, which further comprises isolating an antibody of interest from the mouse and determining the heavy chain CDR3 sequence thereof.
